SEQUENCE LISTING (208 sequences)

SEQ ID NO: 1 - protein, 10 aa, synthetic construct
GYTFTNYWMH

SEQ ID NO: 2 - protein, 17 aa, synthetic construct
IIHPNSGSTNYNEKFQG

SEQ ID NO: 3 - protein, 9 aa, synthetic construct
GIYDYPFAY

SEQ ID NO: 4 - protein, 15 aa, synthetic construct
RASESVSIHGTHLMH

SEQ ID NO: 5 - protein, 7 aa, synthetic construct
AASSLQS

SEQ ID NO: 6 - protein, 9 aa, synthetic construct
QQSIEDPYT

SEQ ID NO: 7 - protein, 7 aa, synthetic construct
AASNLES

SEQ ID NO: 8 - protein, 7 aa, synthetic construct
AASNLQS

SEQ ID NO: 9 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 10 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 11 - protein, 365 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 12 - protein, 377 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 118 aa, synthetic construct
QVQLVQSGAEVKKPGASVKVSCKASGYTFTNYWMHWVRQAPGQGLEWMGIIHPNSGSTNYNEKFQGRVTMTRDTSTSTVYMELSSLRSEDTAVYYCARGIYDYPFAYWGQGTLVTVSS

SEQ ID NO: 14 - protein, 118 aa, synthetic construct
QVQLVQSGAEVKKPGASVKVSCKASGYTFTNYWMHWVRQAPGQGLEWMGIIHPNSGSTNYNEKFQGRVTMTVDKSTSTVYMELSSLRSEDTAVYYCARGIYDYPFAYWGQGTLVTVSS

SEQ ID NO: 15 - protein, 111 aa, synthetic construct
DIQMTQSPSSLSASVGDRVTITCRASESVSIHGTHLMHWYQQKPGKAPKLLIYAASSLQSGVPSRFSGSGSETDFTLTISSLQPEDFATYYCQQSIEDPYTFGGGTKVEIK

SEQ ID NO: 16 - protein, 111 aa, synthetic construct
DIQMTQSPSSLSASVGDRVTITCRASESVSIHGTHLMHWYQQKPGKAPKLLIYAASNLESGVPSRFSGSGSGTDFTLTISSLQPEDFATYYCQQSIEDPYTFGGGTKVEIK

SEQ ID NO: 17 - protein, 111 aa, synthetic construct
DIQMTQSPSSLSASVGDRVTITCRASESVSIHGTHLMHWYQQKPGKAPKLLIYAASNLESGVPSRFSGSGSETDFTLTISSLQPEDFATYYCQQSIEDPYTFGGGTKVEIK

SEQ ID NO: 18 - protein, 111 aa, synthetic construct
DIQMTQSPSSLSASVGDRVTITCRASESVSIHGTHLMHWYQQKPGKAPKLLIYAASNLQSGVPSRFSGSGSGTDFTLTISSLQPEDFATYYCQQSIEDPYTFGGGTKVEIK

SEQ ID NO: 19 - protein, 111 aa, synthetic construct
DIQMTQSPSSLSASVGDRVTITCRASESVSIHGTHLMHWYQQKPGKAPKLLIYAASNLQSGVPSRFSGSGSETDFTLTISSLQPEDFATYYCQQSIEDPYTFGGGTKVEIK

SEQ ID NO: 20 - protein, 111 aa, synthetic construct
DIQMTQSPSSLSASVGDRVTITCRASESVSIHGTHLMHWYQQKPGKAPKLLIYAASSLQSGVPSRFSGSGSGTDFTLTISSLQPEDFATYYCQQSIEDPYTFGGGTKVEIK

SEQ ID NO: 21 - protein, 706 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 290 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 446 aa, synthetic construct
QVQLVQSGAEVKKPGASVKVSCKASGYTFTNYWMHWVRQAPGQGLEWMGIIHPNSGSTNYNEKFQGRVTMTRDTSTSTVYMELSSLRSEDTAVYYCARGIYDYPFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 24 - protein, 218 aa, synthetic construct
DIQMTQSPSSLSASVGDRVTITCRASESVSIHGTHLMHWYQQKPGKAPKLLIYAASSLQSGVPSRFSGSGSETDFTLTISSLQPEDFATYYCQQSIEDPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 218 aa, synthetic construct
DIQMTQSPSSLSASVGDRVTITCRASESVSIHGTHLMHWYQQKPGKAPKLLIYAASNLESGVPSRFSGSGSGTDFTLTISSLQPEDFATYYCQQSIEDPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 10 aa, synthetic construct
GFTFSNAWMD

SEQ ID NO: 27 - protein, 19 aa, synthetic construct
QITAKSNNYATYYADSVKG

SEQ ID NO: 28 - protein, 4 aa, synthetic construct
DGYH

SEQ ID NO: 29 - protein, 19 aa, synthetic construct
QITAKSNNYATYYAAPVKG

SEQ ID NO: 30 - protein, 11 aa, synthetic construct
RASEDIRNGLA

SEQ ID NO: 31 - protein, 7 aa, synthetic construct
NANSLHT

SEQ ID NO: 32 - protein, 9 aa, synthetic construct
EDTSKYPYT

SEQ ID NO: 33 - protein, 115 aa, synthetic construct
EVQLLESGGGLVQPGGSLRLSCAASGFTFSNAWMDWVRQAPGKGLEWVSQITAKSNNYATYYADSVKGRFTISRDNSKNTLYLQMNSLRAEDTAVYYCTDDGYHWGQGTLVTVSS

SEQ ID NO: 34 - protein, 107 aa, synthetic construct
DIQMTQSPSSLSASVGDRVTITCRASEDIRNGLAWYQQKPGKAPKLLIYNANSLHTGVPSRFSGSGSGTDFTLTISSLQPEDFATYYCEDTSKYPYTFGQGTKLEIK

SEQ ID NO: 35 - protein, 115 aa, synthetic construct
EVQLVESGGGLVKPGGSLRLSCAASGFTFSNAWMDWVRQAPGKGLEWIAQITAKSNNYATYYAAPVKGRFTISRDDSKNTLYLQMNSLKTEDTAVYYCTDDGYHWGQGTLVTVSS

SEQ ID NO: 36 - protein, 107 aa, synthetic construct
AIQMTQSPSSLSASVGDRVTITCRASEDIRNGLAWYQQKPGKAPKLLIYNANSLHTGVPSRFSGSGSGTDFTLTISSLQPEDFATYYCEDTSKYPYTFGQGTKLEIK

SEQ ID NO: 37 - protein, 443 aa, synthetic construct
EVQLLESGGGLVQPGGSLRLSCAASGFTFSNAWMDWVRQAPGKGLEWVSQITAKSNNYATYYADSVKGRFTISRDNSKNTLYLQMNSLRAEDTAVYYCTDDGYHWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 38 - protein, 214 aa, synthetic construct
DIQMTQSPSSLSASVGDRVTITCRASEDIRNGLAWYQQKPGKAPKLLIYNANSLHTGVPSRFSGSGSGTDFTLTISSLQPEDFATYYCEDTSKYPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 443 aa, synthetic construct
EVQLVESGGGLVKPGGSLRLSCAASGFTFSNAWMDWVRQAPGKGLEWIAQITAKSNNYATYYAAPVKGRFTISRDDSKNTLYLQMNSLKTEDTAVYYCTDDGYHWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 40 - protein, 214 aa, synthetic construct
AIQMTQSPSSLSASVGDRVTITCRASEDIRNGLAWYQQKPGKAPKLLIYNANSLHTGVPSRFSGSGSGTDFTLTISSLQPEDFATYYCEDTSKYPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 118 aa, synthetic construct
QVQLQQPGAELVKPGASVKLSCKASGYTFTNYWMHWVKQRPGQGLEWIGIIHPNSGSTNYNEKFKSKATLTVDKSSSTAYMQLSSLTSEDSAVYYCARGIYDYPFAYWGQGTLVTVSS

SEQ ID NO: 42 - protein, 111 aa, synthetic construct
DIVLTQSPASLAVSLGQRATISCRASESVSIHGTHLMHWYQQKPGQPPKLLIYAASNLESGVPARFSGSGSETDFTLNIHPVEEEDAATYFCQQSIEDPYTFGGGTKLEIK

SEQ ID NO: 43 - protein, 15 aa, Homo sapiens
YFDYWGQGTLVTVSS

SEQ ID NO: 44 - protein, 12 aa, Homo sapiens
LTFGGGTKVEIK

SEQ ID NO: 45 - protein, 111 aa, synthetic construct
DIQMTQSPSSLSASVGDRVTITCRASESVSIHGTHLMHWYQQKPGKAPKLLIYAASNLESGVPSRFSGSGSGTDFTLTISSLQPEDFATYFCQQSIEDPYTFGGGTKVEIK

SEQ ID NO: 46 - protein, 111 aa, synthetic construct
DIQMTQSPSSLSASVGDRVTITCRASESVSIHGTHLMHWYQQKPGKAPKLLIYAASNLESGVPSRFSGSGSETDFTLTISSLQPEDFATYFCQQSIEDPYTFGGGTKVEIK

SEQ ID NO: 47 - protein, 115 aa, synthetic construct
EVQLVESGGGLVKPGESLRLSCAASGFTFSNAWMDWVRQAPGKRLEWIAQITAKSNNYATYYAAPVKGRFTISRDDSKNTLYLQMNSLKKEDTAVYYCTDDGYHWGQGTLVTVSS

SEQ ID NO: 48 - protein, 107 aa, synthetic construct
AIQMTQSPSSLSASVGDRVTIACRASEDIRNGLAWYQQKPGKAPKLLIYNANSLHTGVPSRFSGSGSGTDFTLTISSLQPEDEAIYYCEDTSKYPYTFGQGTKLEIK

SEQ ID NO: 49 - protein, 17 aa, Homo sapiens
AEYFQHWGQGTLVTVSS

SEQ ID NO: 50 - protein, 12 aa, Homo sapiens
YTFGQGTKLEIK

SEQ ID NO: 51 - protein, 19 aa, synthetic construct
RIKSKTDGGTTDYAAPVKG

SEQ ID NO: 52 - protein, 7 aa, synthetic construct
AASSLQS

SEQ ID NO: 53 - protein, 115 aa, synthetic construct
EVQLVESGGGLVKPGGSLRLSCAASGFTFSNAWMDWVRQAPGKGLEWVGQITAKSNNYATYYAAPVKGRFTISRDDSKNTLYLQMNSLKTEDTAVYYCTDDGYHWGQGTLVTVSS

SEQ ID NO: 54 - protein, 115 aa, synthetic construct
EVQLVESGGGLVKPGGSLRLSCAASGFTFSNAWMDWVRQAPGKGLEWVGQITAKSNNYATDYAAPVKGRFTISRDDSKNTLYLQMNSLKTEDTAVYYCTDDGYHWGQGTLVTVSS

SEQ ID NO: 55 - protein, 115 aa, synthetic construct
EVQLVESGGGLVKPGGSLRLSCAASGFTFSNAWMDWVRQAPGKGLEWVGRIKSKTDGGTTDYAAPVKGRFTISRDDSKNTLYLQMNSLKTEDTAVYYCTDDGYHWGQGTLVTVSS

SEQ ID NO: 56 - protein, 115 aa, synthetic construct
EVQLVESGGGLVKPGGSLRLSCAASGFTFSNAYMDWVRQAPGKGLEWVGQITAKSNNYATYYAAPVKGRFTISRDDSKNTLYLQMNSLKTEDTAVYYCTDDGYHWGQGTLVTVSS

SEQ ID NO: 57 - protein, 107 aa, synthetic construct
AIQMTQSPSSLSASVGDRVTITCRASEDIRNGLAWYQQKPGKAPKLLIYAASSLQSGVPSRFSGSGSGTDFTLTISSLQPEDFATYYCEDTSKYPYTFGQGTKLEIK

SEQ ID NO: 58 - protein, 107 aa, synthetic construct
DIQMTQSPSSLSASVGDRVTITCRASEDIRNGLAWYQQKPGKAPKLLIYAASSLQSGVPSRFSGSGSGTDFTLTISSLQPEDFATYYCEDTSKYPYTFGQGTKLEIK

SEQ ID NO: 59 - protein, 107 aa, synthetic construct
AIQMTQSPSSLSASVGDRVTITCRASEDIRNDLGWYQQKPGKAPKLLIYNANSLHTGVPSRFSGSGSGTDFTLTISSLQPEDFATYYCEDTSKYPYTFGQGTKLEIK

SEQ ID NO: 60 - protein, 107 aa, synthetic construct
AIQMTQSPSSLSASVGDRVTITCRASEDIRNGLAWYQQKPGKAPKLLIYAANSLHTGVPSRFSGSGSGTDFTLTISSLQPEDFATYYCEDTSKYPYTFGQGTKLEIK

SEQ ID NO: 61 - protein, 107 aa, synthetic construct
AIQMTQSPSSLSASVGDRVTITCRASEDIRNGLAWYQQKPGKAPKLLIYAASSLHTGVPSRFSGSGSGTDFTLTISSLQPEDFATYYCEDTSKYPYTFGQGTKLEIK

SEQ ID NO: 62 - protein, 107 aa, synthetic construct
AIQMTQSPSSLSASVGDRVTITCRASEDIRNGLAWYQQKPGKAPKLLIYNANSLQTGVPSRFSGSGSGTDFTLTISSLQPEDFATYYCEDTSKYPYTFGQGTKLEIK

SEQ ID NO: 63 - protein, 107 aa, synthetic construct
AIQMTQSPSSLSASVGDRVTITCRASEDIRNGLAWYQQKPGKAPKLLIYNANSLHSGVPSRFSGSGSGTDFTLTISSLQPEDFATYYCEDTSKYPYTFGQGTKLEIK

SEQ ID NO: 64 - protein, 107 aa, synthetic construct
AIQMTQSPSSLSASVGDRVTITCRASEDIRNGLAWYQQKPGKAPKLLIYNANSLQSGVPSRFSGSGSGTDFTLTISSLQPEDFATYYCEDTSKYPYTFGQGTKLEIK

SEQ ID NO: 65 - protein, 443 aa, synthetic construct
EVQLVESGGGLVKPGGSLRLSCAASGFTFSNAWMDWVRQAPGKGLEWVGQITAKSNNYATYYAAPVKGRFTISRDDSKNTLYLQMNSLKTEDTAVYYCTDDGYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 66 - protein, 214 aa, synthetic construct
AIQMTQSPSSLSASVGDRVTITCRASEDIRNDLGWYQQKPGKAPKLLIYNANSLHTGVPSRFSGSGSGTDFTLTISSLQPEDFATYYCEDTSKYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 225 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 68 - protein, 443 aa, synthetic construct
EVQLVESGGGLVKPGGSLRLSCAASGFTFSNAWMDWVRQAPGKGLEWVGQITAKSNNYATDYAAPVKGRFTISRDDSKNTLYLQMNSLKTEDTAVYYCTDDGYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 69 - protein, 214 aa, synthetic construct
AIQMTQSPSSLSASVGDRVTITCRASEDIRNGLAWYQQKPGKAPKLLIYNANSLHTGVPSRFSGSGSGTDFTLTISSLQPEDFATYYCEDTSKYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 214 aa, synthetic construct
AIQMTQSPSSLSASVGDRVTITCRASEDIRNGLAWYQQKPGKAPKLLIYNANSLHSGVPSRFSGSGSGTDFTLTISSLQPEDFATYYCEDTSKYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 214 aa, synthetic construct
DIQMTQSPSSLSASVGDRVTITCRASEDIRNGLAWYQQKPGKAPKLLIYNANSLHTGVPSRFSGSGSGTDFTLTISSLQPEDFATYYCEDTSKYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 443 aa, synthetic construct
EVQLVESGGGLVKPGGSLRLSCAASGFTFSNAWMDWVRQAPGKGLEWIAQITAKSNNYATYYAAPVKGRFTISRDDSKNTLYLQMNSLKTEDTAVYYCTDDGYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 73 - protein, 214 aa, synthetic construct
AIQMTQSPSSLSASVGDRVTITCRASEDIRNGLAWYQQKPGKAPKLLIYAANSLHTGVPSRFSGSGSGTDFTLTISSLQPEDFATYYCEDTSKYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 214 aa, synthetic construct
AIQMTQSPSSLSASVGDRVTITCRASEDIRNGLAWYQQKPGKAPKLLIYAASSLHTGVPSRFSGSGSGTDFTLTISSLQPEDFATYYCEDTSKYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 443 aa, synthetic construct
EVQLLESGGGLVQPGGSLRLSCAASGFTFSNAWMDWVRQAPGKGLEWVSQITAKSNNYATYYADSVKGRFTISRDNSKNTLYLQMNSLRAEDTAVYYCTDDGYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 76 - protein, 443 aa, synthetic construct
EVQLVESGGGLVKPGESLRLSCAASGFTFSNAWMDWVRQAPGKRLEWIAQITAKSNNYATYYAAPVKGRFTISRDDSKNTLYLQMNSLKKEDTAVYYCTDDGYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 77 - protein, 214 aa, synthetic construct
AIQMTQSPSSLSASVGDRVTIACRASEDIRNGLAWYQQKPGKAPKLLIYNANSLHTGVPSRFSGSGSGTDFTLTISSLQPEDEAIYYCEDTSKYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 446 aa, synthetic construct
QVQLVQSGAEVKKPGASVKVSCKASGYTFTNYWMHWVRQAPGQGLEWMGIIHPNSGSTNYNEKFQGRVTMTRDTSTSTVYMELSSLRSEDTAVYYCARGIYDYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 79 - protein, 218 aa, synthetic construct
DIQMTQSPSSLSASVGDRVTITCRASESVSIHGTHLMHWYQQKPGKAPKLLIYAASNLESGVPSRFSGSGSGTDFTLTISSLQPEDFATYYCQQSIEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 218 aa, synthetic construct
DIQMTQSPSSLSASVGDRVTITCRASESVSIHGTHLMHWYQQKPGKAPKLLIYAASNLESGVPSRFSGSGSETDFTLTISSLQPEDFATYYCQQSIEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 218 aa, synthetic construct
DIQMTQSPSSLSASVGDRVTITCRASESVSIHGTHLMHWYQQKPGKAPKLLIYAASNLQSGVPSRFSGSGSETDFTLTISSLQPEDFATYYCQQSIEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 218 aa, synthetic construct
DIQMTQSPSSLSASVGDRVTITCRASESVSIHGTHLMHWYQQKPGKAPKLLIYAASSLQSGVPSRFSGSGSGTDFTLTISSLQPEDFATYYCQQSIEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 218 aa, synthetic construct
DIQMTQSPSSLSASVGDRVTITCRASESVSIHGTHLMHWYQQKPGKAPKLLIYAASSLQSGVPSRFSGSGSETDFTLTISSLQPEDFATYYCQQSIEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 218 aa, synthetic construct
DIQMTQSPSSLSASVGDRVTITCRASESVSIHGTHLMHWYQQKPGKAPKLLIYAASNLESGVPSRFSGSGSGTDFTLTISSLQPEDFATYFCQQSIEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 218 aa, synthetic construct
DIQMTQSPSSLSASVGDRVTITCRASESVSIHGTHLMHWYQQKPGKAPKLLIYAASNLESGVPSRFSGSGSETDFTLTISSLQPEDFATYFCQQSIEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 446 aa, synthetic construct
QVQLVQSGAEVKKPGASVKVSCKASGYTFTNYWMHWVRQAPGQGLEWMGIIHPNSGSTNYNEKFQGRVTMTVDKSTSTVYMELSSLRSEDTAVYYCARGIYDYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 87 - protein, 446 aa, synthetic construct
QVQLQQPGAELVKPGASVKLSCKASGYTFTNYWMHWVKQRPGQGLEWIGIIHPNSGSTNYNEKFKSKATLTVDKSSSTAYMQLSSLTSEDSAVYYCARGIYDYPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 88 - protein, 218 aa, synthetic construct
DIVLTQSPASLAVSLGQRATISCRASESVSIHGTHLMHWYQQKPGQPPKLLIYAASNLESGVPARFSGSGSETDFTLNIHPVEEEDAATYFCQQSIEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 715 aa, synthetic construct
EVQLLESGGGLVQPGGSLRLSCAASGFTFSDYGLSWVRQAPGKGLEWVSLIDSSGSSTFYADSVKGRFTISRDNSKNTLYLQMNSLRAEDTAVYYCAKEHGLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGSGSGSGSGSGSGSGSQSVLTQPPSASGTPGRRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 90 - protein, 214 aa, synthetic construct
EIVLTQSPGTLSLSPGERATLSCKASQDIDDAINWYQQKPGQAPRLLIYDASLRATGIPDRFSGSGSGTDFTLTISRLEPEDFAVYYCQQSLRTPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 443 aa, synthetic construct
EVQLLESGGGLVQPGGSLRLSCAASGFTFSSYAMNWVRQAPGKGLEWVSAITASGGSTYYADSVKGRFTISRDNSKNTLYLQMNSLRAEDTAVYYCARYWPMSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 92 - protein, 213 aa, synthetic construct
EIVLTQSPGTLSLSPGERATLSCRASQSVSAYYLAWYQQKPGQAPRLLMYDASIRATGIPDRFSGSGSGTDFTLTISRLEPEDFAVYYCQQYERWPLTFGQGTKVEIKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 94 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 95 - protein, 107 aa, Homo sapiens [sequence omitted]

SEQ ID NO: 96 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 97 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 98 - protein, 5 aa, synthetic construct
EPKSC

SEQ ID NO: 99 - protein, 10 aa, synthetic construct
  VARIANT 8: note = X is S or P
DKTHTCPXCP

SEQ ID NO: 100 - protein, 7 aa, synthetic construct
  VARIANT 5: note = X is S or P
HTCPXCP

SEQ ID NO: 101 - protein, 5 aa, synthetic construct
  VARIANT 3: note = X is S or P
CPXCP

SEQ ID NO: 102 - protein, 107 aa, Homo sapiens
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 103 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 330 aa, Homo sapiens [sequence omitted]

SEQ ID NO: 105 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 107 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 108 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 110 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 111 - protein, 5 aa, synthetic construct
GGGGS

SEQ ID NO: 112 - protein, 9 aa, synthetic construct
GGGGSGGGG

SEQ ID NO: 113 - protein, 10 aa, synthetic construct
GGGGSGGGGS

SEQ ID NO: 114 - protein, 10 aa, synthetic construct
SGGGGSGGGG

SEQ ID NO: 115 - protein, 14 aa, synthetic construct
GGGGSGGGGSGGGG

SEQ ID NO: 116 - protein, 10 aa, synthetic construct
GSPGSSSSGS

SEQ ID NO: 117 - protein, 15 aa, synthetic construct
GGGGSGGGGSGGGGS

SEQ ID NO: 118 - protein, 20 aa, synthetic construct
GGGGSGGGGSGGGGSGGGGS

SEQ ID NO: 119 - protein, 8 aa, synthetic construct
GSGSGSGS

SEQ ID NO: 120 - protein, 8 aa, synthetic construct
GSGSGNGS

SEQ ID NO: 121 - protein, 8 aa, synthetic construct
GGSGSGSG

SEQ ID NO: 122 - protein, 6 aa, synthetic construct
GGSGSG

SEQ ID NO: 123 - protein, 4 aa, synthetic construct
GGSG

SEQ ID NO: 124 - protein, 8 aa, synthetic construct
GGSGNGSG

SEQ ID NO: 125 - protein, 8 aa, synthetic construct
GGNGSGSG

SEQ ID NO: 126 - protein, 6 aa, synthetic construct
GGNGSG

SEQ ID NO: 127 - protein, 54 aa, Homo sapiens
MLQMAGQCSQNEYFDSLLHACIPCQLRCSSNTPPLTCQRYCNASVTNSVKGTNA

SEQ ID NO: 128 - protein, 183 aa, Macaca fascicularis
MLQMARQCSQNEYFDSLLHDCKPCQLRCSSTPPLTCQRYCNASMTNSVKGMNAILWTCLGLSLIISLAVFVLTFLLRKMSSEPLKDEFKNTGSGLLGMANIDLEKGRTGDEIVLPRGLEYTVEECTCEDCIKNKPKVDSDHCFPLPAMEEGATILVTTKTNDYCNSLSAALSVTEIEKSISAR

SEQ ID NO: 129 - protein, 53 aa, Macaca fascicularis
MLQMARQCSQNEYFDSLLHDCKPCQLRCSSTPPLTCQRYCNASMTNSVKGMNA

SEQ ID NO: 130 - protein, 111 aa, synthetic construct; identical to SEQ ID NO: 45

SEQ ID NO: 131 - protein, 111 aa, synthetic construct; identical to SEQ ID NO: 46

SEQ ID NO: 132 - protein, 445 aa, synthetic construct
EVQLLESGGGLVQPGGSLRLSCAASGFTFSKYAMAWVRQAPGKGLEWVASISTGGVNTYYADSVKGRFTISRDNSKNTLYLQMNSLRAEDTAVYYCATHTGDYFDYWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 133 - protein, 218 aa, synthetic construct
EIVLTQSPGTLSLSPGERATLSCRASQSVSISGINLMNWYQQKPGQQPKLLIYHASILASGIPDRFSGSGSGTDFTLTISRLEPEDFAVYYCQQTRESPLTFGQGTRLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 670 aa, synthetic construct
EVQLLESGGGLVQPGGSLRLSCAASGFTFSKYAMAWVRQAPGKGLEWVASISTGGVNTYYADSVKGRFTISRDNSKNTLYLQMNSLRAEDTAVYYCATHTGDYFDYWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 135 - protein, 232 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 446 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 137 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 439 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 139 - protein, 232 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 5 aa, synthetic construct
KYAMA

SEQ ID NO: 141 - protein, 17 aa, synthetic construct
SISTGGVNTYYADSVKG

SEQ ID NO: 142 - protein, 8 aa, synthetic construct
HTGDYFDY

SEQ ID NO: 143 - protein, 15 aa, synthetic construct
RASQSVSISGINLMN

SEQ ID NO: 144 - protein, 7 aa, synthetic construct
HASILAS

SEQ ID NO: 145 - protein, 9 aa, synthetic construct
QQTRESPLT

SEQ ID NO: 146 - protein, 117 aa, synthetic construct
EVQLLESGGGLVQPGGSLRLSCAASGFTFSKYAMAWVRQAPGKGLEWVASISTGGVNTYYADSVKGRFTISRDNSKNTLYLQMNSLRAEDTAVYYCATHTGDYFDYWGQGTMVTVSS

SEQ ID NO: 147 - protein, 111 aa, synthetic construct
EIVLTQSPGTLSLSPGERATLSCRASQSVSISGINLMNWYQQKPGQQPKLLIYHASILASGIPDRFSGSGSGTDFTLTISRLEPEDFAVYYCQQTRESPLTFGQGTRLEIK

SEQ ID NO: 148 - protein, 117 aa, synthetic construct
EVQLLESGGGLVQPGGSLRLSCAASGFTFSKYAMAWVRQAPGKGLEWVASISTGGVNTYYRDSVKARFTISRDNSKNTLYLQMNSLRAEDTAVYYCATHTGDYFDYWGQGTMVTVSS

SEQ ID NO: 149 - protein, 117 aa, synthetic construct
ELQLLESGGGLVQPGGSLRLSCAASGFTFSKYAMAWVRQAPGKGLEWVASISTGGVNTYYRDSVKARFTISRDNAKNTLYLQMNSLRAEDTAVYYCATHTGDYFDYWGQGTMVTVSS

SEQ ID NO: 150 - protein, 117 aa, synthetic construct
ELQLLESGGGLVQPGGSLRLSCAASGFTFSKYAMAWVRQAPGKGLEWVASISTGGVNTYYADSVKGRFTISRDNAKNTLYLQMNSLRAEDTAVYYCATHTGDYFDYWGQGTMVTVSS

SEQ ID NO: 151 - protein, 111 aa, synthetic construct
DIVMTQSPDSLAVSLGERATINCRASQSVSISGINLMNWYQQKPGQQPKLLIYHASILASGVPDRFSGSGSGTDFTLTISSLQAEDVAVYYCQQTRESPLTFGQGTRLEIK

SEQ ID NO: 152 - protein, 111 aa, synthetic construct
DIVMTQSPDSLAVSLGERATINCKSSQSVSISGINLMNWYQQKPGQQPKLLIYHASILASGVPDRFSGSGSGTDFTLTISSLQAEDVAVYYCQQTRESPLTFGQGTRLEIK

SEQ ID NO: 153 - protein, 111 aa, synthetic construct
EIVLTQSPGTLSLSPGERATLSCRASQSVSISGINLMNWYQQKPGQQPRLLIYHASILASGIPDRFSGSGSGTDFTLTISRLEPEDFAVYYCQQTRESPLTFGQGTRLEIK

SEQ ID NO: 154 - protein, 111 aa, synthetic construct
DIQMTQSPSSLSASVGDRVTITCRASQSVSISGINLMNWYQQKPGKQPKLLIYHASILASGVPSRFSGSGSGTDFTLTISSLQPEDFATYYCQQTRESPLTFGQGTRLEIK

SEQ ID NO: 155 - protein, 111 aa, synthetic construct
DIQMTQSPSSLSASVGDRVTITCRASQSVSISGINLMNWYQQKPGQQPKLLIYHASILASGVPSRFSGSGSGTDFTLTISSLQPEDFATYYCQQTRESPLTFGQGTRLEIK

SEQ ID NO: 156 - protein, 5 aa, synthetic construct
NYGMA

SEQ ID NO: 157 - protein, 17 aa, synthetic construct
SISTGGGNTYYRDSVKG

SEQ ID NO: 158 - protein, 7 aa, synthetic construct
HDRGGLY

SEQ ID NO: 159 - protein, 16 aa, synthetic construct
RSSKSLLHSNGITYVY

SEQ ID NO: 160 - protein, 7 aa, synthetic construct
RMSNRAS

SEQ ID NO: 161 - protein, 9 aa, synthetic construct
GQLLENPYT

SEQ ID NO: 162 - protein, 116 aa, synthetic construct
QVQLVESGGGVVQPGRSLRLSCAASGFSFSNYGMAWVRQAPGKGLEWVASISTGGGNTYYRDSVKGRFTISRDNSKNTLYLQMNSLRAEDTAVYYCTRHDRGGLYWGQGTMVTVSS

SEQ ID NO: 163 - protein, 116 aa, synthetic construct
EVQLVESGGGVVQPGRSLRLSCAASGFSFSNYGMAWVRQAPGKGLEWVASISTGGGNTYYRDSVKGRFTISRDNAKNTLYLQMNSLRAEDTAVYYCTRHDRGGLYWGQGTMVTVSS

SEQ ID NO: 164 - protein, 116 aa, synthetic construct
QVQLVESGGGVVQPGRSLRLSCAASGFSFSNYGMAWVRQAPGKGLEWVASISTGGGNTYYADSVKGRFTISRDNSKNTLYLQMNSLRAEDTAVYYCTRHDRGGLYWGQGTMVTVSS

SEQ ID NO: 165 - protein, 116 aa, synthetic construct
EVQLVESGGGVVQPGRSLRLSCAASGFSFSNYGMAWVRQAPGKGLEWVASISTGGGNTYYADSVKGRFTISRDNAKNTLYLQMNSLRAEDTAVYYCTRHDRGGLYWGQGTMVTVSS

SEQ ID NO: 166 - protein, 116 aa, synthetic construct
EVQLVESGGGLVQPGGSLRLSCAASGFSFSNYGMAWVRQAPGKGLEWVASISTGGGNTYYRDSVKGRFTISRDNAKNTLYLQMNSLRAEDTAVYYCTRHDRGGLYWGQGTMVTVSS

SEQ ID NO: 167 - protein, 116 aa, synthetic construct
EVQLVESGGGLVQPGGSLRLSCAASGFSFSNYGMAWVRQAPGKGLEWVASISTGGGNTYYADSVKGRFTISRDNAKNTLYLQMNSLRAEDTAVYYCTRHDRGGLYWGQGTMVTVSS

SEQ ID NO: 168 - protein, 112 aa, synthetic construct
DIVMTQSPLSLPVTPGEPASISCRSSKSLLHSNGITYVYWYLQKPGQSPQVLIYRMSNLASGVPDRFSGSGSGTDFTLKISRVEAEDVGVYHCGQLLENPYTFGQGTKLEIK

SEQ ID NO: 169 - protein, 112 aa, synthetic construct
DIVMTQSPLSLPVTPGEPASISCRSSKSLLHSNGITYVYWYLQKPGKSPQVLIYRMSNLASGVPDRFSGSGSGTDFTLKISRVEAEDVGVYHCGQLLENPYTFGQGTKLEIK

SEQ ID NO: 170 - protein, 112 aa, synthetic construct
DIVMTQSPLSLPVTPGEPASISCRSSKSLLHSNGITYVYWYLQKPGQSPQLLIYRMSNRASGVPDRFSGSGSGTDFTLKISRVEAEDVGVYHCGQLLENPYTFGQGTKLEIK

SEQ ID NO: 171 - protein, 112 aa, synthetic construct
DIVMTQSPDSLAVSLGERATINCKSSKSLLHSNGITYVYWYQQKPGQPPKLLIYRMSNLASGVPDRFSGSGSGTDFTLTISSLQAEDVAVYHCGQLLENPYTFGQGTKLEIK

SEQ ID NO: 172 - protein, 112 aa, synthetic construct
EIVLTQSPGTLSLSPGERATLSCRASKSLLHSNGITYVYWYQQKPGQAPRLLIYRMSNLASGIPDRFSGSGSGTDFTLTISRLEPEDFAVYHCGQLLENPYTFGQGTKLEIK

SEQ ID NO: 173 - protein, 5 aa, synthetic construct
SYAMN

SEQ ID NO: 174 - protein, 19 aa, synthetic construct
RIRSKYNNYATYYADSVKG

SEQ ID NO: 175 - protein, 14 aa, synthetic construct
HTTFPSSYVSYYGY

SEQ ID NO: 176 - protein, 14 aa, synthetic construct
GSSTGAVTTSNYAN

SEQ ID NO: 177 - protein, 7 aa, synthetic construct
GTNKRAP

SEQ ID NO: 178 - protein, 9 aa, synthetic construct
ALWYSNLWV

SEQ ID NO: 179 - protein, 125 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

SEQ ID NO: 180 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 181 - protein, 10 aa, synthetic construct
GYSFTGYTMN

SEQ ID NO: 182 - protein, 17 aa, synthetic construct
LINPYKGVSTYNQKFKD

SEQ ID NO: 183 - protein, 13 aa, synthetic construct
SGYYGDSDWYFDV

SEQ ID NO: 184 - protein, 11 aa, synthetic construct
RASQDIRNYLN

SEQ ID NO: 185 - protein, 7 aa, synthetic construct
YTSRLES

SEQ ID NO: 186 - protein, 9 aa, synthetic construct
QQGNTLPWT

SEQ ID NO: 187 - protein, 122 aa, synthetic construct [sequence omitted]

SEQ ID NO: 188 - protein, 107 aa, synthetic construct [sequence omitted]

SEQ ID NO: 189 - protein, 366 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 190 - protein, 378 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 191 - protein, 446 aa, Synthetic construct
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 192 - protein, 216 aa, synthetic construct
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 671 aa, synthetic construct
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 444 aa, synthetic construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 196 - protein, 215 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 443 aa, synthetic construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 228 aa, synthetic construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 200 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 201 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 202 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 203 - protein, 215 aa, Homo sapiens
MLQMAGQCSQNEYFDSLLHACIPCQLPCSSNTPPLTCQRYCNASVTNSVKGTNAILWTCLGLSLIISLAVFVLMFLLRKISSEPLEDEFKNTGSGLLGMANIDLEKSRTGDEIILPRGLEYTVEECTCEDCIKSKPKVDSDHCFPLPAMEEGATILVTTKTNDYCKSLPAALSATEIEKSISARTRTRPLEQKLISEEDLAANDILDYKDDDDKV

SEQ ID NO: 204 - protein, 214 aa, Homo sapiens
MLQMAGQCSQNEYFDSLLHACIPCQLRCSNTPPLTCQRYCNASVTNSVKGTNAILWTCLGLSLIISLAVFVLMFLLRKISSEPLEDEFKNTGSGLLGMANIDLEKSRTGDEIILPRGLEYTVEECTCEDCIKSKPKVDSDHCFPLPAMEEGATILVTTKTNDYCKSLPAALSATEIEKSISARTRTRPLEQKLISEEDLAANDILDYKDDDDKV

SEQ ID NO: 205 - protein, 215 aa, Homo sapiens
MLQMAGQCSQNEYFDSLLHACIPCQLRCSSNTSPLTCQRYCNASVTNSVKGTNAILWTCLGLSLIISLAVFVLMFLLRKISSEPLEDEFKNTGSGLLGMANIDLEKSRTGDEIILPRGLEYTVEECTCEDCIKSKPKVDSDHCFPLPAMEEGATILVTTKTNDYCKSLPAALSATEIEKSISARTRTRPLEQKLISEEDLAANDILDYKDDDDKV

SEQ ID NO: 206 - protein, 214 aa, Homo sapiens
MLQMAGQCSQNEYFDSLLHACIPCQLRCSSNTPLTCQRYCNASVTNSVKGTNAILWTCLGLSLIISLAVFVLMFLLRKISSEPLEDEFKNTGSGLLGMANIDLEKSRTGDEIILPRGLEYTVEECTCEDCIKSKPKVDSDHCFPLPAMEEGATILVTTKTNDYCKSLPAALSATEIEKSISARTRTRPLEQKLISEEDLAANDILDYKDDDDKV

SEQ ID NO: 207 - protein, 447 aa, synthetic construct
QVQLVQSGAEVKKPGASVKVSCKASGYTFTNYWMHWVRQAPGQGLEWMGIIHPNSGSTNYNEKFQGRVTMTRDTSTSTVYMELSSLRSEDTAVYYCARGIYDYPFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 208 - protein, 707 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0285998 A1
(43) Pub. Date: Sep. 24, 2026

(54) IMMUNOSTIMULATORY ANTIGEN BINDING MOLECULES THAT SPECIFICALLY BIND TO BCMA

(71) Applicant: Hoffmann-La Roche Inc., Little Falls, NJ (US)

(72) Inventors: Marlene Biehl, Zurich (CH); Alexander Christian Bujotzek, München (DE); Alejandro Carpy Gutierrez Cirlos, San Francisco, CA (US); Jan Eckmann, München (DE); Tanja Fauti, Zurich (CH); Algirdas Grevys, Oslo (NO); Carina Hage, Penzberg (DE); Christian Klein, Bonstetten (CH); Julia Krueger, München (DE); Stefan Lorenz, Iffeldorf (DE); Céline Marban-Doran, Wettolsheim (FR); Pablo Umaña, Lachen (CH)

(73) Assignee: Hoffmann-La Roche Inc., Little Falls, NJ (US)

(21) Appl. No.: 19/402,720

(22) Filed: Nov. 26, 2025

Related U.S. Application Data
(63) Continuation of application No. PCT/EP2024/064692, filed on May 29, 2024.

(30) Foreign Application Priority Data
Jun. 1, 2023 (EP) 23176584.3
Aug. 4, 2023 (EP) 23189804.0

(51) Int. Cl. C07K 16/28; A61K 38/00; A61K 39/00; A61K 45/06; A61P 35/00; A61P 37/04; C07K 14/705

(52) U.S. Cl. CPC C07K 16/2878; A61K 45/06; A61P 35/00; A61P 37/04; C07K 14/70575; C07K 16/2809; C07K 16/2866; A61K 38/00; A61K 2039/507; C07K 2317/31; C07K 2317/52; C07K 2317/55; C07K 2317/565; C07K 2317/71; C07K 2319/00

(57) ABSTRACT

The present invention relates to new humanized antibodies that bind to B-cell maturation antigen (BCMA), and to immunostimulatory antigen binding molecules compromising these BCMA antibodies, in particular to BCMA targeting 4-1BBL trimer-containing antigen binding molecules, methods for their production, as well as their use in the treatment of cancer.

### CROSS-REFERENCE TO RELATED APPLICATIONS

This application is a continuation of International Application No. PCT/EP2024/064692, filed May 29, 2024, which claims benefit of priority to European Patent Application No. 23176584.3, filed Jun. 1, 2023, and European Patent Application No. 23189804.0, filed Aug. 4, 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Nov. 24, 2025, is named P38061-US_SL.xml and is 213,860 bytes in size.

### FIELD OF THE INVENTION

The present invention relates to new humanized antibodies that bind to B-cell maturation antigen (BCMA), and to immunostimulatory antigen binding molecules compromising these BCMA antibodies, in particular to BCMA targeting 4-1BBL trimer-containing antigen binding molecules, methods for their production, as well as their use in the treatment of cancer.

### BACKGROUND

Within the group of hematologic malignancies, multiple myeloma (MM) represents a large, dynamic, competitive disease area, and a disease, which presents significant continuing unmet medical need, still not being considered curable. Multiple myeloma (MM) is a plasma cell malignancy and the second most common hematologic disorder, mainly affecting an elderly population. Despite dramatic improvements in the diagnostics, classification and treatment of MM, patients with high-risk disease have not yet benefited from therapeutic advances and are often primary refractory to treatment or relapse early. Additionally, the majority of low-risk MM patients ultimately develop drug-resistant clones, become refractory to treatment and transition to high-risk disease. Thus, novel therapies with different mechanisms of action, as well as the optimization of treatment efficacy is key to reduce the risk of disease relapse and deepen response durability (Caraccio C, Krishna S., Phillips DJ., and Schürch C M, Front Immunol. 2020, 11, 501).

CAR-T cells are one of the promising novel treatment modalities: early data from these agents show significant efficacy, and promise to bring major improvements to the treatment of MM. Nevertheless, as already seen with CAR-T therapies in the treatment of non-Hodgkin lymphoma (NHL), these treatment modalities are associated with similar liabilities to agents already seen for the treatment of NHL—especially early toxicity from cytokine release syndrome and/or associated neurotoxicity as well as limitations with regard to accessibility.

T cell bispecific antibodies (TCBs) offer another promising immunotherapeutic approach by binding and stimulating CD3 independently from antigen presentation on major histocompatibility complex (MHC) class I. Via simultaneous binding to tumor-specific antigens (TA), T-cells are redirected to TA-positive MM cells to induce their specific lysis. Although some of these TCBs have demonstrated significant antitumor efficacy in patients, their activity remains to be optimized to ultimately induce robust long lasting cure.

New classes of bispecific antibodies mimicking signal 2 comprise molecules that target a tumor-specific antigen (TA) and a co-stimulatory receptor (e.g. OX40, GITR, CD137, ICOS or CD28). Combining these bispecific antibodies with the emerging class of TCBs may provide potent “synthetic off-the-shelf CAR T cell-like” antibody therapies to further deepen and prolong the TCB-mediated anti-tumor responses in patients.

B cell maturation antigen (BCMA), a transmembrane glycoprotein in the tumor necrosis factor receptor superfamily 17 (TNFRSF17), is expressed at significantly higher levels in all patient MM cells but not on other normal tissues except normal plasma cells. BCMA-chimeric antigen receptor (CAR) T-cells have already shown significant clinical activities in patients with relapsed/refractory MM (RRMM) who have undergone at least three prior treatments, including with a proteasome inhibitor and an immunomodulatory drug (IMiD). Additional modalities, including an anti-BCMA antibody-drug conjugate also have achieved significant clinical responses in patients who failed at least three prior lines of therapy, including an anti-CD38 antibody, a proteasome inhibitor, and an immunomodulatory drug (Cho et al, Front Immunolog. 2018, 9, 1821). However, there is still a need for better treatment options of Multiple Myeloma.

4-1BB (CD137), a member of the TNF receptor superfamily, was first identified as an inducible molecule expressed by activated T cells (Kwon and Weissman, 1989, Proc Natl Acad Sci USA 86, 1963-1967). Subsequent studies demonstrated that many other immune cells also express 4-1BB, including NK cells, B cells, NKT cells, monocytes, neutrophils, mast cells, dendritic cells (DCs) and cells of non-hematopoietic origin such as endothelial and smooth muscle cells (Vinay and Kwon, 2011, Cell Mol Immunol 8, 281-284). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., 2002, J Immunol 168, 3755-3762; Zhang et al., 2010, Clin Cancer Res 13, 2758-2767).

4-1BB ligand (4-1BBL or CD137L) was identified in 1993 (Goodwin et al., 1993, Eur J Immunol 23, 2631-2641). It has been shown that expression of 4-1BBL was restricted on professional antigen presenting cells (APC) such as B-cells, DCs and macrophages. Inducible expression of 4-1BBL is characteristic for T-cells, including both and T-cell subsets, and endothelial cells (Shao and Schwarz, 2011, J Leukoc Biol 89, 21-29).

Co-stimulation through the 4-1BB receptor (for example by 4-1BBL ligation) activates multiple signaling cascades within the T cell (both CD4+ and CD8+ subsets), powerfully augmenting T cell activation. In combination with TCR triggering, agonistic 4-1BB-specific antibodies enhance proliferation of T-cells, stimulate lymphokine secretion and decrease sensitivity of T-lymphocytes to activation-induced cells death (Snell et al., 2011, Immunol Rev 244, 197-217). This mechanism was further advanced as the first proof of concept in cancer immunotherapy. In a preclinical model administration of an agonistic antibody against 4-1BB in tumor bearing mice led to potent anti-tumor effect (Melero et al., 1997, Nat Med 3, 682-685). Also, in hematological tumors, the mono-immunotherapy of an anti-CD137 antibody could rescue 40-50% of mice in a preclinical model of multiple myeloma (Murillo et al, 2008, Clin. Cancer Res. 2008, 14(21), 6895). Later, accumulating evidence indicated that 4-1BB usually exhibits its potency as an anti-tumor agent mainly when administered in combination with other immunomodulatory compounds, chemotherapeutic reagents, tumor-specific vaccination or radiotherapy (Bartkowiak and Curran, 2015, Front Oncol 5, 117).

Signaling of the TNFR-superfamily needs cross-linking of the trimerized ligands to engage with the receptors, so does the 4-1BB agonistic antibodies which require wild type Fc-binding (Li and Ravetch, 2011, Science 333, 1030-1034). However, systemic administration of 4-1BB-specific agonistic antibodies with the functionally active Fc domain resulted in influx of CD8+ T-cells associated with liver toxicity (Dubrot et al., 2010, Cancer Immunol Immunother 59, 1223-1233). In line with that, an Fc-competent 4-1BB agonistic Ab (BMS-663513) (NCT00612664) caused a grade 4 hepatitis in the clinic, leading to termination of the trial (Simeone and Ascierto, 2012, J Immunotoxicol 9, 241-247). Therefore, a targeted delivery of an 4-1BB agonist to the tumor side may be an option for eliciting antitumor efficacy and avoiding a systemic toxicity. There is a strong need to provide BCMA-targeted 4-1BB agonists with advantageous properties.

### SUMMARY

The present invention describes new BCMA-targeted 4-1BB agonists which achieve a tumor-dependent T cell activation without a systemic toxicity. The BCMA-4-1BBL antigen binding molecules of the invention were constructed with an IgG fusion protein composed of a split trimeric 4-1BB ligand comprising the amino acid sequence of SEQ ID NO:9 or SEQ ID NO:10 (which corresponds to the natural ligand), and a tumor antigen targeting moiety recognizing BCMA fused to a an Fc domain composed of a first and a second subunit capable of stable association comprising one or more amino acid substitution that reduces the binding affinity of the antigen binding molecule to an Fc receptor and/or effector function (silent Fc domain). Fc receptor-mediated cross-linking is thereby abrogated and tumor-specific activation is achieved by cross-linking through binding of the second antigen binding domain capable of specific binding to BCMA. Thus, unspecific FcγR mediated crosslinking responsible for Fc-mediated toxicity is replaced by BCMA-targeted MM cell specific crosslinking. The desired mode of action for these molecules is to enhance the effector function of tumor-infiltrating T cells or NK cells upon activation by a tumor-targeted T cell bispecific antibody or ADCC antibody, respectively. As such these molecules are considered to have the potency to treat multiple myeloma.

In particular, the invention relates to new antigen binding molecules that bind to B cell maturation antigen (BCMA), in particular to new optimized respective humanized BCMA antibodies and to BCMA targeting 4-1BBL trimer-containing antigen binding molecules comprising these BCMA antibodies as well as their use in the treatment of cancer. The invention further relates to methods of producing these molecules and to methods of using the same.

The BCMA targeting 4-1BBL trimer-containing antigen binding molecules are active in presence of BCMA-expressing tumors, comprise the natural human 4-1BB ligand and should thus impose less safety issues compared to conventional 4-1BB agonistic antibodies or more artificial fusion proteins. The new antigen binding molecules combine an anti-BCMA antigen binding domain with a moiety that is capable of forming a costimulatory 4-1BBL trimer and that is sufficiently stable to be pharmaceutically useful. The immunostimulatory antigen binding molecules of the invention provide a trimeric and thus biologically active human 4-1BB ligand, although one of the trimerizing 4-1BBL ectodomains is located on another polypeptide than the other two 4-1BBL ectodomains of the molecule.

Thus, provided herein is an immunostimulatory antigen binding molecule that specifically binds to B cell maturation agent (BCMA), comprising
(a) a Fab molecule comprising
   (i) a heavy chain variable region (V_{H} BCMA) comprising a heavy chain complementary determining region CDR-H1 of SEQ ID NO:1 (GYTFTNYWMH), a CDR-H2 of SEQ ID NO:2 (IIHPNSGSTNYNEKFQG), and a CDR-H3 of SEQ ID NO:3 (GIYDYPFAY), and
   (ii) a light chain variable region (V_{L} BCMA) selected from the group consisting of
      (a) a VL comprising a light chain complementary determining region CDR-L1 of SEQ ID NO:4 (RASESVSIHGTHLMH), a CDR-L2 of SEQ ID NO:5 (AASSLQS) and a CDR-L3 of SEQ ID NO:6 (QQSIEDPYT); and
      (b) a VL comprising a light chain complementary determining region CDR-L1 of SEQ ID NO:4 (RASESVSIHGTHLMH), a CDR-L2 of SEQ ID NO:7 (AASNLES) and a CDR-L3 of SEQ ID NO:6 (QQSIEDPYT), and
      (c) a VL comprising a light chain complementary determining region CDR-L1 of SEQ ID NO:4 (RASESVSIHGTHLMH), a CDR-L2 of SEQ ID NO:8 (AASNLQS) and a CDR-L3 of SEQ ID NO:6 (QQSIEDPYT),
(b) a first and a second polypeptide that are linked to each other by a disulfide bond, wherein the antigen binding molecule is characterized in that the first polypeptide comprises two ectodomains of 4-1BBL each comprising the amino acid sequence of SEQ ID NO:9 or SEQ ID NO:10 that are connected to each other by a peptide linker, and in that the second polypeptide comprises one ectodomain of 4-1BBL comprising the amino acid sequence of SEQ ID NO:9 or SEQ ID NO:10, and
(c) an IgG Fc domain.

In one aspect, provided is an immunostimulatory antigen binding molecule as defined above, wherein the ectodomain of 4-1BBL or a fragment thereof comprises the amino acid sequence of SEQ ID NO:9 or SEQ ID NO:10. In one particular aspect, the ectodomain of 4-1BBL or a fragment thereof comprises the amino acid sequence of SEQ ID NO:9. In one aspect, the immunostimulatory antigen binding molecule comprises three ectodomains of 4-1BBL each comprising the amino acid sequence of SEQ ID NO:9 or SEQ ID NO:10, particularly the amino acid sequence of SEQ ID NO:9.

In one aspect, provided is an immunostimulatory antigen binding molecule as defined above, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10. In one particular aspect, the immunostimulatory antigen binding molecule is characterized in that the first polypeptide comprises the amino acid sequence of SEQ ID NO:11 and in that the second polypeptide comprises the amino acid sequence of SEQ ID NO:9.

In one aspect, provided is an immunostimulatory antigen binding molecule as defined above, wherein the antigen binding domain comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16.

In one aspect, the antibody that specifically binds to BCMA comprises an IgG Fc domain composed of a first and a second subunit. In one aspect, the IgG Fc domain composed of a first and a second subunit is an IgG1 Fc domain. In one aspect, the IgG Fc domain is a human Fc domain.

In one aspect, the IgG Fc domain comprises a modification promoting the association of the first and the second subunit of the Fc domain. In one aspect, the Fc domain comprises knobs into hole modifications. In one aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In one further aspect, the IgG Fc domain comprises one or more amino acid substitution that reduces binding to an Fc receptor and/or effector function. In one particular aspect, the IgG Fc domain is of human IgG1 subclass and comprises the amino acid mutations L234A, L235A and P329G (numbering according to Kabat EU index).

In one aspect, the immunostimulatory antigen binding molecule as described herein comprises
(a) a first polypeptide, comprising (ai) a first ectodomain of 4-1BBL comprising the amino acid sequence of SEQ ID NO:9 or SEQ ID NO:10, fused at its C-terminus to the N-terminus of the second ectodomain of 4-1BBL or fragment thereof, (aii) a second ectodomain of 4-1BBL comprising the amino acid sequence of SEQ ID NO:9 or SEQ ID NO:10, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL comprising the amino acid sequence of SEQ ID NO:9 or SEQ ID NO:10, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
   (d) a fourth polypeptide, comprising the light chain of the Fab molecule.

In one aspect, the immunostimulatory antigen binding molecule as described above is a molecule, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one particular aspect, the immunostimulatory antigen binding molecule as described herein comprises
(a) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:24, or
(b) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:25.

In one particular aspect, the immunostimulatory antigen binding molecule comprises a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:24. In one particular aspect, the immunostimulatory antigen binding molecule comprises (a) a first polypeptide comprising the amino acid sequence of SEQ ID NO:208, a second polypeptide comprising the amino acid sequence of SEQ ID NO:22, a third polypeptide comprising the amino acid sequence of SEQ ID NO:207 and a fourth polypeptide comprising the amino acid sequence of SEQ ID NO:24. In one aspect, the immunostimulatory antigen binding molecule consists of (a) a first polypeptide comprising the amino acid sequence of SEQ ID NO:208, a second polypeptide comprising the amino acid sequence of SEQ ID NO:22, a third polypeptide comprising the amino acid sequence of SEQ ID NO:207 and a fourth polypeptide comprising the amino acid sequence of SEQ ID NO:24.

Provided herein is also an immunostimulatory antigen binding molecule that specifically binds to B cell maturation agent (BCMA), comprising
(a) a Fab molecule comprising
   (i) a heavy chain variable region (V_{L} BCMA) selected from the group consisting of
      (a) a VH comprising a heavy chain complementary determining region CDR-H1 of SEQ ID NO:26 (GFTFSNAWMD), a CDR-H2 of SEQ ID NO:27 (QITAKSNNYATYYADSVKG), and a CDR-H3 of SEQ ID NO:28 (DGYH); and
      (b) a VH comprising a heavy chain complementary determining region CDR-H1 of SEQ ID NO:26 (GFTFSNAWMD), a CDR-H2 of SEQ ID NO:29 (QITAKSNNYATYYAAPVKG), and a CDR-H3 of SEQ ID NO:28 (DGYH); and
   (ii) a light chain variable region (V_{L} BCMA) comprising a light chain complementary determining region CDR-L1 of SEQ ID NO:30 (RASEDIRNGLA), a CDR-L2 of SEQ ID NO:31 (NANSLHT) and a CDR-L3 of SEQ ID NO:32 (EDTSKYPYT),
(b) a first and a second polypeptide that are linked to each other by a disulfide bond, wherein the antigen binding molecule is characterized in that the first polypeptide comprises two ectodomains of 4-1BBL each comprising the amino acid sequence of SEQ ID NO:9 or SEQ ID NO:10 that are connected to each other by a peptide linker, and in that the second polypeptide comprises one ectodomain of 4-1BBL comprising the amino acid sequence of SEQ ID NO:9 or SEQ ID NO:10, and
(c) an IgG Fc domain.

In one aspect, provided is the immunostimulatory antigen binding molecule as defined above, wherein the ectodomain of 4-1BBL or a fragment thereof comprises the amino acid sequence of SEQ ID NO:9 or SEQ ID NO:10. In one particular aspect, the ectodomain of 4-1BBL or a fragment thereof comprises the amino acid sequence of SEQ ID NO:9. In one aspect, the immunostimulatory antigen binding molecule comprises three ectodomains of 4-1BBL each comprising the amino acid sequence of SEQ ID NO:9 or SEQ ID NO:10, particularly the amino acid sequence of SEQ ID NO:9.

In one aspect, provided is the immunostimulatory antigen binding molecule as defined above, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10. In one particular aspect, the immunostimulatory antigen binding molecule is characterized in that the first polypeptide comprises the amino acid sequence of SEQ ID NO:11 and in that the second polypeptide comprises the amino acid sequence of SEQ ID NO:9.

In one aspect, provided is an immunostimulatory antigen binding molecule as defined herein before, wherein the antigen binding domain comprises
(a) a V_{H} BCMA comprising an amino acid sequence of SEQ ID NO:33 (VH2a) and V_{L} BCMA comprising an amino acid sequence of SEQ ID NO:34 (VL2a), or
(b) a V_{H} BCMA comprising an amino acid sequence of SEQ ID NO:35 (VH1b) and V_{L} BCMA comprising an amino acid sequence of SEQ ID NO:36 (VL1a).

In one particular aspect, the antigen binding domain comprises a V_{H} BCMA comprising an amino acid sequence of SEQ ID NO:33 (VH2a) and V_{L} BCMA comprising an amino acid sequence of SEQ ID NO:34 (VL2a).

In one aspect, the antibody that specifically binds to BCMA comprises an IgG Fc domain. In one aspect, the Fc domain is an IgG1 Fc domain. In one aspect, the Fc domain is a human Fc domain.

In one aspect, the IgG Fc domain comprises a modification promoting the association of the first and the second subunit of the Fc domain. In one aspect, the IgG Fc domain comprises knobs into hole modifications. In one aspect, the first subunit of the IgG Fc domain comprises the amino acid substitutions S354C and T366W (EU numbering) and the second subunit of the IgG Fc domain comprises the amino acid substitutions Y349C, T366S and Y407V (numbering according to Kabat EU index).

In one further aspect, the IgG Fc domain comprises one or more amino acid substitution that reduces binding to an Fc receptor and/or effector function. In one particular aspect, the IgG Fc domain is of human IgG1 subclass and comprises the amino acid mutations L234A, L235A and P329G (numbering according to Kabat EU index).

In one aspect, the immunostimulatory antigen binding molecule as described herein comprises
(a) a first polypeptide, comprising (ai) a first ectodomain of 4-1BBL comprising the amino acid sequence of SEQ ID NO:9 or SEQ ID NO:10, fused at its C-terminus to the N-terminus of the second ectodomain of 4-1BBL or fragment thereof, (aii) a second ectodomain of 4-1BBL comprising the amino acid sequence of SEQ ID NO:9 or SEQ ID NO:10, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL comprising the amino acid sequence of SEQ ID NO:9 or SEQ ID NO:10, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
   (d) a fourth polypeptide, comprising the light chain of the Fab molecule.

In one aspect, the immunostimulatory antigen binding molecule as described above is a molecule, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one particular aspect, the immunostimulatory antigen binding molecule as described herein comprises
(a) a first polypeptide comprising the amino acid sequence of SEQ ID NO:21, a second polypeptide comprising the amino acid sequence of SEQ ID NO:22, a third polypeptide comprising the amino acid sequence of SEQ ID NO:37 and a fourth polypeptide comprising the amino acid sequence of SEQ ID NO:38, or
(b) a first polypeptide comprising the amino acid sequence of SEQ ID NO:21, a second polypeptide comprising the amino acid sequence of SEQ ID NO:22, a third polypeptide comprising the amino acid sequence of SEQ ID NO:39 and a fourth polypeptide comprising the amino acid sequence of SEQ ID NO:40.

In one particular aspect, the immunostimulatory antigen binding molecule as described herein comprises a first polypeptide comprising the amino acid sequence of SEQ ID NO:21, a second polypeptide comprising the amino acid sequence of SEQ ID NO:22, a third polypeptide comprising the amino acid sequence of SEQ ID NO:37 and a fourth polypeptide comprising the amino acid sequence of SEQ ID NO:38.

In a further aspect, the invention relates to an antibody that specifically binds to BCMA, wherein the antibody comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16.

In yet another aspect, provided is an antibody that specifically binds to BCMA, wherein the antibody comprises
(a) a V_{H} BCMA comprising an amino acid sequence of SEQ ID NO:33 (VH2a) and V_{L} BCMA comprising an amino acid sequence of SEQ ID NO:34 (VL2a), or
(b) a V_{H} BCMA comprising an amino acid sequence of SEQ ID NO:35 (VH1b) and V_{L} BCMA comprising an amino acid sequence of SEQ ID NO:36 (VL1a).

According to another aspect of the invention, there is provided one or more isolated polynucleotide encoding the immunostimulatory antigen binding molecules or antibodies as described herein before. The invention further provides a vector, particularly an expression vector, comprising the isolated polynucleotide of the invention and a host cell comprising the isolated nucleic acid or the expression vector of the invention. In some aspects, the host cell is an eukaryotic cell, particularly a mammalian cell. In another aspect, provided is a method of producing an immunostimulatory antigen binding molecule that specifically binds to BCMA as described herein before or an antibody as described herein before, comprising the steps of a) culturing the host cell as described above under conditions suitable for the expression of the antigen binding molecule, and optionally b) recovering the antigen binding molecule. The invention also encompasses the immunostimulatory antigen binding molecule that specifically binds to BCMA as produced by the method of the invention.

Further provided is a pharmaceutical composition comprising an immunostimulatory antigen binding molecule that specifically binds to BCMA as described herein before and at least one pharmaceutically acceptable excipient. In one aspect, the pharmaceutical composition comprises an additional therapeutic agent.

Also encompassed is the immunostimulatory antigen binding molecule that specifically binds to BCMA as described herein before, or the pharmaceutical composition comprising the bispecific BCMA antibody, for use as a medicament.

In one aspect, provided is the immunostimulatory antigen binding molecule that specifically binds to BCMA as described herein before or the pharmaceutical composition, for use in enhancing (a) T cell activation or (b) T cell effector functions.

In one aspect, provided is the immunostimulatory antigen binding molecule that specifically binds to BCMA as described herein before or the pharmaceutical composition, for use in the treatment of a disease. In one aspect the disease is cancer, in particular multiple myeloma (MM).

In one specific aspect, provided is the immunostimulatory antigen binding molecule that specifically binds to BCMA as described herein before or the pharmaceutical composition, for use in the treatment of cancer, in particular multiple myeloma. In another specific aspect, provided is the immunostimulatory antigen binding molecule that specifically binds to BCMA as described herein before for use in the treatment of cancer, wherein the use is for administration in combination with a chemotherapeutic agent, radiation therapy and/or other agents for use in cancer immunotherapy. In one aspect, the immunostimulatory antigen binding molecule that specifically binds to BCMA as described herein before is for use in the treatment of cancer, wherein the use is for administration in combination with a T-cell activating anti-CD3 bispecific antibody. In one particular aspect, the T-cell activating anti-CD3 bispecific antibody is an anti-GPRC5D/anti-CD3 antibody.

In a further aspect, the invention provides a method of inhibiting the growth of tumor cells in an individual comprising administering to the individual an effective amount of the immunostimulatory antigen binding molecule that specifically binds to BCMA as described herein before, or the pharmaceutical composition of the invention, to inhibit the growth of the tumor cells. In another aspect, the invention provides a method of treating or delaying cancer in an individual comprising administering to the individual an effective amount of the immunostimulatory antigen binding molecule that specifically binds to BCMA as described herein before, or the pharmaceutical composition of the invention.

Also provided is the use of the immunostimulatory antigen binding molecule that specifically binds to BCMA as described herein before for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the immunostimulatory antigen binding molecule that specifically binds to BCMA of the invention in a pharmaceutically acceptable form. In a specific aspect, the disease is cancer. In any of the above aspects the individual is a mammal, particularly a human.

### BRIEF DESCRIPTION OF THE DRAWINGS

In FIG. 1A and FIG. 1B schematic illustrations of exemplary molecules as described herein are shown. FIG. 1A shows a schematic illustration of the BCMA antibody variants as monovalent hu IgG1 PGLALA isotype (“Fc silent”). FIG. 1B illustrates schematically the structure of the monovalent BCMA-targeting split trimeric 4-1BBL Fc (kih) P329G LALA fusion antigen binding molecule comprising CH1-CL cross in the arm wherein the two and one 4-1BBL are fused to, respectively. The thick black point stands for the knob-into-hole modification. Certain amino acids (so-called charged variants) are included in the CH1 and CL domain of the Fab molecule comprising the BCMA antigen binding domain to allow better pairing with the light chain.

FIG. 2A and FIG. 2B show the dose-dependent binding of various BCMA-4-1BBL antigen binding molecules and the comparator molecule P1AH1062 to cells expressing either human BCMA (FIG. 2A) or human 4-1BB (FIG. 2B). The untargeted 4-1BBL molecule only binds to 4-1BB expressing cells but not to BCMA expressing cells.

FIG. 3A and FIG. 3B show that all exemplified BCMA-4-1BBL antigen binding molecules have a similar binding behaviour towards BCMA-expressing CHO cells (CHO-huBCMA cells) as well as to human 4-1BB expressing Jurkat reporter cells, whereas the comparator molecule P1AH1062 did bind much less.

FIG. 3C to FIG. 3H show binding of two BCMA-4-1BBL antigen binding molecules P1AG7400 and P1AG7397 as well as the BCMA-targeted CD3 T cell engager Alnuctamab, to CHO cells expressing human BCMA with indicated point mutations. Shown is the binding in case no BCMA is present (FIG. 3C) and to human wt BCMA (FIG. 3D) as well as to BCMA variants human BCMA P33S (FIG. 3E), human BCMA P34del (FIG. 3F), human BCMA R27P (FIG. 3G) and human BCMA S30del (FIG. 3H). Another BCMA-targeted CD3 T cell engager Teclistamab does not bind to two mutated BCMA variants, namely R27P (FIG. 3G) and S30del (FIG. 3H) and Elranatamab only weakly binds to BCMA with the R27P mutation at high concentrations. Neither of the tested molecules bind to BCMA-negative CHOK1 cells (FIG. 3C).

FIG. 4A to FIG. 4C show the dose-dependent activation of NFκB in the Jurkat reporter assay when titrated amounts of (600.0-0.02 nM) of BCMA-4-1BBL antigen binding molecules were added and bridging target cells with effector cells. Obtained data are shown as mean±s.d. of one independent experiment performed in triplicates out of three. In FIG. 4A the concentration of BCMA-4-1BBL molecules or its controls is blotted against the units of released light (RLU), FIG. 4B and FIG. 4C provide a graphical summary of the EC₅₀ values and Emax values as calculated from three independent experiments performed in triplicate and shown together as mean±s.d.

FIG. 5A and FIG. 5B show that the dose-dependent activation of CD8+ T cells through BCMA-4-1BBL antigen binding molecules occurs only in presence of a first signal (TCB) and BCMA expression. The BCMA-expressing MM cell line NCI-H929 (FIG. 5A) was co-cultured with healthy donor PBMCs (ratio of 1:1). Alternatively, NCI-H929 BCMAko cells (knock-out cells that do not express BCMA) were used as target cells (FIG. 5B). Co-cultures were treated with GPRC5D-TCB (providing 1st signal) alone or in combination with BCMA-4-1BBL or an untargeted 4-1BBL (neg. ref.) which were titrated from 500 nM to 0.14 nM (1:3 dilution steps) and incubated for 4 days. Controls were left untreated or were treated with the BCMA-4-1BBL molecules alone (3 highest concentrations). Data are shown as mean of triplicates±s.d. of one donor (representative for 2-4 donors tested; BCMA ko N=1).

FIG. 5C to FIG. 5H show binding of two BCMA-4-1BBL antigen binding molecules P1AG7400 and P1AG7397 to CHO cells expressing human BCMA with indicated point mutations. Shown is the binding in case no BCMA is present (FIG. 5C) and to human wt BCMA (FIG. 5D) as well as to BCMA variants human BCMA R27P (FIG. 5E), human BCMA P34del (FIG. 5F), human BCMA P33S (FIG. 5G) and human BCMA S30del (FIG. 5H). Both molecules displayed a similar concentration-dependent boosting of T cell activation on top of the CD3 IgG-mediated T cell activation (as illustrated by the upregulation of CD25 on CD8 T cells) in presence of either the mutated or the wildtype BCMA expressed on CHO transfectants.

FIG. 5I to FIG. 5P show that BCMA-4-1BBL (P1AG7400) was able to boost T cell activation and tumor cell lysis on top of an anti-FcRH5/anti-CD3 bispecific antibody (FcRH5 x CD3). BCMA-4-1BBL was added at a fixed concentration to different concentrations of a FcRH5 x CD3 in a co-culture assay of healthy donor PBMCs in presence of BCMA-expressing MM cell lines. FIG. 5I, FIG. 5J, FIG. 5K and FIG. 5L show the increase in frequency of CD137-positive CD8 T cells in 4 donors, respectively, when BCMA-4-1BBL is added. Lysis of tumor cells is shown in FIG. 5M, FIG. 5N, FIG. 5O and FIG. 5P, respectively. T cell activation and tumor cell lysis were significantly boosted in the combination.

FIG. 6A to FIG. 6E show results of an exemplary ex vivo test, using primary MM patients' bone marrow samples. Tumor Cell Lysis (TCL) was determined by flow cytometry, defining maleimide positive cells with low FSC as dead cells (FIG. 6A). T-cell activation was determined by flow cytometry, assessing the upregulation of CD25 on CD8 (FIG. 6B) or CD4 (FIG. 6C) T cells upon incubation with 1 nM GPRC5D-TCB in absence or presence of the indicated concentrations of BCMA-4-1BBL (P1AG7422) or negative reference molecules for 96 hours. Degranulation was determined by flow cytometry, assessing the upregulation of CD107a on CD8 (FIG. 6D) or CD4 (FIG. 6E) T cells upon incubation with 1 nM GPRC5D-TCB in absence or presence of the indicated concentrations of BCMA-4-1BBL or negative reference molecules for 96 hours. All data shown refer to single tube measurement per condition.

FIG. 7A to FIG. 7E show results of another exemplary ex vivo test, using primary MM patients' bone marrow samples. Tumor Cell Lysis (TCL) was determined by flow cytometry, defining maleimide positive cells with low FSC as dead cells (FIG. 7A). T-cell activation was determined by flow cytometry, assessing the upregulation of CD25 on CD8 (FIG. 7B and FIG. 7C) or CD4 (FIG. 7D and FIG. 7E) T cells upon incubation with 1 nM GPRC5D-TCB in absence or presence of the indicated concentrations of BCMA-4-1BBL (P1AG7400 or P1AG7409) or negative reference molecules for 96 hours. All data shown refer to single tube measurement per condition.

FIG. 8A to FIG. 8D show results of an ex vivo test, using BM MNCs of primary MM patients' bone marrow samples. Tumor Cell lysis was determined by flow cytometry, assessing the percent of viable MM PCs upon incubation with 0.025 nM GPRC5D-TCB in absence or presence of 40 nM of BCMA-4-1BBL (P1AG7400) or reference molecules for 96 hours. All data shown refer to single tube measurement per condition.

FIG. 9A to FIG. 9H depict the results from an in vivo experiment testing the efficacy of GPRC5D x CD3 monotherapy and its combination with BCMA-4-1BBL antigen binding molecules (10 mg/kg and 3 mg/kg P1AG7409 and 10 mg/kg and 3 mg/kg P1AG7422) in the NCI-H929 tumor model. FIG. 9A to FIG. 9F show the tumor growth inhibition in single animals and FIG. 9G shows the tumor volume as Median (+/−IQR) per treatment group. Animals with a terminal tumor load below the size at treatment start were defined as responders. Tumor volume at termination (study day 41) is shown for GPRC5D x CD3 monotherapy group and combinations as Median (+/−IQR) is shown in FIG. 9H.

FIG. 10A to FIG. 10H show the results from an in vivo experiment testing the efficacy of GPRC5D x CD3 monotherapy and its combination with BCMA-4-1BBL antigen binding molecules P1AG7397 (10 and 20 mg/kg) and P1AG7400 (10 and 20 mg/kg) in the NCI-H929 tumor model. Tumor growth inhibition is shown in single animals (10A to FIG. 10F) and as Median (+/−IQR) per treatment group (FIG. 10G). Animals with a terminal tumor load below the size at treatment start were defined as responders. Tumor volume at termination (study day 57) is shown for GPRC5D x CD3 monotherapy and combination groups as Median (+/−IQR) (FIG. 10H).

FIG. 11A to FIG. 11F show the results of an in vivo experiment testing the efficacy of GPRC5D x CD3 monotherapy (0.05 mg/kg) and combination with BCMA-4-1BBL antigen binding molecule P1AG7400 (40, 4 and 0.4 mg/kg) in the NCI-H929 tumor model. 16A to 16E show the tumor growth inhibition in single animals. FIG. 11A shows the tumor volume as Median (+/−IQR) per treatment group. FIG. 11B to FIG. 11F show the tumor growth inhibition in single animals. Animals with a terminal tumor load below the size at treatment start were defined as responders.

FIG. 12 shows a comparison of the plasma concentration-time profiles of BCMA-4-1BBL antigen binding molecules P1AG7400, P1AG7397, P1AG7409 and P1AG7422 as measured in HuFcRN transgenic mice (PK study).

FIG. 13A to FIG. 13D show heatMAPPS representations of the results observed in the MAPPs assay (Example 6.3.1) for the bispecific antibodies P1AG7409 (FIG. 13A), P1AG7422 (FIG. 13B), P1AG7400 (FIG. 13C) and P1AG7397 (FIG. 13D). Clusters found in each domain are highlighted.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, multispecific antibodies (e.g., bispecific antibodies), antibody fragments and scaffold antigen binding proteins.

As used herein, the term “antigen binding domain that binds to a tumor-associated antigen” or “moiety capable of specific binding to a tumor-associated antigen” refers to a polypeptide molecule that specifically binds to the tumor-associated antigen BCMA. In one aspect, the antigen binding domain is able to activate signaling through BCMA. In a particular aspect, the antigen binding domain is able to direct the entity to which it is attached (e.g. the 4-1BBL trimer) to a BCMA-expressing cell, for example to a specific type of tumor cell. Antigen binding domains capable of specific binding to BCMA include antibodies and fragments thereof as further defined herein. In addition, antigen binding domains capable of specific binding to a tumor-associated antigen may include scaffold antigen binding proteins as further defined herein, e.g. binding domains which are based on designed repeat proteins or designed repeat domains (see e.g. WO 2002/020565).

In relation to an antigen binding molecule, i.e. an antibody or fragment thereof, the term “antigen binding domain” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH). In another aspect, the “antigen binding domain capable of specific binding to a tumor-associated antigen” can also be a Fab fragment or a crossFab fragment. As used herein, the terms “first”, “second” or “third” with respect to antigen binding domains etc., are used for convenience of distinguishing when there is more than one of each type of moiety. Use of these terms is not intended to confer a specific order or orientation of the moiety unless explicitly so stated.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. However, a bispecific antigen binding molecule may also comprise additional antigen binding sites which bind to further antigenic determinants. In certain aspects, the bispecific antigen binding molecule is capable of simultaneously binding two antigenic determinants, particularly two antigenic determinants expressed on two distinct cells or on the same cell. The term “bispecific” in accordance with the present invention thus may also include a trispecific molecule, e.g. a bispecific molecule comprising a 4-1BBL trimer and two antigen binding domains directed to two different target cell antigens.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (lgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, crossFab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′) 2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” or “Fab molecule” refers to an antibody fragment comprising a light chain fragment comprising a variable light chain (VL) domain and a constant domain of a light chain (CL), and a variable heavy chain (VH) domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments in which the cysteine residue(s) of the constant domains bear a free thiol group. Pepsin treatment yields an F(ab′) 2 fragment that has two antigen-combining sites (two Fab fragments) and a part of the Fc region. A “conventional Fab fragment” is comprised of a VL-CL light chain and a VH-CH1 heavy chain.

The term “crossFab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable (VL) domain and the heavy chain constant domain (CH1), and a peptide chain composed of the heavy chain variable domain (VH) and the light chain constant domain (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable domain (VH) and the light chain constant domain (CL), and a peptide chain composed of the light chain variable domain (VL) and the heavy chain constant domain (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), VNAR fragments, a fibronectin (AdNectin), a C-type lectin domain (Tetranectin); a variable domain of a new antigen receptor beta-lactamase (VNAR fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details, see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details, see Biochim Biophys Acta 1482:337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details, see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details, see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details, see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details, see J. Mol. Biol. 332, 489-503 (2003), PNAS 100 (4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the beta-sandwich can be engineered to enable an Adnectin to specifically recognize a therapeutic target of interest. For further details, see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details, see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can beengineered to include upto 25 amino acids without affecting the overall fold of the microprotein. For further details of engineered knottin domains, see WO2008098796.

An “antigen binding domain that binds to the same epitope” as a reference molecule refers to an antigen binding domain that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable domain (VL) and an antibody heavy chain variable domain (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

An “activating T cell antigen” as used herein refers to an antigenic determinant expressed on the surface of a T lymphocyte, particularly a cytotoxic T lymphocyte, which is capable of inducing T cell activation upon interaction with an antibody. Specifically, interaction of an antibody with an activating T cell antigen may induce T cell activation by triggering the signaling cascade of the T cell receptor complex. In a particular embodiment the activating T cell antigen is CD3, particularly the epsilon subunit of CD3 (see UniProt no. P07766 (version 189), NCBI RefSeq no. NP_000724.1, SEQ ID NO:167 for the human sequence; or UniProt no. Q95LI5 (version 49), NCBI GenBank no. BAB71849.1, SEQ ID NO:168 for the cynomolgus [Macaca fascicularis] sequence).

“T cell activation” as used herein refers to one or more cellular response of a T lymphocyte, particularly a cytotoxic T lymphocyte, selected from proliferation, differentiation, cytokine secretion, cytotoxic effector molecule release, cytotoxic activity, and expression of activation markers. Suitable assays to measure T cell activation are known in the art and described herein.

The term “T cell effector functions” refers to the activities of T cells that play a key role in the adaptive immune system. T cells are responsible for initiating and coordinating the body's immune response against foreign invaders, such as viruses or bacteria as well as tumor cells. Effector functions refer to the various activities carried out by T cells to eliminate these offenders, which include releasing cytokines, stimulating other cells, and directly attacking and eliminating infected cells.

A “tumor-associated antigen” or TAA as used herein refers to an antigenic determinant presented on the surface of a target cell, for example a cell in a tumor such as a cancer cell, a cell of the tumor stroma, a malignant B lymphocyte or a melanoma cell. In certain aspects, the target cell antigen is an antigen on the surface of a tumor cell. In one particular aspect, TAA is BCMA.

The term “BCMA” refers to B cell maturation antigen, also termed tumor necrosis factor receptor superfamily member 17 (TNFRS17) or CD269, and is a type III transmembrane protein without a signal-peptide and containing cysteine-rich extracellular domains. Ligands for BCMA include B cell activating factor (BAFF) and a proliferation-inducing ligand (APRIL), of which APRIL has a higher affinity for BCMA. BCMA is preferentially expressed by mature B lymphocytes, with minimal expression in hematopoietic stem cells or non-hematopoietic tissue, and is essential for the survival of long-lived bone marrow plasma cells. Membrane-bound BCMA can undergo gamma-secretase mediated shedding from the cell surface, leading to circulation of soluble BCMA (sBCMA) and reduced activation of surface BCMA by APRIL and BAFF. BCMA is overexpressed at significantly higher levels in all patient MM cells but not on other normal tissues except normal plasma cells. BCMA, along with two related TNFR superfamily B-cell activation factor receptor (BAFF-R) and transmembrane activator and calcium modulator and cyclophilin ligand interactor (TACI), critically regulate B cell proliferation and survival, as well as maturation and differentiation into plasma cells. These three functionally related receptors support long-term survival of B cells at different stages of development by binding to BAFF and/or APRIL, their cognate ligands. BCMA as used herein refers to any BCMA protein from any vertebrate source, including mammals such as primates (e.g. humans) non-human primates (e.g. cynomolgus monkeys) and rodents (e.g. mice and rats), unless otherwise indicated. The amino acid sequence of human BCMA is shown in UniProt (www.uniprot.org) accession no. Q02223 (SEQ ID NO:93).

The term “CD28” (Cluster of differentiation 28, Tp44) refers to any CD28 protein from any vertebrate source, including mammals such as primates (e.g. humans) non-human primates (e.g. cynomolgus monkeys) and rodents (e.g. mice and rats), unless otherwise indicated. CD28 is expressed on T cells and provides co-stimulatory signals required for T cell activation and survival. T cell stimulation through CD28 in addition to the T-cell receptor (TCR) can provide a potent signal for the production of various interleukins. CD28 is the receptor for CD80 (B7.1) and CD86 (B7.2) proteins and is the only B7 receptor constitutively expressed on naive T cells. The amino acid sequence of human CD28 is shown in UniProt (www.uniprot.org) accession no. P10747 (SEQ ID NO:94).

An “agonistic antibody” refers to an antibody that comprises an agonistic function against a given receptor. In general, when an agonist ligand (factor) binds to a receptor, the tertiary structure of the receptor protein changes, and the receptor is activated (when the receptor is a membrane protein, a cell growth signal or such is usually transducted). If the receptor is a dimer-forming type, an agonistic antibody can dimerize the receptor at an appropriate distance and angle, thus acting similarly to a ligand. An appropriate anti-receptor antibody can mimic dimerization of receptors performed by ligands, and thus can become an agonistic antibody.

A “CD28 agonistic antibody” or “CD28 conventional agonistic antibody” is an antibody that mimics CD28 natural ligands (CD80 or CD86) in their role to enhance T cell activation in presence of a T cell receptor signal (“signal 2”). A T cell needs two signals to become fully activated. Under physiological conditions “signal 1” arises from the interaction of T cell receptor (TCR) molecules with peptide/major histocompatibility complex (MHC) complexes on antigen presenting cells (APCs) and “signal 2” is provided by engagement of a costimulatory receptor, e.g. CD28. A CD28 agonistic antibody is able to costimulate T cells (signal 2). It is also able to induce T cell proliferation and cytokine secretion in combination with a molecule with specificity for the TCR complex, however the CD28 agonistic antibody is not capable of fully activating T cells without additional stimulation of the TCR. There is however a subclass of CD28 specific antigen binding molecules, the so-called CD28 superagonistic antibodies. A “CD28 superagonistic antibody” is a CD28 antibody which is capable of fully activating T cells without additional stimulation of the TCR. A CD28 superagonistic anitbody is capable to induce T cell proliferation and cytokine secretion without prior T cell activation (signal 1).

The term “variable domain” or “variable region” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antigen binding variable domain which are hypervariable in sequence and which determine antigen binding specificity, for example “complementarity determining regions” (“CDRs”). Generally, antigen binding domains comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature. Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Particularly, a “human” or “humanized” antibody comprises a constant region of human origin, particularly of the IgG isotype, more particularly of the IgG1 isotype, comprising a human CH1, CH2, CH3 and/or CL domain.

The term “CL domain” denotes the constant part of an antibody light chain polypeptide. Exemplary sequences of human constant domains are given in SEQ ID Nos: 95 and 96 (human kappa and lambda CL domains, respectively).

The term “CH1 domain” denotes the part of an antibody heavy chain polypeptide that extends approximately from EU position 118 to EU position 215 (EU numbering system according to Kabat). In one aspect, a CH1 domain has the amino acid sequence of ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKV (SEQ ID NO:97). Usually, a segment having the amino acid sequence of EPKSC (SEQ ID NO:98) is following to link the CH1 domain to the hinge region.

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083).

In one aspect, the hinge region has the amino acid sequence DKTHTCPXCP (SEQ ID NO: 99), wherein X is either S or P. In one aspect, the hinge region has the amino acid sequence HTCPXCP (SEQ ID NO: 100), wherein X is either S or P. In one aspect, the hinge region has the amino acid sequence CPXCP (SEQ ID NO:101), wherein X is either S or P.

The term “Fc domain” or “Fc region” herein is used to define a C-terminal region of an antibody heavy chain that contains at least a portion of the constant region. The term includes native sequence Fc regions and variant Fc regions. In the context of a molecule already defined by a Fab fragment (including the CH1 domain), the term “Fc domain” may only refer to an IgG CH2 and an IgG CH3 domain.

The “CH2 domain” of a human IgG Fc region usually extends from an amino acid residue at about EU position 231 to an amino acid residue at about EU position 340 (EU numbering system according to Kabat). In one aspect, a CH2 domain has the amino acid sequence of APELLGGPSV FLFPPKPKDT LMISRTPEVT CVWDVSHEDP EVKENWYVDG VEVHNAKTKP REEQESTYRW SVLTVLHQDW LNGKEYKCKV SNKALPAPIE KTISKAK (SEQ ID NO:102). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one embodiment, a carbohydrate chain is attached to the CH2 domain. The CH2 domain herein may be a native sequence CH2 domain or variant CH2 domain.

The “CH3 domain” comprises the stretch of residues C-terminal to a CH2 domain in an Fc region denotes the part of an antibody heavy chain polypeptide that extends approximately from EU position 341 to EU position 446 (EU numbering system according to Kabat). In one aspect, the CH3 domain has the amino acid sequence of GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTOKS LSLSPGK (SEQ ID NO:103). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “wild-type Fc domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. Wild-type Fc-regions are denoted in SEQ ID NO: 104 (IgG1, caucasian allotype), SEQ ID NO: 105 (IgG1, afroamerican allotype), SEQ ID NO: 106 (IgG2), SEQ ID NO: 107 (IgG3) and SEQ ID NO:108 (IgG4).

The term “variant (human) Fc domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region. A specific variant Fc domain disclosed herein is the human IgG1 heavy chain constant region with mutations L234A, L235A and P329G comprising the amino acid sequence of SEQ ID NO:109.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is an immune mechanism leading to lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831). For example, the capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (SEQ ID NO:110, UniProt accession no. P08637, version 141).

An “ectodomain” is the domain of a membrane protein that extends into the extracellular space (i.e. the space outside the target cell). Ectodomains are usually the parts of proteins that initiate contact with surfaces, which leads to signal transduction.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 5, typically between 2 and 4, in particular 2, i.e. the peptides selected from the group consisting of GGGGS (SEQ ID NO:111), GGGGSGGGG (SEQ ID NO:112), GGGGSGGGGS (SEQ ID NO:113), SGGGGSGGGG (SEQ ID NO:114) and GGGGSGGGGSGGGG (SEQ ID NO:115), but also include the sequences GSPGSSSSGS (SEQ ID NO:116), (G4S)₃ (SEQ ID NO:117), (G4S)₄ (SEQ ID NO:118), GSGSGSGS (SEQ ID NO:119), GSGSGNGS (SEQ ID NO:120), GGSGSGSG (SEQ ID NO:121), GGSGSG (SEQ ID NO:122), GGSG (SEQ ID NO:123), GGSGNGSG (SEQ ID NO:124), GGNGSGSG (SEQ ID NO:125) and GGNGSG (SEQ ID NO:126). Peptide linkers of particular interest are (G4S) (SEQ ID NO:111), (G4S)₂, GGGGSGGGG (SEQ ID NO:112) or GGGGSGGGGS (SEQ ID NO:113), (G4S)₃ (SEQ ID NO:117), and (G4S)₄ (SEQ ID NO:118).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the BCMA antibodies or immunostimulatory antigen binding molecules provided herein are contemplated. For example, it may be desirable to improve the binding affinity and/or other biological properties of the BCMA antibodies or immunostimulatory antigen binding molecules. Amino acid sequence variants of the BCMA antibodies or immunostimulatory antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antigen binding molecule. Any combination of deletion, insertion, and substitution can be made to arrive at the final construct, provided that the final construct possesses the desired characteristics, e.g., antigen-binding. Sites of interest for substitutional mutagenesis include the CDRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more CDRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of insertions include immunostimulatory antigen binding molecules with a fusion to the N- or C-terminus to a polypeptide which increases the serum half-life of the immunostimulatory antigen binding molecule.

In certain aspects, the immunostimulatory antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the agonistic ICOS-binding molecule comprises an Fc domain, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in BCMA antibodies or bispecific BCMA antibodies may be made in order to create variants with certain improved properties. In one aspect, variants of BCMA antibodies or bispecific BCMA antibodies are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the BCMA antibodies or bispecific BCMA antibodies include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the immunostimulatory antigen binding molecules described herein, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the immunostimulatory antigen binding molecule to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the immunostimulatory antigen binding molecules provided herein may be further modified to contain additional non-proteinaceous moieties that are known in the art and readily available. The moieties suitable for derivatization of the antigen binding molecule include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one aspect, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed. In another aspect, immunoconjugates of the BCMA antibodies or bispecific BCMA antibodies provided herein may be obtained. An “immunoconjugate” is an antigen binding molecule conjugated to one or more heterologous molecule(s), for instance a small molecule agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al. (2017) Nature Medicine 23:815-817, or EP 2 101 823 B1).

By “isolated” nucleic acid molecule or polynucleotide is meant a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts, as well as positive and negative strand forms, and double-stranded forms. Isolated polynucleotides or nucleic acids according to the present disclosure further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette as disclosed herein comprises polynucleotide sequences that encode the immunostimulatory antigen binding molecule or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector as disclosed herein comprises an expression cassette that comprises polynucleotide sequences that encode an immunostimulatory antigen binding molecule or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER. C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “therapy” refers to any protocol, method and/or agent that can be used in the prevention, management, treatment and/or amelioration of a disease (or symptom related thereto) or cancer. In certain aspects, the terms “therapies” and “therapy” refer to a biological therapy, supportive therapy, and/or other therapies useful in the prevention, management, treatment and/or amelioration of a disease or cancer known to one of skill in the art such as a physician.

The term “combination treatment” or “co-administration” as noted herein encompasses combined administration (where two or more therapeutic agents are included in the same or separate formulations), and separate administration, in which case, administration of an antibody as reported herein can occur prior to, simultaneously, and/or following, administration of the additional therapeutic agent or agents, preferably an antibody or antibodies.

The term “cancer” refers to or describes the physiological condition in mammals that is typically characterized by unregulated cell growth/proliferation. Thus, the term cancer as used herein refers to proliferative diseases, such as carcinoma, lymphomas (e.g., Hodgkin's and non-Hodgkin's lymphoma), blastoma, sarcoma, and leukemia. In particular, the term cancer includes lymphocytic leukemias, lung cancer, non-small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers. In one aspect, the cancer is a solid tumor. In another aspect, the cancer is a haematological cancer, particularly leukemia, most particularly acute lymphoblastic leukemia (ALL) or acute myelogenous leukemia (AML). In a preferred aspect, the term cancer refers to any cancer in which BCMA is expressed. More preferably, the cancer is multiple myeloma (MM).

### Exemplary New BCMA Antibodies

Provided herein are novel antibodies and/or antibody fragments that specifically bind to B cell maturation antigen (BCMA). Provided are novel antibodies and/or antibody fragments that specifically bind to the extracellular domain of human BCMA comprising the amino acid sequence of SEQ ID NO:127. Thus, these antibodies specifically bind to human BCMA.

These antibodies are able to bind to human BCMA and to cynomolgus BCMA. Thus, they also bind to the extracellular domain of cynomolgus BCMA comprising the amino acid sequence of SEQ ID NO:129.

They bind to the human BCMA extracellular domain (ECD of the amino acid sequence of SEQ ID NO:127) with an K_{D} value of less than 5 nM as measured by surface plasmon resonance (SPR) (see Example 1.3).

As is demonstrated in Example 3, the new antibodies are also able to bind to mutated human BCMA variants hu BCMA_R27P (SEQ ID NO:203), Hu BCMA_S30del (SEQ ID NO:204), hu BCMA_P33S (SEQ ID NO:205) and hu BCMA_P34del (SEQ ID NO:206). They are therefore not affected by point mutations in the ECD of BCMA and do not lose the therapeutic activity as has been observed with other BCMA-targeted molecules such as Elranatamab and Teclistamab ((Lee et al., Nature Medicine 2023, 29, 2295-2306).

The new antibodies are further characterized in that they are producable in high amounts and with high titers, that they show high thermal stability (as measured by the aggregation temperature T_{agg}), or in that they possess a high degree of humanness and may therefore be less immunogenic in the human body. The percentage of humanness of the VH and VL sequences as compared to the human germline sequences can be determined by the methods described in Abhinandan, K. R. and Martin, Andrew C. R. 2007, J. Mol. Biol. 2007, 369, 852-862.

In one aspect, provided herein is an a antibody that specifically binds to B cell maturation agent (BCMA), wherein the antibody comprises
(i) a heavy chain variable region (V_{H} BCMA) comprising a heavy chain complementary determining region CDR-H1 of SEQ ID NO:1 (GYTFTNYWMH), a CDR-H2 of SEQ ID NO:2 (IIHPNSGSTNYNEKFQG), and a CDR-H3 of SEQ ID NO:3 (GIYDYPFAY), and
(ii) a light chain variable region (V_{L} BCMA) selected from the group consisting of
   (a) a VL comprising a light chain complementary determining region CDR-L1 of SEQ ID NO:4 (RASESVSIHGTHLMH), a CDR-L2 of SEQ ID NO:5 (AASSLQS) and a CDR-L3 of SEQ ID NO:6 (QQSIEDPYT); or
   (b) a VL comprising a light chain complementary determining region CDR-L1 of SEQ ID NO:4 (RASESVSIHGTHLMH), a CDR-L2 of SEQ ID NO:7 (AASNLES) and a CDR-L3 of SEQ ID NO:6 (QQSIEDPYT), or
   (c) a VL comprising a light chain complementary determining region CDR-L1 of SEQ ID NO:4 (RASESVSIHGTHLMH), a CDR-L2 of SEQ ID NO:8 (AASNLQS) and a CDR-L3 of SEQ ID NO:6 (QQSIEDPYT).

In one aspect, provided is a BCMA antibody (or an antigen binding domain that specifically binds to BCMA), wherein said antibody comprises a V_{H} BCMA comprising an amino acid sequence selected from the group consisting of SEQ ID NO:13 (VH1a) and SEQ ID NO:14 (VH1b), and/or a V_{L} BCMA comprising an amino acid sequence selected from the group consisting of SEQ ID NO:15 (VL1f), SEQ ID NO:16 (VL1a), SEQ ID NO:17 (VL1b), SEQ ID NO:18 (VL1c), SEQ ID NO:19 (VL1d), and SEQ ID NO:20 (VL1e).

In one aspect, provided is a BCMA antibody (or an antigen binding domain that specifically binds to BCMA), wherein said antibody (or antigen binding domain) comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16; or
(c) a V_{H} BCMA comprising an amino acid sequence of SEQ ID NO:13 and V_{L} BCMA comprising an amino acid sequence of SEQ ID NO:17, or
(d) a V_{H} BCMA comprising an amino acid sequence of SEQ ID NO:13 and V_{L} BCMA comprising an amino acid sequence of SEQ ID NO:18; or
(e) a V_{H} BCMA comprising an amino acid sequence of SEQ ID NO:13 and V_{L} BCMA comprising an amino acid sequence of SEQ ID NO:19, or
(f) a V_{H} BCMA comprising an amino acid sequence of SEQ ID NO:13 and V_{L} BCMA comprising an amino acid sequence of SEQ ID NO:20; or
(g) a V_{H} BCMA comprising an amino acid sequence of SEQ ID NO:13 and V_{L} BCMA comprising an amino acid sequence of SEQ ID NO:130, or
(h) a V_{H} BCMA comprising an amino acid sequence of SEQ ID NO:13 and V_{L} BCMA comprising an amino acid sequence of SEQ ID NO:131; or
(i) a V_{H} BCMA comprising an amino acid sequence of SEQ ID NO:14 and V_{L} BCMA comprising an amino acid sequence of SEQ ID NO:15, or
(j) a V_{H} BCMA comprising an amino acid sequence of SEQ ID NO:14 and V_{L} BCMA comprising an amino acid sequence of SEQ ID NO:16; or
(k) a V_{H} BCMA comprising an amino acid sequence of SEQ ID NO:14 and V_{L} BCMA comprising an amino acid sequence of SEQ ID NO:17, or
(l) a V_{H} BCMA comprising an amino acid sequence of SEQ ID NO:14 and V_{L} BCMA comprising an amino acid sequence of SEQ ID NO:18; or
(m) a V_{H} BCMA comprising an amino acid sequence of SEQ ID NO:14 and V_{L} BCMA comprising an amino acid sequence of SEQ ID NO:19, or
(n) a V_{H} BCMA comprising an amino acid sequence of SEQ ID NO:14 and V_{L} BCMA comprising an amino acid sequence of SEQ ID NO:20; or
(o) a V_{H} BCMA comprising an amino acid sequence of SEQ ID NO:14 and V_{L} BCMA comprising an amino acid sequence of SEQ ID NO:130, or
(p) a V_{H} BCMA comprising an amino acid sequence of SEQ ID NO:14 and V_{L} BCMA comprising an amino acid sequence of SEQ ID NO:131.

In one aspect, the BCMA antibody (or the antigen binding domain that specifically binds to BCMA) comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16; or
(c) a V_{H} BCMA comprising an amino acid sequence of SEQ ID NO:13 and V_{L} BCMA comprising an amino acid sequence of SEQ ID NO:17, or
(d) a V_{H} BCMA comprising an amino acid sequence of SEQ ID NO:13 and V_{L} BCMA comprising an amino acid sequence of SEQ ID NO:18; or
(e) a V_{H} BCMA comprising an amino acid sequence of SEQ ID NO:13 and V_{L} BCMA comprising an amino acid sequence of SEQ ID NO:19, or
(f) a V_{H} BCMA comprising an amino acid sequence of SEQ ID NO:13 and V_{L} BCMA comprising an amino acid sequence of SEQ ID NO:20; or
(g) a V_{H} BCMA comprising an amino acid sequence of SEQ ID NO:13 and V_{L} BCMA comprising an amino acid sequence of SEQ ID NO:130, or
(h) a V_{H} BCMA comprising an amino acid sequence of SEQ ID NO:13 and V_{L} BCMA comprising an amino acid sequence of SEQ ID NO:131.

In one aspect, the BCMA antibody (or the antigen binding domain that specifically binds to BCMA) comprises
(i) a V_{H} BCMA comprising an amino acid sequence of SEQ ID NO:14 and V_{L} BCMA comprising an amino acid sequence of SEQ ID NO:15, or
(j) a V_{H} BCMA comprising an amino acid sequence of SEQ ID NO:14 and V_{L} BCMA comprising an amino acid sequence of SEQ ID NO:16; or
(k) a V_{H} BCMA comprising an amino acid sequence of SEQ ID NO:14 and V_{L} BCMA comprising an amino acid sequence of SEQ ID NO:17, or
(l) a V_{H} BCMA comprising an amino acid sequence of SEQ ID NO:14 and V_{L} BCMA comprising an amino acid sequence of SEQ ID NO:18; or
(m) a V_{H} BCMA comprising an amino acid sequence of SEQ ID NO:14 and V_{L} BCMA comprising an amino acid sequence of SEQ ID NO:19, or
(n) a V_{H} BCMA comprising an amino acid sequence of SEQ ID NO:14 and V_{L} BCMA comprising an amino acid sequence of SEQ ID NO:20; or
(o) a V_{H} BCMA comprising an amino acid sequence of SEQ ID NO:14 and V_{L} BCMA comprising an amino acid sequence of SEQ ID NO:130, or
(p) a V_{H} BCMA comprising an amino acid sequence of SEQ ID NO:14 and V_{L} BCMA comprising an amino acid sequence of SEQ ID NO:131.

In one particular aspect, provided is a BCMA antibody (or an antigen binding domain that specifically binds to BCMA), wherein said antibody (or antigen binding domain) comprises
(a) a V_{H} BCMA comprising an amino acid sequence of SEQ ID NO:13 and a V_{L} BCMA comprising an amino acid sequence of SEQ ID NO:15, or
(b) a V_{H} BCMA comprising an amino acid sequence of SEQ ID NO:13 and a V_{L} BCMA comprising an amino acid sequence of SEQ ID NO:16.

More particularly, the BCMA antibody (or the antigen binding domain that specifically binds to BCMA) comprises a V_{H} BCMA comprising an amino acid sequence of SEQ ID NO:13 and a V_{L} BCMA comprising an amino acid sequence of SEQ ID NO:15.

In another aspect, provided herein is an a antibody that specifically binds to B cell maturation agent (BCMA), wherein the antibody comprises
(i) a heavy chain variable region (V_{H} BCMA) selected from the group consisting of
   (a) a VH comprising a heavy chain complementary determining region CDR-H1 of SEQ ID NO:26 (GFTFSNAWMD), a CDR-H2 of SEQ ID NO:27 (QITAKSNNYATYYADSVKG), and a CDR-H3 of SEQ ID NO:28 (DGYH), and
   (b) a VH comprising a heavy chain complementary determining region CDR-H1 of SEQ ID NO:26 (GFTFSNAWMD), a CDR-H2 of SEQ ID NO:29 (QITAKSNNYATYYAAPVKG), and a CDR-H3 of SEQ ID NO:29 (DGYH), and
(ii) a light chain variable region (V_{L} BCMA) comprising a light chain complementary determining region CDR-L1 of SEQ ID NO:30 (RASEDIRNGLA), a CDR-L2 of SEQ ID NO:31 (NANSLHT) and a CDR-L3 of SEQ ID NO:32 (EDTSKYPYT).

In one aspect, provided is a BCMA antibody (or an antigen binding domain that specifically binds to BCMA), wherein said antibody (or antigen binding domain) comprises a heavy chain variable region (V_{H} BCMA) comprising a CDR-H1 of SEQ ID NO:26 (GFTFSNAWMD), a CDR-H2 of SEQ ID NO:27 (QITAKSNNYATYYADSVKG), and a CDR-H3 of SEQ ID NO:28 (DGYH), and a light chain variable region (V_{L} BCMA) comprising a CDR-L1 of SEQ ID NO:30 (RASEDIRNGLA), a CDR-L2 of SEQ ID NO:31 (NANSLHT) and a CDR-L3 of SEQ ID NO:32 (EDTSKYPYT).

In one aspect, provided is a BCMA antibody (or an antigen binding domain that specifically binds to BCMA), wherein said antibody (or antigen binding domain) comprises a heavy chain variable region (V_{H} BCMA) comprising a CDR-H1 of SEQ ID NO:26 (GFTFSNAWMD), a CDR-H2 of SEQ ID NO:29 (QITAKSNNYATYYAAPVKG), and a CDR-H3 of SEQ ID NO:28 (DGYH), and a light chain variable region (V_{L} BCMA) comprising a CDR-L1 of SEQ ID NO:30 (RASEDIRNGLA), a CDR-L2 of SEQ ID NO:31 (NANSLHT) and a CDR-L3 of SEQ ID NO:32 (EDTSKYPYT).

In one aspect, provided is a BCMA antibody (or an antigen binding domain that specifically binds to BCMA), wherein said antibody (or antigen binding domain) comprises a V_{H} BCMA comprising an amino acid sequence selected from the group consisting of SEQ ID NO:33 (VH2a), SEQ ID NO:35 (VH1b), SEQ ID NO:53 (VH1a), SEQ ID NO:54 (VH1a_Y292D), SEQ ID NO:55 (VHlc_hu CDR2) and SEQ ID NO:56 (VH1a_W197Y), and/or the V_{L} BCMA comprising an amino acid sequence selected from the group consisting of SEQ ID NO:34 (VL2a), SEQ ID NO:36 (VL1a), SEQ ID NO:57 (VL1a_L2_GL), SEQ ID NO:58 (VL2a_L2_GL), SEQ ID NO:59 (VL1a_L1_pGL), SEQ ID NO:60 (VL1a_N651A), SEQ ID NO:61 (VL1a_N651A_N695S), SEQ ID NO:62 (VL1a_H698Q), SEQ ID NO:63 (VL1a_T699S) and SEQ ID NO:64 (VL1a_H698Q_T699S).

In one aspect, provided is a BCMA antibody (or an antigen binding domain that specifically binds to BCMA), wherein said antibody (or antigen binding domain) comprises a V_{H} BCMA comprising an amino acid sequence selected from the group consisting of SEQ ID NO:33 (VH2a) and SEQ ID NO:35 (VH1b), and/or the V_{L} BCMA comprising an amino acid sequence selected from the group consisting of SEQ ID NO:34 (VL2a) and SEQ ID NO:36 (VL1a).

In one aspect, provided is a BCMA antibody (or an antigen binding domain that specifically binds to BCMA), wherein said antibody (or antigen binding domain) comprises
(a) a V_{H} BCMA comprising an amino acid sequence of SEQ ID NO:33 and a V_{L} BCMA comprising an amino acid sequence of SEQ ID NO:34, or
(b) a V_{H} BCMA comprising an amino acid sequence of SEQ ID NO:35 and a V_{L} BCMA comprising an amino acid sequence of SEQ ID NO:36; or
(c) a V_{H} BCMA comprising an amino acid sequence of SEQ ID NO:53 and V_{L} BCMA comprising an amino acid sequence of SEQ ID NO:57, or
(d) a V_{H} BCMA comprising an amino acid sequence of SEQ ID NO:54 and V_{L} BCMA comprising an amino acid sequence of SEQ ID NO:36; or
(e) a V_{H} BCMA comprising an amino acid sequence of SEQ ID NO:54 and V_{L} BCMA comprising an amino acid sequence of SEQ ID NO:57, or
(f) a V_{H} BCMA comprising an amino acid sequence of SEQ ID NO:54 and V_{L} BCMA comprising an amino acid sequence of SEQ ID NO:63; or
(g) a V_{H} BCMA comprising an amino acid sequence of SEQ ID NO:54 and V_{L} BCMA comprising an amino acid sequence of SEQ ID NO:34, or
(h) a V_{H} BCMA comprising an amino acid sequence of SEQ ID NO:35 and V_{L} BCMA comprising an amino acid sequence of SEQ ID NO:59; or
(i) a V_{H} BCMA comprising an amino acid sequence of SEQ ID NO:35 and V_{L} BCMA comprising an amino acid sequence of SEQ ID NO:60, or
(j) a V_{H} BCMA comprising an amino acid sequence of SEQ ID NO:35 and V_{L} BCMA comprising an amino acid sequence of SEQ ID NO:61; or
(k) a V_{H} BCMA comprising an amino acid sequence of SEQ ID NO:35 and V_{L} BCMA comprising an amino acid sequence of SEQ ID NO:63, or
(l) a V_{H} BCMA comprising an amino acid sequence of SEQ ID NO:35 and V_{L} BCMA comprising an amino acid sequence of SEQ ID NO:34; or
(m) a V_{H} BCMA comprising an amino acid sequence of SEQ ID NO:33 and V_{L} BCMA comprising an amino acid sequence of SEQ ID NO:36, or
(n) a V_{H} BCMA comprising an amino acid sequence of SEQ ID NO:33 and V_{L} BCMA comprising an amino acid sequence of SEQ ID NO:59; or
(o) a V_{H} BCMA comprising an amino acid sequence of SEQ ID NO:33 and V_{L} BCMA comprising an amino acid sequence of SEQ ID NO:63.

In one particular aspect, provided is a BCMA antibody (or an antigen binding domain that specifically binds to BCMA), wherein said antibody (or antigen binding domain) comprises
(a) a V_{H} BCMA comprising an amino acid sequence of SEQ ID NO:33 and a V_{L} BCMA comprising an amino acid sequence of SEQ ID NO:34, or
(b) a V_{H} BCMA comprising an amino acid sequence of SEQ ID NO:35 and a V_{L} BCMA comprising an amino acid sequence of SEQ ID NO:36.

More particularly, the BCMA antibody comprises a V_{H} BCMA comprising an amino acid sequence of SEQ ID NO:33 and a V_{L} BCMA comprising an amino acid sequence of SEQ ID NO:34.

In one aspect, the antibody that specifically binds to BCMA is a full-length antibody, in particular of human IgG1 subclass. In one particular aspect, it comprises the amino acid mutations L234A, L235A and P329G (numbering according to Kabat EU index).

### Immunostimulatory Antigen Binding Molecules Comprising the New BCMA Antibodies

The patent application provides new BCMA targeting 4-1BBL antigen binding molecules that comprise three 4-1 BB ligands or fragments thereof that are fused to the antigen binding molecule whereby two of the 4-1 BB ligands or fragments thereof are fused to each other via a linker and one 4-1BB ligand is present on a different chain of the molecule (split 4-1BBL trimer). These BCMA targeting 4-1BBL antigen binding molecules possess advantageous properties such as excellent producibility, stability, binding affinity, biological activity, targeting efficiency, reduced toxicity, an extended dosage range that can be given to a patient and thereby a possibly an enhanced efficacy can be observed. The new BCMA targeting 4-1BBL antigen binding molecules comprise an Fc domain composed of a first and a second subunit capable of stable association comprising one or more amino acid substitution that reduces the binding affinity of the antigen binding molecule to an Fc receptor and/or effector function (Fc silent) and thus unspecific cross-linking via Fc receptors is avoided. Instead, they comprise specific antigen binding domain capable of specific binding to BCMA which causes cross-linking at the tumor site. Surprisingly, the inventors have found that based on their binding properties the BCMA antigen binding domains as described herein have advantageous properties that makes them more usable in this format as other BCMA antigen binding domains. This can be seen for instance in Jurkat NFκB/4-1BB Reporter cell assay (Example 4.1) wherein a 4-1BBL antigen binding molecule comprising BCMA antibody PR was less able to induce T cell activation compared to the 4-1BBL antigen binding molecules comprising the new BCMA antibodies described herein. It has been found that the a 4-1BBL antigen binding molecules comprising these BCMA antigen binding domains possess an improved functionality and ability to increase T cell activation, particularly in the presence of T-cell activating anti-CD3 bispecific antibodies. Thus, an enhanced tumor-specific T cell activation is achieved.

In one aspect, provided herein is an immunostimulatory antigen binding molecule that specifically binds to B cell maturation agent (BCMA), comprising
a) an antigen binding domain comprising
   (i) a heavy chain variable region (V_{H} BCMA) comprising a heavy chain complementary determining region CDR-H1 of SEQ ID NO:1 (GYTFTNYWMH), a CDR-H2 of SEQ ID NO:2 (IIHPNSGSTNYNEKFQG), and a CDR-H3 of SEQ ID NO:3 (GIYDYPFAY), and
   (ii) a light chain variable region (V_{L} BCMA) selected from the group consisting of
      (a) a VL comprising a light chain complementary determining region CDR-L1 of SEQ ID NO:4 (RASESVSIHGTHLMH), a CDR-L2 of SEQ ID NO:5 (AASSLQS) and a CDR-L3 of SEQ ID NO:6 (QQSIEDPYT); and
      (b) a VL comprising a light chain complementary determining region CDR-L1 of SEQ ID NO:4 (RASESVSIHGTHLMH), a CDR-L2 of SEQ ID NO:7 (AASNLES) and a CDR-L3 of SEQ ID NO:6 (QQSIEDPYT), and
      (c) a VL comprising a light chain complementary determining region CDR-L1 of SEQ ID NO:4 (RASESVSIHGTHLMH), a CDR-L2 of SEQ ID NO:8 (AASNLQS) and a CDR-L3 of SEQ ID NO:6 (QQSIEDPYT),
(b) a first and a second polypeptide that are linked to each other by a disulfide bond, wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) an Fc domain composed of a first and a second subunit.

In one aspect, provided is an immunostimulatory antigen binding molecule as defined above, wherein the ectodomain of 4-1BBL or a fragment thereof comprises the amino acid sequence of SEQ ID NO:9 or SEQ ID NO:10. In one particular aspect, the ectodomain of 4-1BBL or a fragment thereof comprises the amino acid sequence of SEQ ID NO:9. In one aspect, the immunostimulatory antigen binding molecule comprises three ectodomains of 4-1BBL each comprising the amino acid sequence of SEQ ID NO:9 or SEQ ID NO:10, particularly the amino acid sequence of SEQ ID NO:9.

In one aspect, provided is an immunostimulatory antigen binding molecule as defined above, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10. In one particular aspect, the immunostimulatory antigen binding molecule is characterized in that the first polypeptide comprises the amino acid sequence of SEQ ID NO:11 and in that the second polypeptide comprises the amino acid sequence of SEQ ID NO:9.

In one aspect, the peptide linker that connects the ectodomains of 4-1BBL or a fragment thereof with each other comprises the amino acid sequence of SEQ ID NO:112 or SEQ ID NO:113. In one particular aspect, the peptide linker comprises the amino acid sequence of SEQ ID NO:112.

In one aspect, provided is an immunostimulatory antigen binding molecule as defined above, wherein said antigen binding molecule comprises a V_{H} BCMA comprising an amino acid sequence selected from the group consisting of SEQ ID NO:13 (VH1a) and SEQ ID NO: 14 (VH1b), and/or a V_{L} BCMA comprising an amino acid sequence selected from the group consisting of SEQ ID NO:15 (VL1f), SEQ ID NO:16 (VL1a), SEQ ID NO:17 (VL1b), SEQ ID NO:18 (VL1c), SEQ ID NO:19 (VL1d), and SEQ ID NO:20 (VL1e).

In one aspect, provided is an immunostimulatory antigen binding molecule as defined above, wherein said antigen binding molecule comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16; or
(c) a V_{H} BCMA comprising an amino acid sequence of SEQ ID NO:13 and V_{L} BCMA comprising an amino acid sequence of SEQ ID NO:17, or
(d) a V_{H} BCMA comprising an amino acid sequence of SEQ ID NO:13 and V_{L} BCMA comprising an amino acid sequence of SEQ ID NO:18; or
(e) a V_{H} BCMA comprising an amino acid sequence of SEQ ID NO:13 and V_{L} BCMA comprising an amino acid sequence of SEQ ID NO:19, or
(f) a V_{H} BCMA comprising an amino acid sequence of SEQ ID NO:13 and V_{L} BCMA comprising an amino acid sequence of SEQ ID NO:20; or
(g) a V_{H} BCMA comprising an amino acid sequence of SEQ ID NO:13 and V_{L} BCMA comprising an amino acid sequence of SEQ ID NO:130, or
(h) a V_{H} BCMA comprising an amino acid sequence of SEQ ID NO:13 and V_{L} BCMA comprising an amino acid sequence of SEQ ID NO:131; or
(i) a V_{H} BCMA comprising an amino acid sequence of SEQ ID NO:14 and V_{L} BCMA comprising an amino acid sequence of SEQ ID NO:15, or
(j) a V_{H} BCMA comprising an amino acid sequence of SEQ ID NO:14 and V_{L} BCMA comprising an amino acid sequence of SEQ ID NO:16; or
(k) a V_{H} BCMA comprising an amino acid sequence of SEQ ID NO:14 and V_{L} BCMA comprising an amino acid sequence of SEQ ID NO:17, or
(l) a V_{H} BCMA comprising an amino acid sequence of SEQ ID NO:14 and V_{L} BCMA comprising an amino acid sequence of SEQ ID NO:18; or
(m) a V_{H} BCMA comprising an amino acid sequence of SEQ ID NO:14 and V_{L} BCMA comprising an amino acid sequence of SEQ ID NO:19, or
(n) a V_{H} BCMA comprising an amino acid sequence of SEQ ID NO:14 and V_{L} BCMA comprising an amino acid sequence of SEQ ID NO:20; or
(o) a V_{H} BCMA comprising an amino acid sequence of SEQ ID NO:14 and V_{L} BCMA comprising an amino acid sequence of SEQ ID NO:130, or
(p) a V_{H} BCMA comprising an amino acid sequence of SEQ ID NO:14 and V_{L} BCMA comprising an amino acid sequence of SEQ ID NO:131.

In one aspect, the immunostimulatory antigen binding molecule as defined above comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16; or
(c) a V_{H} BCMA comprising an amino acid sequence of SEQ ID NO:13 and V_{L} BCMA comprising an amino acid sequence of SEQ ID NO:17, or
(d) a V_{H} BCMA comprising an amino acid sequence of SEQ ID NO:13 and V_{L} BCMA comprising an amino acid sequence of SEQ ID NO:18; or
(e) a V_{H} BCMA comprising an amino acid sequence of SEQ ID NO:13 and V_{L} BCMA comprising an amino acid sequence of SEQ ID NO:19, or
(f) a V_{H} BCMA comprising an amino acid sequence of SEQ ID NO:13 and V_{L} BCMA comprising an amino acid sequence of SEQ ID NO:20; or
(g) a V_{H} BCMA comprising an amino acid sequence of SEQ ID NO:13 and V_{L} BCMA comprising an amino acid sequence of SEQ ID NO:130, or
(h) a V_{H} BCMA comprising an amino acid sequence of SEQ ID NO:13 and V_{L} BCMA comprising an amino acid sequence of SEQ ID NO:131.

In one aspect, the immunostimulatory antigen binding molecule as defined above comprises
(i) a V_{H} BCMA comprising an amino acid sequence of SEQ ID NO:14 and V_{L} BCMA comprising an amino acid sequence of SEQ ID NO:15, or
(j) a V_{H} BCMA comprising an amino acid sequence of SEQ ID NO:14 and V_{L} BCMA comprising an amino acid sequence of SEQ ID NO:16; or
(k) a V_{H} BCMA comprising an amino acid sequence of SEQ ID NO:14 and V_{L} BCMA comprising an amino acid sequence of SEQ ID NO:17, or
(l) a V_{H} BCMA comprising an amino acid sequence of SEQ ID NO:14 and V_{L} BCMA comprising an amino acid sequence of SEQ ID NO:18; or
(m) a V_{H} BCMA comprising an amino acid sequence of SEQ ID NO:14 and V_{L} BCMA comprising an amino acid sequence of SEQ ID NO:19, or
(n) a V_{H} BCMA comprising an amino acid sequence of SEQ ID NO:14 and V_{L} BCMA comprising an amino acid sequence of SEQ ID NO:20; or
(o) a V_{H} BCMA comprising an amino acid sequence of SEQ ID NO:14 and V_{L} BCMA comprising an amino acid sequence of SEQ ID NO:130, or
(p) a V_{H} BCMA comprising an amino acid sequence of SEQ ID NO:14 and V_{L} BCMA comprising an amino acid sequence of SEQ ID NO:131.

In one particular aspect, provided is an immunostimulatory antigen binding molecule as defined above, wherein said antigen binding molecule comprises
(a) a V_{H} BCMA comprising an amino acid sequence of SEQ ID NO:13 and a V_{L} BCMA comprising an amino acid sequence of SEQ ID NO:15, or
(b) a V_{H} BCMA comprising an amino acid sequence of SEQ ID NO:13 and a V_{L} BCMA comprising an amino acid sequence of SEQ ID NO:16.

More particularly, the immunostimulatory antigen binding molecule comprises a V_{H} BCMA comprising an amino acid sequence of SEQ ID NO:13 and a V_{L} BCMA comprising an amino acid sequence of SEQ ID NO:15.

In one particular aspect, the antigen binding domain is a Fab molecule.

In one aspect, the immunostimulatory antigen binding molecule as defined above comprises an Fc domain composed of a first and a second subunit. In one aspect, the Fc domain composed of a first and a second subunit is an IgG, particularly an IgG1 Fc domain. In one aspect, the Fc domain is a human Fc domain.

In one aspect, the Fc domain comprises a modification promoting the association of the first and the second subunit of the Fc domain. In one aspect, the Fc domain comprises knobs into hole modifications. In one aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In one aspect, the immunostimulatory antigen binding molecule as described herein comprises
(a) a first polypeptide, comprising (ai) a first ectodomain of 4-1BBL or fragment thereof, fused at its C-terminus to the N-terminus of the second ectodomain of 4-1BBL or fragment thereof, (aii) a second ectodomain of 4-1BBL or fragment thereof, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL or fragment thereof, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
   (d) a fourth polypeptide, comprising the light chain of the Fab molecule.

In one aspect, immunostimulatory antigen binding molecule as described above is a molecule, wherein n the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, the immunostimulatory antigen binding molecule as described herein comprises a polypeptide that is at least 95%, 96%, 97%, 98%, or 99% identical to the sequence of SEQ ID NO:21, a peptide that is at least 95%, 96%, 97%, 98%, or 99% identical to the sequence of SEQ ID NO:22, a polypeptide that is at least 95%, 96%, 97%, 98%, or 99% identical to the sequence of SEQ ID NO:23, and a polypeptide that is at least 95%, 96%, 97%, 98%, or 99% identical to the sequence of SEQ ID NO:24. In a further aspect, the immunostimulatory antigen binding molecule as described herein comprises a polypeptide that is at least 95%, 96%, 97%, 98%, or 99% identical to the sequence of SEQ ID NO:21, a polypeptide that is at least 95%, 96%, 97%, 98%, or 99% identical to the sequence of SEQ ID NO:22, a polypeptide that is at least 95%, 96%, 97%, 98%, or 99% identical to the sequence of SEQ ID NO:23, and a polypeptide that is at least 95%, 96%, 97%, 98%, or 99% identical to the sequence of SEQ ID NO:25.

In one particular aspect, the immunostimulatory antigen binding molecule as described herein comprises
(a) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:24, or
(b) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:25.

In one particular aspect, the immunostimulatory antigen binding molecule comprises a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:24. In one aspect, the immunostimulatory antigen binding molecule comprises (a) a first polypeptide comprising the amino acid sequence of SEQ ID NO:208, a second polypeptide comprising the amino acid sequence of SEQ ID NO:22, a third polypeptide comprising the amino acid sequence of SEQ ID NO:207 and a fourth polypeptide comprising the amino acid sequence of SEQ ID NO:24. In one particular aspect, the immunostimulatory antigen binding molecule consists of (a) a first polypeptide comprising the amino acid sequence of SEQ ID NO:208, a second polypeptide comprising the amino acid sequence of SEQ ID NO:22, a third polypeptide comprising the amino acid sequence of SEQ ID NO:207 and a fourth polypeptide comprising the amino acid sequence of SEQ ID NO:24.

Provided herein is also an immunostimulatory antigen binding molecule that specifically binds to B cell maturation agent (BCMA), comprising
(a) an antigen binding domain comprising
   (i) a heavy chain variable region (V_{L} BCMA) selected from the group consisting of
      (a) a VH comprising a heavy chain complementary determining region CDR-H1 of SEQ ID NO:26 (GFTFSNAWMD), a CDR-H2 of SEQ ID NO:27 (QITAKSNNYATYYADSVKG), and a CDR-H3 of SEQ ID NO:28 (DGYH); and
      (b) a VH comprising a heavy chain complementary determining region CDR-H1 of SEQ ID NO:26 (GFTFSNAWMD), a CDR-H2 of SEQ ID NO:29 (QITAKSNNYATYYAAPVKG), and a CDR-H3 of SEQ ID NO:28 (DGYH); and
   (ii) a light chain variable region (V_{L} BCMA) comprising a light chain complementary determining region CDR-L1 of SEQ ID NO:30 (RASEDIRNGLA), a CDR-L2 of SEQ ID NO:31 (NANSLHT) and a CDR-L3 of SEQ ID NO:32 (EDTSKYPYT),
(b) a first and a second polypeptide that are linked to each other by a disulfide bond, wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) an Fc domain composed of a first and a second subunit.

In one aspect, provided is the immunostimulatory antigen binding molecule as defined above, wherein the ectodomain of 4-1BBL or a fragment thereof comprises the amino acid sequence of SEQ ID NO:9 or SEQ ID NO:10. In one particular aspect, the ectodomain of 4-1BBL or a fragment thereof comprises the amino acid sequence of SEQ ID NO:9. In one aspect, the immunostimulatory antigen binding molecule comprises three ectodomains of 4-1BBL each comprising the amino acid sequence of SEQ ID NO:9 or SEQ ID NO:10, particularly the amino acid sequence of SEQ ID NO:9.

In one aspect, provided is the immunostimulatory antigen binding molecule as defined above, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10. In one particular aspect, the immunostimulatory antigen binding molecule is characterized in that the first polypeptide comprises the amino acid sequence of SEQ ID NO:11 and in that the second polypeptide comprises the amino acid sequence of SEQ ID NO:9.

In one aspect, the peptide linker that connects the ectodomains of 4-1BBL or a fragment thereof with each other comprises the amino acid sequence of SEQ ID NO:112 or SEQ ID NO:113. In one particular aspect, the peptide linker comprises the amino acid sequence of SEQ ID NO:112.

In one aspect, provided is an immunostimulatory antigen binding molecule as defined herein before, wherein said antigen binding molecule comprises a V_{H} BCMA comprising an amino acid sequence selected from the group consisting of SEQ ID NO:33 (VH2a), SEQ ID NO:35 (VH1b), SEQ ID NO:53 (VH1a), SEQ ID NO:54 (VH1a_Y292D), SEQ ID NO:55 (VH1c_hu CDR2) and SEQ ID NO:56 (VH1a_W197Y), and/or the V_{L} BCMA comprising an amino acid sequence selected from the group consisting of SEQ ID NO:34 (VL2a), SEQ ID NO:36 (VL1a), SEQ ID NO:57 (VL1a_L2_GL), SEQ ID NO:58 (VL2a_L2_GL), SEQ ID NO:59 (VL1a_L1_pGL), SEQ ID NO:60 (VL1a_N651A), SEQ ID NO:61 (VL1a_N651A_N695S), SEQ ID NO:62 (VL1a_H698Q), SEQ ID NO:63 (VL1a_T699S) and SEQ ID NO:64 (VL1a_H698Q_T699S).

In one aspect, provided is an immunostimulatory antigen binding molecule as described above, wherein said antigen binding molecule comprises a V_{H} BCMA comprising an amino acid sequence selected from the group consisting of SEQ ID NO:33 (VH2a) and SEQ ID NO: 35 (VH1b), and/or the V_{L} BCMA comprising an amino acid sequence selected from the group consisting of SEQ ID NO:34 (VL2a) and SEQ ID NO:36 (VL1a).

In one aspect, provided is an immunostimulatory antigen binding molecule, wherein said antigen binding molecule comprises
(a) a V_{H} BCMA comprising an amino acid sequence of SEQ ID NO:33 and a V_{L} BCMA comprising an amino acid sequence of SEQ ID NO:34, or
(b) a V_{H} BCMA comprising an amino acid sequence of SEQ ID NO:35 and a V_{L} BCMA comprising an amino acid sequence of SEQ ID NO:36; or
(c) a V_{H} BCMA comprising an amino acid sequence of SEQ ID NO:53 and V_{L} BCMA comprising an amino acid sequence of SEQ ID NO:57, or
(d) a V_{H} BCMA comprising an amino acid sequence of SEQ ID NO:54 and V_{L} BCMA comprising an amino acid sequence of SEQ ID NO:36; or
(e) a V_{H} BCMA comprising an amino acid sequence of SEQ ID NO:54 and V_{L} BCMA comprising an amino acid sequence of SEQ ID NO:57, or
(f) a V_{H} BCMA comprising an amino acid sequence of SEQ ID NO:54 and V_{L} BCMA comprising an amino acid sequence of SEQ ID NO:63; or
(g) a V_{H} BCMA comprising an amino acid sequence of SEQ ID NO:54 and V_{L} BCMA comprising an amino acid sequence of SEQ ID NO:34, or
(h) a V_{H} BCMA comprising an amino acid sequence of SEQ ID NO:35 and V_{L} BCMA comprising an amino acid sequence of SEQ ID NO:59; or
(i) a V_{H} BCMA comprising an amino acid sequence of SEQ ID NO:35 and V_{L} BCMA comprising an amino acid sequence of SEQ ID NO:60, or
(j) a V_{H} BCMA comprising an amino acid sequence of SEQ ID NO:35 and V_{L} BCMA comprising an amino acid sequence of SEQ ID NO:61; or
(k) a V_{H} BCMA comprising an amino acid sequence of SEQ ID NO:35 and V_{L} BCMA comprising an amino acid sequence of SEQ ID NO:63, or
(l) a V_{H} BCMA comprising an amino acid sequence of SEQ ID NO:35 and V_{L} BCMA comprising an amino acid sequence of SEQ ID NO:34; or
(m) a V_{H} BCMA comprising an amino acid sequence of SEQ ID NO:33 and V_{L} BCMA comprising an amino acid sequence of SEQ ID NO:36, or
(n) a V_{H} BCMA comprising an amino acid sequence of SEQ ID NO:33 and V_{L} BCMA comprising an amino acid sequence of SEQ ID NO:59; or
(o) a V_{H} BCMA comprising an amino acid sequence of SEQ ID NO:33 and V_{L} BCMA comprising an amino acid sequence of SEQ ID NO:63.

In one particular aspect, provided is an immunostimulatory antigen binding molecule as described above, wherein said antigen binding molecule comprises
(a) a V_{H} BCMA comprising an amino acid sequence of SEQ ID NO:33 and a V_{L} BCMA comprising an amino acid sequence of SEQ ID NO:34, or
(b) a V_{H} BCMA comprising an amino acid sequence of SEQ ID NO:35 and a V_{L} BCMA comprising an amino acid sequence of SEQ ID NO:36.

More particularly, the immunostimulatory antigen binding molecule comprises a VH BCMA comprising an amino acid sequence of SEQ ID NO:33 and a V_{L} BCMA comprising an amino acid sequence of SEQ ID NO:34.

In one particular aspect, the antigen binding domain is a Fab molecule.

In one aspect, the antigen binding molecule that specifically binds to BCMA comprises an Fc domain composed of a first and a second subunit. In one aspect, the Fc domain composed of a first and a second subunit is an IgG, particularly an IgG1 Fc domain. In one aspect, the Fc domain is a human Fc domain.

In one aspect, the Fc domain comprises a modification promoting the association of the first and the second subunit of the Fc domain. In one aspect, the Fc domain comprises knobs into hole modifications. In one aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In one aspect, the immunostimulatory antigen binding molecule as described herein comprises
(a) a first polypeptide, comprising (ai) a first ectodomain of 4-1BBL or fragment thereof, fused at its C-terminus to the N-terminus of the second ectodomain of 4-1BBL or fragment thereof, (aii) a second ectodomain of 4-1BBL or fragment thereof, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL or fragment thereof, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
   (d) a fourth polypeptide, comprising the light chain of the Fab molecule.

In one aspect, immunostimulatory antigen binding molecule as described above is a molecule, wherein the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, the immunostimulatory antigen binding molecule as described herein comprises a polypeptide that is at least 95%, 96%, 97%, 98%, or 99% identical to the sequence of SEQ ID NO: 21, a polypeptide that is at least 95%, 96%, 97%, 98%, or 99% identical to the sequence of SEQ ID NO: 22, a polypeptide that is at least 95%, 96%, 97%, 98%, or 99% identical to the sequence of SEQ ID NO: 37, and a polypeptide that is at least 95%, 96%, 97%, 98%, or 99% identical to the sequence of SEQ ID NO: 38. In a further aspect, the immunostimulatory antigen binding molecule as described herein comprises a polypeptide that is at least 95%, 96%, 97%, 98%, or 99% identical to the sequence of SEQ ID NO: 21, a polypeptide that is at least 95%, 96%, 97%, 98%, or 99% identical to the sequence of SEQ ID NO: 22, a polypeptide that is at least 95%, 96%, 97%, 98%, or 99% identical to the sequence of SEQ ID NO: 39, and a polypeptide that is at least 95%, 96%, 97%, 98%, or 99% identical to the sequence of SEQ ID NO: 40.

In one particular aspect, the immunostimulatory antigen binding molecule as described herein comprises
(a) a first polypeptide comprising the amino acid sequence of SEQ ID NO: 21, a second polypeptide comprising the amino acid sequence of SEQ ID NO:22, a third polypeptide comprising the amino acid sequence of SEQ ID NO:37 and a fourth polypeptide comprising the amino acid sequence of SEQ ID NO:38, or
(b) a first polypeptide comprising the amino acid sequence of SEQ ID NO: 21, a second polypeptide comprising the amino acid sequence of SEQ ID NO:22, a third polypeptide comprising the amino acid sequence of SEQ ID NO:39 and a fourth polypeptide comprising the amino acid sequence of SEQ ID NO:40.

In one particular aspect, the immunostimulatory antigen binding molecule as described herein comprises a first polypeptide comprising the amino acid sequence of SEQ ID NO: 21, a second polypeptide comprising the amino acid sequence of SEQ ID NO:22, a third polypeptide comprising the amino acid sequence of SEQ ID NO:37 and a fourth polypeptide comprising the amino acid sequence of SEQ ID NO:38.

In any of the aspects mentioned above, the immunostimulatory antigen binding molecule comprises a Fc domain. In one aspect, the Fc domain is an IgG, particularly an IgG1 Fc domain. In one particular aspect, the Fc domain is a human Fc domain, particularly a human IgG1 Fc domain. In any of the aspects described above, the Fc domain comprises one or more amino acid substitution that reduces binding to an Fc receptor and/or effector function. In one particular aspect, it comprises the amino acid mutations L234A, L235A and P329G (numbering according to Kabat EU index).

Fc Domain Modifications Reducing Fc Receptor Binding and/or Effector Function

The Fc domain of the immunostimulatory antigen binding molecule described herein consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. The Fc domain confers favorable pharmacokinetic properties to the antigen binding molecules of the invention, including a long serum half-life which contributes to good accumulation in the target tissue and a favorable tissue-blood distribution ratio. On the other side, it may, however, lead to undesirable targeting of the antigen binding molecules to cells expressing Fc receptors rather than to the preferred antigen-bearing cells.

Accordingly, the Fc domain of the immunostimulatory antigen binding molecule that binds to BCMA exhibits reduced binding affinity to an Fc receptor and/or reduced effector function, as compared to a native IgG1 Fc domain. In one aspect, the Fc domain does not substantially bind to an Fc receptor and/or does not induce effector function. In a particular aspect, the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of an antigen binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In one particular aspect, provided is an immunostimulatory antigen binding molecule that specifically binds to BCMA, wherein the Fc region comprises one or more amino acid substitution that reduces binding to an Fc receptor, in particular towards Fcγ receptor. In one aspect, provided is an immunostimulatory antigen binding molecule that specifically binds to BCMA, wherein the Fc region comprises one or more amino acid substitution and wherein the ADCC induced by the antigen binding molecule is reduced to 0-20% of the ADCC induced by an antibody comprising the wild-type human IgG1 Fc region.

In one aspect, the Fc domain of the antigen binding molecule described herein comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is an immunostimulatory antigen binding molecule that specifically binds to BCMA which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or cell activating antibodies comprising an Fc domain for Fc receptors may be evaluated using cell lines known to express particular Fc receptors, such as human NK cells expressing FcγIIIa receptor.

Effector function of an Fc domain, or antigen binding molecules as described herein comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

In some aspects, binding of the Fc domain to a complement component, specifically to C1q, is reduced. Accordingly, in some aspects wherein the Fc domain is engineered to have reduced effector function, said reduced effector function includes reduced CDC. C1q binding assays may be carried out to determine whether the bispecific antibodies of the invention are able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

In one particular aspect, the Fc domain exhibiting reduced binding affinity to an Fc receptor and/or reduced effector function, as compared to a native IgG1 Fc domain, is a human IgG1 Fc domain comprising the amino acid substitutions L234A, L235A and optionally P329G, or a human IgG4 Fc domain comprising the amino acid substitutions S228P, L235E and optionally P329G (numberings according to Kabat EU index). More particularly, it is a human IgG1 Fc domain comprising the amino acid substitutions L234A, L235A and P329G (numbering according to Kabat EU index).

### Fc Domain Modifications Promoting Heterodimerization

The immunostimulatory antigen binding molecule that specifically binds to BCMA comprises different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antigen binding molecules disclosed herein in recombinant production, it will thus be advantageous to introduce in the Fc domain of the antibody described herein a modification promoting the association of the desired polypeptides.

Accordingly, in particular aspects, provided is an immunostimulatory antigen binding molecule that specifically binds to BCMA comprising a Fc domain composed of a first and a second subunit capable of stable association, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, provided is an immunostimulatory antigen binding molecule that specifically binds to BCMA, wherein the antibody comprises a Fc domain composed of a first and a second subunit capable of stable association comprising one or more amino acid substitution that reduces the binding affinity of the antigen binding molecule to an Fc receptor and/or effector function, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the immunostimulatory antigen binding molecule that specifically binds to BCMA an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending P. In one preferred aspect, the C-terminus of the heavy chain is a shortened C-terminus ending PG. In one aspect of all aspects as reported herein, an antigen binding molecule comprising a heavy chain including a C-terminal CH3 domain as specified herein, comprises the C-terminal glycine-lysine dipeptide (G446 and K447, numbering according to Kabat EU index). In one aspect of all aspects as reported herein, an antigen binding molecule comprising a heavy chain including a C-terminal CH3 domain, as specified herein, comprises a C-terminal glycine residue (G446, numbering according to Kabat EU index).

### Modifications in the Fab Domains

In one aspect, provided is an immunostimulatory antigen binding molecule that specifically binds to BCMA characterized by monovalent binding to BCMA comprising (a) a first antigen binding domain capable of specific binding to BCMA, (b) a first and a second polypeptide that are linked to each other by a disulfide bond, wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and (c) a Fc domain composed of a first and a second subunit capable of stable association comprising one or more amino acid substitution that reduces the binding affinity of the antigen binding molecule to an Fc receptor and/or effector function, wherein the two ectodomains of 4-1BBL and the one ectodomain of 4-1BBL are fused, optionally via a peptide linker, to constant domains CL and CH1, and these are exchanged against each other, so that CH1 is part of the shorter polypeptide (“light chain”) and CL is part of the longer polypeptide (“heavy chain”). The constant domains CH1 and CL are exchanged according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In another aspect, and to further improve correct pairing, the immunostimulatory antigen binding molecule that specifically binds to BCMA contains different charged amino acid substitutions (so-called “charged residues”). These modifications are introduced in the crossed or non-crossed CH1 and CL domains. In a particular aspect, the invention relates to an immunostimulatory antigen binding molecule that specifically binds to BCMA, wherein in the CL domain of the Fab molecule that binds to BCMA the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E). In one particular aspect, in the CL domain of the Fab molecule capable of specific binding to BCMA the amino acid at position 123 (EU numbering) has been replaced by arginine (R) and the amino acid at position 124 (EU numbering) has been substituted by lysine (K) and in the CH1 domain of the Fab molecule capable of specific binding to BCMA the amino acids at position 147 (EU numbering) and at position 213 (EU numbering) have been substituted by glutamic acid (E).

### Polynucleotides

The invention further provides isolated polynucleotides encoding a BCMA antibody or an immunostimulatory antigen binding molecule that specifically binds to BCMA as described herein. The one or more isolated polynucleotides encoding the BCMA antibody or the immunostimulatory antigen binding molecule that specifically binds to BCMA may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin. In some aspects, the isolated polynucleotide encodes the entire immunostimulatory antigen binding molecule that specifically binds to BCMA as described herein. In other aspects, the isolated polynucleotide encodes a polypeptide comprised in the immunostimulatory antigen binding molecule that specifically binds to BCMA as described herein. In certain aspects the polynucleotide or nucleic acid is DNA. In other aspects, a polynucleotide of the present invention is RNA, for example, in the form of messenger RNA (mRNA). RNA of the present invention may be single stranded or double stranded.

### Recombinant Methods

BCMA antibodies or immunostimulatory antigen binding molecules that specifically binds to BCMA as described herein may be obtained, for example, by solid-state peptide synthesis (e.g. Merrifield solid phase synthesis) or recombinant production. For recombinant production one or more polynucleotide encoding the antigen binding molecule that specifically binds to BCMA or polypeptide fragments thereof, e.g., as described above, is isolated and inserted into one or more vectors for further cloning and/or expression in a host cell. Such polynucleotide may be readily isolated and sequenced using conventional procedures. In one aspect, a vector, preferably an expression vector, comprising one or more of the polynucleotides as described herein is provided. Methods which are well known to those skilled in the art can be used to construct expression vectors containing the coding sequence of the antibody (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the antibody or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antibody of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions as described herein may be associated with additional coding regions which encode secretory or signal peptides, which direct the secretion of a polypeptide encoded by a polynucleotide of the present invention. For example, if secretion of the antibody or polypeptide fragments thereof is desired, DNA encoding a signal sequence may be placed upstream of the nucleic acid encoding an antibody as described herein or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain aspects, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase. DNA encoding a short protein sequence that could be used to facilitate later purification (e.g. a histidine tag) or assist in labeling the antibody as described herein may be included within or at the ends of the polynucleotide encoding the antibody as described herein or polypeptide fragments thereof.

In a further aspect, a host cell comprising one or more polynucleotides as described herein is provided. In certain aspects, a host cell comprising one or more vectors as described herein is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) an antibody as disclosed herein. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antibody for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing an immunostimulatory antigen binding molecule that specifically binds to BCMA or polypeptide fragments thereof is provided, wherein the method comprises culturing a host cell comprising polynucleotides encoding the antibody or polypeptide fragments thereof, as provided herein, under conditions suitable for expression of the antibody or polypeptide fragments thereof, and recovering the antibody as described herein or polypeptide fragments thereof from the host cell (or host cell culture medium).

In certain aspects the antigen binding domain capable of specific binding to BCMA (e.g. a Fab fragment) forming part of the immunostimulatory antigen binding molecule that specifically binds to BCMA comprises at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used for the methods described herein. Non-limiting immunoglobulins useful can be of murine, primate, or human origin. If the antibody is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31 (3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, NJ, 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the antigen binding domains comprised in the antigen binding molecules described herein are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antibodies of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain aspects, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, NJ). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch. 14 (Cold Spring Harbor Laboratory, Cold Spring Harbor, NY).

Antigen binding molecules that specifically bind to BCMA prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the antigen binding molecule binds. For example, for affinity chromatography purification of antigen binding molecules of the invention, a matrix with protein A or protein G may be used. Sequential Protein A or G affinity chromatography and size exclusion chromatography can be used to isolate an antigen binding molecule essentially as described in the Examples. The purity of the antibody or fragments thereof can be determined by any of a variety of well-known analytical methods including gel electrophoresis, high pressure liquid chromatography, and the like. For example, the bispecific antibody expressed as described in the Examples was shown to be intact and properly assembled as demonstrated by reducing and non-reducing SDS-PAGE.

### Assays

The BCMA antibody or the immunostimulatory antigen binding molecule that specifically binds to BCMA provided herein may be identified, screened for, or characterized for its physical/chemical properties and/or biological activities by various assays known in the art.

### 1. Affinity Assays

The affinity of the antigen binding molecule provided herein for the corresponding target can be determined in accordance with the methods set forth in the Examples by surface plasmon resonance (SPR), using standard instrumentation such as a Proteon instrument (Bio-rad), and receptors or target proteins such as may be obtained by recombinant expression. The affinity of the antigen binding molecule for the target cell antigen can also be determined by surface plasmon resonance (SPR), using standard instrumentation such as a Proteon instrument (Bio-rad), and receptors or target proteins such as may be obtained by recombinant expression. According to one aspect, K_{D} is measured by surface plasmon resonance using a Proteon® machine (Bio-Rad) at 25° C.

### 2. Binding Assays and Other Assays

Binding of the antibody or the immunostimulatory antigen binding molecule that specifically binds to BCMA provided herein to the corresponding receptor expressing cells may be evaluated using cell lines expressing the particular receptor or target antigen, for example by flow cytometry (FACS). In one aspect, CHO-K1 cell lines expressing BCMA extracellular domain and non-truncating mutants thereof are used in the binding assay.

In a further aspect, Jurkat-NFκB-luc2_4-1BB cells were used to demonstrate the binding of the antigen binding molecules to 4-1BBL.

### 3. Activity Assays

In one aspect, assays are provided for identifying the biological activity of the BCMA-4-1BBL antigen binding molecule. Biological activity may include, e.g. T cell proliferation and cytokine secretion as measured with the methods as described in Example 4. Antigen binding molecules having such biological activity in vivo and/or in vitro are also provided.

### Pharmaceutical Compositions, Formulations and Routes of Administration

In a further aspect, the invention provides pharmaceutical compositions comprising any of the immunostimulatory antigen binding molecules that specifically bind to BCMA provided herein, e.g., for use in any of the below therapeutic methods. In one aspect, a pharmaceutical composition comprises an immunostimulatory antigen binding molecule that specifically binds to BCMA provided herein and at least one pharmaceutically acceptable excipient. In another aspect, a pharmaceutical composition comprises an immunostimulatory antigen binding molecule that specifically binds to BCMA provided herein and at least one additional therapeutic agent, e.g., as described below.

Pharmaceutical compositions as disclosed herein comprise a therapeutically effective amount of one or more antigen binding molecules dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one immunostimulatory antigen binding molecule that specifically binds to BCMA and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the 4-1BBL trimer-containing antigen binding molecules may be formulated in aqueous solutions, preferably in physiologically compatible buffers such as Hanks' solution, Ringer's solution, or physiological saline buffer. The solution may contain formulation agents such as suspending, stabilizing and/or dispersing agents. Alternatively, the immunostimulatory antigen binding molecule that specifically binds to BCMA may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof. Exemplary pharmaceutically acceptable excipients herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. In addition to the compositions described previously, the immunostimulatory antigen binding molecule that specifically binds to BCMA may also be formulated as a depot preparation. Such long acting formulations may be administered by implantation (for example subcutaneously or intramuscularly) or by intramuscular injection. Thus, for example, the immunostimulatory antigen binding molecule that specifically binds to BCMA may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the immunostimulatory antigen binding molecule that specifically binds to BCMA may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen. The immunostimulatory antigen binding molecule that specifically binds to BCMA may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms. The composition herein may also contain more than one active ingredient as necessary for the particular indication being treated, preferably those with complementary activities that do not adversely affect each other. Such active ingredients are suitably present in combination in amounts that are effective for the purpose intended. The formulations to be used for in vivo administration are generally sterile. Sterility may be readily accomplished, e.g., by filtration through sterile filtration membranes.

### Therapeutic Methods and Compositions

Any of the immunostimulatory antigen binding molecule that specifically binds to BCMA provided herein may be used in therapeutic methods, either alone or in combination.

In one aspect, an immunostimulatory antigen binding molecule that specifically binds to BCMA for use as a medicament is provided. In further aspects, an antigen binding molecule that specifically binds to BCMA and 4-1BB for use in treating cancer is provided. In one particular aspect, an immunostimulatory antigen binding molecule that specifically binds to BCMA for use in treating hematologic malignancies is provided. The term “hematologic malignancies” includes diseases selected from the group consisting of multiple myeloma (MM), chronic lymphocytic leukemia, acute B-lymphoblastic leukemia, non-Hodgkin lymphoma (NHL), and Hodgkin lymphoma, but also acute myeloid leukemia and acute lymphoblastic leukemia. In one particular aspect, an antigen binding molecule that specifically binds to BCMA and 4-1BB for use in treating multiple myeloma (MM) is provided.

In certain aspects, an immunostimulatory antigen binding molecule that specifically binds to BCMA for use in a method of treatment is provided. In certain aspects, herein is provided an immunostimulatory antigen binding molecule that specifically binds to BCMA for use in a method of treating an individual having cancer comprising administering to the individual an effective amount of the immunostimulatory antigen binding molecule that specifically binds to BCMA. In one such aspect, the method further comprises administering to the individual an effective amount of at least one additional therapeutic agent.

In one aspect, the immunostimulatory antigen binding molecule that specifically binds to BCMA as described herein is for use in inhibiting the growth of BCMA-expressing cancer cells.

In certain aspects, an immunostimulatory antigen binding molecule that specifically binds to BCMA for use in a method of treatment is provided. In certain aspects, herein is provided an immunostimulatory antigen binding molecule that specifically binds to BCMA for use in a method of treating an individual having cancer comprising administering to the individual an effective amount of the immunostimulatory antigen binding molecule that specifically binds to BCMA. In another aspect, provided is an immunostimulatory antigen binding molecule that specifically binds to BCMA for use in a method of treating an individual having BCMA-expressing cancer, in particular hematologic malignancies selected from the group consisting of multiple myeloma (MM), chronic lymphocytic leukemia, acute B-lymphoblastic leukemia, non-Hodgkin lymphoma (NHL), Hodgkin lymphoma, acute myeloid leukemia and acute lymphoblastic leukemia, comprising administering to the individual an effective amount of the immunostimulatory antigen binding molecule that specifically binds to BCMA. In one such aspect, the method further comprises administering to the individual an effective amount of at least one additional therapeutic agent.

In a further aspect, herein is provided for the use of an immunostimulatory antigen binding molecule that specifically binds to BCMA as described herein in the manufacture or preparation of a medicament. In one embodiment, the medicament is for treatment of cancer, particularly BCMA-expressing cancer. In a further aspect, the medicament is for use in a method of treating cancer comprising administering to an individual having cancer an effective amount of said medicament. In one such aspect, the method further comprises administering to the individual an effective amount of at least one additional therapeutic agent, e.g., as described below. In another aspect, the medicament is for treatment of BCMA-expressing cancer. In a further aspect, the medicament is for use in a method of treating cancer, in particular BCMA-expressing cancer, comprising administering to an individual having cancer an effective amount of the medicament. In a further aspect, herein is provided a method for treating a cancer, in particular BCMA-expressing cancer. In one aspect, the method comprises administering to an individual having cancer an effective amount of an immunostimulatory antigen binding molecule that specifically binds to BCMA. In one such aspect, the method further comprises administering to the individual an effective amount of at least one additional therapeutic agent, as described below. An “individual” according to any of the above aspects may be a human.

In a further aspect, herein are provided pharmaceutical formulations comprising any of the immunostimulatory antigen binding molecules that specifically bind to BCMA as reported herein, e.g., for use in any of the above therapeutic methods. In one aspect, a pharmaceutical formulation comprises an immunostimulatory antigen binding molecule that specifically binds to BCMA as reported herein and a pharmaceutically acceptable carrier. In another aspect, a pharmaceutical formulation comprises an immunostimulatory antigen binding molecule that specifically binds to BCMA as reported herein and at least one additional therapeutic agent.

An immunostimulatory antigen binding molecule that specifically binds to BCMA as reported herein can be used either alone or in combination with other agents in a therapy. For instance, an immunostimulatory antigen binding molecule that specifically binds to BCMA as reported herein may be co-administered with at least one additional therapeutic agent. Thus, an immunostimulatory antigen binding molecule that specifically binds to BCMA as described herein for use in cancer immunotherapy is provided. In certain aspects, an immunostimulatory antigen binding molecule that specifically binds to BCMA for use in a method of cancer immunotherapy is provided. An “individual” according to any of the above aspects is preferably a human.

Such combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In one aspect, administration of the immunostimulatory antigen binding molecule that specifically binds to BCMA and administration of an additional therapeutic agent occur within about one month, or within about one, two or three weeks, or within about one, two, three, four, five, or six days, of each other.

An antigen binding molecule as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Immunostimulatory antigen binding molecules that specifically bind to BCMA as described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunostimulatory antigen binding molecule that specifically binds to BCMA needs not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an immunostimulatory antigen binding molecule that specifically binds to BCMA as described herein (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The immunostimulatory antigen binding molecule that specifically binds to BCMA is suitably administered to the patient at one time or over a series of treatments. Depending on the type and severity of the disease, about 1 μg/kg to 15 mg/kg (e.g. 0.5 mg/kg-10 mg/kg) of the immunostimulatory antigen binding molecule that specifically binds to BCMA can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antigen binding molecule would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

### Other Agents and Treatments

As described before, the immunostimulatory antigen binding molecule that specifically binds to BCMA may be administered in combination with one or more other agents in therapy. For instance, an antigen binding molecule as described herein may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain aspects, an additional therapeutic agent is another anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In certain aspects, an additional therapeutic agent is an immunomodulatory agent, a cytostatic agent, an inhibitor of cell adhesion, a cytotoxic or cytostatic agent, an activator of cell apoptosis, or an agent that increases the sensitivity of cells to apoptotic inducers.

Thus, provided are immunostimulatory antigen binding molecules that specifically bind to BCMA as disclosed herein or pharmaceutical compositions comprising them for use in the treatment of cancer, wherein the bispecific antibody is administered in combination with a chemotherapeutic agent, radiation and/or other agents for use in cancer immunotherapy.

Such other agents are suitably present in combination in amounts that are effective for the purpose intended. The effective amount of such other agents depends on the amount of fusion protein used, the type of disorder or treatment, and other factors discussed above. The bispecific antigen binding molecule or antibody of the invention are generally used in the same dosages and with administration routes as described herein, or about from 1 to 99% of the dosages described herein, or in any dosage and by any route that is empirically/clinically determined to be appropriate. Such combination therapies noted above encompass combined administration (where two or more therapeutic agents are included in the same or separate compositions), and separate administration, in which case, administration of the bispecific antigen binding molecule or antibody of the invention can occur prior to, simultaneously, and/or following, administration of the additional therapeutic agent and/or adjuvant.

In a further aspect, provided is an immunostimulatory antigen binding molecule that specifically binds to BCMA as described herein for use in the treatment of cancer, in particular BCMA-expressing cancer, wherein the bispecific antigen binding molecule is administered in combination with another immunomodulator. The term “immunomodulator” refers to any substance including a monoclonal antibody that effects the immune system. The molecules of the inventions can be considered immunomodulators. Immunomodulators can be used as anti-neoplastic agents for the treatment of cancer. In one aspect, immunomodulators include, but are not limited to anti-CTLA4 antibodies (e.g. ipilimumab), anti-PD1 antibodies (e.g. nivolumab or pembrolizumab), PD-L1 antibodies (e.g. atezolizumab, avelumab or durvalumab), OX-40 antibodies, 4-1BB antibodies and GITR antibodies. Such combination therapies noted above encompass combined administration (where two or more therapeutic agents are included in the same or separate compositions), and separate administration, in which case, administration of the bispecific antigen binding molecule can occur prior to, simultaneously, and/or following, administration of the additional therapeutic agent and/or adjuvant.

Combination with T Cell Bispecific Antibodies

In one aspect, the immunostimulatory antigen binding molecules that specifically bind to BCMA may be administered in combination with T-cell activating anti-CD3 bispecific antibodies. The T-cell activating anti-CD3 bispecific antibodies are specific for a tumor-associated antigen, for example GPRC5D, CD38 or BCMA. In one aspect, the T-cell activating anti-CD3 bispecific antibody is an anti-GPRC5D/anti-CD3 bispecific antibody.

In one aspect, the anti-GPRC5D/anti-CD3 bispecific antibody comprises at least one antigen binding domain capable of specific binding to GPRC5D which comprises a heavy chain variable region (V_{H}GPRC5D) comprising (i) CDR-H1 comprising the amino acid sequence of SEQ ID NO:140, (ii) CDR-H2 comprising the amino acid sequence of SEQ ID NO:141, and (iii) CDR-H3 comprising the amino acid sequence of SEQ ID NO:142, and a light chain variable region (V_{L}GPRC5D) comprising (iv) CDR-L1 comprising the amino acid sequence of SEQ ID NO:143, (v) CDR-L2 comprising the amino acid sequence of SEQ ID NO:144, and (vi) CDR-L3 comprising the amino acid sequence of SEQ ID NO:145. In one aspect, the anti-GPRC5D/anti-CD3 bispecific antibody comprises at least one antigen binding domain capable of specific binding to GPRC5D which comprises a heavy chain variable region (V_{H}GPRC5D) comprising an amino acid sequence selected from the group consisting of SEQ ID NO:146, SEQ ID NO:148, SEQ ID NO:149 and SEQ ID NO:150 and a light chain variable region (V_{L}GPRC5D) comprising an amino acid sequence selected from the group consisting of SEQ ID NO:147, SEQ ID NO:151, SEQ ID NO:152, SEQ ID NO:153, SEQ ID NO:154 and SEQ ID NO:155.

In one aspect, the anti-GPRC5D/anti-CD3 bispecific antibody comprises at least one antigen binding domain capable of specific binding to GPRC5D which comprises a heavy chain variable region (V_{H}GPRC5D) comprising an amino acid sequence that is at least about 95%, 96%, 97%, 98%, 99% or 100% identical to the amino acid sequence of SEQ ID NO:146, and a light chain variable region (V_{L}GPRC5D) comprising an amino acid sequence that is at least about 95%, 96%, 97%, 98%, 99% or 100% identical to the amino acid sequence of SEQ ID NO:147. Particularly, the antigen binding domain capable of specific binding to GPRC5D comprises a heavy chain variable region (V_{H}GPRC5D) comprising the amino acid sequence of SEQ ID NO:146 and a light chain variable region (V_{L}GPRC5D) comprising the amino acid sequence of SEQ ID NO:147.

In another aspect, the anti-GPRC5D/anti-CD3 bispecific antibody comprises at least one antigen binding domain capable of specific binding to GPRC5D which comprises a heavy chain variable region (V_{H}GPRC5D) comprising (i) CDR-H1 comprising the amino acid sequence of SEQ ID NO:156, (ii) CDR-H2 comprising the amino acid sequence of SEQ ID NO:157, and (iii) CDR-H3 comprising the amino acid sequence of SEQ ID NO:158, and a light chain variable region (V_{L}GPRC5D) comprising (iv) CDR-L1 comprising the amino acid sequence of SEQ ID NO:159, (v) CDR-L2 comprising the amino acid sequence of SEQ ID NO:160, and (vi) CDR-L3 comprising the amino acid sequence of SEQ ID NO:161. In one aspect, the anti-GPRC5D/anti-CD3 bispecific antibody comprises at least one antigen binding domain capable of specific binding to GPRC5D which comprises a heavy chain variable region (V_{H}GPRC5D) comprising an amino acid sequence selected from the group consisting of SEQ ID NO:162, SEQ ID NO:163, SEQ ID NO:164, SEQ ID NO:165, SEQ ID NO:166 and SEQ ID NO:167 and a light chain variable region (V_{L}GPRC5D) comprising an amino acid sequence selected from the group consisting of SEQ ID NO:168, SEQ ID NO:169, SEQ ID NO:170, SEQ ID NO:171 and SEQ ID NO:172. In another aspect, the anti-GPRC5D/anti-CD3 bispecific antibody comprises at least one antigen binding domain capable of specific binding to GPRC5D which comprises
(a) a heavy chain variable region (V_{H}GPRC5D) comprising the amino acid sequence of SEQ ID NO:146 and a light chain variable region (V_{L}GPRC5D) comprising the amino acid sequence of SEQ ID NO:147, or
(b) a heavy chain variable region (V_{H}GPRC5D) comprising the amino acid sequence of SEQ ID NO:150 and a light chain variable region (V_{L}GPRC5D) comprising the amino acid sequence of SEQ ID NO:153, or
(c) a heavy chain variable region (V_{H}GPRC5D) comprising the amino acid sequence of SEQ ID NO:146 and a light chain variable region (V_{L}GPRC5D) comprising the amino acid sequence of SEQ ID NO:153, or
(d) a heavy chain variable region (V_{H}GPRC5D) comprising the amino acid sequence of SEQ ID NO:162 and a light chain variable region (V_{L}GPRC5D) comprising the amino acid sequence of SEQ ID NO:170, or
(e) a heavy chain variable region (V_{H}GPRC5D) comprising the amino acid sequence of SEQ ID NO:164 and a light chain variable region (V_{L}GPRC5D) comprising the amino acid sequence of SEQ ID NO:169.

In one aspect, the anti-GPRC5D/anti-CD3 bispecific antibody comprises an antigen binding domain capable of specific binding to CD3 which comprises a heavy chain variable region (V_{H}CD3) comprising (i) CDR-H1 comprising the amino acid sequence of SEQ ID NO:173, (ii) CDR-H2 comprising the amino acid sequence of SEQ ID NO:174, and (iii) CDR-H3 comprising the amino acid sequence of SEQ ID NO:175, and a light chain variable region (V_{L}CD3) comprising (iv) CDR-L1 comprising the amino acid sequence of SEQ ID NO:176, (v) CDR-L2 comprising the amino acid sequence of SEQ ID NO:177, and (vi) CDR-L3 comprising the amino acid sequence of SEQ ID NO:178. In one aspect, the anti-GPRC5D/anti-CD3 bispecific antibody comprises an antigen binding domain capable of specific binding to CD3 which comprises a heavy chain variable region (V_{H}CD3) comprising an amino acid sequence of SEQ ID NO:179 and a light chain variable region (V_{L}CD3) comprising an amino acid sequence of SEQ ID NO:180.

In another aspect, the anti-GPRC5D/anti-CD3 bispecific antibody comprises an antigen binding domain capable of specific binding to CD3 which comprises a heavy chain variable region (V_{H}CD3) comprising (i) CDR-H1 comprising the amino acid sequence of SEQ ID NO:181, (ii) CDR-H2 comprising the amino acid sequence of SEQ ID NO:182, and (iii) CDR-H3 comprising the amino acid sequence of SEQ ID NO:183, and a light chain variable region (V_{L}CD3) comprising (iv) CDR-L1 comprising the amino acid sequence of SEQ ID NO:184, (v) CDR-L2 comprising the amino acid sequence of SEQ ID NO:185, and (vi) CDR-L3 comprising the amino acid sequence of SEQ ID NO:186. In one aspect, the anti-GPRC5D/anti-CD3 bispecific antibody comprises an antigen binding domain capable of specific binding to CD3 which comprises a heavy chain variable region (V_{H}CD3) comprising an amino acid sequence of SEQ ID NO:187 and a light chain variable region (V_{L}CD3) comprising an amino acid sequence of SEQ ID NO:188.

In one aspect, the anti-GPRC5D/anti-CD3 bispecific antibody comprises an antigen binding domain capable of specific binding to CD3 which comprises a heavy chain variable region (V_{H}CD3) comprising an amino acid sequence of SEQ ID NO:179 and a light chain variable region (V_{L}CD3) comprising an amino acid sequence of SEQ ID NO:180, and two antigen binding domains capable of specifically binding to GPRC5D, wherein the antigen binding domains capable of specifically binding to GPRC5D each comprise a heavy chain variable region (V_{H}GPRC5D) comprising the amino acid sequence of SEQ ID NO:146 and a light chain variable region (V_{L}GPRC5D) comprising the amino acid sequence of SEQ ID NO:147. In one particular aspect, the anti-GPRC5D/anti-CD3 bispecific antibody comprises a polypeptide that is at least 95%, 96%, 97%, 98%, or 99% identical to the sequence of SEQ ID NO:132, two polypeptides that are at least 95%, 96%, 97%, 98%, or 99% identical to the sequence of SEQ ID NO:133, a polypeptide that is at least 95%, 96%, 97%, 98%, or 99% identical to the sequence of SEQ ID NO:134, and a polypeptide that is at least 95%, 96%, 97%, 98%, or 99% identical to the sequence of SEQ ID NO:135. In a further particular aspect, the bispecific antibody comprises a polypeptide sequence of SEQ ID NO:132, two polypeptide sequences of SEQ ID NO:133, a polypeptide sequence of SEQ ID NO:134 and a polypeptide sequence of SEQ ID NO:135 (GPRC5D CD3 TCB). In one particular aspect, the anti-GPRC5D/anti-CD3 bispecific antibody is formatting.

In another aspect, the anti-GPRC5D/anti-CD3 bispecific antibody comprises a polypeptide that is at least 95%, 96%, 97%, 98%, or 99% identical to the sequence of SEQ ID NO: 136, a polypeptide that is at least 95%, 96%, 97%, 98%, or 99% identical to the sequence of SEQ ID NO: 137, a polypeptide that is at least 95%, 96%, 97%, 98%, or 99% identical to the sequence of SEQ ID NO: 138, and a polypeptide that is at least 95%, 96%, 97%, 98%, or 99% identical to the sequence of SEQ ID NO: 139. In one aspect, the bispecific antibody comprises a polypeptide sequence of SEQ ID NO: 136, a polypeptide sequence of SEQ ID NO: 137, a polypeptide sequence of SEQ ID NO: 138 and a polypeptide sequence of SEQ ID NO: 139 (GPRC5D CD3 1+1 bispecific antibody).

In a further aspect, the T-cell activating anti-CD3 bispecific antibody is an anti-BCMA/anti-CD3 bispecific antibody. In one aspect, the anti-BCMA/anti-CD3 bispecific antibody is in the 2+1 format. In one aspect, the anti-BCMA/anti-CD3 bispecific antibody comprises an amino acid sequence of SEQ ID NO:191, two amino acid sequences of SEQ ID NO:192, an amino acid sequence of SEQ ID NO:193 and amino acid sequence of SEQ ID NO:194. In another aspect, the anti-BCMA/anti-CD3 bispecific antibody is in the 1+1 format. In one aspect, the anti-BCMA/anti-CD3 bispecific antibody comprises an amino acid sequence of SEQ ID NO:195, an amino acid sequence of SEQ ID NO:196, an amino acid sequence of SEQ ID NO:197 and amino acid sequence of SEQ ID NO:198. In one aspect, the anti-BCMA/anti-CD3 bispecific antibody comprises an amino acid sequence of SEQ ID NO:199, an amino acid sequence of SEQ ID NO:200, an amino acid sequence of SEQ ID NO:201 and amino acid sequence of SEQ ID NO:202.

In another aspect, the T-cell activating anti-CD3 bispecific antibody is an anti-FcRH5/anti-CD3 bispecific antibody. Anti-FcRH5/anti-CD3 bispecific antibodies are for instance disclosed in WO 2016/205520 A1.

Such combination therapies noted above encompass combined administration (where two or more therapeutic agents are included in the same or separate formulations), and separate administration, in which case, administration of the therapeutic agent can occur prior to, simultaneously, and/or following, administration of an additional therapeutic agent or agents. In one embodiment, administration of the therapeutic agent and administration of an additional therapeutic agent occur within about one month, or within about one, two or three weeks, or within about one, two, three, four, five, or six days, of each other.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an immunostimulatory antigen binding molecule that specifically binds to BCMA as disclosed herein. The label or package insert indicates that the composition is used for treating the condition of choice. Moreover, the article of manufacture may comprise (a) a first container with a composition contained therein, wherein the composition comprises an immunostimulatory antigen binding molecule that specifically binds to BCMA; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID NO: | Name | Sequence |
| 1 | BCMA (E04_VH1a)-CDR- | GYTFTNYWMH |
| | H1 | |
| 2 | BCMA (E04_VH1a)-CDR- | IIHPNSGSTNYNEKFQG |
| | H2 | |
| 3 | BCMA (E04_VH1a)-CDR- | GIYDYPFAY |
| | H3 | |
| 4 | BCMA (E04_VL1f)-CDR- | RASESVSIHGTHLMH |
| | L1 | |
| 5 | BCMA (E04_VL1f)-CDR- | AASSLQS |
| | L2 | |
| 6 | BCMA (E04_VL1f)-CDR- | QQSIEDPYT |
| | L3 | |
| 7 | BCMA (E04_VL1a)-CDR- | AASNLES |
| | L2 | |
| 8 | BCMA (E04_VL1c)-CDR- | AASNLQS |
| | L2 | |
| 9 | Human (hu) 4-1BBL (71- | REGPELSPDDPAGLLDLRQGMFAQLVAQNVLLIDGPLSW |
| | 248) | YSDPGLAGVSLTGGLSYKEDTKELVVAKAGVYYVFFQLE |
| | | LRRVVAGEGSGSVSLALHLQPLRSAAGAAALALTVDLPP |
| | | ASSEARNSAFGFQGRLLHLSAGQRLGVHLHTEARARHAW |
| | | QLTQGATVLGLFRVTPEIPAGL |
| 10 | Human (hu) 4-1BBL (71- | REGPELSPDDPAGLLDLRQGMFAQLVAQNVLLIDGPLSW |
| | 254) | YSDPGLAGVSLTGGLSYKEDTKELVVAKAGVYYVFFQLE |
| | | LRRVVAGEGSGSVSLALHLQPLRSAAGAAALALTVDLPP |
| | | ASSEARNSAFGFQGRLLHLSAGQRLGVHLHTEARARHAW |
| | | QLTQGATVLGLFRVTPEIPAGLPSPRSE |
| 11 | dimeric hu 4-1BBL (71-248) | REGPELSPDDPAGLLDLRQGMFAQLVAQNVLLIDGPLSW |
| | connected by G4SG4 linker | YSDPGLAGVSLTGGLSYKEDTKELVVAKAGVYYVFFQLE |
| | | LRRVVAGEGSGSVSLALHLQPLRSAAGAAALALTVDLPP |
| | | ASSEARNSAFGFQGRLLHLSAGQRLGVHLHTEARARHAW |
| | | QLTQGATVLGLFRVTPEIPAGLGGGGSGGGGREGPELSP |
| | | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGL |
| 12 | dimeric hu 4-1BBL (71-254) | REGPELSPDDPAGLLDLRQGMFAQLVAQNVLLIDGPLSW |
| | connected by G4SG4 linker | YSDPGLAGVSLTGGLSYKEDTKELVVAKAGVYYVFFQLE |
| | | LRRVVAGEGSGSVSLALHLQPLRSAAGAAALALTVDLPP |
| | | ASSEARNSAFGFQGRLLHLSAGQRLGVHLHTEARARHAW |
| | | QLTQGATVLGLFRVTPEIPAGLPSPRSEGGGGSGGGGRE |
| | | GPELSPDDPAGLLDLRQGMFAQLVAQNVLLIDGPLSWYS |
| | | DPGLAGVSLTGGLSYKEDTKELVVAKAGVYYVFFQLELR |
| | | RVVAGEGSGSVSLALHLQPLRSAAGAAALALTVDLPPAS |
| | | SEARNSAFGFQGRLLHLSAGQRLGVHLHTEARARHAWQL |
| | | TQGATVLGLFRVTPEIPAGLPSPRSE |
| 13 | BCMA (E04_VH1a) VH | QVQLVQSGAEVKKPGASVKVSCKASGYTFTNYWMHWV |
| | | RQAPGQGLEWMGIIHPNSGSTNYNEKFQGRVTMTRDT |
| | | STSTVYMELSSLRSEDTAVYYCARGIYDYPFAYWGQG |
| | | TLVTVSS |
| 14 | BCMA (E04_VH1b) VH | QVQLVQSGAEVKKPGASVKVSCKASGYTFTNYWMHWV |
| | | RQAPGQGLEWMGIIHPNSGSTNYNEKFQGRVTMTVDK |
| | | STSTVYMELSSLRSEDTAVYYCARGIYDYPFAYWGQG |
| | | TLVTVSS |
| 15 | BCMA (E04_VL1f) VL | DIQMTQSPSSLSASVGDRVTITCRASESVSIHGTHLM |
| | | HWYQQKPGKAPKLLIYAASSLQSGVPSRFSGSGSETD |
| | | FTLTISSLQPEDFATYYCQQSIEDPYTFGGGTKVEIK |
| 16 | BCMA (E04_VL1a) VL | DIQMTQSPSSLSASVGDRVTITCRASESVSIHGTHLM |
| | | HWYQQKPGKAPKLLIYAASNLESGVPSRFSGSGSGTD |
| | | FTLTISSLQPEDFATYYCQQSIEDPYTFGGGTKVEIK |
| 17 | BCMA (E04_VL1b) VL | DIQMTQSPSSLSASVGDRVTITCRASESVSIHGTHLM |
| | | HWYQQKPGKAPKLLIYAASNLESGVPSRFSGSGSETD |
| | | FTLTISSLQPEDFATYYCQQSIEDPYTFGGGTKVEIK |
| 18 | BCMA (E04_VL1c) VL | DIQMTQSPSSLSASVGDRVTITCRASESVSIHGTHLM |
| | | HWYQQKPGKAPKLLIYAASNLQSGVPSRFSGSGSGTD |
| | | FTLTISSLQPEDFATYYCQQSIEDPYTFGGGTKVEIK |
| 19 | BCMA (E04_VL1d) VL | DIQMTQSPSSLSASVGDRVTITCRASESVSIHGTHLM |
| | | HWYQQKPGKAPKLLIYAASNLQSGVPSRFSGSGSETD |
| | | FTLTISSLQPEDFATYYCQQSIEDPYTFGGGTKVEIK |
| 20 | BCMA (E04_VL1e) VL | DIQMTQSPSSLSASVGDRVTITCRASESVSIHGTHLM |
| | | HWYQQKPGKAPKLLIYAASSLQSGVPSRFSGSGSGTD |
| | | FTLTISSLQPEDFATYYCQQSIEDPYTFGGGTKVEIK |
| 21 | Dimeric 4-1BB ligand (71- | REGPELSPDDPAGLLDLRQGMFAQLVAQNVLLIDGPL |
| | 248)-CL Fc knob chain | SWYSDPGLAGVSLTGGLSYKEDTKELVVAKAGVYYVF |
| | | FQLELRRVVAGEGSGSVSLALHLQPLRSAAGAAALAL |
| | | TVDLPPASSEARNSAFGFQGRLLHLSAGQRLGVHLHT |
| | | EARARHAWQLTQGATVLGLFRVTPEIPAGLGGGGSGG |
| | | GGREGPELSPDDPAGLLDLRQGMFAQLVAQNVLLIDG |
| | | PLSWYSDPGLAGVSLTGGLSYKEDTKELVVAKAGVYY |
| | | VFFQLELRRVVAGEGSGSVSLALHLQPLRSAAGAAAL |
| | | ALTVDLPPASSEARNSAFGFQGRLLHLSAGQRLGVHL |
| | | HTEARARHAWQLTQGATVLGLFRVTPEIPAGLGGGGS |
| | | GGGGRTVAAPSVFIFPPSDEQLKSGTASVVCLLNNFY |
| | | PREAKVQWKVDNALQSGNSQESVTEQDSKDSTYSLSS |
| | | TLTLSKADYEKHKVYACEVTHQGLSSPVTKSENRGEC |
| | | DKTHTCPPCPAPEAAGGPSVFLFPPKPKDTLMISRTP |
| | | EVTCVVVDVSHEDPEVKENWYVDGVEVHNAKTKPREE |
| | | QYNSTYRVVSVLTVLHQDWLNGKEYKCKVSNKALGAP |
| | | IEKTISKAKGQPREPQVYTLPPCRDELTKNQVSLWCL |
| | | VKGFYPSDIAVEWESNGQPENNYKTTPPVLDSDGSFF |
| | | LYSKLTVDKSRWQQGNVFSCSVMHEALHNHYTQKSLS |
| | | LSP |
| 22 | Monomeric 4-1BB ligand | REGPELSPDDPAGLLDLRQGMFAQLVAQNVLLIDGPL |
| | (71-248)-CH1 | SWYSDPGLAGVSLTGGLSYKEDTKELVVAKAGVYYVF |
| | | FQLELRRVVAGEGSGSVSLALHLQPLRSAAGAAALAL |
| | | TVDLPPASSEARNSAFGFQGRLLHLSAGQRLGVHLHT |
| | | EARARHAWQLTQGATVLGLFRVTPEIPAGLGGGGSGG |
| | | GGASTKGPSVFPLAPSSKSTSGGTAALGCLVKDYFPE |
| | | PVTVSWNSGALTSGVHTFPAVLQSSGLYSLSSVVTVP |
| | | SSSLGTQTYICNVNHKPSNTKVDKKVEPKSC |
| 23 | BCMA (E04_VH1a) VH | QVQLVQSGAEVKKPGASVKVSCKASGYTFTNYWMHWV |
| | CH1 (EE)-Fc hole | RQAPGQGLEWMGIIHPNSGSTNYNEKFQGRVTMTRDT |
| | PGLALA | STSTVYMELSSLRSEDTAVYYCARGIYDYPFAYWGQG |
| | | TLVTVSSASTKGPSVFPLAPSSKSTSGGTAALGCLVE |
| | | DYFPEPVTVSWNSGALTSGVHTFPAVLQSSGLYSLSS |
| | | VVTVPSSSLGTQTYICNVNHKPSNTKVDEKVEPKSCD |
| | | KTHTCPPCPAPEAAGGPSVFLFPPKPKDTLMISRTPE |
| | | VTCVVVDVSHEDPEVKFNWYVDGVEVHNAKTKPREEQ |
| | | YNSTYRVVSVLTVLHQDWLNGKEYKCKVSNKALGAPI |
| | | EKTISKAKGQPREPQVCTLPPSRDELTKNQVSLSCAV |
| | | KGFYPSDIAVEWESNGQPENNYKTTPPVLDSDGSFFL |
| | | VSKLTVDKSRWQQGNVFSCSVMHEALHNHYTQKSLSL |
| | | SP |
| 24 | BCMA (E04_VLIf) VL-CL | DIQMTQSPSSLSASVGDRVTITCRASESVSIHGTHLM |
| | (P1AG7400) | HWYQQKPGKAPKLLIYAASSLQSGVPSRFSGSGSETD |
| | | FTLTISSLQPEDFATYYCQQSIEDPYTFGGGTKVEIK |
| | | RTVAAPSVFIFPPSDRKLKSGTASVVCLLNNFYPREA |
| | | KVQWKVDNALQSGNSQESVTEQDSKDSTYSLSSTLTL |
| | | SKADYEKHKVYACEVTHQGLSSPVTKSENRGEC |
| 25 | BCMA (E04_VL1a) VL- | DIQMTQSPSSLSASVGDRVTITCRASESVSIHGTHLMHW |
| | CL (P1AG7409) | YQQKPGKAPKLLIYAASNLESGVPSRFSGSGSGTDFTLT |
| | | ISSLQPEDFATYYCQQSIEDPYTFGGGTKVEIKRTVAAP |
| | | SVFIFPPSDRKLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 26 | BCMA (54_VH2a)-CDR- | GFTFSNAWMD |
| | H1 | |
| 27 | BCMA (54_VH2a)-CDR- | QITAKSNNYATYYADSVKG |
| | H2 | |
| 28 | BCMA (54_VH2a)-CDR- | DGYH |
| | H3 | |
| 29 | BCMA (54_VH1b)-CDR- | QITAKSNNYATYYAAPVKG |
| | H2 | |
| 30 | BCMA (54_VL2a)-CDR- | RASEDIRNGLA |
| | L1 | |
| 31 | BCMA (54 VL2a)-CDR- | NANSLHT |
| | L2 | |
| 32 | BCMA (54_VL2a)-CDR- | EDTSKYPYT |
| | L3 | |
| 33 | BCMA (54_VH2a)-VH | EVQLLESGGGLVQPGGSLRLSCAASGFTFSNAWMDWV |
| | | RQAPGKGLEWVSQITAKSNNYATYYADSVKGRFTISR |
| | | DNSKNTLYLQMNSLRAEDTAVYYCTDDGYHWGQGTLV |
| | | TVSS |
| 34 | BCMA (54_VL2a)-VL | DIQMTQSPSSLSASVGDRVTITCRASEDIRNGLAWYQ |
| | | QKPGKAPKLLIYNANSLHTGVPSRFSGSGSGTDFTLT |
| | | ISSLQPEDFATYYCEDTSKYPYTFGQGTKLEIK |
| 35 | BCMA (54_VH1b)-VH | EVQLVESGGGLVKPGGSLRLSCAASGFTFSNAWMDWV |
| | | RQAPGKGLEWIAQITAKSNNYATYYAAPVKGRFTISR |
| | | DDSKNTLYLQMNSLKTEDTAVYYCTDDGYHWGQGTLV |
| | | TVSS |
| 36 | BCMA (54_VL1a)-VL | AIQMTQSPSSLSASVGDRVTITCRASEDIRNGLAWYQQK |
| | | PGKAPKLLIYNANSLHTGVPSRFSGSGSGTDFTLTISSL |
| | | QPEDFATYYCEDTSKYPYTFGQGTKLEIK |
| 37 | BCMA (54_VH2a) VH CH1 | EVQLLESGGGLVQPGGSLRLSCAASGFTFSNAWMDWVRQ |
| | (EE)-Fc hole PGLALA | APGKGLEWVSQITAKSNNYATYYADSVKGRFTISRDNSK |
| | (P1AG7397) | NTLYLQMNSLRAEDTAVYYCTDDGYHWGQGTLVTVSSAS |
| | | TKGPSVFPLAPSSKSTSGGTAALGCLVEDYFPEPVTVSW |
| | | NSGALTSGVHTFPAVLQSSGLYSLSSVVTVPSSSLGTQT |
| | | YICNVNHKPSNTKVDEKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVCTLPP |
| | | SRDELTKNQVSLSCAVKGFYPSDIAVEWESNGQPENNYK |
| | | TTPPVLDSDGSFFLVSKLTVDKSRWQQGNVFSCSVMHEA |
| | | LHNHYTQKSLSLSP |
| 38 | BCMA (54_VL2a) VL-CL | DIQMTQSPSSLSASVGDRVTITCRASEDIRNGLAWYQQK |
| | (RK) (P1AG7397) | PGKAPKLLIYNANSLHTGVPSRFSGSGSGTDFTLTISSL |
| | | QPEDFATYYCEDTSKYPYTFGQGTKLEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 39 | BCMA (54_VH1b) VH | EVQLVESGGGLVKPGGSLRLSCAASGFTFSNAWMDWVRQ |
| | CH1 (EE)-Fc hole | APGKGLEWIAQITAKSNNYATYYAAPVKGRFTISRDDSK |
| | PGLALA (P1AG7422) | NTLYLQMNSLKTEDTAVYYCTDDGYHWGQGTLVTVSSAS |
| | | TKGPSVFPLAPSSKSTSGGTAALGCLVEDYFPEPVTVSW |
| | | NSGALTSGVHTFPAVLQSSGLYSLSSVVTVPSSSLGTQT |
| | | YICNVNHKPSNTKVDEKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVCTLPP |
| | | SRDELTKNQVSLSCAVKGFYPSDIAVEWESNGQPENNYK |
| | | TTPPVLDSDGSFFLVSKLTVDKSRWQQGNVFSCSVMHEA |
| | | LHNHYTQKSLSLSP |
| 40 | BCMA (54_VL1a) VL-CL | AIQMTQSPSSLSASVGDRVTITCRASEDIRNGLAWYQQK |
| | (RK) (P1AG7422) | PGKAPKLLIYNANSLHTGVPSRFSGSGSGTDFTLTISSL |
| | | QPEDFATYYCEDTSKYPYTFGQGTKLEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 41 | Parental BCMA (E04) VH | QVQLQQPGAELVKPGASVKLSCKASGYTFTNYWMHWVKQ |
| | | RPGQGLEWIGIIHPNSGSTNYNEKFKSKATLTVDKSSST |
| | | AYMQLSSLTSEDSAVYYCARGIYDYPFAYWGQGTLVTVS |
| | | S |
| 42 | Parental BCMA (E04) VL | DIVLTQSPASLAVSLGQRATISCRASESVSIHGTHLMHW |
| | | YQQKPGQPPKLLIYAASNLESGVPARFSGSGSETDETLN |
| | | IHPVEEEDAATYFCQQSIEDPYTFGGGTKLEIK |
| 43 | human IGHJ germline | YFDYWGQGTLVTVSS |
| | IGHJ4*01 post-CDR3 | |
| | framework | |
| 44 | human IGKJ germline | LTFGGGTKVEIK |
| | IGKJ4*01 post-CDR3 | |
| | framework | |
| 45 | BCMA (E04_VL1g) VL | DIQMTQSPSSLSASVGDRVTITCRASESVSIHGTH |
| | | LMHWYQQKPGKAPKLLIYAASNLESGVPSRESGSG |
| | | SGTDFTLTISSLQPEDFATYFCQQSIEDPYTFGGG |
| | | TKVEIK |
| 46 | BCMA (E04_VL1h) VL | DIQMTQSPSSLSASVGDRVTITCRASESVSIHGTH |
| | | LMHWYQQKPGKAPKLLIYAASNLESGVPSRESGSG |
| | | SETDFTLTISSLQPEDFATYFCQQSIEDPYTFGGG |
| | | TKVEIK |
| 47 | BCMA-54 VH | EVQLVESGGGLVKPGESLRLSCAASGFTFSNAWMDWVRQ |
| | | APGKRLEWIAQITAKSNNYATYYAAPVKGRFTISRDDSK |
| | | NTLYLQMNSLKKEDTAVYYCTDDGYHWGQGTLVTVSS |
| 48 | BCMA-54 VL | AIQMTQSPSSLSASVGDRVTIACRASEDIRNGLAWYQQK |
| | | PGKAPKLLIYNANSLHTGVPSRFSGSGSGTDFTLTISSL |
| | | QPEDEAIYYCEDTSKYPYTFGQGTKLEIK |
| 49 | human IGHJ germline | AEYFQHWGQGTLVTVSS |
| | IGHJ1*01 post-CDR3 | |
| | framework | |
| 50 | human IGKJ germline | YTFGQGTKLEIK |
| | IGKJ2*01 post-CDR3 | |
| | framework | |
| 51 | BCMA CDR-H2 | RIKSKTDGGTTDYAAPVKG |
| 52 | BCMA CDR-L2 | AASSLQS |
| 53 | BCMA (54_VH1a) | EVQLVESGGGLVKPGGSLRLSCAASGFTFSNAWMDWV |
| | | RQAPGKGLEWVGQITAKSNNYATYYAAPVKGRFTISR |
| | | DDSKNTLYLQMNSLKTEDTAVYYCTDDGYHWGQGTLV |
| | | TVSS |
| 54 | BCMA (54_VH1a_Y292D) | EVQLVESGGGLVKPGGSLRLSCAASGFTFSNAWMDWV |
| | | RQAPGKGLEWVGQITAKSNNYATDYAAPVKGRFTISR |
| | | DDSKNTLYLQMNSLKTEDTAVYYCTDDGYHWGQGTLV |
| | | TVSS |
| 55 | BCMA | EVQLVESGGGLVKPGGSLRLSCAASGFTFSNAWMDWV |
| | (54_VH1c_huCDR2) | RQAPGKGLEWVGRIKSKTDGGTTDYAAPVKGRFTISR |
| | | DDSKNTLYLQMNSLKTEDTAVYYCTDDGYHWGQGTLV |
| | | TVSS |
| 56 | BCMA (54_VH1a_W197Y) | EVQLVESGGGLVKPGGSLRLSCAASGFTFSNAYMDWV |
| | | RQAPGKGLEWVGQITAKSNNYATYYAAPVKGRFTISR |
| | | DDSKNTLYLQMNSLKTEDTAVYYCTDDGYHWGQGTLV |
| | | TVSS |
| 57 | BCMA (54_VL1a_L2_GL) | AIQMTQSPSSLSASVGDRVTITCRASEDIRNGLAWYQ |
| | | QKPGKAPKLLIYAASSLQSGVPSRFSGSGSGTDFTLT |
| | | ISSLQPEDFATYYCEDTSKYPYTFGQGTKLEIK |
| 58 | BCMA (54_VL2a_L2_GL) | DIQMTQSPSSLSASVGDRVTITCRASEDIRNGLAWYQ |
| | | QKPGKAPKLLIYAASSLQSGVPSRFSGSGSGTDFTLT |
| | | ISSLQPEDFATYYCEDTSKYPYTFGQGTKLEIK |
| 59 | BCMA (54_VL1a_L1_pGL) | AIQMTQSPSSLSASVGDRVTITCRASEDIRNDLGWYQ |
| | | QKPGKAPKLLIYNANSLHTGVPSRFSGSGSGTDFTLT |
| | | ISSLQPEDFATYYCEDTSKYPYTFGQGTKLEIK |
| 60 | BCMA (54_VL1a_N651A) | AIQMTQSPSSLSASVGDRVTITCRASEDIRNGLAWYQ |
| | | QKPGKAPKLLIYAANSLHTGVPSRFSGSGSGTDFTLT |
| | | ISSLQPEDFATYYCEDTSKYPYTFGQGTKLEIK |
| 61 | BCMA | AIQMTQSPSSLSASVGDRVTITCRASEDIRNGLAWYQ |
| | (54_VL1a_N651A_N695S) | QKPGKAPKLLIYAASSLHTGVPSRFSGSGSGTDFTLT |
| | | ISSLQPEDFATYYCEDTSKYPYTFGQGTKLEIK |
| 62 | BCMA (54_VL1a_H698Q) | AIQMTQSPSSLSASVGDRVTITCRASEDIRNGLAWYQ |
| | | QKPGKAPKLLIYNANSLQTGVPSRFSGSGSGTDFTLT |
| | | ISSLQPEDFATYYCEDTSKYPYTFGQGTKLEIK |
| 63 | BCMA (54_VL1a_T699S) | AIQMTQSPSSLSASVGDRVTITCRASEDIRNGLAWYQ |
| | | QKPGKAPKLLIYNANSLHSGVPSRFSGSGSGTDFTLT |
| | | ISSLQPEDFATYYCEDTSKYPYTFGQGTKLEIK |
| 64 | BCMA | AIQMTQSPSSLSASVGDRVTITCRASEDIRNGLAWYQ |
| | (54_VL1a_H698Q_T699S) | QKPGKAPKLLIYNANSLQSGVPSRFSGSGSGTDFTLT |
| | | ISSLQPEDFATYYCEDTSKYPYTFGQGTKLEIK |
| 65 | BCMA (54_VH1a) VH- | EVQLVESGGGLVKPGGSLRLSCAASGFTFSNAWMDWVRQ |
| | CH1-Fc knob PGLALA | APGKGLEWVGQITAKSNNYATYYAAPVKGRFTISRDDSK |
| | | NTLYLQMNSLKTEDTAVYYCTDDGYHWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVYTLPP |
| | | CRDELTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSP |
| 66 | BCMA (54_VL1a_L1_pGL) | AIQMTQSPSSLSASVGDRVTITCRASEDIRNDLGWYQQK |
| | VL-Ckappa | PGKAPKLLIYNANSLHTGVPSRFSGSGSGTDFTLTISSL |
| | | QPEDFATYYCEDTSKYPYTFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 67 | Fc fragment | DKTHTCPPCPAPEAAGGPSVFLFPPKPKDTLMISRTPEV |
| | | TCVVVDVSHEDPEVKENWYVDGVEVHNAKTKPREEQYNS |
| | | TYRVVSVLTVLHQDWLNGKEYKCKVSNKALGAPIEKTIS |
| | | KAKGQPREPQVCTLPPSRDELTKNQVSLSCAVKGFYPSD |
| | | IAVEWESNGQPENNYKTTPPVLDSDGSFFLVSKLTVDKS |
| | | RWQQGNVFSCSVMHEALHNRFTQKSLSLSP |
| 68 | BCMA (54_VH1a_Y292D) | EVQLVESGGGLVKPGGSLRLSCAASGFTFSNAWMDWVRQ |
| | VH-CH1-Fc knob PGLALA | APGKGLEWVGQITAKSNNYATDYAAPVKGRFTISRDDSK |
| | | NTLYLQMNSLKTEDTAVYYCTDDGYHWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVYTLPP |
| | | CRDELTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSP |
| 69 | BCMA (54_VL1a) VL- | AIQMTQSPSSLSASVGDRVTITCRASEDIRNGLAWYQQK |
| | Ckappa | PGKAPKLLIYNANSLHTGVPSRFSGSGSGTDFTLTISSL |
| | | QPEDFATYYCEDTSKYPYTFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 70 | BCMA (54_VL1a_T699S) | AIQMTQSPSSLSASVGDRVTITCRASEDIRNGLAWYQQK |
| | VL-Ckappa | PGKAPKLLIYNANSLHSGVPSRFSGSGSGTDFTLTISSL |
| | | QPEDFATYYCEDTSKYPYTFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 71 | BCMA (54_VL2a) VL- | DIQMTQSPSSLSASVGDRVTITCRASEDIRNGLAWYQQK |
| | Ckappa | PGKAPKLLIYNANSLHTGVPSRFSGSGSGTDFTLTISSL |
| | | QPEDFATYYCEDTSKYPYTFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 72 | BCMA (54_VH1b) VH- | EVQLVESGGGLVKPGGSLRLSCAASGFTFSNAWMDWVRQ |
| | CH1-Fc knob PGLALA | APGKGLEWIAQITAKSNNYATYYAAPVKGRFTISRDDSK |
| | | NTLYLQMNSLKTEDTAVYYCTDDGYHWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVYTLPP |
| | | CRDELTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSP |
| 73 | BCMA (54_VL1a_N651A) | AIQMTQSPSSLSASVGDRVTITCRASEDIRNGLAWYQQK |
| | VL-Ckappa | PGKAPKLLIYAANSLHTGVPSRFSGSGSGTDFTLTISSL |
| | | QPEDFATYYCEDTSKYPYTFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 74 | BCMA | AIQMTQSPSSLSASVGDRVTITCRASEDIRNGLAWYQQK |
| | (54_VL1a_N651A_N695S) | PGKAPKLLIYAASSLHTGVPSRFSGSGSGTDFTLTISSL |
| | VL-Ckappa | QPEDFATYYCEDTSKYPYTFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 75 | BCMA (54_VH2a) VH- | EVQLLESGGGLVQPGGSLRLSCAASGFTFSNAWMDWVRQ |
| | CH1-Fc knob PGLALA | APGKGLEWVSQITAKSNNYATYYADSVKGRFTISRDNSK |
| | | NTLYLQMNSLRAEDTAVYYCTDDGYHWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVYTLPP |
| | | CRDELTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSP |
| 76 | BCMA (54) heavy chain (Fc | EVQLVESGGGLVKPGESLRLSCAASGFTFSNAWMDWVRQ |
| | knob PGLALA) | APGKRLEWIAQITAKSNNYATYYAAPVKGRFTISRDDSK |
| | | NTLYLQMNSLKKEDTAVYYCTDDGYHWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQT |
| | | YICNVNHKPSNTKVDKKVEPKSCDKTHTCPPCPAPEAAG |
| | | GPSVFLFPPKPKDTLMISRTPEVTCVVVDVSHEDPEVKE |
| | | NWYVDGVEVHNAKTKPREEQYNSTYRVVSVLTVLHQDWL |
| | | NGKEYKCKVSNKALGAPIEKTISKAKGQPREPQVYTLPP |
| | | CRDELTKNQVSLWCLVKGFYPSDIAVEWESNGQPENNYK |
| | | TTPPVLDSDGSFFLYSKLTVDKSRWQQGNVFSCSVMHEA |
| | | LHNHYTQKSLSLSP |
| 77 | BCMA (54) light chain | AIQMTQSPSSLSASVGDRVTIACRASEDIRNGLAWYQQK |
| | | PGKAPKLLIYNANSLHTGVPSRFSGSGSGTDFTLTISSL |
| | | QPEDEAIYYCEDTSKYPYTFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 78 | BCMA (E04_VH1a) VH- | QVQLVQSGAEVKKPGASVKVSCKASGYTFTNYWMHWVRQ |
| | CH1-Fc knob PGLALA | APGQGLEWMGIIHPNSGSTNYNEKFQGRVTMTRDTSTST |
| | | VYMELSSLRSEDTAVYYCARGIYDYPFAYWGQGTLVTVS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALGAPIEKTISKAKGQPREPQVYT |
| | | LPPCRDELTKNQVSLWCLVKGFYPSDIAVEWESNGQPEN |
| | | NYKTTPPVLDSDGSFFLYSKLTVDKSRWQQGNVFSCSVM |
| | | HEALHNHYTQKSLSLSP |
| 79 | BCMA (E04_VL1a) VL- | DIQMTQSPSSLSASVGDRVTITCRASESVSIHGTHLMHW |
| | Ckappa | YQQKPGKAPKLLIYAASNLESGVPSRFSGSGSGTDFTLT |
| | | ISSLQPEDFATYYCQQSIEDPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 80 | BCMA (E04_VL1b) VL- | DIQMTQSPSSLSASVGDRVTITCRASESVSIHGTHLMHW |
| | Ckappa | YQQKPGKAPKLLIYAASNLESGVPSRFSGSGSETDFTLT |
| | | ISSLQPEDFATYYCQQSIEDPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 81 | BCMA (E04_VL1d) VL- | DIQMTQSPSSLSASVGDRVTITCRASESVSIHGTHLMHW |
| | Ckappa | YQQKPGKAPKLLIYAASNLQSGVPSRFSGSGSETDFTLT |
| | | ISSLQPEDFATYYCQQSIEDPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 82 | BCMA (E04_VL1e) VL- | DIQMTQSPSSLSASVGDRVTITCRASESVSIHGTHLMHW |
| | Ckappa | YQQKPGKAPKLLIYAASSLQSGVPSRFSGSGSGTDFTLT |
| | | ISSLQPEDFATYYCQQSIEDPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 83 | BCMA (E04_VLIf) VL- | DIQMTQSPSSLSASVGDRVTITCRASESVSIHGTHLMHW |
| | Ckappa | YQQKPGKAPKLLIYAASSLQSGVPSRFSGSGSETDFTLT |
| | | ISSLQPEDFATYYCQQSIEDPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 84 | BCMA (E04_VL1g) VL- | DIQMTQSPSSLSASVGDRVTITCRASESVSIHGTHLMHW |
| | Ckappa | YQQKPGKAPKLLIYAASNLESGVPSRFSGSGSGTDFTLT |
| | | ISSLQPEDFATYFCQQSIEDPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 85 | BCMA (E04_VL1h) VL- | DIQMTQSPSSLSASVGDRVTITCRASESVSIHGTHLMHW |
| | Ckappa | YQQKPGKAPKLLIYAASNLESGVPSRFSGSGSETDFTLT |
| | | ISSLQPEDFATYFCQQSIEDPYTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 86 | BCMA (E04_VH1b) VH- | QVQLVQSGAEVKKPGASVKVSCKASGYTFTNYWMHWVRQ |
| | CH1-Fc knob PGLALA | APGQGLEWMGIIHPNSGSTNYNEKFQGRVTMTVDKSTST |
| | | VYMELSSLRSEDTAVYYCARGIYDYPFAYWGQGTLVTVS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALGAPIEKTISKAKGQPREPQVYT |
| | | LPPCRDELTKNQVSLWCLVKGFYPSDIAVEWESNGQPEN |
| | | NYKTTPPVLDSDGSFFLYSKLTVDKSRWQQGNVFSCSVM |
| | | HEALHNHYTQKSLSLSP |
| 87 | BCMA (E04) heavy chain | QVQLQQPGAELVKPGASVKLSCKASGYTFTNYWMHWVKQ |
| | (Fc knob PGLALA) | RPGQGLEWIGIIHPNSGSTNYNEKFKSKATLTVDKSSST |
| | | AYMQLSSLTSEDSAVYYCARGIYDYPFAYWGQGTLVTVS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALGAPIEKTISKAKGQPREPQVYT |
| | | LPPCRDELTKNQVSLWCLVKGFYPSDIAVEWESNGQPEN |
| | | NYKTTPPVLDSDGSFFLYSKLTVDKSRWQQGNVFSCSVM |
| | | HEALHNHYTQKSLSLSP |
| 88 | BCMA (E04) light chain | DIVLTQSPASLAVSLGQRATISCRASESVSIHGTHLMHW |
| | | YQQKPGQPPKLLIYAASNLESGVPARFSGSGSETDFTLN |
| | | IHPVEEEDAATYFCQQSIEDPYTFGGGTKLEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 89 | Heavy chain of 5D5 LM4 | EVQLLESGGGLVQPGGSLRLSCAASGFTFSDYGLSWVRQ |
| | (P1AH1062) | APGKGLEWVSLIDSSGSSTFYADSVKGRFTISRDNSKNT |
| | | LYLQMNSLRAEDTAVYYCAKEHGLFDSWGQGTLVTVSSA |
| | | STKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELL |
| | | GGPSVFLFPPKPKDTLMISRTPEVTCVVVDVSHEDPEVK |
| | | FNWYVDGVEVHNAKTKPREEQYNSTYRVVSVLTVLHQDW |
| | | LNGKEYKCKVSNKALPAPIEKTISKAKGQPREPQVYTLP |
| | | PSREEMTKNQVSLTCLVKGFYPSDIAVEWESNGQPENNY |
| | | KTTPPVLDSDGSFFLYSKLTVDKSRWQQGNVFSCSVMHE |
| | | ALHNHYTQKSLSLSPGKGSGSGSGSGSGSGSGSGSQSVL |
| | | TQPPSASGTPGRRVTISCSGSSSNIGNNYVTWYQQLPGT |
| | | APKLLIYADSHRPSGVPDRESGSKSGTSASLAISGLRSE |
| | | DEADYYCATWDYSLSGYVFGCGTKLTVLGGGGSGGGGSG |
| | | GGGSGGGGSEVQLLESGGGLVQPGGSLRLSCAASGFTFS |
| | | SYDMSWVRQAPGKCLEWVSWISYSGGSIYYADSVKGRFT |
| | | ISRDNSKNTLYLQMNSLRAEDTAVYYCARDAQRNSMREF |
| | | DYWGQGTLVTVSS |
| 90 | Light Chain of 5D5 LM4 | EIVLTQSPGTLSLSPGERATLSCKASQDIDDAINWYQQK |
| | (P1AH1062) | PGQAPRLLIYDASLRATGIPDRFSGSGSGTDFTLTISRL |
| | | EPEDFAVYYCQQSLRTPITFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 91 | BCMA (PR) VH CH1-Fc | EVQLLESGGGLVQPGGSLRLSCAASGFTFSSYAMNWVRQAPGK |
| | hole PGLALA (P1AF7814) | GLEWVSAITASGGSTYYADSVKGRFTISRDNSKNTLYLQMNSL |
| | | RAEDTAVYYCARYWPMSLWGQGTLVTVSSASTKGPSVFPLAPS |
| | | SKSTSGGTAALGCLVKDYFPEPVTVSWNSGALTSGVHTFPAVL |
| | | QSSGLYSLSSVVTVPSSSLGTQTYICNVNHKPSNTKVDKKVEP |
| | | KSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALGAPIEKTISKAKGQPREP |
| | | QVCTLPPSRDELTKNQVSLSCAVKGFYPSDIAVEWESNGQPEN |
| | | NYKTTPPVLDSDGSFFLVSKLTVDKSRWQQGNVFSCSVMHEAL |
| | | HNHYTQKSLSLSP |
| 92 | BCMA (PR) VL-CL | EIVLTQSPGTLSLSPGERATLSCRASQSVSAYYLAWYQQKPGQ |
| | (P1AF7814) | APRLLMYDASIRATGIPDRFSGSGSGTDFTLTISRLEPEDFAV |
| | | YYCQQYERWPLTFGQGTKVEIKVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSENRGEC |
| 93 | Human BCMA Uniprot Acc, | MLQMAGQCSQNEYFDSLLHACIPCQLRCSSNTPPLTCQRYCNA |
| | No. Q02223 | SVTNSVKGTNAILWTCLGLSLIISLAVFVLMFLLRKINSEPLK |
| | | DEFKNTGSGLLGMANIDLEKSRTGDEIILPRGLEYTVEECTCE |
| | | DCIKSKPKVDSDHCFPLPAMEEGATILVTTKTNDYCKSLPAAL |
| | | SATEIEKSISAR |
| 94 | Human CD28 Uniprot Acc. | MLRLLLALNLFPSIQVTGNKILVKQSPMLVAYDNAVNLSCKYS |
| | No. P10747 | YNLFSREFRASLHKGLDSAVEVCVVYGNYSQQLQVYSKTGENC |
| | | DGKLGNESVTFYLQNLYVNQTDIYFCKIEVMYPPPYLDNEKSN |
| | | GTIIHVKGKHLCPSPLFPGPSKPFWVLVVVGGVLACYSLLVTV |
| | | AFIIFWVRSKRSRLLHSDYMNMTPRRPGPTRKHYQPYAPPRDF |
| | | AAYRS |
| 95 | Human kappa CL domain | RTVAAPSVFIFPPSDEQLKSGTASVVCLLNNFYPR |
| | | EAKVQWKVDNALQSGNSQESVTEQDSKDSTYSLSS |
| | | TLTLSKADYEKHKVYACEVTHQGLSSPVTKSENRG |
| | | EC |
| 96 | Human lambda CL domain | QPKAAPSVTLFPPSSEELQANKATLVCLISDFYPG |
| | | AVTVAWKADSSPVKAGVETTTPSKQSNNKYAASSY |
| | | LSLTPEQWKSHRSYSCQVTHEGSTVEKTVAPTECS |
| 97 | IgG CH1 domain | ASTKGPSVFPLAPSSKSTSGGTAALGCLVKDYFPE |
| | | PVTVSWNSGALTSGVHTFPAVLQSSGLYSLSSVVT |
| | | VPSSSLGTQTYICNVNHKPSNTKVDKKV |
| 98 | CH1 domain connector | EPKSC |
| 99 | Hinge full | DKTHTCPXCP with X being S or P |
| 100 | Hinge middle | HTCPXCP with X being S or P |
| 101 | Hinge short | CPXCP with X being S or P |
| 102 | IgG CH2 domain | APELLGGPSVFLFPPKPKDTLMISRTPEVTCVWDVSHED |
| | | PEVKFNWYVDGVEVHNAKTKPREEQESTYRWSVLTVLHQ |
| | | DWLNGKEYKCKVSNKALPAPIEKTISKAK |
| 103 | IgG CH3 domain | GQPREPQVYTLPPSRDELTKNQVSLTCLVKGFYPSDIAV |
| | | EWESNGQPENNYKTTPPVLDSDGSFFLYSKLTVDKSRWQ |
| | | QGNVFSCSVMHEALHNHYTQKSLSLSPGK |
| 104 | IgG1, caucasian allotype | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPEL |
| | | LGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 105 | IgG1, afroamerican allotype | ASTKGPSVFPLAPSSKSTSGGTAALGCLVKDYFPEPVTV |
| | | SWNSGALTSGVHTFPAVLQSSGLYSLSSVVTVPSSSLGT |
| | | QTYICNVNHKPSNTKVDKKVEPKSCDKTHTCPPCPAPEL |
| | | LGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 106 | IgG2 | ASTKGPSVFPLAPCSRSTSESTAALGCLVKDYFPEPVTV |
| | | SWNSGALTSGVHTFPAVLQSSGLYSLSSVVTVPSSNFGT |
| | | QTYTCNVDHKPSNTKVDKTVERKCCVECPPCPAPPVAGP |
| | | SVFLFPPKPKDTLMISRTPEVTCVVVDVSHEDPEVQENW |
| | | YVDGVEVHNAKTKPREEQFNSTFRVVSVLTVVHQDWING |
| | | KEYKCKVSNKGLPAPIEKTISKTKGQPREPQVYTLPPSR |
| | | EEMTKNQVSLTCLVKGFYPSDISVEWESNGQPENNYKTT |
| | | PPMLDSDGSFFLYSKLTVDKSRWQQGNVFSCSVMHEALH |
| | | NHYTQKSLSLSPGK |
| 107 | IgG3 | ASTKGPSVFPLAPCSRSTSGGTAALGCLVKDYFPEPVTV |
| | | SWNSGALTSGVHTFPAVLQSSGLYSLSSVVTVPSSSLGT |
| | | QTYTCNVNHKPSNTKVDKRVELKTPLGDTTHTCPRCPEP |
| | | KSCDTPPPCPRCPEPKSCDTPPPCPRCPEPKSCDTPPPC |
| | | PRCPAPELLGGPSVFLFPPKPKDTLMISRTPEVTCVVVD |
| | | VSHEDPEVQFKWYVDGVEVHNAKTKPREEQYNSTFRVVS |
| | | VLTVLHQDWLNGKEYKCKVSNKALPAPIEKTISKTKGQP |
| | | REPQVYTLPPSREEMTKNQVSLTCLVKGFYPSDIAVEWE |
| | | SSGQPENNYNTTPPMLDSDGSFFLYSKLTVDKSRWQQGN |
| | | IFSCSVMHEALHNRFTQKSLSLSPGK |
| 108 | IgG4 | ASTKGPSVFPLAPCSRSTSESTAALGCLVKDYFPEPVTV |
| | | SWNSGALTSGVHTFPAVLQSSGLYSLSSVVTVPSSSLGT |
| | | KTYTCNVDHKPSNTKVDKRVESKYGPPCPSCPAPEFLGG |
| | | PSVFLFPPKPKDTLMISRTPEVTCVVVDVSQEDPEVQEN |
| | | WYVDGVEVHNAKTKPREEQFNSTYRVVSVLTVLHQDWLN |
| | | GKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 109 | hu IgG1 heavy chain | ASTKGPSVFPLAPSSKSTSGGTAALGCLVKDYFPE |
| | constant region with | PVTVSWNSGALTSGVHTFPAVLQSSGLYSLSSVVT |
| | mutations L234A, L235A | VPSSSLGTQTYICNVNHKPSNTKVDKKVEPKSCDK |
| | and P329G | THTCPPCPAPEAAGGPSVELFPPKPKDTLMISRTP |
| | | EVTCVVVDVSHEDPEVKENWYVDGVEVHNAKTKPR |
| | | EEQYNSTYRVVSVLTVLHQDWLNGKEYKCKVSNKA |
| | | LGAPIEKTISKAKGQPREPQVYTLPPSRDELTKNQ |
| | | VSLTCLVKGFYPSDIAVEWESNGQPENNYKTTPPV |
| | | LDSDGSFFLYSKLTVDKSRWQQGNVFSCSVMHEAL |
| | | HNHYTQKSLSLSP |
| 110 | human FcyRIIIa, Uniprot | MWQLLLPTALLLLVSAGMRTEDLPKAVVFLEPQWYRVLE |
| | Acc No. P08637 | KDSVTLKCQGAYSPEDNSTQWFHNESLISSQASSYFIDA |
| | | ATVDDSGEYRCQTNLSTLSDPVQLEVHIGWLLLQAPRWV |
| | | FKEEDPIHLRCHSWKNTALHKVTYLQNGKGRKYFHHNSD |
| | | FYIPKATLKDSGSYFCRGLFGSKNVSSETVNITITQGLA |
| | | VSTISSFFPPGYQVSFCLVMVLLFAVDTGLYFSVKTNIR |
| | | SSTRDWKDHKFKWRKDPQDK |
| 111 | Peptide linker (G4S) | GGGGS |
| 112 | Peptide linker G4SG4 | GGGGSGGGG |
| 113 | Peptide linker (G4S)2 | GGGGSGGGGS |
| 114 | Peptide linker (SG4)2 | SGGGGSGGGG |
| 115 | Peptide linker G4(SG4)2 | GGGGSGGGGSGGGG |
| 116 | peptide linker | GSPGSSSSGS |
| 117 | (G4S)3 peptide linker | GGGGSGGGGSGGGGS |
| 118 | (G4S)4 peptide linker | GGGGSGGGGSGGGGSGGGGS |
| 119 | peptide linker | GSGSGSGS |
| 120 | peptide linker | GSGSGNGS |
| 121 | peptide linker | GGSGSGSG |
| 122 | peptide linker | GGSGSG |
| 123 | peptide linker | GGSG |
| 124 | peptide linker | GGSGNGSG |
| 125 | peptide linker | GGNGSGSG |
| 126 | peptide linker | GGNGSG |
| 127 | Human BCMA extracellular | MLQMAGQCSQNEYFDSLLHACIPCQLRCSSNTPPLTCQR |
| | domain | YCNASVTNSVKGTNA |
| 128 | Cynomolgus BCMA | MLQMARQCSQNEYFDSLLHDCKPCQLRCSSTPPLTCQRY |
| | (Macaca fascicularis) NCBI | CNASMTNSVKGMNAILWTCLGLSLIISLAVFVLTFLLRK |
| | XP_005591343.1 | MSSEPLKDEFKNTGSGLLGMANIDLEKGRTGDEIVLPRG |
| | | LEYTVEECTCEDCIKNKPKVDSDHCFPLPAMEEGATILV |
| | | TTKTNDYCNSLSAALSVTEIEKSISAR |
| 129 | Cyno BCMA extracellular | MLQMARQCSQNEYFDSLLHDCKPCQLRCSSTPPLTCQRY |
| | domain | CNASMTNSVKGMNA |
| 130 | BCMA (E04_VL1g) | DIQMTQSPSSLSASVGDRVTITCRASESVSIHGTH |
| | | LMHWYQQKPGKAPKLLIYAASNLESGVPSRFSGSG |
| | | SGTDFTLTISSLQPEDFATYFCQQSIEDPYTFGGG |
| | | TKVEIK |
| 131 | BCMA (E04_VL1h) | DIQMTQSPSSLSASVGDRVTITCRASESVSIHGTH |
| | | LMHWYQQKPGKAPKLLIYAASNLESGVPSRESGSG |
| | | SETDFTLTISSLQPEDFATYFCQQSIEDPYTFGGG |
| | | TKVEIK |
| 132 | GPRC5D (5E11) hu IgG1 | EVQLLESGGGLVQPGGSLRLSCAASGFTFSKYAMAWVRQ |
| | VH-CH1 “EE” Fc hole | APGKGLEWVASISTGGVNTYYADSVKGRFTISRDNSKNT |
| | PGLALA (P1AE6625) | LYLQMNSLRAEDTAVYYCATHTGDYFDYWGQGTMVTVSS |
| | | ASTKGPSVFPLAPSSKSTSGGTAALGCLVEDYFPEPVTV |
| | | SWNSGALTSGVHTFPAVLQSSGLYSLSSVVTVPSSSLGT |
| | | QTYICNVNHKPSNTKVDEKVEPKSCDKTHTCPPCPAPEA |
| | | AGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALGAPIEKTISKAKGQPREPQVCTL |
| | | PPSRDELTKNQVSLSCAVKGFYPSDIAVEWESNGQPENN |
| | | YKTTPPVLDSDGSFFLVSKLTVDKSRWQQGNVFSCSVMH |
| | | EALHNHYTQKSLSLSP |
| 133 | GPRC5D (5E11)VL-Ckappa | EIVLTQSPGTLSLSPGERATLSCRASQSVSISGINLMNW |
| | “RK” (P1AE6625) | YQQKPGQQPKLLIYHASILASGIPDRFSGSGSGTDFTLT |
| | | ISRLEPEDFAVYYCQQTRESPLTFGQGTRLEIKRTVAAP |
| | | SVFIFPPSDRKLKSGTASVVCLLNNFYPREAKVQWKVDN |
| | | ALQSGNSQESVTEQDSKDSTYSLSSTLTLSKADYEKHKV |
| | | YACEVTHQGLSSPVTKSENRGEC |
| 134 | GPRC5D (5E11) VH- | EVQLLESGGGLVQPGGSLRLSCAASGFTFSKYAMAWVRQ |
| | CH1(EE)-CD3 (C122) VL- | APGKGLEWVASISTGGVNTYYADSVKGRFTISRDNSKNT |
| | CH1-Fc (knob, P329G LALA) | LYLQMNSLRAEDTAVYYCATHTGDYFDYWGQGTMVTVSS |
| | (P1AE6625) | ASTKGPSVFPLAPSSKSTSGGTAALGCLVEDYFPEPVTV |
| | | SWNSGALTSGVHTFPAVLQSSGLYSLSSVVTVPSSSLGT |
| | | QTYICNVNHKPSNTKVDEKVEPKSCDGGGGSGGGGSQAV |
| | | VTQEPSLTVSPGGTVTLTCGSSTGAVTTSNYANWVQEKP |
| | | GQAFRGLIGGTNKRAPGTPARFSGSLLGGKAALTLSGAQ |
| | | PEDEAEYYCALWYSNLWVFGGGTKLTVLSSASTKGPSVE |
| | | PLAPSSKSTSGGTAALGCLVKDYFPEPVTVSWNSGALTS |
| | | GVHTFPAVLQSSGLYSLSSVVTVPSSSLGTQTYICNVNH |
| | | KPSNTKVDKKVEPKSCDKTHTCPPCPAPEAAGGPSVELF |
| | | PPKPKDTLMISRTPEVTCVVVDVSHEDPEVKENWYVDGV |
| | | EVHNAKTKPREEQYNSTYRVVSVLTVLHQDWLNGKEYKC |
| | | KVSNKALGAPIEKTISKAKGQPREPQVYTLPPCRDELTK |
| | | NQVSLWCLVKGFYPSDIAVEWESNGQPENNYKTTPPVLD |
| | | SDGSFFLYSKLTVDKSRWQQGNVFSCSVMHEALHNHYTQ |
| | | KSLSLSP |
| 135 | CD3 (C122) VH-CL | EVQLLESGGGLVQPGGSLRLSCAASGFQFSSYAMNWVRQ |
| | (P1AE6625) | APGKGLEWVSRIRSKYNNYATYYADSVKGRFTISRDDSK |
| | | NTLYLQMNSLRAEDTAVYYCVRHTTFPSSYVSYYGYWGQ |
| | | GTLVTVSSASVAAPSVFIFPPSDEQLKSGTASVVCLLNN |
| | | FYPREAKVQWKVDNALQSGNSQESVTEQDSKDSTYSLSS |
| | | TLTLSKADYEKHKVYACEVTHQGLSSPVTKSENRGEC |
| 136 | GPRC5D hu IgG1 VH-CH1 | QVQLVQSGAEVKKPGASVKVSCKASGYSFTGYTMNWVRQ |
| | “EE” Fc hole PGLALA | APGQGLEWMGLINPYNSDTNYAQKLQGRVTMTTDTSTST |
| | (P1AE3357) | AYMELRSLRSDDTAVYYCARVALRVALDYWGQGTLVTVS |
| | | SASTKGPSVFPLAPSSKSTSGGTAALGCLVEDYFPEPVT |
| | | VSWNSGALTSGVHTFPAVLQSSGLYSLSSVVTVPSSSLG |
| | | TQTYICNVNHKPSNTKVDEKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALGAPIEKTISKAKGQPREPQVCT |
| | | LPPSRDELTKNQVSLSCAVKGFYPSDIAVEWESNGQPEN |
| | | NYKTTPPVLDSDGSFFLVSKLTVDKSRWQQGNVFSCSVM |
| | | HEALHNHYTQKSLSLSP |
| 137 | GPRC5D VL-Ckappa ″RK″ | DIQMTQSPSSLSASVGDRVTITCKASQNVATHVGWYQQK |
| | (P1AE3357) | PGKAPKRLIYSASYRYSGVPSRFSGSGSGTEFTLTISNL |
| | | QPEDFATYYCQQYNRYPYTFGQGTKLEIKRTVAAPSVFI |
| | | FPPSDRKLKSGTASVVCLLNNFYPREAKVQWKVDNALQS |
| | | GNSQESVTEQDSKDSTYSLSSTLTLSKADYEKHKVYACE |
| | | VTHQGLSSPVTKSENRGEC |
| 138 | CD3 VL-CH1-Fc (knob, | QTVVTQEPSLTVSPGGTVTLTCRSSTGAVTTSNYANWVQ |
| | P329G LALA) (P1AE3357) | QKPGQAPRGLIGGTNKRAPGTPARFSGSLLGGKAALTLS |
| | | GVQPEDEAEYYCALWYSNLWVFGGGTKLTVLSSASTKGP |
| | | SVFPLAPSSKSTSGGTAALGCLVKDYFPEPVTVSWNSGA |
| | | LTSGVHTFPAVLQSSGLYSLSSVVTVPSSSLGTQTYICN |
| | | VNHKPSNTKVDKKVEPKSCDKTHTCPPCPAPEAAGGPSV |
| | | FLFPPKPKDTLMISRTPEVTCVVVDVSHEDPEVKENWYV |
| | | DGVEVHNAKTKPREEQYNSTYRVVSVLTVLHQDWLNGKE |
| | | YKCKVSNKALGAPIEKTISKAKGQPREPQVYTLPPCRDE |
| | | LTKNQVSLWCLVKGFYPSDIAVEWESNGQPENNYKTTPP |
| | | VLDSDGSFFLYSKLTVDKSRWQQGNVFSCSVMHEALHNH |
| | | YTQKSLSLSP |
| 139 | CD3 VH-CL (P1AE3357) | EVQLVESGGGLVQPGGSLRLSCAASGFTENTYAMNWVRQ |
| | | APGKGLEWVARIRSKYNNYATYYAASVKGRFTISRDDSK |
| | | NSLYLQMNSLKTEDTAVYYCARHGNFGNSYVSWFAYWGQ |
| | | GTLVTVSSASVAAPSVFIFPPSDEQLKSGTASVVCLLNN |
| | | FYPREAKVQWKVDNALQSGNSQESVTEQDSKDSTYSLSS |
| | | TLTLSKADYEKHKVYACEVTHQGLSSPVTKSENRGEC |
| 140 | GPRC5D (5E11)-CDR-H1 | KYAMA |
| 141 | GPRC5D (5E11)-CDR-H2 | SISTGGVNTYYADSVKG |
| 142 | GPRC5D (5E11)-CDR-H3 | HTGDYFDY |
| 143 | GPRC5D (5E11)-CDR-L1 | RASQSVSISGINLMN |
| 144 | GPRC5D (5E11)-CDR-L2 | HASILAS |
| 145 | GPRC5D (5E11)-CDR-L3 | QQTRESPLT |
| 146 | GPRC5D (5E11) VH1c | EVQLLESGGGLVQPGGSLRLSCAASGFTFSKYAMAWVRQ |
| | | APGKGLEWVASISTGGVNTYYADSVKGRFTISRDNSKNT |
| | | LYLQMNSLRAEDTAVYYCATHTGDYFDYWGQGTMVTVSS |
| 147 | GPRC5D (5E11) VL2b | EIVLTQSPGTLSLSPGERATLSCRASQSVSISGINLMNW |
| | | YQQKPGQQPKLLIYHASILASGIPDRFSGSGSGTDFTLT |
| | | ISRLEPEDFAVYYCQQTRESPLTFGQGTRLEIK |
| 148 | GPRC5D (5E11) VH1a | EVQLLESGGGLVQPGGSLRLSCAASGFTFSKYAMAWVRQ |
| | | APGKGLEWVASISTGGVNTYYRDSVKARFTISRDNSKNT |
| | | LYLQMNSLRAEDTAVYYCATHTGDYFDYWGQGTMVTVSS |
| 149 | GPRC5D (5E11) VH1b | ELQLLESGGGLVQPGGSLRLSCAASGFTFSKYAMAWVRQ |
| | | APGKGLEWVASISTGGVNTYYRDSVKARFTISRDNAKNT |
| | | LYLQMNSLRAEDTAVYYCATHTGDYFDYWGQGTMVTVSS |
| 150 | GPRC5D (5E11) VH1d | ELQLLESGGGLVQPGGSLRLSCAASGFTFSKYAMAWVRQ |
| | | APGKGLEWVASISTGGVNTYYADSVKGRFTISRDNAKNT |
| | | LYLQMNSLRAEDTAVYYCATHTGDYFDYWGQGTMVTVSS |
| 151 | GPRC5D (5E11) VL1a | DIVMTQSPDSLAVSLGERATINCRASQSVSISGINLMNW |
| | | YQQKPGQQPKLLIYHASILASGVPDRFSGSGSGTDFTLT |
| | | ISSLQAEDVAVYYCQQTRESPLTFGQGTRLEIK |
| 152 | GPRC5D (5E11) VL1c | DIVMTQSPDSLAVSLGERATINCKSSQSVSISGINLMNW |
| | | YQQKPGQQPKLLIYHASILASGVPDRFSGSGSGTDFTLT |
| | | ISSLQAEDVAVYYCQQTRESPLTFGQGTRLEIK |
| 153 | GPRC5D (5E11) VL2a | EIVLTQSPGTLSLSPGERATLSCRASQSVSISGINLMNW |
| | | YQQKPGQQPRLLIYHASILASGIPDRFSGSGSGTDFTLT |
| | | ISRLEPEDFAVYYCQQTRESPLTFGQGTRLEIK |
| 154 | GPRC5D (5E11) VL3a | DIQMTQSPSSLSASVGDRVTITCRASQSVSISGINLMNW |
| | | YQQKPGKQPKLLIYHASILASGVPSRFSGSGSGTDFTLT |
| | | ISSLQPEDFATYYCQQTRESPLTFGQGTRLEIK |
| 155 | GPRC5D (5E11) VL3b | DIQMTQSPSSLSASVGDRVTITCRASQSVSISGINLMNW |
| | | YQQKPGQQPKLLIYHASILASGVPSRFSGSGSGTDFTLT |
| | | ISSLQPEDFATYYCQQTRESPLTFGQGTRLEIK |
| 156 | GPRC5D (5F11)-CDR-H1 | NYGMA |
| 157 | GPRC5D (5F11)-CDR-H2 | SISTGGGNTYYRDSVKG |
| 158 | GPRC5D (5F11)-CDR-H3 | HDRGGLY |
| 159 | GPRC5D (5F11)-CDR-L1 | RSSKSLLHSNGITYVY |
| 160 | GPRC5D (5F11)-CDR-L2 | RMSNRAS |
| 161 | GPRC5D (5F11)-CDR-L3 | GQLLENPYT |
| 162 | GPRC5D (5F11) VH1a | QVQLVESGGGVVQPGRSLRLSCAASGFSFSNYGMAWVRQ |
| | | APGKGLEWVASISTGGGNTYYRDSVKGRFTISRDNSKNT |
| | | LYLQMNSLRAEDTAVYYCTRHDRGGLYWGQGTMVTVSS |
| 163 | GPRC5D (5F11) VH1b | EVQLVESGGGVVQPGRSLRLSCAASGFSFSNYGMAWVRQ |
| | | APGKGLEWVASISTGGGNTYYRDSVKGRFTISRDNAKNT |
| | | LYLQMNSLRAEDTAVYYCTRHDRGGLYWGQGTMVTVSS |
| 164 | GPRC5D (5F11) VH1c | QVQLVESGGGVVQPGRSLRLSCAASGFSFSNYGMAWVRQ |
| | | APGKGLEWVASISTGGGNTYYADSVKGRFTISRDNSKNT |
| | | LYLQMNSLRAEDTAVYYCTRHDRGGLYWGQGTMVTVSS |
| 165 | GPRC5D (5F11) VH1d | EVQLVESGGGVVQPGRSLRLSCAASGESFSNYGMAWVRQ |
| | | APGKGLEWVASISTGGGNTYYADSVKGRFTISRDNAKNT |
| | | LYLQMNSLRAEDTAVYYCTRHDRGGLYWGQGTMVTVSS |
| 166 | GPRC5D (5F11) VH2b | EVQLVESGGGLVQPGGSLRLSCAASGFSFSNYGMAWVRQ |
| | | APGKGLEWVASISTGGGNTYYRDSVKGRFTISRDNAKNT |
| | | LYLQMNSLRAEDTAVYYCTRHDRGGLYWGQGTMVTVSS |
| 167 | GPRC5D (5F11) VH2d | EVQLVESGGGLVQPGGSLRLSCAASGFSFSNYGMAWVRQ |
| | | APGKGLEWVASISTGGGNTYYADSVKGRFTISRDNAKNT |
| | | LYLQMNSLRAEDTAVYYCTRHDRGGLYWGQGTMVTVSS |
| 168 | GPRC5D (5F11) VL1a | DIVMTQSPLSLPVTPGEPASISCRSSKSLLHSNGITYVY |
| | | WYLQKPGQSPQVLIYRMSNLASGVPDRFSGSGSGTDFTL |
| | | KISRVEAEDVGVYHCGQLLENPYTFGQGTKLEIK |
| 169 | GPRC5D (5F11) VL1b | DIVMTQSPLSLPVTPGEPASISCRSSKSLLHSNGITYVY |
| | | WYLQKPGKSPQVLIYRMSNLASGVPDRFSGSGSGTDFTL |
| | | KISRVEAEDVGVYHCGQLLENPYTFGQGTKLEIK |
| 170 | GPRC5D (5F11) VL2a | DIVMTQSPLSLPVTPGEPASISCRSSKSLLHSNGITYVY |
| | | WYLQKPGQSPQLLIYRMSNRASGVPDRFSGSGSGTDFTL |
| | | KISRVEAEDVGVYHCGQLLENPYTFGQGTKLEIK |
| 171 | GPRC5D (5F11) VL2b | DIVMTQSPDSLAVSLGERATINCKSSKSLLHSNGITYVY |
| | | WYQQKPGQPPKLLIYRMSNLASGVPDRFSGSGSGTDFTL |
| | | TISSLQAEDVAVYHCGQLLENPYTFGQGTKLEIK |
| 172 | GPRC5D (5F11) VL2c | EIVLTQSPGTLSLSPGERATLSCRASKSLLHSNGITYVY |
| | | WYQQKPGQAPRLLIYRMSNLASGIPDRFSGSGSGTDFTL |
| | | TISRLEPEDFAVYHCGQLLENPYTFGQGTKLEIK |
| 173 | CD3 (C122) CDR-H1 | SYAMN |
| 174 | CD3 (C122) CDR-H2 | RIRSKYNNYATYYADSVKG |
| 175 | CD3 (C122) CDR-H3 | HTTFPSSYVSYYGY |
| 176 | CD3 (C122) CDR-L1 | GSSTGAVTTSNYAN |
| 177 | CD3 (C122) CDR-L2 | GTNKRAP |
| 178 | CD3 (C122) CDR-L3 | ALWYSNLWV |
| 179 | CD3 (C122) VH | EVQLLESGGGLVQPGGSLRLSCAASGFQFSSYAMNWVRQ |
| | | APGKGLEWVSRIRSKYNNYATYYADSVKGRFTISRDDSK |
| | | NTLYLQMNSLRAEDTAVYYCVRHTTFPSSYVSYYGYWGQ |
| | | GTLVTVSS |
| 180 | CD3 (C122) VL | QAVVTQEPSLTVSPGGTVTLTCGSSTGAVTTSNYANWVQ |
| | | EKPGQAFRGLIGGTNKRAPGTPARFSGSLLGGKAALTLS |
| | | GAQPEDEAEYYCALWYSNLWVFGGGTKLTVL |
| 181 | CD3 (V9) CDR-H1 | GYSFTGYTMN |
| 182 | CD3 (V9) CDR-H2 | LINPYKGVSTYNQKFKD |
| 183 | CD3 (V9) CDR-H3 | SGYYGDSDWYFDV |
| 184 | CD3 (V9) CDR-L1 | RASQDIRNYLN |
| 185 | CD3 (V9) CDR-L2 | YTSRLES |
| 186 | CD3 (V9) CDR-L3 | QQGNTLPWT |
| 187 | CD3 (V9) VH | EVQLVESGGGLVQPGGSLRLSCAASGYSFTGYTMNWVRQ |
| | | APGKGLEWVALINPYKGVSTYNQKFKDRFTISVDKSKNT |
| | | AYLQMNSLRAEDTAVYYCARSGYYGDSDWYFDVWGQGTL |
| | | VTVSS |
| 188 | CD3 (V9) VL | DIQMTQSPSSLSASVGDRVTITCRASQDIRNYLNWYQQK |
| | | PGKAPKLLIYYTSRLESGVPSRFSGSGSGTDYTLTISSL |
| | | QPEDFATYYCQQGNTLPWTFGQGTKVEIK |
| 189 | dimeric hu 4-1BBL (71-248) | REGPELSPDDPAGLLDLRQGMFAQLVAQNVLLIDGPLSW |
| | connected by (G4S)2 linker | YSDPGLAGVSLTGGLSYKEDTKELVVAKAGVYYVFFQLE |
| | | LRRVVAGEGSGSVSLALHLQPLRSAAGAAALALTVDLPP |
| | | ASSEARNSAFGFQGRLLHLSAGQRLGVHLHTEARARHAW |
| | | QLTQGATVLGLFRVTPEIPAGLGGGGSGGGGSREGPELS |
| | | PDDPAGLLDLRQGMFAQLVAQNVLLIDGPLSWYSDPGLA |
| | | GVSLTGGLSYKEDTKELVVAKAGVYYVFFQLELRRVVAG |
| | | EGSGSVSLALHLQPLRSAAGAAALALTVDLPPASSEARN |
| | | SAFGFQGRLLHLSAGQRLGVHLHTEARARHAWQLTQGAT |
| | | VLGLFRVTPEIPAGL |
| 190 | dimeric hu 4-1BBL (71-254) | REGPELSPDDPAGLLDLRQGMFAQLVAQNVLLIDGPLSW |
| | connected by (G4S)2 linker | YSDPGLAGVSLTGGLSYKEDTKELVVAKAGVYYVFFQLE |
| | | LRRVVAGEGSGSVSLALHLQPLRSAAGAAALALTVDLPP |
| | | ASSEARNSAFGFQGRLLHLSAGQRLGVHLHTEARARHAW |
| | | QLTQGATVLGLFRVTPEIPAGLPSPRSEGGGGSGGGGSR |
| | | EGPELSPDDPAGLLDLRQGMFAQLVAQNVLLIDGPLSWY |
| | | SDPGLAGVSLTGGLSYKEDTKELVVAKAGVYYVFFQLEL |
| | | RRVVAGEGSGSVSLALHLQPLRSAAGAAALALTVDLPPA |
| | | SSEARNSAFGFQGRLLHLSAGQRLGVHLHTEARARHAWQ |
| | | LTQGATVLGLFRVTPEIPAGLPSPRSE |
| 191 | Alnuctamab hu IgG1 Fc | EVQLLESGGGLVQPGGSLRLSCAASGFTFSDNAMGWVRQ |
| | hole PGLALA (P1AF0105) | APGKGLEWVSAISGPGSSTYYADSVKGRFTISRDNSKNT |
| | | LYLQMNSLRAEDTAVYYCAKVLGWFDYWGQGTLVTVSSA |
| | | STKGPSVFPLAPSSKSTSGGTAALGCLVEDYFPEPVTVS |
| | | WNSGALTSGVHTFPAVLQSSGLYSLSSVVTVPSSSLGTQ |
| | | TYICNVNHKPSNTKVDEKVEPKSCDKTHTCPPCPAPEAA |
| | | GGPSVFLFPPKPKDTLMISRTPEVTCVVVDVSHEDPEVK |
| | | FNWYVDGVEVHNAKTKPREEQYNSTYRVVSVLTVLHQDW |
| | | LNGKEYKCKVSNKALGAPIEKTISKAKGQPREPQVCTLP |
| | | PSRDELTKNQVSLSCAVKGFYPSDIAVEWESNGQPENNY |
| | | KTTPPVLDSDGSFFLVSKLTVDKSRWQQGNVFSCSVMHE |
| | | ALHNHYTQKSLSLSPGK |
| 192 | Alnuctamab VL-Ckappa | EIVLTQSPGTLSLSPGERATLSCRASQSVSDEYLSWYQQ |
| | TNFRSF17 (P1AF0105) | KPGQAPRLLIHSASTRATGIPDRFSGSGSGTDFTLAISR |
| | | LEPEDFAVYYCQQYGYPPDFTFGQGTKVEIKRTVAAPSV |
| | | FIFPPSDRKLKSGTASVVCLLNNFYPREAKVQWKVDNAL |
| | | QSGNSQESVTEQDSKDSTYSLSSTLTLSKADYEKHKVYA |
| | | CEVTHQGLSSPVTKSENRGEC |
| 193 | Alnuctamab hu IgG1_Fc | EVQLLESGGGLVQPGGSLRLSCAASGFTFSDNAMGWVRQ |
| | knob PGLALA (P1AF0105) | APGKGLEWVSAISGPGSSTYYADSVKGRFTISRDNSKNT |
| | | LYLQMNSLRAEDTAVYYCAKVLGWFDYWGQGTLVTVSSA |
| | | STKGPSVFPLAPSSKSTSGGTAALGCLVEDYFPEPVTVS |
| | | WNSGALTSGVHTFPAVLQSSGLYSLSSVVTVPSSSLGTQ |
| | | TYICNVNHKPSNTKVDEKVEPKSCDGGGGSGGGGSQAVV |
| | | TQEPSLTVSPGGTVTLTCGSSTGAVTTSNYANWVQEKPG |
| | | QAFRGLIGGTNKRAPGTPARFSGSLLGGKAALTLSGAQP |
| | | EDEAEYYCALWYSNLWVFGGGTKLTVLSSASTKGPSVFP |
| | | LAPSSKSTSGGTAALGCLVKDYFPEPVTVSWNSGALTSG |
| | | VHTFPAVLQSSGLYSLSSVVTVPSSSLGTQTYICNVNHK |
| | | PSNTKVDKKVEPKSCDKTHTCPPCPAPEAAGGPSVFLFP |
| | | PKPKDTLMISRTPEVTCVVVDVSHEDPEVKENWYVDGVE |
| | | VHNAKTKPREEQYNSTYRVVSVLTVLHQDWLNGKEYKCK |
| | | VSNKALGAPIEKTISKAKGQPREPQVYTLPPCRDELTKN |
| | | QVSLWCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 194 | Alnuctamab VL-Ckappa | EVQLLESGGGLVQPGGSLRLSCAASGFTFSTYAMNWVRQ |
| | CD3 (P1AF0105) | APGKGLEWVSRIRSKYNNYATYYADSVKGRFTISRDDSK |
| | | NTLYLQMNSLRAEDTAVYYCVRHGNFGNSYVSWFAYWGQ |
| | | GTLVTVSSASVAAPSVFIFPPSDEQLKSGTASVVCLLNN |
| | | FYPREAKVQWKVDNALQSGNSQESVTEQDSKDSTYSLSS |
| | | TLTLSKADYEKHKVYACEVTHQGLSSPVTKSENRGEC |
| 195 | Elranatamab hu IgG1_Fc | EVQLLESGGGLVQPGGSLRLSCAASGFTFSSYPMSWVRQ |
| | hole PGLALA (P1AH5054) | APGKGLEWVSAIGGSGGSLPYADIVKGRFTISRDNSKNT |
| | | LYLQMNSLRAEDTAVYYCARYWPMDIWGQGTLVTVSSAS |
| | | TKGPSVFPLAPSSKSTSGGTAALGCLVEDYFPEPVTVSW |
| | | NSGALTSGVHTFPAVLQSSGLYSLSSVVTVPSSSLGTQT |
| | | YICNVNHKPSNTKVDEKVEPKSCDKTHTCPPCPAPEAAG |
| | | GPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALGAPIEKTISKAKGQPREPQVCTLPP |
| | | SRDELTKNQVSLSCAVKGFYPSDIAVEWESNGQPENNYK |
| | | TTPPVLDSDGSFFLVSKLTVDKSRWQQGNVFSCSVMHEA |
| | | LHNHYTQKSLSLSPG |
| 196 | Elranatamab VL-Ckappa | EIVLTQSPGTLSLSPGERATLSCRASQSVSSSYLAWYQQ |
| | TNFRSF17 (P1AH5054) | KPGQAPRLLMYDASIRATGIPDRFSGSGSGTDFTLTISR |
| | | LEPEDFAVYYCQQYQSWPLTFGQGTKVEIKRTVAAPSVE |
| | | IFPPSDRKLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSENRGEC |
| 197 | Elranatamab hu IgG1_Fc | DIVMTQSPDSLAVSLGERATINCKSSQSLENVRSRKNYL |
| | knob PGLALA (P1AH5054) | AWYQQKPGQPPKLLISWASTRESGVPDRFSGSGSGTDFT |
| | | LTISSLQAEDVAVYYCKQSYDLFTFGSGTKLEIKSSAST |
| | | KGPSVFPLAPSSKSTSGGTAALGCLVKDYFPEPVTVSWN |
| | | SGALTSGVHTFPAVLQSSGLYSLSSVVTVPSSSLGTQTY |
| | | ICNVNHKPSNTKVDKKVEPKSCDKTHTCPPCPAPEAAGG |
| | | PSVFLFPPKPKDTLMISRTPEVTCVVVDVSHEDPEVKEN |
| | | WYVDGVEVHNAKTKPREEQYNSTYRVVSVLTVLHQDWLN |
| | | GKEYKCKVSNKALGAPIEKTISKAKGQPREPQVYTLPPC |
| | | RDELTKNQVSLWCLVKGFYPSDIAVEWESNGQPENNYKT |
| | | TPPVLDSDGSFFLYSKLTVDKSRWQQGNVFSCSVMHEAL |
| | | HNHYTQKSLSLSPG |
| 198 | Elranatamab VL-Ckappa | EVQLVESGGGLVQPGGSLRLSCAASGFTFSDYYMTWVRQ |
| | CD3 (P1AH5054) | APGKGLEWVAFIRNRARGYTSDHNPSVKGRFTISRDNAK |
| | | NSLYLQMNSLRAEDTAVYYCARDRPSYYVLDYWGQGTTV |
| | | TVSSASVAAPSVFIFPPSDEQLKSGTASVVCLLNNFYPR |
| | | EAKVQWKVDNALQSGNSQESVTEQDSKDSTYSLSSTLTL |
| | | SKADYEKHKVYACEVTHQGLSSPVTKSENRGEC |
| 199 | Teclistamab anti- | QLQLQESGPGLVKPSETLSLTCTVSGGSISSGSYFWGWI |
| | TNFRSF17 heavy chain | RQPPGKGLEWIGSIYYSGITYYNPSLKSRVTISVDTSKN |
| | | QFSLKLSSVTAADTAVYYCARHDGAVAGLFDYWGQGTLV |
| | | TVSSASTKGPSVFPLAPCSRSTSESTAALGCLVKDYFPE |
| | | PVTVSWNSGALTSGVHTFPAVLQSSGLYSLSSVVTVPSS |
| | | SLGTKTYTCNVDHKPSNTKVDKRVESKYGPPCPPCPAPE |
| | | AAGGPSVFLFPPKPKDTLMISRTPEVTCVVVDVSQEDPE |
| | | VQFNWYVDGVEVHNAKTKPREEQFNSTYRVVSVLTVLHQ |
| | | DWLNGKEYKCKVSNKGLPSSIEKTISKAKGQPREPQVYT |
| | | LPPSQEEMTKNQVSLTCLVKGFYPSDIAVEWESNGQPEN |
| | | NYKTTPPVLDSDGSFFLYSRLTVDKSRWQEGNVFSCSVM |
| | | HEALHNHYTQKSLSLSLGK |
| 200 | Teclistamab anti- | SYVLTQPPSVSVAPGQTARITCGGNNIGSKSVHWYQQPP |
| | TNFRSF17 light chain | GQAPVVVVYDDSDRPSGIPERFSGSNSGNTATLTISRVE |
| | | AGDEAVYYCQVWDSSSDHVVFGGGTKLTVLGQPKAAPSV |
| | | TLFPPSSEELQANKATLVCLISDFYPGAVTVAWKGDSSP |
| | | VKAGVETTTPSKQSNNKYAASSYLSLTPEQWKSHRSYSC |
| | | QVTHEGSTVEKTVAPTECS |
| 201 | Teclistamab anti-CD3 | EVQLVESGGGLVQPGGSLRLSCAASGFTENTYAMNWVRQ |
| | heavy chain | APGKGLEWVARIRSKYNNYATYYAASVKGRFTISRDDSK |
| | | NSLYLQMNSLKTEDTAVYYCARHGNFGNSYVSWFAYWGQ |
| | | GTLVTVSSASTKGPSVFPLAPCSRSTSESTAALGCLVKD |
| | | YFPEPVTVSWNSGALTSGVHTFPAVLQSSGLYSLSSVVT |
| | | VPSSSLGTKTYTCNVDHKPSNTKVDKRVESKYGPPCPPC |
| | | PAPEAAGGPSVFLFPPKPKDTLMISRTPEVTCVVVDVSQ |
| | | EDPEVQFNWYVDGVEVHNAKTKPREEQFNSTYRVVSVLT |
| | | VLHQDWLNGKEYKCKVSNKGLPSSIEKTISKAKGQPREP |
| | | QVYTLPPSQEEMTKNQVSLTCLVKGFYPSDIAVEWESNG |
| | | QPENNYKTTPPVLDSDGSFLLYSKLTVDKSRWQEGNVES |
| | | CSVMHEALHNHYTQKSLSLSLGK |
| 202 | Teclistamab anti-CD3 light | QTVVTQEPSLTVSPGGTVTLTCRSSTGAVTTSNYANWVQ |
| | chain | QKPGQAPRGLIGGTNKRAPGTPARFSGSLLGGKAALTLS |
| | | GVQPEDEAEYYCALWYSNLWVFGGGTKLTVLGQPKAAPS |
| | | VTLFPPSSEELQANKATLVCLISDFYPGAVTVAWKADSS |
| | | PVKAGVETTTPSKQSNNKYAASSYLSLTPEQWKSHRSYS |
| | | CQVTHEGSTVEKTVAPTECS |
| 203 | huBCMA R27P | MLQMAGQCSQNEYFDSLLHACIPCQLPCSSNTPPLTCQR |
| | | YCNASVTNSVKGTNAILWTCLGLSLIISLAVFVLMFLLR |
| | | KISSEPLEDEFKNTGSGLLGMANIDLEKSRTGDEIILPR |
| | | GLEYTVEECTCEDCIKSKPKVDSDHCFPLPAMEEGATIL |
| | | VTTKTNDYCKSLPAALSATEIEKSISARTRTRPLEQKLI |
| | | SEEDLAANDILDYKDDDDKV |
| 204 | huBCMA S30del | MLQMAGQCSQNEYFDSLLHACIPCQLRCSNTPPLTCQR |
| | | YCNASVTNSVKGTNAILWTCLGLSLIISLAVFVLMFLLR |
| | | KISSEPLEDEFKNTGSGLLGMANIDLEKSRTGDEIILPR |
| | | GLEYTVEECTCEDCIKSKPKVDSDHCFPLPAMEEGATIL |
| | | VTTKTNDYCKSLPAALSATEIEKSISARTRTRPLEQKLI |
| | | SEEDLAANDILDYKDDDDKV |
| 205 | huBCMA P33S | MLQMAGQCSQNEYFDSLLHACIPCQLRCSSNTSPLTCQR |
| | | YCNASVTNSVKGTNAILWTCLGLSLIISLAVFVLMELLR |
| | | KISSEPLEDEFKNTGSGLLGMANIDLEKSRTGDEIILPR |
| | | GLEYTVEECTCEDCIKSKPKVDSDHCFPLPAMEEGATIL |
| | | VTTKTNDYCKSLPAALSATEIEKSISARTRTRPLEQKLI |
| | | SEEDLAANDILDYKDDDDKV |
| 206 | huBCMA P34del | MLQMAGQCSQNEYFDSLLHACIPCQLRCSSNTPLTCQR |
| | | YCNASVTNSVKGTNAILWTCLGLSLIISLAVFVLMFLLR |
| | | KISSEPLEDEFKNTGSGLLGMANIDLEKSRTGDEIILPR |
| | | GLEYTVEECTCEDCIKSKPKVDSDHCFPLPAMEEGATIL |
| | | VTTKTNDYCKSLPAALSATEIEKSISARTRTRPLEQKLI |
| | | SEEDLAANDILDYKDDDDKV |
| 207 | BCMA (E04_VH1a) VH | QVQLVQSGAEVKKPGASVKVSCKASGYTFTNYWMHWVRQ |
| | CH1(EE)-Fchole | APGQGLEWMGIIHPNSGSTNYNEKFQGRVTMTRDTSTST |
| | PGLALA(P1AG7400) | VYMELSSLRSEDTAVYYCARGIYDYPFAYWGQGTLVTVS |
| | | SASTKGPSVFPLAPSSKSTSGGTAALGCLVEDYFPEPVT |
| | | VSWNSGALTSGVHTFPAVLQSSGLYSLSSVVTVPSSSLG |
| | | TQTYICNVNHKPSNTKVDEKVEPKSCDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALGAPIEKTISKAKGQPREPQVCT |
| | | LPPSRDELTKNQVSLSCAVKGFYPSDIAVEWESNGQPEN |
| | | NYKTTPPVLDSDGSFFLVSKLTVDKSRWQQGNVFSCSVM |
| | | HEALHNHYTQKSLSLSPG |
| 208 | Dimeric 4-1BB ligand (71- | REGPELSPDDPAGLLDLRQGMFAQLVAQNVLLIDGPLSW |
| | 248)-CL Fc knob chain | YSDPGLAGVSLTGGLSYKEDTKELVVAKAGVYYVFFQLE |
| | (P1AG7400) | LRRVVAGEGSGSVSLALHLQPLRSAAGAAALALTVDLPP |
| | | ASSEARNSAFGFQGRLLHLSAGQRLGVHLHTEARARHAW |
| | | QLTQGATVLGLFRVTPEIPAGLGGGGSGGGGREGPELSP |
| | | DDPAGLLDLRQGMFAQLVAQNVLLIDGPLSWYSDPGLAG |
| | | VSLTGGLSYKEDTKELVVAKAGVYYVFFQLELRRVVAGE |
| | | GSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGLGGGGSGGGGRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGL |
| | | SSPVTKSFNRGECDKTHTCPPCPAPEAAGGPSVFLFPPK |
| | | PKDTLMISRTPEVTCVVVDVSHEDPEVKFNWYVDGVEVH |
| | | NAKTKPREEQYNSTYRVVSVLTVLHQDWLNGKEYKCKVS |
| | | NKALGAPIEKTISKAKGQPREPQVYTLPPCRDELTKNQV |
| | | SLWCLVKGFYPSDIAVEWESNGQPENNYKTTPPVLDSDG |
| | | SFFLYSKLTVDKSRWQQGNVFSCSVMHEALHNHYTQKSL |
| | | SLSPG |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, MD (1991)) as defined above.

The following numbered paragraphs (paras) describe aspects of the present invention:

1. An immunostimulatory antigen binding molecule that specifically binds to B cell maturation agent (BCMA), comprising
(a) an antigen binding domain comprising
(i) a heavy chain variable region (VH BCMA) comprising a heavy chain complementary determining region CDR-H1 of SEQ ID NO:1 (GYTFTNYWMH), a CDR-H2 of SEQ ID NO:2 (IIHPNSGSTNYNEKFQG), and a CDR-H3 of SEQ ID NO:3 (GIYDYPFAY), and
(ii) a light chain variable region (VL BCMA) selected from the group consisting of
(a) a VL comprising a light chain complementary determining region CDR-L1 of SEQ ID NO:4 (RASESVSIHGTHLMH), a CDR-L2 of SEQ ID NO:5 (AASSLQS) and a CDR-L3 of SEQ ID NO:6 (QQSIEDPYT); and
(b) a VL comprising a light chain complementary determining region CDR-L1 of SEQ ID NO:4 (RASESVSIHGTHLMH), a CDR-L2 of SEQ ID NO:7 (AASNLES) and a CDR-L3 of SEQ ID NO:6 (QQSIEDPYT), and
(c) a VL comprising a light chain complementary determining region CDR-L1 of SEQ ID NO:4 (RASESVSIHGTHLMH), a CDR-L2 of SEQ ID NO:8 (AASNLQS) and a CDR-L3 of SEQ ID NO:6 (QQSIEDPYT),
(b) a first and a second polypeptide that are linked to each other by a disulfide bond,
wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) an Fc domain composed of a first and a second subunit.

2. The immunostimulatory antigen binding molecule of para 1, wherein the ectodomain of 4-1BBL or a fragment thereof comprises the amino acid sequence of SEQ ID NO:9 or SEQ ID NO:10.

3. The immunostimulatory antigen binding molecule of paras 1 or 2, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10.

4. The immunostimulatory antigen binding molecule of any one of paras 1 to 3, wherein the antigen binding domain comprises
(a) a VH BCMA comprising an amino acid sequence of SEQ ID NO:13 and VL BCMA comprising an amino acid sequence of SEQ ID NO:15, or
(b) a VH BCMA comprising an amino acid sequence of SEQ ID NO:13 and VL BCMA comprising an amino acid sequence of SEQ ID NO:16.

5. The immunostimulatory antigen binding molecule of any one of paras 1 to 4, wherein the antigen binding domain is a Fab molecule.

6. The immunostimulatory antigen binding molecule of any one of paras 1 to 5, wherein the Fc domain composed of a first and a second subunit is an IgG, particularly an IgG1 Fc domain.

7. The immunostimulatory antigen binding molecule of any one of paras 1 to 6, wherein the Fc domain is a human Fc domain.

8. The immunostimulatory antigen binding molecule of any one of paras 1 to 7, wherein the Fc comprises a modification promoting the association of the first and the second subunit of the Fc domain.

9. The immunostimulatory antigen binding molecule of para 8, wherein the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

10. The immunostimulatory antigen binding molecule of any one of paras 1 to 9, wherein the Fc domain comprises one or more amino acid substitution that reduces binding to an Fc receptor and/or effector function.

11. The immunostimulatory antigen binding molecule of any one of paras 1 to 10, wherein the Fc domain is of human IgG1 subclass and comprises the amino acid mutations L234A, L235A and P329G (numbering according to Kabat EU index).

12. The immunostimulatory antigen binding molecule of any one of paras 1 to 11, comprising
(a) a first polypeptide, comprising (ai) a first ectodomain of 4-1BBL or fragment thereof, fused at its C-terminus to the N-terminus of the second ectodomain of 4-1BBL or fragment thereof, (aii) a second ectodomain of 4-1BBL or fragment thereof, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL or fragment thereof, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
(d) a fourth polypeptide, comprising the light chain of the Fab molecule.

13. The immunostimulatory antigen binding molecule of para 12, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

14. The immunostimulatory antigen binding molecule of any one of claims 1 to 13, comprising
(a) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:24, or
(b) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:25.

15. An immunostimulatory antigen binding molecule that specifically binds to B cell maturation agent (BCMA), comprising
(a) an antigen binding domain comprising
(i) a heavy chain variable region (VL BCMA) selected from the group consisting of
(a) a VH comprising a heavy chain complementary determining region CDR-H1 of SEQ ID NO: 26 (GFTFSNAWMD), a CDR-H2 of SEQ ID NO: 27 (QITAKSNNYATYYADSVKG), and a CDR-H3 of SEQ ID NO: 28 (DGYH); and
(b) a VH comprising a heavy chain complementary determining region CDR-H1 of SEQ ID NO:26 (GFTFSNAWMD), a CDR-H2 of SEQ ID NO: 29 (QITAKSNNYATYYAAPVKG), and a CDR-H3 of SEQ ID NO: 28 (DGYH); and
(ii) a light chain variable region (VL BCMA) comprising a light chain complementary determining region CDR-L1 of SEQ ID NO: 30 (RASEDIRNGLA), a CDR-L2 of SEQ ID NO: 31 (NANSLHT) and a CDR-L3 of SEQ ID NO: 32 (EDTSKYPYT),
(b) a first and a second polypeptide that are linked to each other by a disulfide bond,
wherein the antigen binding molecule is characterized in that the first polypeptide comprises two ectodomains of 4-1BBL or a fragment thereof that are connected to each other by a peptide linker and in that the second polypeptide comprises one ectodomain of 4-1BBL or a fragment thereof, and
(c) an Fc domain composed of a first and a second subunit.

16. The immunostimulatory antigen binding molecule of para 15, wherein the ectodomain of 4-1BBL or a fragment thereof comprises the amino acid sequence of SEQ ID NO:9 or SEQ ID NO:10.

17. The immunostimulatory antigen binding molecule of paras 15 or 16, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10.

18. The immunostimulatory antigen binding molecule of any one of paras 15 to 17, wherein the antigen binding domain comprises
(a) a VH BCMA comprising an amino acid sequence of SEQ ID NO:33 (VH2a) and VL BCMA comprising an amino acid sequence of SEQ ID NO:34 (VL2a), or
(b) a VH BCMA comprising an amino acid sequence of SEQ ID NO:35 (VH1b) and VL BCMA comprising an amino acid sequence of SEQ ID NO:36 (VL1a).

19. The immunostimulatory antigen binding molecule of any one of paras 15 to 18, wherein the antigen binding domain is a Fab molecule.

20. The immunostimulatory antigen binding molecule of any one of paras 15 to 19, wherein the Fc domain composed of a first and a second subunit is an IgG, particularly an IgG1 Fc domain.

21. The immunostimulatory antigen binding molecule of any one of paras 15 to 20, wherein the Fc domain is a human Fc domain.

22. The immunostimulatory antigen binding molecule of any one of paras 15 to 21, wherein the Fc comprises a modification promoting the association of the first and the second subunit of the Fc domain.

23. The immunostimulatory antigen binding molecule of para 22, wherein the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

24. The immunostimulatory antigen binding molecule of any one of paras 15 to 23, wherein the Fc domain comprises one or more amino acid substitution that reduces binding to an Fc receptor and/or effector function.

25. The immunostimulatory antigen binding molecule of any one of paras 15 to 24, wherein the Fc domain is of human IgG1 subclass and comprises the amino acid mutations L234A, L235A and P329G (numbering according to Kabat EU index).

26. The immunostimulatory antigen binding molecule of any one of paras 15 to 25, comprising
(a) a first polypeptide, comprising (ai) a first ectodomain of 4-1BBL or fragment thereof, fused at its C-terminus to the N-terminus of the second ectodomain of 4-1BBL or fragment thereof, (aii) a second ectodomain of 4-1BBL or fragment thereof, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL or fragment thereof, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
(d) a fourth polypeptide, comprising the light chain of the Fab molecule.

27. The immunostimulatory antigen binding molecule of para 26, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

28. The immunostimulatory antigen binding molecule of any one of paras 15 to 27, comprising
(a) a first polypeptide comprising the amino acid sequence of SEQ ID NO: 21, a second polypeptide comprising the amino acid sequence of SEQ ID NO:22, a third polypeptide comprising the amino acid sequence of SEQ ID NO:37 and a fourth polypeptide comprising the amino acid sequence of SEQ ID NO:38, or
(b) a first polypeptide comprising the amino acid sequence of SEQ ID NO: 21, a second polypeptide comprising the amino acid sequence of SEQ ID NO:22, a third polypeptide comprising the amino acid sequence of SEQ ID NO:39 and a fourth polypeptide comprising the amino acid sequence of SEQ ID NO:40.

29. One or more isolated polynucleotide encoding the immunostimulatory antigen binding molecule of any one of claims 1 to 28.

30. One or more vector, particularly expression vector, comprising the polynucleotide(s) of claim 29.

31. A host cell comprising the polynucleotide(s) of para 29 or the vector(s) of para 30.

32. A method of producing an immunostimulatory antigen binding molecule that specifically binds to BCMA, comprising the steps of a) culturing the host cell of para 31 under conditions suitable for the expression of the immunostimulatory antigen binding molecule and optionally b) recovering the immunostimulatory antigen binding molecule.

33. An immunostimulatory antigen binding molecule that specifically binds to BCMA produced by the method of para 32.

34. A pharmaceutical composition comprising the immunostimulatory antigen binding molecule of any one of paras 1 to 28 or 33 and at least one pharmaceutically acceptable excipient.

35. The immunostimulatory antigen binding molecule of any one of paras 1 to 28 or 33, or the pharmaceutical composition of para 34, for use as a medicament.

36. The immunostimulatory antigen binding molecule of any one of paras 1 to 28 or 33, or the pharmaceutical composition of para 34, for use in enhancing (a) T cell activation or (b) T cell effector functions.

37. The immunostimulatory antigen binding molecule of any one of paras 1 to 28 or 33, or the pharmaceutical composition of para 34, for use in the treatment of a disease.

38. The immunostimulatory antigen binding molecule or the pharmaceutical composition for use of para 37, wherein the disease is cancer.

39. The immunostimulatory antigen binding molecule of any one of paras 1 to 28 or 33, or the pharmaceutical composition of para 34, for use in the treatment of cancer, wherein the use is for administration in combination with a chemotherapeutic agent, radiation therapy and/or other agents for use in cancer immunotherapy.

40. The immunostimulatory antigen binding molecule of any one of paras 1 to 28 or 33, or the pharmaceutical composition of para 34, for use in the treatment of cancer, wherein the use is for administration in combination with a T-cell activating anti-CD3 bispecific antibody.

41. The immunostimulatory antigen binding molecule or the pharmaceutical composition for use of para 40, wherein the T-cell activating anti-CD3 bispecific antibody is an anti-GPRC5D/anti-CD3 antibody.

42. Use of the immunostimulatory antigen binding molecule of any one of paras 1 to 28 or 33, or the pharmaceutical composition of para 34, in the manufacture of a medicament for the treatment of a disease, particularly for the treatment of cancer.

43. A method of treating a disease, particularly cancer, in an individual, comprising administering to said individual an effective amount of the immunostimulatory antigen binding molecule of paras 1 to 28 or 33, or the pharmaceutical composition of claim 34.

44. The method of para 43, further comprising administration in combination with a chemotherapeutic agent, radiation therapy and/or other agents for use in cancer immunotherapy, particularly in combination with a T-cell activating anti-CD3 bispecific antibody.

### EXAMPLES

The following are examples of methods and compositions of the invention. It is understood that various other aspects may be practiced, given the general description provided above.

Recombinant DNA techniques: Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA sequencing: DNA sequences were determined by double strand sequencing.

Gene synthesis: Desired gene segments, where required, were either generated by PCR using appropriate templates or were synthesized at Geneart AG (Regensburg, Germany) or Genscript (New Jersey, USA) from synthetic oligonucleotides and PCR products by automated gene synthesis. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow subcloning into the respective expression vectors. All constructs were designed with a 5′-end DNA sequence coding for a leader peptide which targets proteins for secretion in eukaryotic cells.

Production of IgG and bispecific antibodies: The DNA sequences encoding the variable heavy and light chain regions of the BCMA antibodies were cloned into mammalian expression vectors using conventional cloning techniques. The antibodies described herein were produced using shaking flasks with FedBatch mode. The recombinant production was performed by transient transfection of Expi293™ Cells in a defined, serum-free medium. For transfection ExpiFectamine™ 293 Transfection Kit was used (Gibco). Cell culture supernatants were harvested 7-12 days after transfection.

Quantification of protein titer: The protein titer of supernatant samples was determined by affinity chromatography using a POROS A 20 μm column, 2.1×30 mm (Life Technologies, Carlsbad, CA, USA) on a High Performance Liquid Chromatography system (Ultimate 3000 HPLC system, Thermo Scientific, Waltham, MA, USA). The supernatant was loaded onto the column equilibrated with 0.2 M Na₂HPO₄, pH 7.4, followed by elution with 0.1 M citric acid, 0.2 M NaCl, pH 2.5. Titers were quantified by measuring absorption at 280 nm, and subsequently calculating the protein concentration by comparing the elution peak area (under the curve) of the analyte with a reference standard curve.

Purification of IgG and bispecific antibodies: Proteins were purified from cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5 or PBS; elution buffer: 20 mM, 25 mM or 50 mM sodium citrate, pH 3.0). Elution was achieved at pH 3.0 followed by immediate pH neutralization of the sample. The protein was concentrated by centrifugation (Millipore Amicon® ULTRA-15, #UFC903096), and aggregated protein was separated from monomeric protein by size exclusion chromatography in 20 mM histidine, 140 mM sodium chloride, pH 6.0

Analytics of IgG and bispecific antibodies: The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer). Determination of the aggregate content was performed by HPLC chromatography at 25° C. using analytical size-exclusion column (TSKgel G3000 SW XL or UP-SW3000, Tosoh Bioscience) equilibrated in running buffer (200 mM KH₂PO₄, 250 mM KCl pH 6.2, 0.02% NaN₃). The final quality was good for all molecules with varying monomer content from around 70% to almost 100% monomer content and >90% purity on CE-SDS. In conclusion, all IgGs and bispecific antibodies were produced in good quality.

Mass spectrometry: This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs. The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

Determination of binding and binding affinity of multispecific antibodies to the respective antigens using surface plasmon resonance (SPR) (BIACORE): Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Generation and Production of Optimal Anti-BCMA Antibodies

### 1.1 Generation of Humanized Variants of Anti-BCMA Antibody E04

### 1.1.1 Methodology

Anti-BCMA antibody E04 is disclosed in WO 2012/163805 and has the VH domain of SEQ ID NO:41 and the VL domain of SEQ ID NO:42. Optimized variants thereof were created as described in the following. For the identification of a suitable human acceptor framework during the humanization, a combination of two methodologies was used. On the one hand, a classical approach was taken by querying a BLASTp database of human V- and J-region sequences for the murine input sequences (cropped to the variable part). Selective criteria for the choice of human acceptor framework were sequence homology, same or similar CDR lengths, and the estimated frequency of the human germline, but also the conservation of certain amino acids at the VH-VL domain interface. Following the germline identification step, the CDRs of the murine input sequences were grafted onto the human acceptor framework regions. Each amino acid difference between these initial CDR grafts and the parental antibody was rated for possible impact on the structural integrity of the respective variable region, and “back mutations” towards the parental sequence were introduced whenever deemed appropriate. The structural assessment was based on Fv region homology models of both the parental antibody and the humanization variants, created with an in-house antibody structure homology modeling protocol implemented using the BIOVIA Discovery Studio Environment, version 17R2. In some humanization variants, “forward mutations” were included, i.e., amino acid exchanges that change the original amino acid occurring at a given CDR position of the parental binder to the amino acid found at the equivalent position of the human acceptor germline. The aim is to increase the overall human character of the humanization variants (beyond the framework regions) to further reduce the immunogenicity risk.

On the other hand, an in silico tool developed in-house was used to predict the VH-VL domain orientation of the paired VH and VL humanization variants (WO 2016/062734). The results were compared to the predicted VH-VL domain orientation of the parental binder to select for framework combinations which are close in geometry to the original antibody. The rational is to detect possible amino acid exchanges in the VH-VL interface region that might lead to disruptive changes in the pairing of the two domains that in turn might have detrimental effects on the binding properties.

### 1.1.2 Choice of Acceptor Framework and Adaptations Thereof

The acceptor framework was chosen as described in Table 1 below:

TABLE 1
Acceptor framework

| | Murine (mus | Choice of |
|---|---|---|
| | musculus) | human acceptor |
| | V-region | V-region |
| | germline | germline |
| VH1ab | IGHV1-64*01 | IGHV1-46*01 |
| VL1abcdef | IGKV3-3*01 | IGKV1-39*01 |

Post-CDR3 framework regions were adapted from human IGHJ germline IGHJ4*01 (YFDYWGQGTLVTVSS, SEQ ID NO: 41) and human IGKJ germline IGKJ4*01 (LTFGGGTKVEIK, SEQ ID NO: 42). The part relevant for the acceptor framework is indicated as underlined.

Based on structural considerations, back mutations from the human acceptor framework to the amino acid in the parental clone were introduced at certain positions of the E04 humanization variants. Furthermore, some positions were identified as promising candidates for forward mutations, where the amino acid in a CDR of the parental binder is substituted by the amino acid found in the human acceptor germline. The changes are detailed in Table 2 below.

TABLE 2
List of Variants

| | | Identity to human V-region |
|---|---|---|
| Variant Name | Mutations | germline (BLASTp) |
| VH1a | fK64Q, fS65G | 91.8 |
| VH1b | fK64Q, fS65G, bR71V, bT73K | 89.8 |
| VL1a | — | 84.8 |
| VL1b | bG68E | 83.8 |
| VL1c | fE55Q | 85.9 |
| VL1d | fE55Q, bG68E | 84.8 |
| VL1e | fN53S, fE55Q | 86.9 |
| VL1f | fN53S, fE55Q, bG68E | 85.9 |

Note:
Back mutations are prefixed with b, forward mutations with f, e.g., bS49A refers to a back mutation (human germline amino acid to parental antibody amino acid) from serine to alanine at position 49. All residue indices are given in Kabat numbering.

### 1.1.3 T-Cell Epitope Prediction

To assess the occurrence of potential T-cell epitopes in the humanized sequences, the NetMHCIIpan 4.0 predictor (Reynisson B et. al: NetMHCpan-4.1 and NetMHCIIpan-4.0: improved predictions of MHC antigen presentation by concurrent motif deconvolution and integration of MS MHC eluted ligand data, Nucl. Acids Res., 48(W1): W449-W454 (2020)) was employed. The predictions were made for the following human MHC class II alleles: DRB1*01:01, DRB1*03:01, DRB1*04:01, DRB1*07:01, DRB1*08:01, DRB1*09:01, DRB1*11:01, DRB1*13:01, and DRB1*15:01.

The thresholds for strong and weak binding 15mer peptides were set to a percentile rank of 1 and 5, respectively. Binding 15mer peptides with a percentile rank above 5 were not considered. Likewise, all binding 15mer peptides with a 9mer core peptide occurring in 10 or more human V-region germlines were not considered. The germline sequences were obtained from the IMGT database (Giudicelli, V. et al.: IMGT/LIGM-DB, the IMGT® comprehensive database of immunoglobulin and T cell receptor nucleotide sequences. Nucl. Acids Res., 34(S1): D781-D784 (2006)). As many of the predicted 15mer binders are sharing the same 9mer core peptide, Table 3 below also details the number of unique 9mer cores present in the respective sequence and predicted to bind in the percentile rank range ≤5.

TABLE 3
T cell epitopes

| | #Strong | #Weak | #Total | #Unique |
|---|---|---|---|---|
| | binding | binding | binding | 9mer |
| | 15mer | 15mer | 15mer | peptide |
| Sequence | peptides | peptides | peptides | cores |
| E04_VH_parental | 6 | 53 | 59 | 9 |
| VH1a | 0 | 12 | 12 | 5 |
| VH1b | 0 | 17 | 17 | 7 |
| E04_VL_parental | 10 | 37 | 47 | 8 |
| VL1a | 10 | 29 | 39 | 7 |
| VL1b | 10 | 29 | 39 | 7 |
| VL1c | 11 | 28 | 39 | 7 |
| VL1d | 11 | 28 | 39 | 7 |
| VL1e | 8 | 21 | 29 | 5 |
| VL1f | 8 | 21 | 29 | 5 |

### 1.1.4 VH and VL Domains of the Resulting Humanized BCMA Antibodies

The resulting VH domains of the humanized BCMA antibodies can be found in Table 4 below and the resulting VL domains of the humanized BCMA antibodies are listed in Table 5 below.

TABLE 4
Amino acid sequences of the
VH domains of humanized BCMA antibodies

| | | Seq ID |
|---|---|---|
| Description | Sequence | No |
| BCMA | QVQLVQSGAEVKKPGASVKVSCKAS | 13 |
| (E04_VH1a) | GYTFTNYWMHWVRQAPGQGLEWMGI | |
| | IHPNSGSTNYNEKFQGRVTMTRDTS | |
| | TSTVYMELSSLRSEDTAVYYCARGI | |
| | YDYPFAYWGQGTLVTVSS | |
| BCMA | QVQLVQSGAEVKKPGASVKVSCKAS | 14 |
| (E04_VH1b) | GYTFTNYWMHWVRQAPGQGLEWMGI | |
| | IHPNSGSTNYNEKFQGRVTMTVDKS | |
| | TSTVYMELSSLRSEDTAVYYCARGI | |
| | YDYPFAYWGQGTLVTVSS | |

TABLE 5
Amino acid sequences of the
VL domains of humanized BCMA antibodies

| | | Seq ID |
|---|---|---|
| Description | Sequence | No |
| BCMA | DIQMTQSPSSLSASVGDRVTITCRA | 16 |
| (E04_VL1a) | SESVSIHGTHLMHWYQQKPGKAPKL | |
| | LIYAASNLESGVPSRFSGSGSGTDF | |
| | TLTISSLQPEDFATYYCQQSIEDPY | |
| | TFGGGTKVEIK | |
| BCMA | DIQMTQSPSSLSASVGDRVTITCRA | 17 |
| (E04_VL1b) | SESVSIHGTHLMHWYQQKPGKAPKL | |
| | LIYAASNLESGVPSRFSGSGSETDF | |
| | TLTISSLQPEDFATYYCQQSIEDPY | |
| | TFGGGTKVEIK | |
| BCMA | DIQMTQSPSSLSASVGDRVTITCRA | 18 |
| (E04_VL1c) | SESVSIHGTHLMHWYQQKPGKAPKL | |
| | LIYAASNLQSGVPSRFSGSGSGTDF | |
| | TLTISSLQPEDFATYYCQQSIEDPY | |
| | TFGGGTKVEIK | |
| BCMA | DIQMTQSPSSLSASVGDRVTITCRA | 19 |
| (E04_VL1d) | SESVSIHGTHLMHWYQQKPGKAPKL | |
| | LIYAASNLQSGVPSRFSGSGSETDF | |
| | TLTISSLQPEDFATYYCQQSIEDPY | |
| | TFGGGTKVEIK | |
| BCMA | DIQMTQSPSSLSASVGDRVTITCRA | 20 |
| (E04_VL1e) | SESVSIHGTHLMHWYQQKPGKAPKL | |
| | LIYAASSLQSGVPSRFSGSGSGTDF | |
| | TLTISSLQPEDFATYYCQQSIEDPY | |
| | TFGGGTKVEIK | |
| BCMA | DIQMTQSPSSLSASVGDRVTITCRA | 15 |
| (E04_VL1f) | SESVSIHGTHLMHWYQQKPGKAPKL | |
| | LIYAASSLQSGVPSRFSGSGSETDF | |
| | TLTISSLQPEDFATYYCQQSIEDPY | |
| | TFGGGTKVEIK | |
| BCMA | DIQMTQSPSSLSASVGDRVTITCRA | 130 |
| (E04_VL1g) | SESVSIHGTHLMHWYQQKPGKAPKL | |
| | LIYAASNLESGVPSRFSGSGSGTDF | |
| | TLTISSLQPEDFATYFCQQSIEDPY | |
| | TFGGGTKVEIK | |
| BCMA | DIQMTQSPSSLSASVGDRVTITCRA | 131 |
| (E04_VL1h) | SESVSIHGTHLMHWYQQKPGKAPKL | |
| | LIYAASNLESGVPSRFSGSGSETDF | |
| | TLTISSLQPEDFATYFCQQSIEDPY | |
| | TFGGGTKVEIK | |

The humanized amino acid sequences for heavy and light chain variable domains of E04 humanized variants were fused to a one-armed human IgG1 backbone/human CH1-Hinge-CH2-CH3 with an effector silent Fc domain (P329G; L234A, L235A) to abrogate binding to Fcγ receptors according to the method described in WO 2012/130831 A1 and into a light chain. For the right assembly of the one-armed IgG1 a human Fc containing an effector silent Fc domain was used. The amino acid sequences were backtranslated in to DNA and the resulting cDNA were synthesized (GeneArt or Twist Biosciences) and then cloned into heavy chain expression vectors as fusion proteins with human IgG1 backbones expression vectors as fusion proteins to human C-kappa. Light chain (LC) and heavy chain (HC) plasmids were then co-transfected into HEK293 cells and purified after 7 days from supernatants by standard methods for antibody purification.

### 1.2 Generation of Humanized Variants of Anti-BCMA Antibody 54

### 1.2.1 Methodology

Anti-BCMA antibody 54 is disclosed in WO 2013/072415 and has the VH domain of SEQ ID NO:47 and the VL domain of SEQ ID NO:48. BCMA-54 is a humanized antibody with 84.8% identity to the most similar human HV germline (IGHV3-15*01) and 83.2% identity to the most similar human KV germline (IGKV1-6*01). Even though the variable region of BCMA-54 is based on framework regions of human origin, there are several positions in these regions which are not corresponding to the human germline amino acid. Examples for this include the positions VH-16 (Ala), VH-44 (Arg), VH-84 (Lys), VL-22 (Ala), VL-83 (Glu), and VL-95 (Ile). Furthermore, there exists the possibility to humanize parts of the CDRs to decrease the immunogenic potential of this binder and reduce the number of potential T-cell epitopes.

To realize this potential for optimization, suitable human acceptor frameworks were identified by querying a BLASTp database of human V- and J-region sequences for the original BCMA-54 sequences. Selective criteria for the choice of human acceptor framework were sequence homology, same or similar CDR lengths, and the estimated frequency of the human germline, but also the conservation of certain amino acids at the VH-VL domain interface. Following the germline identification step, the CDRs of the BCMA-54 input sequences were grafted onto the human acceptor framework regions. Each amino acid difference between these initial CDR grafts and the parental antibodies was rated for possible impact on the structural integrity of the respective variable region, and “back mutations” towards the parental sequence were introduced whenever deemed appropriate. The structural assessment was based on Fv region homology models of both BCMA-54 and the optimization candidate variants, created with an in-house antibody structure homology modeling protocol implemented using the BIOVIA Discovery Studio Environment, version 17R2. In some humanization variants, “forward mutations” were included, i.e., amino acid exchanges that change the original amino acid occurring at a given CDR position of the parental binder to the amino acid found at the equivalent position of the human acceptor germline.

An in silico tool developed in-house was used to predict the VH-VL domain orientation of the paired VH and VL humanization variants (WO 2016/062734). The results were compared to the predicted VH-VL domain orientation of the parental binder to select for framework combinations which are close in geometry to the original antibody. The rationale is to detect possible amino acid exchanges in the VH-VL interface region that might lead to disruptive changes in the pairing of the two domains that in turn might have detrimental effects on the binding properties.

### 1.2.2 Choice of Acceptor Framework and Adaptations Thereof

The following acceptor frameworks were chosen as described in Table 6 below:

TABLE 6
Acceptor framework

| | V-region | Choice of human |
|---|---|---|
| | germline in | acceptor V-region |
| | BCMA-54 | germline |
| VH1ab | IGHV3-15*01 | IGHV3-15*01 |
| VH2a | | IGHV3-23*01 |
| VL1a | IGKV1-6*01 | IGKV1-6*01 |
| VL2a | | IGKV1-39*01 |

Post-CDR3 framework regions were adapted from human IGHJ germline IGHJ1*01 (AEYFQHWGQGTLVTVSS, SEQ ID NO: 49) and human IGKJ germline IGKJ2*01 (YTFGQGTKLEIK, SEQ ID NO: 50). The part relevant for the acceptor framework is indicated as underlined.

Based on structural considerations, back mutations from the human acceptor framework to the amino acid in the original BCMA-54 sequence were introduced at certain positions of the optimization variants. Furthermore, some positions were identified as promising candidates for forward mutations, wherein the amino acid in a CDR of the parental binder is substituted by the amino acid found in the human acceptor germline. On top of the basis variants (VH1a, VH1b, VH2a, VL1a and VL2a) additional sequence variants were defined which typically introduce additional forward mutations (“germlining”), either of individual positions or stretches of the respective basis sequence. One variant (VH1a_W197Y) aimed at ameliorating a predicted hydrophobic surface patch, thereby potentially improving the biophysical properties of the VH region. The changes are detailed in Table 7 below.

TABLE 7
List of Variants

| | | Identity to |
|---|---|---|
| Variant | | human V-region |
| Name | Mutations | germline (BLASTp) |
| VH1a | bT94D | 89.9 |
| VH1b | bV48I, bG49A, bT94D | 87.9 |
| VH2a | fA61D, fP62S, bA93T, bK94D | 87.8 |
| VH1a_Y292D | bT94D, fY58D | 90.9 |
| VH1c_huCDR2 | bT94D, and CDR-H2 germlined | 99.0 |
| | to RIKSKTDGGTTDYAAPVKG | |
| | (SEQ ID NO: 51) (IGHV3-15*01) | |
| VH1a_W197Y | bT94D, fW33Y | 88.9 |
| VL1a | | 86.3 |
| VL2a | | 86.2 |
| VL1a_L2_GL | CDR-L2 germlined to AASSLQS | 90.5 |
| | (SEQ ID NO: 52) (IGKV1-6*01) | |
| VL2a_L2_GL | CDR-L2 germlined to AASSLQS | 90.4 |
| | (SEQ ID NO: 52) (IGKV1-39*01) | |
| VL1a_L1_pGL | fG32D, fA34G | 88.4 |
| VL1a_N651A | fN40A | 87.4 |
| VL1a_N651A_ | fN40A, fN52S | 88.4 |
| N695S | | |
| VL1a_H698Q | fH55Q | 87.4 |
| VL1a_T699S | fT56S | 87.4 |
| VL1a_H698Q_ | fH55Q, fT56S | 88.4 |
| T699S | | |

Note: Back mutations are prefixed with b, forward mutations with f, e.g., bS49A refers to a back mutation (human germline amino acid to parental antibody amino acid) from serine to alanine at position 49. All residue indices are given in Kabat numbering.

### 1.2.3 T-cell Epitope Prediction

To assess the occurrence of potential T-cell epitopes in the humanized sequences, the NetMHCIIpan 4.0 predictor (Reynisson B et. al: NetMHCpan-4.1 and NetMHCIIpan-4.0: improved predictions of MHC antigen presentation by concurrent motif deconvolution and integration of MS MHC eluted ligand data, Nucl. Acids Res., 48 (W1): W449-W454 (2020)) was employed. The predictions were made for the following human MHC class II alleles: DRB1*01:01, DRB1*03:01, DRB1*04:01, DRB1*07:01, DRB1*08:01, DRB1*09:01, DRB1*11:01, DRB1*13:01, and DRB1*15:01.

The thresholds for strong and weak binding 15mer peptides were set to a percentile rank of 1 and 5, respectively. Binding 15mer peptides with a percentile rank above 5 were not considered. Likewise, all binding 15mer peptides with a 9mer core peptide occurring in 10 or more human V-region germlines were not considered. The germline sequences were obtained from the IMGT database (Giudicelli, V. et al.: IMGT/LIGM-DB, the IMGT® comprehensive database of immunoglobulin and T cell receptor nucleotide sequences. Nucl. Acids Res., 34(S1):D781-D784 (2006)). As many of the predicted 15mer binders are sharing the same 9mer core peptide, Table 8 below also details the number of unique 9mer cores present in the respective sequence and predicted to bind in the percentile rank range ≤5.

TABLE 8
T cell epitopes

| | #Strong | #Weak | #Total | #Unique |
|---|---|---|---|---|
| | binding | binding | binding | 9mer |
| | 15mer | 15mer | 15mer | peptide |
| Sequence | peptides | peptides | peptides | cores |
| 54_VH_parental | 15 | 60 | 75 | 8 |
| VH1a | 15 | 52 | 67 | 6 |
| VH1b | 15 | 53 | 68 | 6 |
| VH2a | 7 | 54 | 61 | 6 |
| VH1a_Y292D | 14 | 54 | 68 | 5 |
| VH1c_huCDR2 | 10 | 24 | 34 | 4 |
| VH1a_W197Y | 14 | 53 | 67 | 6 |
| 54_VL_parental | 12 | 17 | 29 | 5 |
| VL1a | 12 | 15 | 27 | 5 |
| VL2a | 12 | 15 | 27 | 5 |
| VL_a_L2_GL | 9 | 12 | 21 | 4 |
| VL2a_L2_GL | 9 | 12 | 21 | 4 |
| VL1a_L1_pGL | 12 | 23 | 35 | 7 |
| VL1a_N651A | 11 | 23 | 34 | 6 |
| VL1a_N651A_N6 | 9 | 19 | 28 | 6 |
| 95S | | | | |
| VL1a_H698Q | 12 | 16 | 28 | 6 |
| VL1a_T699S | 13 | 16 | 29 | 5 |
| VL1a_H698Q_T6 | 14 | 15 | 29 | 6 |
| 99S | | | | |

### 1.2.4 VH and VL Domains of the Resulting Humanized BCMA Antibodies

The resulting VH domains of the humanized BCMA antibodies can be found in Table 9 below and the resulting VL domains of the humanized BCMA antibodies are listed in Table 10 below.

TABLE 9
Amino acid sequences of the
VH domains of humanized BCMA antibodies

| | | Seq ID |
|---|---|---|
| Description | Sequence | No |
| BCMA | EVQLVESGGGLVKPGGSLRLSCA | 53 |
| (54_VH1a) | ASGFTFSNAWMDWVRQAPGKGLE | |
| | WVGQITAKSNNYATYYAAPVKGR | |
| | FTISRDDSKNTLYLQMNSLKTED | |
| | TAVYYCTDDGYHWGQGTLVTVSS | |
| BCMA | EVQLVESGGGLVKPGGSLRLSCA | 35 |
| (54_VH1b) | ASGFTFSNAWMDWVRQAPGKGLE | |
| | WIAQITAKSNNYATYYAAPVKGR | |
| | FTISRDDSKNTLYLQMNSLKTED | |
| | TAVYYCTDDGYHWGQGTLVTVSS | |
| BCMA | EVQLLESGGGLVQPGGSLRLSCA | 33 |
| (54_VH2a) | ASGFTFSNAWMDWVRQAPGKGLE | |
| | WVSQITAKSNNYATYYADSVKGR | |
| | FTISRDNSKNTLYLQMNSLRAED | |
| | TAVYYCTDDGYHWGQGTLVTVSS | |
| BCMA | EVQLVESGGGLVKPGGSLRLSCA | 54 |
| (54_VH1a_ | ASGFTFSNAWMDWVRQAPGKGLE | |
| Y292D) | WVGQITAKSNNYATDYAAPVKGR | |
| | FTISRDDSKNTLYLQMNSLKTED | |
| | TAVYYCTDDGYHWGQGTLVTVSS | |
| BCMA | EVQLVESGGGLVKPGGSLRLSCA | 55 |
| (54_VH1c_ | ASGFTFSNAWMDWVRQAPGKGLE | |
| huCDR2) | WVGRIKSKTDGGTTDYAAPVKGR | |
| | FTISRDDSKNTLYLQMNSLKTED | |
| | TAVYYCTDDGYHWGQGTLVTVSS | |
| BCMA | EVQLVESGGGLVKPGGSLRLSCA | 56 |
| (54_VH1a_ | ASGFTFSNAYMDWVRQAPGKGLE | |
| W197Y) | WVGQITAKSNNYATYYAAPVKGR | |
| | FTISRDDSKNTLYLQMNSLKTED | |
| | TAVYYCTDDGYHWGQGTLVTVSS | |

TABLE 10
Amino acid sequences of the
VL domains of humanized BCMA antibodies

| | | Seq ID |
|---|---|---|
| Description | Sequence | No |
| BCMA | AIQMTQSPSSLSASVGDRVTITC | 36 |
| (54_VL1a) | RASEDIRNGLAWYQQKPGKAPKL | |
| | LIYNANSLHTGVPSRFSGSGSGT | |
| | DFTLTISSLQPEDFATYYCEDTS | |
| | KYPYTFGQGTKLEIK | |
| BCMA | DIQMTQSPSSLSASVGDRVTITC | 34 |
| (54_VL2a) | RASEDIRNGLAWYQQKPGKAPKL | |
| | LIYNANSLHTGVPSRFSGSGSGT | |
| | DFTLTISSLQPEDFATYYCEDTS | |
| | KYPYTFGQGTKLEIK | |
| BCMA | AIQMTQSPSSLSASVGDRVTITC | 57 |
| (54_VL1a_ | RASEDIRNGLAWYQQKPGKAPKL | |
| L2_GL) | LIYAASSLQSGVPSRFSGSGSGT | |
| | DFTLTISSLQPEDFATYYCEDTS | |
| | KYPYTFGQGTKLEIK | |
| BCMA | DIQMTQSPSSLSASVGDRVTITC | 58 |
| (54_VL2a_ | RASEDIRNGLAWYQQKPGKAPKL | |
| L2_GL) | LIYAASSLQSGVPSRFSGSGSGT | |
| | DFTLTISSLQPEDFATYYCEDTS | |
| | KYPYTFGQGTKLEIK | |
| BCMA | AIQMTQSPSSLSASVGDRVTITC | 59 |
| (54_VL1a_ | RASEDIRNDLGWYQQKPGKAPKL | |
| L1_pGL) | LIYNANSLHTGVPSRFSGSGSGT | |
| | DFTLTISSLQPEDFATYYCEDTS | |
| | KYPYTFGQGTKLEIK | |
| BCMA | AIQMTQSPSSLSASVGDRVTITC | 60 |
| (54_VL1a_ | RASEDIRNGLAWYQQKPGKAPKL | |
| N651A) | LIYAANSLHTGVPSRFSGSGSGT | |
| | DFTLTISSLQPEDFATYYCEDTS | |
| | KYPYTFGQGTKLEIK | |
| BCMA | AIQMTQSPSSLSASVGDRVTITC | 61 |
| (54_VL1a_ | RASEDIRNGLAWYQQKPGKAPKL | |
| N651A_N695S) | LIYAASSLHTGVPSRFSGSGSGT | |
| | DFTLTISSLQPEDFATYYCEDTS | |
| | KYPYTFGQGTKLEIK | |
| BCMA | AIQMTQSPSSLSASVGDRVTITC | 62 |
| (54_VL1a_ | RASEDIRNGLAWYQQKPGKAPKL | |
| H698Q) | LIYNANSLQTGVPSRFSGSGSGT | |
| | DFTLTISSLQPEDFATYYCEDTS | |
| | KYPYTFGQGTKLEIK | |
| BCMA | AIQMTQSPSSLSASVGDRVTITC | 63 |
| (54_VL1a_ | RASEDIRNGLAWYQQKPGKAPKL | |
| T699S) | LIYNANSLHSGVPSRFSGSGSGT | |
| | DFTLTISSLQPEDFATYYCEDTS | |
| | KYPYTFGQGTKLEIK | |
| BCMA | AIQMTQSPSSLSASVGDRVTITC | 64 |
| (54_VL1a_ | RASEDIRNGLAWYQQKPGKAPKL | |
| H698Q_T699S) | LIYNANSLQSGVPSRFSGSGSGT | |
| | DFTLTISSLQPEDFATYYCEDTS | |
| | KYPYTFGQGTKLEIK | |

The humanized amino acid sequences for heavy and light chain variable domains of E04 humanized variants were fused to a one-armed human IgG1 backbone/human CH1-Hinge-CH2-CH3 with an effector silent Fc domain (P329G; L234A, L235A) to abrogate binding to Fcγ receptors according to the method described in WO 2012/130831 A1 and containing a knob mutation according to Knobs-into-Holes technology and into a light chain. For the right assembly of the one-armed IgG1 a human Fc containing an effector silent Fc domain was used. The amino acid sequences were backtranslated into DNA and the resulting cDNA were synthesized (GeneArt or Twist Biosciences) and then cloned into heavy chain expression vectors as fusion proteins with human IgG1 backbones expression vectors as fusion proteins to human C-kappa. Light chain (LC) and heavy chain (HC) plasmids were then co-transfected into HEK293 cells and purified after 7 days from supernatants by standard methods for antibody purification.

### 1.3 Characterization of the Humanized Anti-BCMA Variants

In order to characterize the humanized anti-BCMA antibody variants, all clones were expressed as monovalent one-armed IgG-like constructs (FIG. 1A). This format was chosen in order to characterize the binding to BCMA in a 1:1 model.

With the intention of selecting two preferred humanized variants of anti-BCMA antibody E04 and two preferred humanized variants of anti-BCMA antibody 54, 76 variants were produced. From these 76 variants, the 29 variants (15 BCMA 54 variants and 14 BCMA E04 variants) with the highest affinity to huBCMA were pre-selected and further characterized by measuring affinity to cyBCMA.

The corresponding VH/VL pairs, the construct ID (TaPIR ID) and the corresponding SEQ ID NO:s of the 29 variants and the parental antibodies are listed in Table 11 below.

TABLE 11
Summary of expressed monovalent anti-BCMA variants

| | | SEQ | SEQ | SEQ |
|---|---|---|---|---|
| Variants VH/VL (one-armed IgG | | ID | ID | ID |
| construct) | Tapir ID | NO: | NO: | NO: |
| BCMA (54_VH1a)/BCMA | P1AG5067 | 65 | 66 | 67 |
| (54_VL1a_L1_pGL) | | | | |
| BCMA (54_VH1a_Y292D)/ | P1AG5057 | 68 | 69 | 67 |
| BCMA (54_VL1a) | | | | |
| BCMA (54_VH1a_Y292D)/ | P1AG5014 | 68 | 66 | 67 |
| BCMA (54_VL1a_L1_pGL) | | | | |
| BCMA (54_VH1a_Y292D)/ | P1AG5065 | 68 | 70 | 67 |
| BCMA (54_VL1a_T699S) | | | | |
| BCMA (54_VH1a_Y292D)/ | P1AG5080 | 68 | 71 | 67 |
| BCMA (54_VL2a) | | | | |
| BCMA (54_VH1b)/BCMA | P1AG5072 | 72 | 64 | 67 |
| (54_VL1a) | | | | |
| BCMA (54_VH1b)/BCMA | P1AG5027 | 72 | 66 | 67 |
| (54_VL1a_L1_pGL) | | | | |
| BCMA (54_VH1b)/BCMA | P1AG5056 | 72 | 73 | 67 |
| (54_VL1a_N651A) | | | | |
| BCMA (54_VH1b)/BCMA | P1AG5064 | 72 | 74 | 67 |
| (54_VL1a_N651A_N695S) | | | | |
| BCMA (54_VH1b)/BCMA | P1AG5046 | 72 | 70 | 67 |
| (54_VL1a_T699S) | | | | |
| BCMA (54_VH1b)/BCMA | P1AG5030 | 72 | 71 | 67 |
| (54_VL2a) | | | | |
| BCMA (54_VH2a)/BCMA | P1AG5028 | 75 | 64 | 67 |
| (54_VL1a) | | | | |
| BCMA (54_VH2a)/BCMA | P1AG5013 | 75 | 61 | 67 |
| (54_VL1a_L1_pGL) | | | | |
| BCMA (54_VH2a)/BCMA | P1AG5071 | 75 | 65 | 67 |
| (54_VL1a_T699S) | | | | |
| BCMA (54_VH2a)/BCMA | P1AG5031 | 75 | 71 | 67 |
| (54_VL2a) | | | | |
| BCMA (54) parental | P1AG5061 | 76 | 77 | 67 |
| BCMA (E04_VH1a)/BCMA | P1AG5063 | 78 | 79 | 67 |
| (E04_VL1a) | | | | |
| BCMA (E04_VH1a)/BCMA | P1AG5004 | 78 | 80 | 67 |
| (E04_VL1b) | | | | |
| BCMA (E04_VH1a)/BCMA | P1AG5043 | 78 | 81 | 67 |
| (E04_VL1d) | | | | |
| BCMA (E04_VH1a)/BCMA | P1AG5007 | 78 | 82 | 67 |
| (E04_VL1e) | | | | |
| BCMA (E04_VH1a)/BCMA | P1AG5036 | 78 | 83 | 67 |
| (E04_VL1f) | | | | |
| BCMA (E04_VH1a)/BCMA | P1AG5059 | 78 | 84 | 67 |
| (E04_VL1g) | | | | |
| BCMA (E04_VH1a)/BCMA | P1AG5053 | 78 | 85 | 67 |
| (E04_VL1h) | | | | |
| BCMA (E04_VH1b)/BCMA | P1AG5026 | 86 | 79 | 67 |
| (E04_VL1a) | | | | |
| BCMA (E04_VH1b)/BCMA | P1AG5044 | 86 | 80 | 67 |
| (E04_VL1b) | | | | |
| BCMA (E04_VH1b)/BCMA | P1AG5078 | 86 | 81 | 67 |
| (E04_VL1d) | | | | |
| BCMA (E04_VH1b)/BCMA | P1AG5034 | 86 | 82 | 67 |
| (E04_VL1e) | | | | |
| BCMA (E04_VH1b)/BCMA | P1AG5060 | 86 | 83 | 67 |
| ((E04_VL1f) | | | | |
| BCMA (E04_VH1b)/BCMA | P1AG5021 | 86 | 84 | 67 |
| (E04_VL1g) | | | | |
| BCMA (E04_VH1b)/BCMA | P1AG5076 | 86 | 85 | 67 |
| (E04_VL1h) | | | | |
| BCMA (E04) parental | P1AG5062 | 87 | 88 | 67 |

The protein concentration of purified constructs was determined by measuring the optical density (OD) at 280 nm, using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII (Perkin Elmer). Determination of the aggregate content was performed by HPLC chromatography at 25° C. using analytical size-exclusion column (TSKgel G3000 SW XL or UP-SW3000) equilibrated in running buffer (25 mM K₂HPO₄, 125 mM NaCl, 200 mM L-Arginine Monohydrocloride, pH 6.7 or 200 mM KH₂PO₄, 250 mM KCl pH 6.2 respectively). A summary of the product amount and purification parameters of all monovalent anti-BCMA variants is given in Table 12.

The binding kinetics of the monovalent humanized BCMA antibody variants to human BCMA and to cynomolgus BCMA were investigated by surface plasmon resonance (SPR) using a BIACORE T200 instrument (GE Healthcare). All experiments were performed at 25° C. using HBS-P Buffer (10 mM HEPES, 150 mM NaCl pH 7.4, 0.05% surfactant P20) as running buffer and as dilution buffer. An anti-Fc IgG capturing antibody, specific for the PGLALA mutant Fc region, was immobilized on a series S Sensor Chip CM5 (Cytiva) by using standard amine coupling chemistry, resulting in a surface density of approximately 15000 resonance units (RU). Anti-BCMA antibodies were captured on the surface for 30 s at a flow rate of 5 μl/min leading to a capturing response of 50-200 RU. A dilution series of the antigen (human BCMA Fc homodimer (R&D Systems) or cynomolgus BCMA Fc homodimer (R&D Systems), respectively) was injected for 120 s at 30 μl/min with concentrations from 3 up to 300 nM onto the surface (association phase). The dissociation phase was monitored for 300-600 sec by washing with running buffer. The surface was regenerated by injecting 5 mM NaOH (freshly prepared) for 2×30 s. Bulk refractive index differences were corrected by subtracting blank injections and by substracting the response obtained from the reference flow cell without captured antibody. The derived curves were fitted to a 1:1 Langmuir binding model using the BIAevaluation software.

The K_{D} values for the monovalent constructs including the 29 preferred BCMA variants are shown in Table 12 below.

TABLE 12
Production, Purification and Binding Properties
expressed monovalent anti-BCMA variants

| | | Analyt. | CE SDS | | |
|---|---|---|---|---|---|
| | Product | SEC | Main | | |
| | Amount | Monomer | peak | K_{D} [M] | K_{D} [M] |
| Tapir ID | [μg] | [%] | [%] | huBCMA | cyBCMA |
| P1AG5067 | 2231 | 97.2 | 93.7 | 3.4E−09 | 1.7E−09 |
| P1AG5057 | 1184 | 96.7 | 90.6 | 1.7E−09 | 4.0E−09 |
| P1AG5014 | 1246 | 97 | 92.2 | 1.7E−09 | 1.1E−10 |
| P1AG5065 | 1110 | 95.8 | 88.9 | 2.6E−09 | 6.4E−09 |
| P1AG5080 | 2195 | 97 | 89.9 | 1.3E−09 | 4.3E−09 |
| P1AG5072 | 3394 | 97.2 | 93.8 | 1.3E−09 | 3.2E−09 |
| P1AG5027 | 3492 | 98.3 | 95.4 | 1.7E−09 | 1.2E−09 |
| P1AG5056 | 3200 | 97.4 | 94 | 4.2E−09 | 1.3E−08 |
| P1AG5064 | 1760 | 100 | 89.7 | 3.5E−09 | 1.3E−08 |
| P1AG5046 | 2685 | 97.9 | 92.6 | 1.9E−09 | 3.9E−09 |
| P1AG5030 | 3344 | 97.6 | 91 | 1.3E−09 | 2.2E−09 |
| P1AG5028 | 3405 | 98.7 | 96.1 | 2.7E−09 | 3.8E−09 |
| P1AG5013 | 2900 | 97.6 | 94.5 | 2.4E−09 | 1.9E−09 |
| P1AG5071 | 2508 | 96.5 | 91.4 | 3.4E−09 | 6.5E−09 |
| P1AG5031 | 3155 | 97.5 | 91.1 | 2.4E−09 | 4.7E−09 |
| P1AG5061 | 3481 | 95.5 | 82.6 | 1.4E−09 | 1.9E−09 |
| P1AG5063 | 3041 | 99.6 | 96.3 | 1.6E−09 | 2.8E−08 |
| P1AG5004 | 2454 | 98.6 | 95.2 | 2.5E−09 | 1.6E−08 |
| P1AG5043 | 2485 | 100 | 96.7 | 2.6E−09 | 1.4E−08 |
| P1AG5007 | 393 | 98.6 | 100 | 3.0E−09 | 1.2E−08 |
| P1AG5036 | 2735 | 99.6 | 97.4 | 2.9E−09 | 9.5E−09 |
| P1AG5059 | 2520 | 99.1 | 95.4 | 2.3E−09 | 3.0E−08 |
| P1AG5053 | 1222 | 99.6 | 99.3 | 2.4E−09 | 1.3E−08 |
| P1AG5026 | 569 | 98.9 | 93.1 | 2.7E−09 | 3.3E−08 |
| P1AG5044 | 1398 | 97.7 | 91.8 | 1.8E−09 | 2.0E−08 |
| P1AG5078 | 1236 | 99.8 | 96 | 2.4E−09 | 2.2E−08 |
| P1AG5034 | 286 | 100 | 96.5 | 3.2E−09 | 3.2E−08 |
| P1AG5060 | 702 | 96.4 | 86.5 | 3.2E−09 | 1.4E−08 |
| P1AG5021 | 344 | 97.3 | 92.2 | 2.8E−09 | 3.5E−08 |
| P1AG5076 | 944 | 99.7 | 99.5 | 1.9E−09 | 2.3E−08 |
| P1AG5062 | 1995 | 99.8 | 97.2 | 3.1E−09 | 3.3E−08 |

Based on the results of the in-silico assessment of the occurrence of potential T-cell epitopes in the humanized sequences with the NetMHCIIpan 4.0 predictor (see Example 1.2.3), the antibodies P1AG5080, P1AG5072, P1AG5028, P1AG5031, P1AG5063 and P1AG5036 (4 BCMA 54 variants and 2 BCMA E04 variants) were selected as the molecules with the lowest potential T-cell epitopes. In combination with their binding behavior, P1AG5072 and P1AG5031 were choosen as BCMA 54 variants and P1AG5063 and P1AG5036 were selected as the BCMA E04 variant antibodies to be included in the bispecific antibodies.

### Example 2

### Generation and Production of BCMA Targeting 4-1BBL Trimer-Containing Antigen Binding Molecules

### 2.1 Cloning of BCMA Targeting 4-1BBL Trimer-Containing Antigen Binding Molecules

For the generation of the expression plasmids, the sequences of the respective variable domains were used and sub-cloned in frame with the respective constant regions which are pre-inserted in the respective recipient mammalian expression vector. In the Fc domain, Pro329Gly, Leu234Ala and Leu235Ala mutations (PG-LALA) have been introduced in the constant region of the human IgG1 heavy chains to abrogate binding to Fc gamma receptors according to the method described in International Patent Appl. Publ. No. WO 2012/130831. For the generation of the constructs, Fc fragments contained either the “knob” (S354C/T366W mutations, numbering according to Kabat EU index) or “hole” mutations (Y349C/T366S/L368A/Y407V mutations according to Kabat EU index) to avoid mispairing of the heavy chains. In order to improve correct pairing, charges were introduced into the CH1 and Ckappa domains of the BCMA antigen binding moiety as described in International Patent Appl. Publ. No. WO 2015/150447.

A polypeptide containing two ectodomains of 4-1BB ligand, separated by (G4S)2 linkers, and fused to the human IgG1-CL domain, was cloned as follows: human 4-1BB ligand, (G4S)2 connector, human 4-1BB ligand, (G4S)2 connector, human CL, Fc hole IgG1. A polypeptide containing one ectodomain of 4-1BB ligand and fused to the human IgG1-CH domain, was cloned as follows: human 4-1BB ligand, (G4S)2 connector, human CH.

A schematic illustration of the BCMA targeting 4-1BBL trimer-containing antigen binding molecule is shown in FIG. 1B. Table 13 summarizes specific antigen binding molecules made, their identifiers and the sequences of the heavy chains (Fc1 knob/fusion protein 2 and Fc2 hole) and light chains (LC and fusion protein 1).

TABLE 13
Summary of expressed anti-BCMA-4-1BBL antigen binding molecules

| | | SEQ ID | SEQ ID | SEQ ID | SEQ |
|---|---|---|---|---|---|
| | | NO: | NO: (Fc1 | NO: | ID |
| | | (fusion | knob/fusion | (Fc2 | NO: |
| Description | TAPIR ID | prot. 1) | prot 2) | hole) | (LC) |
| BCMA | P1AG7400 | 22 | 21 | 23 | 24 |
| (E04_VH1a/VL1f)-4- | | | | | |
| 1BBL | | | | | |
| BCMA (E04- | P1AG7409 | 22 | 21 | 23 | 25 |
| VH1a/VL1a)-4-1BBL | | | | | |
| BCMA | P1AG7397 | 22 | 21 | 37 | 38 |
| (54_VH2a/VL2a)-4- | | | | | |
| 1BBL | | | | | |
| BCMA | P1AG7422 | 22 | 21 | 39 | 40 |
| (54_VH1b/VL1a)-4- | | | | | |
| 1BBL | | | | | |
| BCMA (PR)-4-1BBL | P1AF7814 | 22 | 21 | 91 | 92 |

### 2.2 Production and Purification of BCMA Targeting 4-1BBL Trimer-Containing Antigen Binding Molecules

The DNA sequences encoding the variable heavy and light chain regions of the BCMA antigen binding domain and the 4-1BBL fusion proteins were cloned into mammalian expression vectors using conventional cloning techniques. The bispecific antibodies described herein were produced using shaking flasks with FedBatch mode. The recombinant production was performed by transient transfection of Expi293™ Cells in a defined, serum-free medium. For transfection ExpiFectamine™ 293 Transfection Kit was used (Gibco). Cell culture supernatants were harvested 7-12 days after transfection.

Quantification of protein titer: The protein titer of supernatant samples was determined by affinity chromatography using a POROS A 20 μm column, 2.1×30 mm (Life Technologies, Carlsbad, CA, USA) on a High Performance Liquid Chromatography system (Ultimate 3000 HPLC system, Thermo Scientific, Waltham, MA, USA). The supernatant was loaded onto the column equilibrated with 0.2 M Na₂HPO₄, pH 7.4, followed by elution with 0.1 M citric acid, 0.2 M NaCl, pH 2.5. Titers were quantified by measuring absorption at 280 nm, and subsequently calculating the protein concentration by comparing the elution peak area (under the curve) of the analyte with a reference standard curve.

Purification of bispecific antibodies: Proteins were purified from cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5 or PBS; elution buffer: 20 mM, 25 mM or 50 mM sodium citrate, pH 3.0). Elution was achieved at pH 3.0 followed by immediate pH neutralization of the sample. The protein was concentrated by centrifugation (Millipore Amicon® ULTRA-15, #UFC903096), and aggregated protein was separated from monomeric protein by size exclusion chromatography in 20 mM histidine, 140 mM sodium chloride, pH 6.0

Analytics of BCMA Targeting 4-1BBL Trimer-Containing Antigen Binding Molecules: The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer). Determination of the aggregate content was performed by HPLC chromatography at 25° C. using analytical size-exclusion column (TSKgel G3000 SW XL or UP-SW3000, Tosoh Bioscience) equilibrated in running buffer (200 mM KH₂PO₄, 250 mM KCl pH 6.2, 0.02% NaN₃).

A summary of the purification parameters of selected molecules is given in Table 14.

TABLE 14
Summary of the production and purification of BCMA
targeting 4-1BBL trimer-containing
antigen binding molecules

| | | | Analytical | Purity |
|---|---|---|---|---|
| | | | SEC | measured |
| | | | (HMW/ | by CE- |
| | Des- | Yield | Monomer/ | SDS |
| TAPIR ID | cription | [mg/l] | LMW) [%] | [%] |
| P1AG7400 | BCMA | | 99 | 100 |
| | (E04_ | | | |
| | VH1a/ | | | |
| | VL1f)-4- | | | |
| | 1BBL | | | |
| P1AG7409 | BCMA | | 99 | 100 |
| | (E04- | | | |
| | VH1a/ | | | |
| | VL1a)- | | | |
| | 4-1BBL | | | |
| P1AG7397 | BCMA | | 99 | 100 |
| | (54_ | | | |
| | VH2a/ | | | |
| | VL2a)-4- | | | |
| | 1BBL | | | |
| P1AG7422 | BCMA | | 99 | 98.5 |
| | (54_ | | | |
| | VH1b/ | | | |
| | VL1a)-4- | | | |
| | 1BBL | | | |

### 2.3 Biophysical and Biochemical Characterization of BCMA Targeting 4-1BBL Trimer-Containing Antigen Binding Molecules

In order to predict the developability of the BCMA-4-1BBL antigen binding molecules, its biophysical and biochemical properties were assessed by calculation methods and assays.

A sequence-based prediction of the net charge of the protein as a function of pH by using standard software (e.g. the EMBOSS tool), and thus it isoelectric point (calc. pI) helped to estimate whether the bispecific antibody is suitable for binding and non-binding to cation and anion exchange chromatography media, and thus suitable for the general purification methods during production. All produced BCMA-4-1BBL constructs had pI values in the range of about 8 which is considered suitable for developability (see Table 15 below).

Thermal stability of the BCMA-4-1BBL constructs prepared was monitored by Dynamic Light Scattering (DLS) and by monitoring of temperature dependent intrinsic protein fluorescence by applying a temperature ramp using an Optim 2 instrument (Avacta Analytical, UK). 10 μg of filtered protein sample with a protein concentration of 1 mg/ml was applied in duplicate to the Optim 2 instrument. The temperature was ramped from 25° C. to 85° C. at 0.1° C./min, with the ratio of fluorescence intensity at 350 nm/330 nm and scattering intensity at 266 nm being collected. The results are shown in Table 15. The aggregation temperature (T_{agg}) of the tested BCMA-4-1BBL constructs P1AG7397 and P1AG7422 was favorable as compared to the constructs P1AG7409 and P1AG7400, however the aggregation temperature for all constructs was considered sufficiently high for further development.

The apparent hydrophobicity of the bispecific antibodies was assessed by hydrophobic interaction chromatography (HIC) as relative retention time compared to hydrophobicity standards (90% of immunoglobulins in IVIG preparation have a relative retention time <0.35). In detail, 20 μg of sample was injected onto a HIC-Ether-5 PW (Tosoh) column equilibrated with 25 mM Na-phosphate, 1.5 M ammonium sulfate, pH 7.0. Elution was performed with a linear gradient from 0 to 100% buffer B (25 mM Na-phosphate, pH 7.0) within 60 minutes. Retention times were then compared to protein standards with known hydrophobicity (e.g. Avastin). All constructs possess a higher hydrophobicity than a typical antibody.

For the FcRn affinity chromatography, FcRn was expressed, purified and biotinylated as described (Schlothauer et al., MAbs 2013, 5 (4), 576-86). For coupling, the prepared receptor was added to streptavidin-sepharose (GE Healthcare). The resulting FcRn-sepharose matrix was packed in a column housing. The column was equilibrated with 20 mM 2-(N-morpholine)-ethanesulfonic acid (MES) and 140 mM NaCl, pH 5.5 (eluent A) at a 0.5 ml/min flow rate. 30 μg of antibody samples were diluted at a volume ratio of 1:1 with eluent A and applied to the FcRn column. The column was washed with 5 column volumes of eluent A followed by elution with a linear gradient from 20 to 100% 20 mM Tris/HCl and 140 mM NaCl, pH 8.8 (eluent B) in 35 column volumes. The analysis was performed with a column oven at 25° C. The elution profile was monitored by continuous measurement of the absorbance at 280 nm. Retention times were compared to protein standards with known affinities.

Heparin affinity was determined by injecting 30-50 μg of sample onto a TSKgel Heparin-5 PW (Tosoh) column equilibrated with 50 mM Tris, pH 7.4. Elution was performed with a linear gradient from 0 to 100% buffer B (50 mM Tris, 1M NaCl, pH 7.4 mM) within 37 minutes. Retention times were compared to protein standards with known affinities. The expected values for the retention times and the measured retention times for particular BCMA-4-1BBL constructs are shown in Table 15.

TABLE 15
In vitro developability assessment of BCMA-4-1BBL antigen binding molecules

| | | Expected value | P1AG7409 | | P1AG7400 | | P1AG7397 | | P1AG7422 | |
|---|---|---|---|---|---|---|---|---|---|---|
| | | for an “ideal” | BCMA E04 1a 1a | | BCMA E04 1a 1f | | BCMA 54 2a 2a | | BCMA 54 1b 1a | |
| Parameter | | antibody | 4-1BBL | | 4-1BBL | | 4-1BBL | | 4-1BBL | |
| Calc. pI, charge | | >8: OK | 7.99 | | 7.99 | | 7.8 | | 8.16 | |
| distribution | | 7 . . . 8: orange flag | | | | | | | | |
| | | <7: red flag | | | | | | | | |
| Thermal stability | | T_{agg} > 64° C. | 55.50 | | 56.30 | | 64.70 | | 65.90 | |
| (T_{agg}) | | | | | | | | | | |
| Apparent | | Rel. ret. <0.34 | 0.54 | | 0.54 | | 0.50 | | 0.50 | |
| hydrophobicity | | (<Avastin) | | | | | | | | |
| (HIC)¹ | | | | | | | | | | |
| FcRn² | Heparin | | 0.07 | 0.19 | 0.02 | 0.28 | −0.21 | 0.57 | −0.23 | 0.54 |

Affinity
chromatography

¹Relative to generic hydrophobicity standards. 90% of immunoglobulins in IVIG preparation have a relative retention time <0.35.
²Relative to generic FcRn affinity standard. Normal IgGs have a relative retention time between 0 and 2.75.

A characterization of binding potency by surface plasmon resonance (SPR) after stress of was carried out with particular BCMA-4-1BBL constructs. The results are shown in Table 16. The reduction in binding potency caused by incubation of the molecules for 14 days at 37° C., pH 7.4 and at 40° C., pH 6 was quantified by surface plasmon resonance using a Biacore T200 instrument (GE Healthcare). Samples stored at −80° C. and pH 6 were used as reference. The reference samples and the samples stressed at 40° C. were in 20 mM Histidine buffer, 140 mM NaCl, pH 6.0, and the samples stressed at 37° C. in PBS buffer, pH 7.4, all at a concentration of 1.0 mg/ml. After the stress period (14 days) samples in PBS buffer were dialyzed back to 20 mM Histidine buffer, 140 mM NaCl, pH 6.0 for further analysis.

All SPR experiments were performed using a BIACORE instrument (GE Healthcare Biosciences AB, Uppsala, Sweden) at 25° C. with HBS-P+ buffer (10 mM HEPES, 150 mM NaCl, pH 7.4, 0.05% Surfactant P20) as running and dilution buffer. Antigen (R&D Systems or in house purified) was added in various concentrations in solution. Biotinylated human BCMA and 4-1BB, as well as biotinylated anti-hu IgG (Capture Select, Thermo Scientific, #7103262100) were immobilized on a Series S Sensor Chip SA (GE Healthcare, #29104992), resulting in surface densities of at least 1000 resonance units (RU). BCMA-4-1BBL constructs with a concentration of 2 μg/ml were injected for 30 s at a flow rate of 5 μl/min, and dissociation was monitored for 120 s. The surface was regenerated by injecting 10 mM glycine buffer, pH 1.5, for 60 s. Bulk refractive index differences were corrected by subtracting blank injections and by subtracting the response obtained from a blank control flow cell. For evaluation, the binding response 5 seconds after injection end was taken.

To normalize the binding signal, the BCMA and 4-1BB binding was divided by the anti-hu IgG response (the signal (RU) obtained upon capture of the BCMA-4-1BBL constructs on the immobilized anti-hu IgG antibody). The relative binding activity was calculated by referencing each temperature stressed sample to the corresponding, non-stressed sample. As shown in Table 16, all BCMA-4-1BBL constructs showed a stabile binding upon stress to BCMA and 4-1BB.

TABLE 16
In vitro developability assessment of bispecific antibodies targeting BCMA and 4-1BB

| | | Expected value | P1AG7409 | | P1AG7400 | | P1AG7397 | | P1AG7422 | |
|---|---|---|---|---|---|---|---|---|---|---|
| | | for an “ideal” | BCMA E04 1a 1a | | BCMA E04 1a 1f | | BCMA 54 2a 2a | | BCMA 54 1b 1a | |
| Parameter | | lead | 4-1BBL | | 4-1BBL | | 4-1BBL | | 4-1BBL | |
| CE-SDS | Ref. | <2% | 97.93 | | 98.11 | | 98.31 | | 94.56 | |
| [%] | PBS | comp. to | 90.42 | | 89.76 | | 85.02 | | 86.83 | |
| monomer | 37° C. | Ref. | | | | | | | | |
| (non-red.) | His | | 88.82 | | 86.19 | | 91.73 | | 90.78 | |
| | 40° C. | | | | | | | | | |
| CE-SDS | Ref. | <2% | 99.87 | | 99.86 | | 99.81 | | 99.82 | |
| [%] | PBS | comp. to | 97.08 | | 98.48 | | 96.63 | | 98.87 | |
| LC + HC | 37° C. | Ref. | | | | | | | | |
| (red.) | His | | 98.75 | | 98.86 | | 98.1 | | 99.55 | |
| | 40° C. | | | | | | | | | |
| SEC | Ref. | <2% | 98.20 | | 97.80 | | 97.20 | | 97.50 | |
| monomer | PBS | comp. to | 93.70 | | 93.00 | | 92.00 | | 91.50 | |
| [%] | 37° C. | Ref. | | | | | | | | |
| | His | | 98.3 | | 98.3 | | 98.1 | | 97.7 | |
| | 40° C. | | | | | | | | | |
| Target | Ref. | >90% | 100 | 100 | 100 | 100 | 100 | 100 | 100 | 100 |
| binding ± | PBS | | 100 | 97 | 101 | 99 | 101 | 98 | 101 | 98 |
| stress. | 37° C. | | | | | | | | | |
| by SPR | His | | 97 | 97 | 99 | 98 | 98 | 94 | 99 | 96 |
| RAC [%] | 40° C. | | | | | | | | | |
| | | | BCMA | 4-1BB | BCMA | 4-1BB | BCMA | 4-1BB | BCMA | 4-1BB |

The BCMA-41BBL antigen binding molecules displayed a lower stability in the in vitro assessment compared to classical antibodies or bispecific antibodies. However, the data are in the range of other fusion proteins that are currently tested in clinical trials and thus considered acceptable.

### 2.4 Production and Purification of Bispecific Antigen Binding Molecules Targeting BCMA and CD3

For comparison, the well-described BCMA-targeted CD3 T cell engagers Alnuctamab and Elranatamab were prepared. Alnuctamab is a BCMA x CD3 bispecific antibody in a 2+1 format, based on an IgG1 Fc with L234A/L235A/P329G (EU numbering) mutations. The sequences are identified in the International Non-Proprietary Nomenclature List (Recommended INN: List 85; WHO Drug Information, Vol. 35, No. 1, 2021). Alnuctamab comprises an amino acid sequence of SEQ ID NO:191, two amino acid sequences of SEQ ID NO: 192, an amino acid sequence of SEQ ID NO:193 and amino acid sequence of SEQ ID NO: 194 (2+1 format).

Elranatamab is a BCMA x CD3 bispecific antibody in a 1+1 format. The sequences are identified in the International Non-Proprietary Nomenclature List (Recommended INN: List 87, WHO Drug Information, Vol. 36, No. 1, 2022). Elranatamab comprises an amino acid sequence of SEQ ID NO: 195, an amino acid sequence of SEQ ID NO: 196, an amino acid sequence of SEQ ID NO: 197 and amino acid sequence of SEQ ID NO: 198.

For the production of Alnuctamab (P1AF0105), the DNA sequences encoding the variable heavy and light chain regions of the respective binding domains were cloned into mammalian expression vectors using conventional cloning techniques. Antibodies were generated by transient transfection of Expi293F cells. Cells were seeded in Expi293 media (Gibco, #1435101) at a density of 2.5×10⁶/mL. Expression plasmids and ExpiFectamine (Gibco, ExpiFectamine transfection kit, #13385544) were separately mixed in OptiMEM (Gibco, #11520386). After 5 minutes both solutions were combined, mixed by pipetting and incubated for 15-20 minutes at room temperature. Cells were added to the plasmid/ExpiFectamine solution and incubated for 24 hours at 37° C. in a shaking incubator with a 5% CO₂ atmosphere. One day post transfection, supplements (Enhancer 1+2, ExpiFectamine transfection kit) were added. Cell supernatants were harvested after 4-5 days by centrifugation and subsequent filtration (0.2 μm filter), and proteins were purified from the harvested supernatant by standard methods as indicated below.

Elranatamab (P1AH5054) was produced and purified by Proteros according to their standard methods and protocols.

The quantification of Fc containing constructs in supernatants was performed by Protein A-HPLC on an Agilent HPLC System with UV detector. Supernatants are injected on POROS 20 A (Applied Biosystems), washed with 10 mM Tris, 50 mM Glycine, 100 mM NaCl, pH 8.0 and eluted in the same buffer at pH 2.0. Titers were quantified by measuring absorption at 280 nm, and subsequently calculating the protein concentration by comparing the elution peak area (under the curve) of the analyte with a reference standard curve.

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation using Millipore Amicon® ULTRA-15 (Merck, #UFC903096), and aggregated protein was separated from monomeric protein by size exclusion chromatography in 20 mM histidine, 140 mM sodium chloride, pH 6.0. The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of the aggregate content was performed by HPLC chromatography at 25° C. using analytical size-exclusion column (TSKgel G3000 SW XL or UP-SW3000) equilibrated in running buffer (200 mM KH₂PO₄, 250 mM KCl pH 6.2, 0.02% NaN₃). A summary of the purification parameters is given in Table 16A.

TABLE 16A
Summary of the production and purification of bispecific
BCMA x CD3 antigen binding molecules

| | | | | Purity |
|---|---|---|---|---|
| | | | Analytical | measured |
| | | | SEC | by |
| | | Yield | (Monomer) | CE-SDS |
| TAPIR ID | Description | [mg/l] | [%] | [%] |
| P1AF0105 | Alnuctamab | 16.8 | 94.8 | 88.7 |
| P1AH5054 | Elranatamab | 7.6 | >99 | >98 |

Teclistamab is a BCMA x CD3 bispecific antibody with an IgG4-F234A/L235A/S228P (EU numbering) Fc. The sequences are identified in the International Non-Proprietary Nomenclature List (Recommended INN: List 82, WHO Drug Information, Vol. 33, No. 3, 2019). It was obtained from a Supplier (FarmaMondo, Lot Nr. AT1334P1). Teclistamab comprises an amino acid sequence of SEQ ID NO:199, an amino acid sequence of SEQ ID NO:200, an amino acid sequence of SEQ ID NO:201 and amino acid sequence of SEQ ID NO:202.

### Example 3

### Binding of BCMA Targeting 4-1BBL Trimer-Containing Antigen Binding Molecules

To measure the binding to BCMA or 4-1BB we performed FACS-based binding assays on CHO transfectants, that were transduced to stably overexpress either human BCMA, or on Jurkat reporter cells expressing human 4-1BB, respectively.

### Generation of CHO-K1 Cell Lines Expressing BCMA Extracellular Domain Non-Truncating Mutants

Full-length cDNA encoding human BCMA (UniProt: Q02223) and its corresponding mutants (R27P, S30del, P33S and P34del) were subcloned into lentiviral transfer vectors controlled by the CMV promoter. Lentiviral particles were prepared by transiently co-transfecting HEK 293-derived viral production cells (Gibco, #A35347) with transfer plasmid and lentiviral packaging mix (pRSV-Rev, pCgpV, and pCMV-VSV-G) using the LV-MAX Transfection Kit (Gibco, #A35346) according to the manufacturer's protocol. Viral supernatants were harvested 48 h after transfection, filtered through a 0.45-μm low protein binding filter and stored at −80° C. until use.

One day prior to transduction, 5×10⁴ CHO-K1 (ATCC CRL-9618) cells were seeded per well in a 24-well plate. The following day, the culture medium was replaced with 300 μL of purified lentiviral supernatant and 100 μL of fresh culture medium DMEM/F-12 (Gibco, #11320033) supplemented with 10% fetal bovine serum (Gibco, #16140063) and 1% GlutaMAX Supplement (Gibco; #31331-028). To facilitate viral transduction, the infectious medium was further supplemented with 2.5 μL of TransDux™ reagent and 100 μL of MAX Enhancer (SBI; #LV860A-1). The cells were then incubated for 24 hours at 37° C. After the incubation period, the viral medium was discarded and the cells were subsequently maintained in fresh culture medium.

Three days post transduction, the culture medium was supplemented with 6 μg/mL puromycin (Invivogen; #ant-pr-1). After initial selection, the cells exhibiting human BCMA surface expression were isolated by BD FACSAria III cell sorter (BD Biosciences) and subsequently cultured to generate stable clones. Following a 4-week stability test, the surface expression and its stability were verified by flow cytometry analysis using mouse PE-conjugated anti-human BCMA (BioLegend, #357503).

The stable CHO transfectants (parental cell line CHO-k1 ATCC #CCL-61) were cultured in DMEM/F-12 (Gibco, #10565018) supplemented with 10% fetal bovine serum (Gibco, #16140063 or Sigma-Aldrich F4135) and 1% GlutaMAX Supplement (Gibco; #31331-028), including 6 μg/ml puromycin (Invivogen; #ant-pr-1). Adherent CHO cells were detached using Cell Dissociation Buffer (Gibco, #13151014) or trypsin (Gibco by ThermoFisher Scientific, TrypLETMExpress Enzyme #2605-010), counted and checked for viability. All subsequent steps were performed at 4° C.

### BCMA Binding Protocol

For the assessment of the binding to BCMA, CHO-huBCMA cells were re-suspended in FACS buffer (PBS, 2% Fetal Bovine Serum; 1% 0.5 M EDTA pH 8; 0.25% NaN₃ Sodium azide) at 1 Mio cells per ml. 0.1 Mio cells were plated per well of a round-bottom 96-well-plate and washed with 150 μl cold FACS buffer per well once. Cells were stained in a total volume of 50 μl per well and increasing concentrations of the indicated BCMA-4-1BBL antigen binding molecules (0.48 μM-2000 nM) for 30 minutes at 4° C. Afterwards, the cells were centrifuged and washed with 150 μl FACS buffer twice.

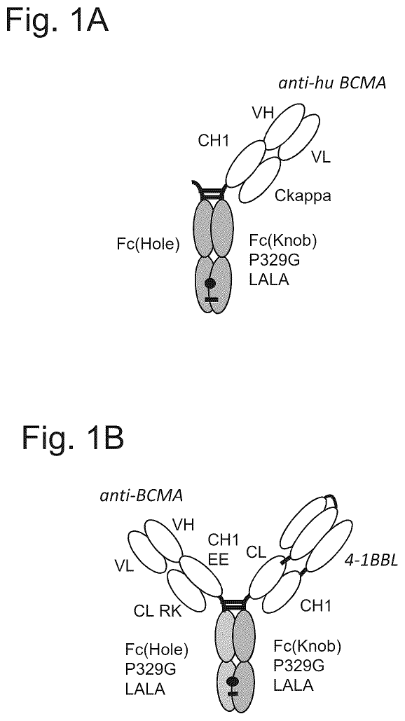

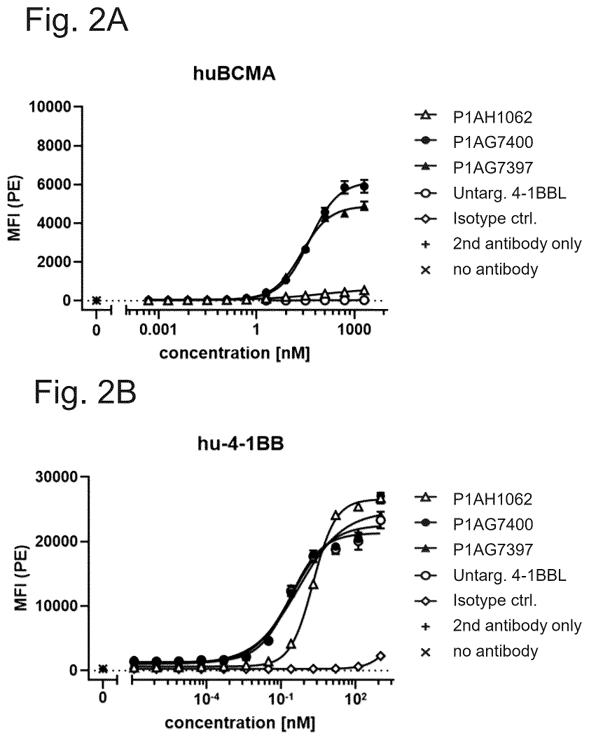

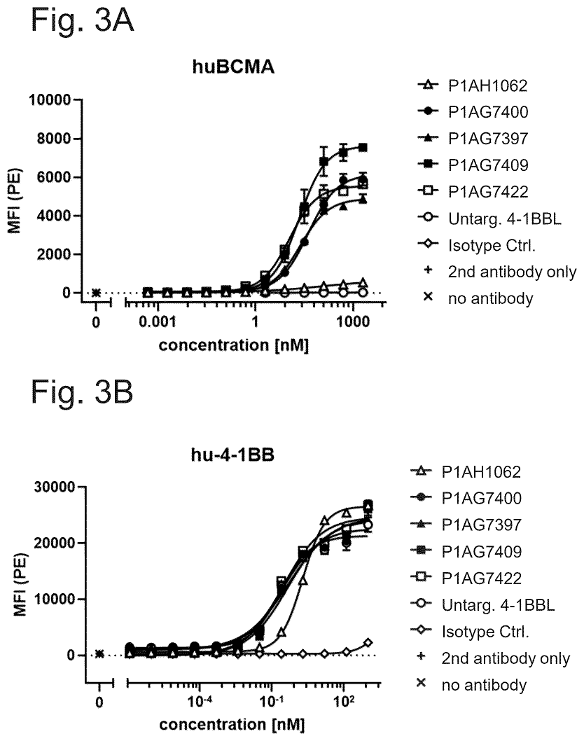

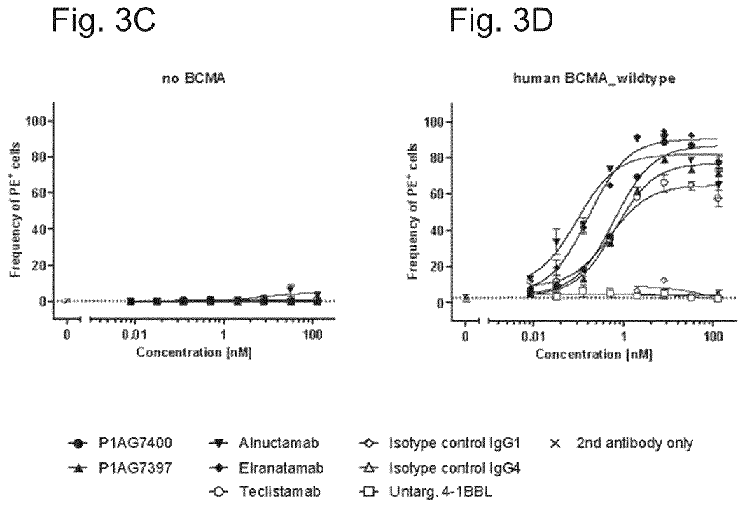

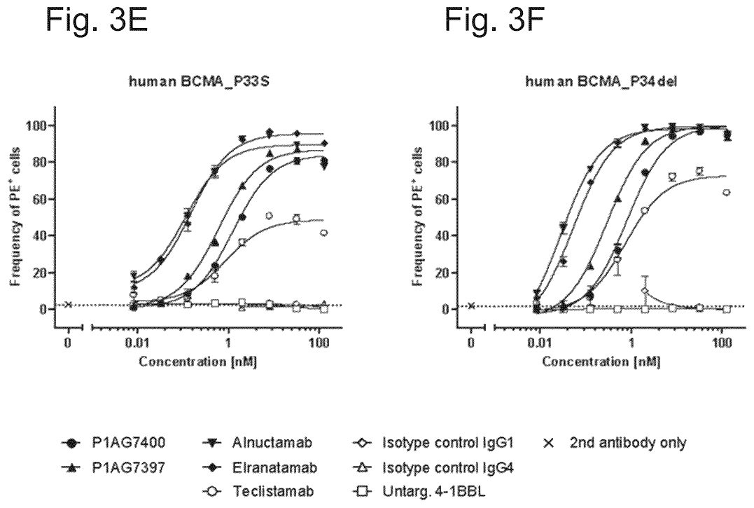

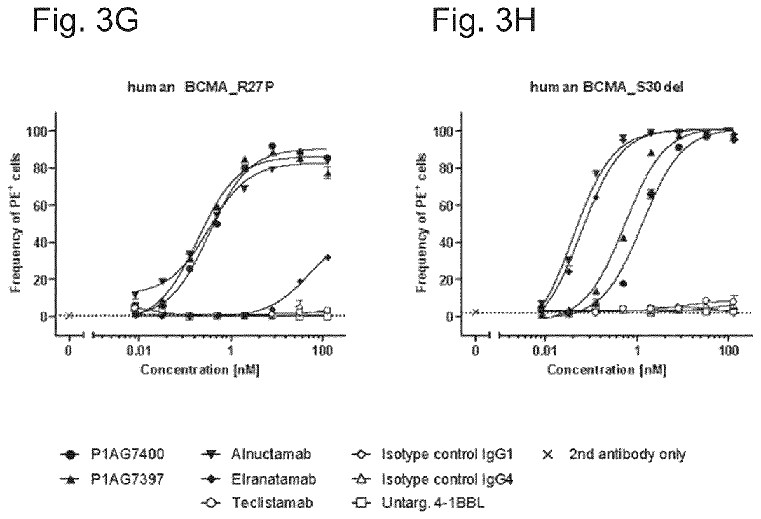

A total of 25 μl per well of pre-diluted secondary antibody (PE-AffiniPure F(ab′)2 Fragment Goat Anti-Human IgG, Fcγ Fragment Specific; Jackson Immunoresearch, 109-116-170, 1:50 diluted in FACS buffer) was added and plates were incubated for 30 minutes at 4° C. Cells were washed twice and then fixed by adding 50 μl of 1% PFA (in PBS) and an incubation of 20 min at RT. Cells were washed once with 150 μl FACS buffer and analyzed on a BD Fortessa flow cytometer, equipped with the software FACS Diva. Binding curves and EC₅₀ values were obtained using GraphPadPrism6. The data as measured for various BCMA-4-1BBL antigen binding molecules and controls (for instance “2^{nd} antibody only refers to samples containing only the flurophore labeled secondary antibody) are shown in FIGS. 2A and 3A.

For the assessment of the binding to BCMA variants with the indicated point mutations in the extracellular domain of hu BCMA, CHO transfectants were resuspended in PBS (Gibco by ThermoFisher Scientific, #20012050) and counted. Live-dead staining of target cells was performed by incubation of cells with 1:1000 diluted Zombie Aqua Viability dye (BioLegend #42310277143) in PBS at 1.5 Mio cells per ml for 10 minutes in the dark, followed by a two washing steps with PBS and centrifugation at 400×g at 4° C. for 4 minutes. CHO-K1 cells were adjusted to 1.5 Mio cells per ml in PBS and 40 μl were seeded per well of a 384-U bottom well plates (ThermoFisher Scientific #264573). Plates were centrifuged once more at 400×g at 4° C. for 4 minutes and 20 μl supernatant was removed.

Cells were stained in a total volume of 20 40 μl per well and by adding 20 μl of 2× concentrated increasing concentrations of the indicated dilutions of BCMA-4-1BBL antigen binding molecules (final concentrations ranged from 0.008- to 125 nM) for 30 minutes at 4° C. in the dark. Afterwards, the cells were centrifuged and washed with 40 μl PBS buffer twice.

A total of 20 ml per well of pre-diluted secondary antibody (R-Phycoerythrin AffiniPure Fab Fragment Goat Anti-Human IgG, Fcγ fragment specific; Jackson Immunoresearch, 109-117-008, 1:100 diluted in PBS) was added and to plates already containing 20 ml (final dilution 1:200). Plates were incubated for 30 minutes at 4° C. Cells were washed twice with 40 μl PBS per well and then fixed by adding 40 μl of 1% PFA (in PBS) overnight at 4° C. Cells were washed twice with 40 μl FACS buffer and analyzed on a BD FACSymphony™ A5 Cell Analyzer. Binding curves and EC50 values were obtained using GraphPadPrism6. The data as measured for various BCMA-4-1BBL antigen binding molecules and controls (for instance “2nd antibody only refers to samples containing only the fluorophore labeled secondary antibody) are shown in FIG. 3C to FIG. 3H.

### 4-1BB Binding Protocol

Agonistic binding of the 4-1BB (CD137) receptor to its ligand (4-1BBL) induces 4-1BB-downstream signaling via activation of nuclear factor kappa B (NFκB) and promotes survival and activity of CD8 T cells (Lee H W, Park S J, Choi B K, Kim H H, Nam K O, Kwon B S, J Immunol 2002; 169, 4882-4888). To monitor this NFκB-activation mediated by 4-1BBL antigen binding molecules, the Jurkat-NFκB-luc2_4-1BB Reporter Cell Line was developed by Promega in collaboration with Roche Diagnostics GmbH and is stably transfected with two plasmids encoding human 4-1BB and a NFκB-driven luciferase reporter gene. The cell line was grown in RPMI1640 medium supplemented with 10% FCS, 25 mM HEPES (Thermofisher), 2 mM GlutaMax, 0.1 mM non-essential amino acid (Thermofisher), and 1 mM sodium pyruvate. In addition, the medium was supplemented with 400 μg/ml of Hygromycin B and 600 μg/mL of Geneticin (Sigma-Aldrich). Jurkat cells were cultured twice per week to maintain a density between 0.2-1.0×10⁶ cells/ml.

All subsequent steps were performed at 4° C. Jurkat-NFκB-luc2_4-1BB cells were resuspended in PBS at 1.0×10⁶ cells/ml. 0.1×10⁶ cells per well were seeded into a 96-round-bottom-well plate and stained with a fixable viability dye (LIVE/DEAD™ Fixable Near-IR Dead Cell Stain Kit (APC/Cy7), L34976 MolecularProbes, 1:1000 diluted in PBS) for 20 min at 4° C. After washing cells with PBS once, they were incubated with 50 μl per well of 1000 nM to 0.0001 μM (serial dilutions of 1:8) of the indicated BCMA-4-1BBL constructs, a negative reference (untargeted 4-1BBL) or an isotype control or no construct for 30 min at 4° C. The cells were washed with cold FACS buffer (PBS, 2% Fetal Bovine Serum; 1% 0.5m EDTA pH 8; 0.25% NaN3 Sodium azide (20%)) twice and then stained with PE-conjugated secondary antibody (PE-AffiniPure F(ab′) 2 Fragment Goat Anti-Human IgG, Fcγ Fragment Specific; Jackson Immunoresearch, 109-116-170) that was diluted 1/100 in cold FACS buffer, for another 30 min at 4° C. After two final washing steps, cells were fixed with 1% PFA for 20 min at RT. Flow cytometric acquisition was performed on a custom-designed BD Biosciences Fortessa and analyzed using BD Diva. EC₅₀ values were calculated, using GraphPad Prism software. The data as measured for various BCMA-4-1BBL antigen binding molecules and controls (for instance “2^{nd} antibody only refers to samples containing only the fluorophore labeled secondary antibody) are shown in FIGS. 2B and 3B.

All BCMA targeting 4-1BBL trimer-containing antigen binding molecules were able to bind human BCMA (FIGS. 2A and 3A) as well as to human 4-1BB (FIGS. 2B and 3B) in a concentration-dependent manner. The BCMA-4-1BBL constructs showed a significantly stronger binding to human BCMA than the construct P1AH1062 (BCMA 5D5 LM4) as prepared according to WO 2021/118246 A1. P1AH1062 is a bivalent BCMA antibody that comprises two scFv fragments binding to 4-1BB at its C-terminus. Thus, P1AH1062 comprises two similar heavy chains of SEQ ID NO:89 and two light chains of SEQ ID NO:90. Binding to human 4-1BB was comparable for all constructs (FIGS. 2B and 3B). The EC₅₀ values are listed in Table 17 below.

TABLE 17
EC₅₀ values (nM) for binding of the indicated antigen binding
molecules to either human 4-1BB or BCMA, expressed on cells

| | | | EC₅₀ | EC₅₀ |
|---|---|---|---|---|
| | | | binding | binding |
| | Moiety | | to | to |
| | binding to | | 4.1BB | BCMA |
| BCMA antibody | 4-1BB | Name | (nM) | (nM) |
| BCMA (5D5 LM4) | scFv (4-1BB) | P1AH1062 | 1.89 | 103 |
| BCMA (E04_1a1f) | 4-1BBL | P1AG7400 | 0.51 | 41.5 |
| BCMA (54_2a2a) | 4-1BBL | P1AG7397 | 0.27 | 23.7 |
| BCMA (E04_1a1a) | 4-1BBL | P1AG7409 | 0.45 | 21.6 |
| BCMA (54_1a1a) | 4-1BBL | P1AG7422 | 0.29 | 10.8 |
| — | 4-1BBL | neg. ref. | 0.23 | — |
| | | (untargeted | | |
| | | 4-1BBL) | | |

In a second assay, the bispecific antigen binding molecules tested comprised the trimeric 4-1BBL and the BCMA binder BCMA (54_2a2a) or BCMA (E04_1a1f). Since one of the relapse mechanisms in MM can be non-truncating, missense mutations or in-frame deletions in the extracellular domain of BCMA, the above mentioned molecules were compared head-to-head to well described BCMA-targeted CD3 T cell engagers, namely Teclistamab, Alnuctamab and Elranatamab (Lee et al., Nature Medicine 2023, 29, 2295-2306).

The binding properties of the various BCMA-targeting molecules are summarized in Table 17A. Both BCMA-targeted 4-1BBL bispecific molecules, as well as Alnuctamab, were able to bind to the wt and to all mutated forms of human BCMA in a concentration-dependent manner. In contrast, Elranatamab displayed a significant reduction of binding to the point mutation R27P, which translated into a weak binding signal at the highest concentrations only. Teclistamab was not able to bind to the point mutation R27P and the S30del. Efficient binding to BCMA is a crucial requisite for the therapeutic activity of BCMA-targeted bispecific molecules, suggesting an advantage for molecules who are not affected by point mutations in the ECD of BCMA. The difference in the maximal values for the wt versus the mutated BCMA transfectants is due to different expression levels of the human BCMA wt and variants on the transfectants and is consistent for all molecules tested.

TABLE 17A
High-level summary of binding properties of the indicated BCMA-targeting
molecules to either human wildtype (wt) BCMA or the indicated point
mutations in the extracellular domain of human BCMA

| | | | CHO-K1- | CHO-K1- | CHO-K1- | CHO-K1- |
|---|---|---|---|---|---|---|
| | | | BCMA | BCMA | BCMA | BCMA |
| BCMA | | | R27P_{—} | P34del_{—} | P33S_{—} | S30del_{—} |
| antibody | Name | wt BCMA | clone_12 | clone_C10 | clone_D5 | clone_H1 |
| BCMA | P1AG7397 | yes | yes | yes | yes | yes |
| (54_2a2a) | | | | | | |
| BCMA | P1AG7400 | yes | yes | yes | yes | yes |
| (E04_1a1f) | | | | | | |
| Elranatamab | P1AH5054 | yes | reduced & | yes | yes | yes |
| | | | only at high | | | |
| | | | concentrations | | | |
| Teclistamab | FarmaMondo | yes | no | yes | yes | no |
| | Lot nr. | | | | | |
| | AT1334P1 | | | | | |
| Alnuctamab | P1AF0105 | yes | yes | yes | yes | yes |

“Yes” means binding to the BCMA variant.

### Example 4

### In Vitro Functional Characterization of BCMA Targeting 4-1BBL Trimer-Containing Antigen Binding Molecules

Several cell-based in vitro and ex vivo assays were performed to evaluate the capability of the BCMA-4-1BBL antigen binding molecules to boost T-cell bispecific-mediated activation. (Jurkat and) T-cell activation, proliferation, cytokine secretion, and tumor cell killing as determined by flow cytometry were main read-outs.

1. The activity of the targeted BCMA-4-1BBL molecules was assessed in a Jurkat-NFκB-luc2_4-1BB Reporter assay, where the molecules simultaneously bind to human 4-1BB on Jurkat-NFκB-luc2_4-1BB cells and BCMA expressed on the indicated MM cell lines leading to hyperclustering of 4-1BB and thereby receptor activation. Subsequent induction of corresponding signaling pathways results in the activation of NFκB which induces the expression of firefly luciferase. The activity of the luciferase reporter is measured by adding luciferase substrate and luminescence readout.

2. In another read-out, the functionality of BCMA-4-1BBL was evaluated in a PBMC co-culture assay in presence of BCMA-expressing MM cell lines and a fixed concentration of a GPRC5D TCB providing the simultaneous 1st signal for T-cell activation. Alternatively, BCMA-4-1BBL was added at a fixed concentration to different concentrations of a FcRH5-targeted CD3 engager (FcRH5 x CD3). FcRH5 x CD3 bispecific antibodies are for instance disclosed in WO 2016/205520 A1. Read-outs were upregulation of surface activation markers on T cells and lysis of tumor cells, as indicated.

3. A similar set-up was used to assess the functional activity of BCMA-4-1BBL antigen binding molecules in presence of a GPRC5D TCB in an ex vivo setting, using unprocessed MM patients bone marrow aspirate samples containing autologous T effector cells and BCMA—as well as GPRC5D-expressing MM target cells.

### 4.1 T Cell Activation—Jurkat NFκB/4-1BB Reporter Cell Assay

Jurkat NFκB reporter assays were used to address how different BCMA-4-1BBL antigen binding molecules can activate T cells. The assay has been performed as described in the following:

Cell cultures: NCI-H929 cells were cultivated in RPMI1640 (Gibco) supplemented with 10% FCS (PAN-biotech), 2 mM L-glutamine (Sigma-Aldrich), 1 mM sodium pyruvate (Thermofisher), and 50 μM 2-Mercaptoethanol (Thermofisher). Cells were cultured twice per week to maintain a density between 0.5-2.0×10⁶ cells/ml. Jurkat IL2 reporter and Jurkat NF-κB/4-1BB reporter cell lines were grown in RPMI1640 medium supplemented with 10% FCS, 25 mM HEPES (Thermofisher), 2 mM L-glutamine, 0.1 mM non-essential amino acid (Thermofisher), and 1 mM sodium pyruvate. In addition, the medium was supplemented with 400 μg/ml of Hygromycin B and 600 μg/mL of Geneticin (Sigma-Aldrich) for Jurkat NFκB/4-1BB reporter cell line. Jurkat cells were cultured twice per week to maintain a density between 0.1-0.5×10⁶ cells/ml.

The target (NCI-H929) and effector (Jurkat NFκB/4-1BB reporter) cells were harvested and re-suspended in assay medium (Jurkat cell medium without antibiotics) to obtain a cell density of 6×10⁶ cells/ml for the target cells and 2×10⁶ cells/ml for the effector cells. Then, the cells were mixed at a ratio E:T 1:3, and 20 μl of effector-target cells mixture were plated per well of a white-walled 384-flat bottom well plate (353988 Falcon™ 384-Well White Flat-Bottom Tissue Culture Treated Microplate). Next, 20 μl of titrated amount (600.0-0.02 nM) of BCMA-4-1BBL antigen binding molecules were added to the plate in triplicates, reaching the final volume of 40 μl per well. In addition, two separate control conditions were prepared. Target and effector cells were mixed alone—to indicate the baseline of effector cell-induced NFκB2 signaling without stimulus. To address possible nonspecific activation of effector cells 4-1BBL antigen binding molecules comprising an Fab against an irrelevant target (untargeted 4-1BBL) were used. Assay medium was used to reach the final volume (40 μl)) for the control wells. Assay plates were centrifuged for 1 min at 350 g and incubated for 4 hrs at 37° C. in a humidified CO₂ incubator. The assay plates were incubated for 5 min at room temperature before adding 20 μl of the ONE-Glo solution (Promega). Furthermore, plates were centrifuged for 1 min at 350 g and incubated for 10 min at room temperature in the dark to reach complete lysis of the cells. Luminescence was measured (read: 1 s per well) by using the Tecan Spark10M.

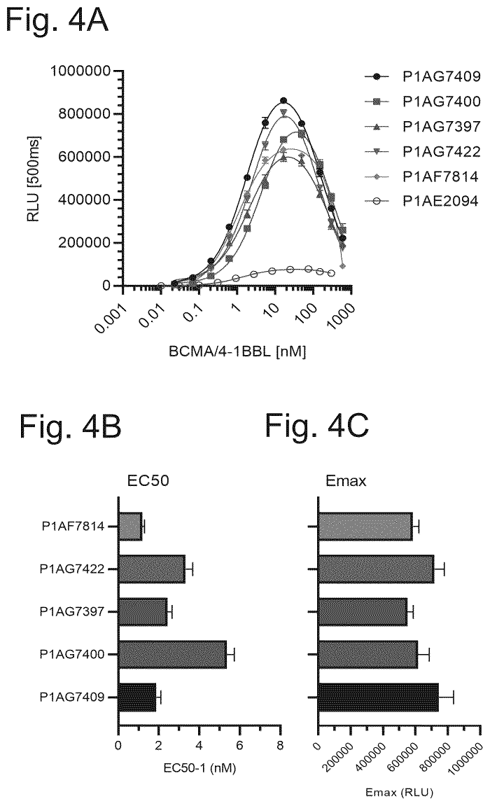

Briefly, the target NCI-H929 cell line expressing high levels of BCMA was mixed at the ratio 1:3 (E:T) with the effector Jurkat NFκB reporter cell line that induces 4-1BB receptor-mediated signaling induces luminescence via the NFκB signaling pathway. Then, titrated (600.0-0.02 nM) BCMA-4-1BBL antigen binding molecules were added and luminescence was measured 4 hrs later. Results showed that all tested BCMA-4-1BBL antigen binding molecules induced T cell activation in a dose-dependent manner (FIG. 4A). Two molecules P1AG7409 and P1AG7422 outperformed other variants and the molecule comprising BCMA antibody PR (P1AF7814) (FIG. 4A to FIG. 4C).

Obtained data are shown as mean #s.d. of one independent experiment performed in triplicates out of three independent experiments in Table 18 below.

TABLE 18
EC50 values (nM) and Emax (%) for T cell activation as
measured in the NFκB/4-1BB Reporter cell assay

| Molecule | EC50 (nM) | Efficacy (Eₘₐₓ (RLU)) |
|---|---|---|
| P1AG7409 | 1.906 | 863586.6 |
| P1AG7400 | 4.829 | 708230.2 |
| P1AG7397 | 2.148 | 600361.9 |
| P1AG7422 | 2.836 | 802822.6 |
| P1AF7814 | 1.053 | 635327.1 |

### 4.2 PBMC Co-Culture Assay in Presence of BCMA-Expressing MM Cell Lines or BCMA-Expressing CHO-k1 Transfectants

To assess the ability of BCMA-4-1BBL antigen binding molecules to function in a PBMC co-culture assay in presence of BCMA-expressing MM cell lines, the following method was carried out.

Target Cells: NCI-H929 & NCI-H929 BCMAko, BCMA-expressing CHO-k1 transfectants or MOLP-2, as indicated respectively.

NCI-H929 (ATCC® CRL-9068™) is a human multiple myeloma cell line that expresses BCMA. In order to assess potential target-independent activity of the BCMA-4-1BBL antigen binding molecules, a BCMA knockout variant of the NCI-H929 cell line (generated using CRISPR/Cas9 technology) was tested as well. The cells were cultivated in RPMI 1640 (Gibco™ 31870074) supplemented with 10% FCS (Gibco™ 16140-071), 10 mM HEPES (Gibco™ 15630056), 2 mM GlutaMAX-I (Gibco™ 35050-038), 1 mM sodium pyruvate (Gibco™ 11360039), and 50 μM 2-Mercapto-ethanol (Gibco™ 31350010). Cells were passaged 2-3 times per week by adding fresh medium to maintain a density between 0.5×10⁶/ml and 2.5×10⁶/ml. Cells were incubated at 37° C. with 5% CO₂. At the day of the assay the target cells were harvested, counted and re-suspended in assay medium (RPMI 1640 Gibco™ 31870074 w/HEPES w/GlutaMax plus 10% FCS, Sigma-Aldrich #F4135).

The stable CHO transfectants (parental cell line CHO-k1 ATCC #CCL-61) were cultured in DMEM/F-12 (Gibco, #10565018) supplemented with 10% fetal bovine serum (Gibco, #16140063 or Sigma-Aldrich F4135) and 1% GlutaMAX Supplement (Gibco; #31331-028), including 6 μg/ml puromycin (Invivogen; #ant-pr-1). Adherent CHO cells were detached using trypsin (Gibco by ThermoFisher Scientific, TrypLETMExpress Enzyme #2605-010). Cells were centrifuged at 300×g for 5 minutes, resuspended in PBS and counted and checked for viability using Cedex HiRes Analyzer. Target cells were centrifuged at 300×g for 5 minutes, PBS was removed except for 1 ml and pellet was not disturbed for the irradiation of the target cells for 1.49 minutes (5000 Rad, 50 Gy) using a RS 2000 Irradiator (RadSource). Afterwards cells were resuspended in PBS for subsequent counting using Cedex HiRes Analyzer. For the assay, the irradiated target cells were adjusted to 1.2 Mio cells per ml in assay medium (RPMI 1640 Gibco™ 72400-21 w/HEPES w/GlutaMax plus 10% FCS, Sigma-Aldrich #F4135).

MOLP-2 (DSMZ ACC607) is a human multiple myeloma cell line that expresses e.g. BCMA, GPRC5D and FcRH5. The cells were cultured in RPMI 1640 with HEPES and GlutaMax (Gibco™ 72400-21) supplemented with 20% FCS. Cells were passaged 2 times per week by adding fresh medium to maintain a density between 0.3×10⁶/ml and 1×10⁶/ml. Cells were incubated at 37° C. with 5% CO₂. For the assay, MOLP-2 cells were counted and checked for viability. Cells were and washed with D-PBS centrifuged for 5 minutes at 280 xg and washed once with PBS. Next, cells were resuspended in PBS to a concentration of 2 Mio cells per ml. Pre-diluted CellTrace Far Red Dye (Invitrogen via Thermo Fisher Scientific, #C34564) in PBS was added to the cell suspension at a final concentration of 0.04 UM and the cells were stained for 15 minutes at 37° C. in the waterbath. Assay medium was added (RPMI 1640 Gibco™ 72400-21 w/HEPES w/GlutaMax plus 10% FCS, Sigma-Aldrich #F4135), cells were centrifuged at 280×g for 5 minutes, counted and checked for viability and adjusted to 1.2 Mio cells per ml in assay medium (RPMI 1640 Gibco™ 72400-21 w/HEPES w/GlutaMax plus 10% FCS, Sigma-Aldrich #F4135).

### Effector Cells: PBMCs

Frozen human peripheral blood mononuclear cells (PBMC) from whole human blood in CPD were received from Cambridge bioscience or Biomex. Alternatively, PBMCs were isolated from buffy coats that were collected from anonymous healthy volunteers through the Zürich blood donation center. PBMCs were isolated from buffy coats by diluting the buffy coat 2:1 with PBS and pipetting the PBS/buffy coat mixture into a Leucosep tube (227290, greiner bio-one) containing 15 ml of Histopaque®-1077 (10771, Sigma) underneath the barrier. Cells were centrifuged for 30 min at 450×g at room temperature without a break (density centrifugation). The interphase consisting of the PBMCs was collected and washed several times with PBS. PBMCs were resuspended in Freezing medium (80023-IBI) at 40-67 Mio cells/ml and frozen using a Cell Freezing Container (CLS432002 Corning) at −80° C. over night and then transferred to nitrogen vapor phase for storage.

PBMCs were stored in nitrogen vapor phase and thawed on the day of the assay. Cells were counted and then labeled with Cell Proliferation Dye eFluor™ 450 (65-0842-90, eBioscience™). Briefly, PBMCs were washed once with DPBS (Gibco), supernatant was discarded and cells resuspended to 2 Mio cells per well using DPBS. 10 ml of the cell suspension was added to 50 ml-falcon tubes and then 10 ml of a 10 μM concentrated Cell Proliferation Dye eFluor™ 450 was added while carefully vortexing (final dye concentration was 5 μM). After an incubation of 10 min at 37° C. (water bath) either 30 ml of warm assay medium was added and cells were incubated for 5 minutes at 4° C. or 30 ml cold medium was added to stop the labeling reaction. Cells were centrifuged, counted and re-suspended in assay medium to obtain a cell density of 1.2×10⁶ PBMCs/ml.

In another assay, human PBMCs were thawed and washed with PBS, including a centrifugation at 300×g for 15 minutes at room temperature. To label the PBMCs with CFSE, CellTrace CFSE dye (Invitrogen via ThermoFischer Scientific, #C34554) was diluted in room temperature PBS (2 mM stock concentration) and cells were incubated at a final concentration of 0.1 μM for 15 minutes at 37° C. in a waterbath. 20 ml medium containing 10% FCS were added and the suspension was centrifuged at 300×g for 15 minutes. The cells were resuspended in assay medium (RPMI 1640 Gibco™ 72400-2 w/HEPES w/GlutaMax plus 10% FCS, Sigma-Aldrich #F4135) at a concentration of 1.2 Mio cells per ml.

### Preparation of Co-Culture

50 μl (0.06×10⁶) NCI-H929 or NCI-H929 BCMAko cells and 50 μl (0.06×10⁶ efluor450-labeled PBMCs) were plated per well in 96-well round bottom plates (TPP #92097) resulting in a 1:1 ratio. Next, 50 μl of titrated amount (0.12-500 nM) of (0.14 nM-300 nM) of BCMA-4-1BBL antigen binding molecules were added to the plate in triplicates. Further, 50 μl of fixed concentration (0.32 μM) GPRC5D-TCB (P1AE6625) was added to the plate, reaching the final volume of 200 μl per well. In addition, three separate control conditions were prepared. Target and effector cells with BCMA-4-1BBL antigen binding molecules and without GPRC5D-TCB were added to address potential activation of effector cells by BCMA-4-1BBL molecules without a first signal. In another control, target, and effector cells were treated with GPRC5D-TCB alone to indicate the baseline. Last, a control with target and effector cells was prepared in which no antibody was added (untreated). Assay medium was used to reach the final volume (200 μl) for the control wells. Assay plates were centrifuged for 1 min at 350 g and incubated for 4 days at 37° C. in a humidified CO₂ incubator.

In another set-up, 50 μl (0.06 Mio) of the respective CHO-hBCMA variant or parental (BCMA-negative) CHO-k1 and 50 μl (0.06 Mio) eFluor450-labeled PBMCs were plated per well in 96-well flat bottom plates (TPP, #92096) resulting in a 1:1 effector to target cell ratio. Next, 50 μl of a fixed concentration (final concentration of 90 μM) anti-CD3 IgG (clone SP34-1, BD Biosciences 551916) was added to the plate. Further, 50 μl of BCMA-4-1BBL antigen binding molecules were added to the plate in triplicates, covering a final concentration of 3.84 μM to 300 nM, with a final volume of 200 μl per well. In addition, a control was plated, containing target and effector cells with anti-CD3 IgG alone to determine the baseline effects induced by the first signal provider only. Last, a control with target and effector cells was prepared in which no antibody was added (untreated). Assay medium was used to reach the final volume (200 μl) for the control wells. Assay plates were centrifuged for 1 min at 350 g and incubated for 4 days at 37° C. in a humidified CO₂ incubator.

Alternatively, 50 μl (0.06 Mio) FarRed-labeled MOLP-2 and 50 μl (0.06 Mio) CFSE-labeled PBMCs were plated per well in 96-well round bottom plates (TPP, #92097) resulting in a 1:1 effector to target cell ratio. Next, 50 μl of different doses of FcRH5 x CD3 were added (final concentration range of 0.03 μM to 32 nM). To some wells, 50 μl of assay medium and to others 50 μl of a fixed concentration of 10 nM of BCMA-4-1BBL antigen binding molecules were added (final volume of 200 μl per well). In addition, a control was plated, containing target and effector cells with 10 nM BCMA-4-1BBL alone. Last, a control with target and effector cells was prepared in which no antibody was added (untreated). Assay medium was used to reach the final volume (200 μl) for the control wells. Assay plates were centrifuged for 1 min at 350 g and then incubated for 4 days at 37° C. in a humidified CO₂ incubator.

### FACS Staining and Read-Out

After incubation, the cells were stained to evaluate T cell activation and proliferation. The cells were first washed once with 200 μL PBS and then stained with LIVE/DEAD™ Fixable Near-IR Dead Cell Stain (1:500) (ThermoFisher Scientific Catalog No. L34976), FITC anti-human CD4 (clone RPA-T4), BV711 anti-human CD8 (clone RPA-T8), APC anti-human CD25 (clone BC96), PerCP-Cy5.5 anti-human CD137 (clone 4B4-1), all from BioLegend in PBS for 30 min at 4° C. Flow cytometry acquisition was performed on a custom-designed BD Biosciences Fortessa and analyzed using FlowJo software (Tree Star, Ashland, OR) and GraphPad Prism software.

In another experiment, after 4 days of incubation plates with co-cultures of PBMCs and variants of CHO-k1 cells were centrifuged at 400×g, 4° C. for 4 minutes. Cells were transferred into 96-well U-bottom plates (TPP #92097), in which they were washed with 150 μL PBS per well. Cells were centrifuged at 400×g, 40 min, 4° C. This washing step was repeated once. Samples were mixed with Eppendorf MixMate at 850 rpm for 15 seconds. Thereafter, 25 μL of an FACS antibody mix (LIVE/DEAD™ Fixable Near-IR Dead Cell Stain, 1:1000, ThermoFisher Scientific, #L34976, FITC anti-human CD4, clone RPA-T4, BV711 anti-human CD8, clone RPA-T8, APC anti-human CD25, clone BC96, BUV395 anti-human CD69, clone FN50, PE anti-human CD137, clone 4B4-1, all from BioLegend and pre-diluted in PBS) were added per well and cells were incubated for 30 minutes at 4° C. in the dark. Thereafter, cells were washed twice with 150 μl FACS Buffer/well, mixed with the Eppendorf MixMate at 850 rpm for 15 seconds and fixed with 100 μl of 1% Formaldehyde in PBS per well overnight at 4° C. On the next day, cells were washed twice with 150 μl/well of FACS Buffer. Finally, cells were resuspended with 150 μl/well of FACS Buffer and 100 μl/sample were acquired using the BD FACSymphony™ A5 Cell Analyzer.

In another experiment, plates with co-cultures of PBMCs and MOLP-2 cells were removed from the incubator after 4 days and centrifuged at 400×g for 4 minutes at 4° C. Cells were washed once with PBS (200 μL/well) and inserted into a shaker for 15 seconds at 850 rpm to resuspend the cells. PBS including Zombie UV™ Fixable Viability Dye (Biolegend 423108,1:1000) was added to the wells (50 μL/well) and incubated for 20 min at RT (in the dark). Next, 150 μl PBS was added per well, cells were centrifuged at 400×g for 4 min, supernatant was flicked and cells were resuspended by using a shaker as described above. Thereafter, 25 μL of an FACS antibody mix (BUV737 anti-human CD4, clone SK3 from BD Biosciences, BV785 anti-human CD8, clone SK1, BV605 anti-human CD25, clone BC96, PE anti-human CD137, clone 4B4-1, PE/Dazzle594 anti-human CD3, clone OKT3, all but anti-human CD4 from BioLegend and pre-diluted in FACS Buffer) were added per well and cells were incubated for 30 minutes at 4° C. in the dark. Cells were washed three times with 200 μl FACS Buffer/well, mixed with the Eppendorf MixMate at 850 rpm for 15 seconds and fixed with 50 μl of 1% Formaldehyde in PBS per well for 15 minutes at room temperature in the dark. Next, cells were washed twice with 150 μl/well of FACS Buffer. Finally, plates were centrifuged, supernatants were flipped off and FACS buffer containing Counting Beads (Life Technologies, C36950) were added to have 5 μl beads (4900 beads per well in 150 μl). Plates were acquired using the BD FACS Fortessa. In order to determine tumor cell lysis of MOLP-2 cells, a pre-gate in FSC-A/SSC-A channel was set to include the larger tumor cells and exclude lymphocytes as well as counting beads. Next was gated for singlets (FSC—H, FSC-A) and from these for FarRed positive/CD3 negative cells. Then Zombie UV positive, dead cells were gated. The dead cell count per μl was calculated by dividing the cell count from this gate with the bead count (separate gate in FSC-A/SSC-A) multiplied by the counting bead concentration.

### Results

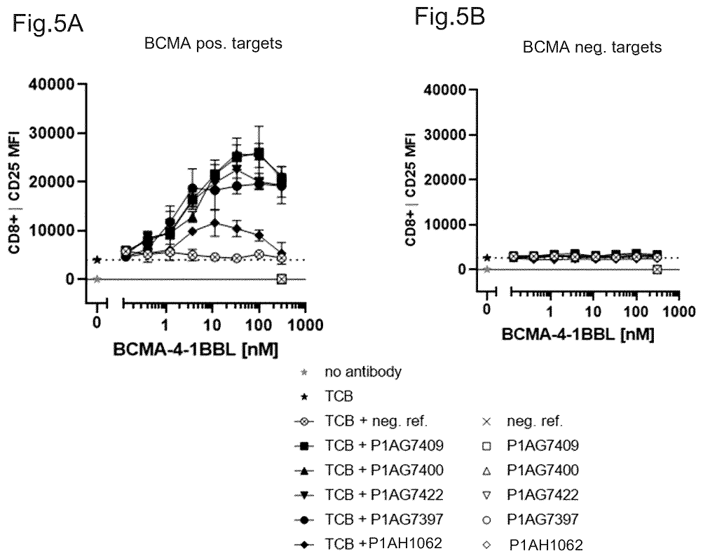

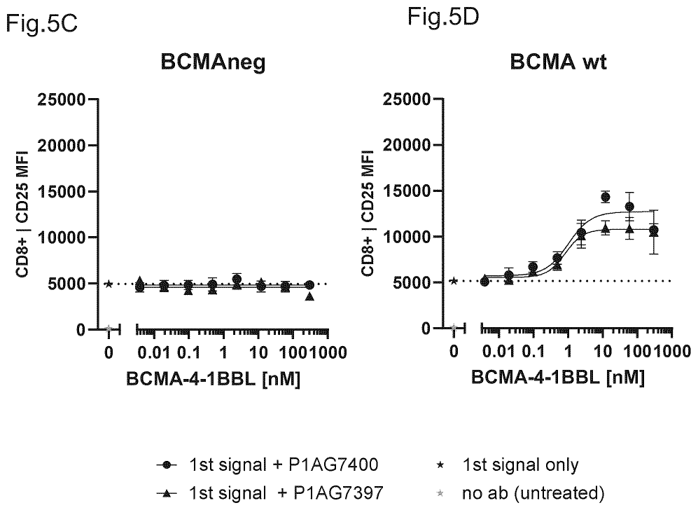

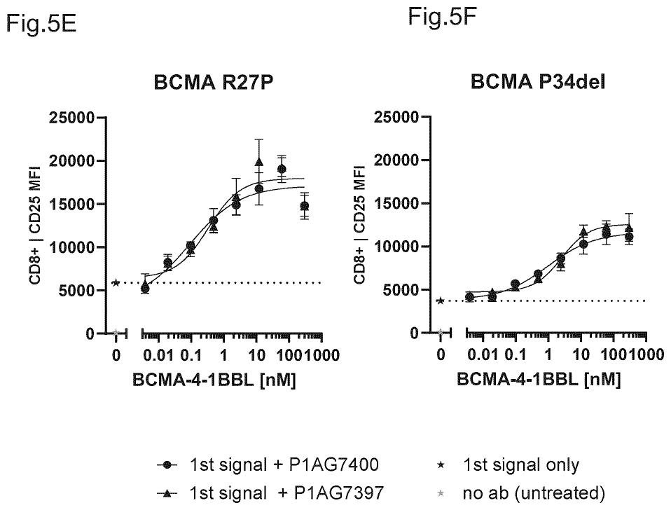

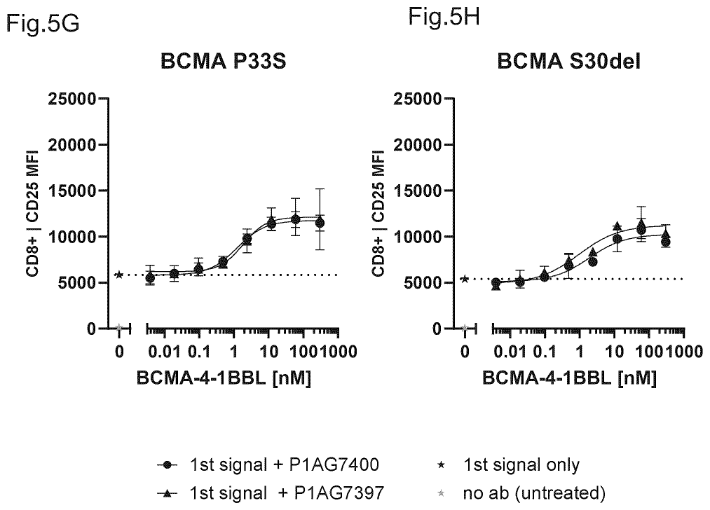

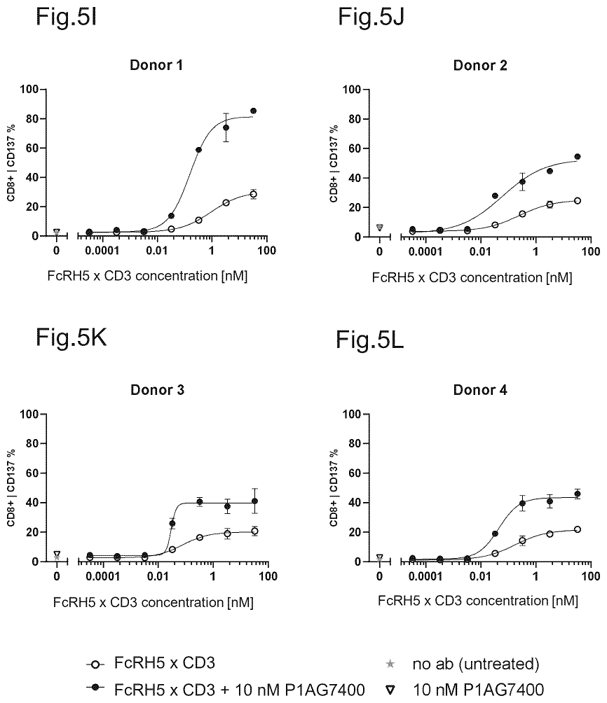

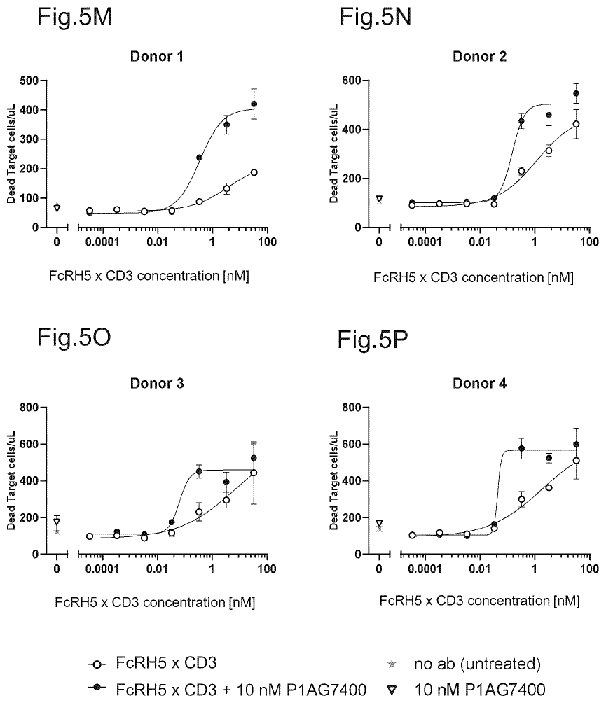

As illustrated in FIG. 5A and FIG. 5B, all BCMA-4-1BBL antigen binding molecules were able to significantly boost activation of CD8+ T cells (CD25 upregulation) in the presence of BCMA-expressing target cells and a first signal provided by a TCB (FIG. 5A). None of the BCMA-4-1BBL molecules is inducing T cell activation in the absence of a first signal/TCB at 300 nM. Furthermore, there is no boosting of TCB-mediated activation of T cells in the absence of BCMA-expressing targets (FIG. 5B). On top, the molecules have a higher efficacy than the bispecific antibody P1AH1062 (5D5 LM4).

Table 19 summarizes the EC₅₀ values, as well as Emax values derived from the data shown in FIG. 5A as well as results obtained with 1-3 additional donors. EC₅₀ values were calculated using GraphPadPrism6.

TABLE 19
EC₅₀ values (nM) and Efficacy of CD25 upregulation on CD8 T cells

| | EC₅₀ (nM) | | | | Efficacy (Emax (MFI)) | | | |
|---|---|---|---|---|---|---|---|---|
| | | | Donor 3 | | | | Donor 3 | |
| | | | (shown | | | | (shown | |
| | | | in Fig. | | | | in Fig. | |
| Molecule | Donor 1 | Donor 2 | 5A) | Donor 4 | Donor 1 | Donor 2 | 5A) | Donor 4 |
| P1AG7409 | 0.980 | 1.063 | 3.095 | 1.053 | 13532 | 22866 | 23932 | 24010 |
| P1AG7400 | 1.272 | 1.477 | 4.958 | 2.306 | 14036 | 25341 | 24254 | 23437 |
| P1AG7422 | 0.819 | 1.197 | 2.315 | 1.064 | 14416 | 26887 | 20680 | 22327 |
| P1AG7397 | 0.481 | 0.889 | 1.240 | 1.036 | 13585 | 25095 | 19191 | 19788 |
| P1AH1062 | not | not | 1.324 | 0.498 | not | not | 9254 | 9094 |
| | tested | tested | | | tested | tested | | |

In another co-culture assay, the functional activity of the bispecific BCMA-4-1BBL molecules P1AG7397 and P1AG7400 was tested in combination with an anti-CD3 IgG as the first signal provider and in presence of CHO transfectants expressing either the wildtype or mutated forms of human BCMA (see also Example 3, binding to transfectants).

In line with the retained binding of both BCMA-4-1BBL molecules to the various point mutations in the ECD of human BCMA as compared to the wildtype form of human BCMA (Example 3), both molecules display a similar concentration-dependent boosting of T cell activation on top of the CD3 IgG-mediated T cell activation (as illustrated by the upregulation of CD25 on CD8 T cells) in presence of either the mutated or the wildtype BCMA expressed on CHO transfectants (FIG. 5C to FIG. 5H, Table 19A). The difference in the maximal activity for the wt versus the mutated BCMA transfectants is due to different expression levels of the human BCMA wt and variants on the transfectants. This data supports the broader use of BCMA-4-1BBL also in patients harboring one of the tested BCMA point mutations without expecting any significant loss of activity.

TABLE 19A
EC₅₀ values (nM) for T cell activation as measured by flow
cytometry and detection of CD25 on CD8 T cells

| | EC₅₀ (nM) | | | | |
|---|---|---|---|---|---|
| | | CHO- | CHO- | CHO- | CHO- |
| | | K1_ | K1_ | K1_ | K1_ |
| | CHO- | BCMA | BCMA | BCMA | BCMA |
| | K1_BCMA | R27P_ | P34del_ | P33S_ | S30del_ |
| Molecule | (wildtype) | clone_12 | clone_C10 | clone_D5 | clone_H1 |
| P1AG7400 | 1.02 | 0.09 | 0.98 | 1.24 | 1.89 |
| P1AG7397 | 0.83 | 0.33 | 2.5 | 1.85 | 0.95 |

To test the combination with another CD3 bispecific molecule in the treatment of Multiple Myeloma, another series of assays was conducted to test the boosting of T cell activation and tumor cell lysis on top of a CD3 engager targeted to FcRH5 (FcRH5 x CD3). nM of BCMA-4-1BBL further boosted FcRH5 x CD3-induced T cell activation and lysis of MOLP-2 tumor cells over a broad range of FcRH5 x CD3 concentrations, namely 32 μM up to 32 nM for T cell activation (frequency of CD137-positive CD8 T cells, FIG. 5I to FIG. 5L) and 320 μM to 32 nM for the lysis of tumor cells (FIG. 5M to FIG. 5P). The data supports the capability of BCMA-4-1BBL to further boost T cell activation and tumor cell lysis induced by various T cell engagers.

### 4.3 Ex Vivo Functional Characterization of 4-1BBL Agonistic Antigen Binding Molecules Targeting BCMA

The therapeutic efficacy of the bispecific BCMA-4-1BBL antigen binding molecules was tested in combination therapy with GPRC5D-TCB in heparinized primary MM patient bone marrow aspirate (BMA) samples obtained within the first 48 hours after extraction.

Cell amounts, their viability and phenotype, as well as percent of diseased MM plasma cells at baseline were analyzed by flow cytometry. Next, at least 0.1 million MM plasma cells (MM PCs) were seeded in 24- or 48-well plates. A fixed concentration of a GPRC5D-CD3 bispecific molecule (GPRC5D TCB, 1 or 10 nM as indicated) was tested alone or in combination with increasing doses of the different BCMA-4-1BBL antigen binding molecules. As negative references, a TCB isotype, respectively an untargeted 4-1BBL molecule was added. After 96 hours cells were transferred into 15 ml falcon tubes. Sterile PBS was added up to 1 mL. Cells were centrifuged at 540 g for 5 min and erythrocytes were lysed for 15 minutes according to standard methods. Cells were centrifuged at 800 g for 10 minutes, washed with sterile PBS once and incubated at room temperature for 20 minutes with maleimide to stain dead versus living cells. Cells were washed with 12 ml of PBS, including 0.09% of NaN3 and 0.5% of BSA to remove unbound maleimide. Thereafter, cell pellets were resuspended in 500 μL PBS and filtered through 5 ml polystyrene Round-bottom tubes with cell-strainer cap to discard cell clots and remove potential membrane aggregates. Cells were centrifuged once more at 540 g for 5 minutes. Finally, cells were stained with the indicated antibodies against different surface markers for 20 min at RT protected from light (Table 20 and Table 21), washed as described above and resuspended in sterile PBS. The total sample was acquired by flow.

TABLE 20
Antibody combination used for the analysis of pre-treatment sample

| BB515 | BB700 | PE | APC | APCR700 | APC/Cy7 | BV421 | BV480 |
|---|---|---|---|---|---|---|---|
| CD38ME | CD138 | BCMA | GPRC5D | CD56 | CD45 | CD137 | CD3 |
| CYT-38F/ | Clone MI15 | clone 19F2 | Clone from | Clone N901 | clone | Clone | clone SK7 |
| CYT-38F2 | BD | Biolegend | Roche | (NKH-1) | 2D1 | 4B4-1 | BD |
| Cytognos | Bioscienes | | | BC | Biolegend | Biolegend | Biosciences |

| BV605 | BV650 | BUV395 | BUV496 |
|---|---|---|---|
| 4-1BBL | CD28 | HLADR | CD8 |
| Clone | Clone | clone Tu39 | Clone HIT8a |
| C65-485 | CD28.2 | BD | BD |
| BD | BD | Biosciences | Biosciences |
| Biosciences | Biosciences | | |

TABLE 21
Antibody combination used to determine MM-PC lysis and effector cell activation

| BB515 | BB700 | PE | PE-CF594 | APC | PC7 | APCR700 | APC/Cy7 |
|---|---|---|---|---|---|---|---|
| CD38ME | CD138, | CD127, | CD57, | CD25, | CD13, | CD56 | CD45, |
| CYT-38F/ | Clone MI15, | clone | clone | Clone 2A4, | cloneL138 , | Clone | clone 2D1, |
| CYT-38F2, | BD | A019D5, | NK-1, | BD | BD | N901 | Biolegend |
| Cytognos | Bioscienes | Biolegend | BD | Biosciences | Biosciences | (NKH-1), | |
| | | | Biosciences | | | BC | |

| BV421 | BV480 | BV605 | BV650 | BUV395 | BUV496 |
|---|---|---|---|---|---|
| CD4 | CD16, | CD3, | PD1, | HLADR, | CD14, |
| B49197 | clone 3G8, | Clone SK7, | Clone EH12, | Clone | clone |
| | BD | BD | BD | Tu39, | MΦP9, |
| | Biosciences | Biosciences | Biosciences | BD | BD |
| | | | | Biosciences | Biosciences |

In another experiment, the therapeutic efficacy of the BCMA-4-1BBL antigen binding molecules was tested in combination therapy with GPRC5D-TCB using frozen and thawed primary MM bone marrow mononuclear cells (BMMNCs) as described below.

On day one, samples were thawed and resuspended in StemSpan SFEMII (StemCell) medium, including 20% human serum, 55 M β-mercaptoethanol (Gibco cat #11528926) and 100 U/ml penicillin and 100 μg/ml streptomycin (Gibco, 100× stock). 3 Mio cells were plated per well of a 12-well-plate, including 100 ng/mLof recombinant human IL-6 and stored in the incubator at 37° C., 5% CO₂ for 24 hours.

Thereafter, samples were harvested and 0.1 Mio cells were used for baseline characterization. The remaining cells were washed twice with sterile PBS and stained with a NIR live/dead stain prior to being sorted for viability using the FACS BD Aria sorter. Living cells were resuspended in medium and 0.1 Mio cells were plated per well of a 96-well-plate. Cells were incubated for 96 hours in the absence or presence of the indicated antibodies in a total volume of 150 μL per well at 37° C., 5% CO₂. Samples were washed twice with sterile PBS and stained as follows: First, a live/dead staining was performed for 20 minutes at room temperature in the dark, using a NIR dye (ThermoFisher). After one washing step, human TruStain Fcx Blocking was added in FACS buffer (PBS with 2% Fetal Bovine Serum, 1% 0.5 M EDTA pH 8, 0.25% NaN3 Sodium azide) and cells were incubated for another 10 minutes at room temperature (RT) in the dark prior to addition of the respective antibody mix (see Table 25).

Surface staining was performed for 20 minutes at RT in the dark. Cells were washed with FACS buffer once and fixed in PBS, containing 2% paraformaldehyde (PFA) for 10 minutes at RT in the dark. Samples were washed with FACS buffer, resuspended in FACS buffer, and analyzed by flow cytometry after addition of counting beads to determine the absolute number of cells.

TABLE 22
Antibody combination used to do the baseline
characterization and effector cell activation

| | Marker | Fluorochrome |
|---|---|---|
| Baseline panel: | | |
| | hCD45 | BUV395 |
| T cells | CD3 | BV510 |
| | CD8 | BUV737 |
| | CD4 | PerCPCy5.5 |
| B cells | CD19 | FITC |
| Activated cells/ | HLA-DR | BV786 |
| myeloid cells | | |
| Activated T cells | 4-1BB | PE-Cy5 |
| | CD25 | Pe-Dazzle594 |
| | CD28 | BV650 |
| | CD69 | PE−Cy7 |
| Multiple Myeloma | CD138 | BV421 |
| (+ level of malignant) | CD38 | BV711 |
| | BCMA | PE |
| | GPR5CD | AF647 |
| NK cells/malignant | CD56 | BV605 |
| cells | | |
| | live/dead | NIR |
| Isotype Panel | | |
| | hCD45 | BUV395 |
| | CD3 | BV510 |
| | CD8 | BUV737 |
| | CD4 | PerCPCy5.5 |
| | CD19 | FITC |
| | isotype | BV786 |
| | isotype | PE-Cy5 |
| | isotype | Pe-Dazzle594 |
| | isotype | BV650 |
| | isotype | PE-Cy7 |
| | CD138 | BV421 |
| | CD38 | BV711 |
| | isotype | PE |
| | isotype | AF647/APC |
| | CD56 | BV605 |
| | live/dead | NIR |
| Activation panel: | | |
| | hCD45 | BUV395 |
| | CD3 | BV510 |
| T cells | CD8 | BUV737 |
| | CD4 | PerCPCy5.5 |
| B cells | CD19 | FITC |
| Activated cells/ | HLA-DR | BV786 |
| myeloid cells | | |
| Activated T cells | 4-1BB | PE-Cy5 |
| | CD25 | Pe-Dazzle594 |
| | CD28 | BV650 |
| | CD69 | PE−Cy7 |
| | PD-1 | PE |
| | CD39 | APC |
| | TIM-3 | BUV615 |
| Multiple Myeloma | CD138 | BV421 |
| (+ level of malignant) | CD38 | BV711 |
| NK cells/malignant | CD56 | BV605 |
| cells | | |
| | live/dead | NIR |

The FACS surface antibodies were purchased from BD Biosciences, Miltenyi or LuBiosciences and used according to the manufacturers' recommendations. The detection antibody for human GPRC5D was generated by Roche.

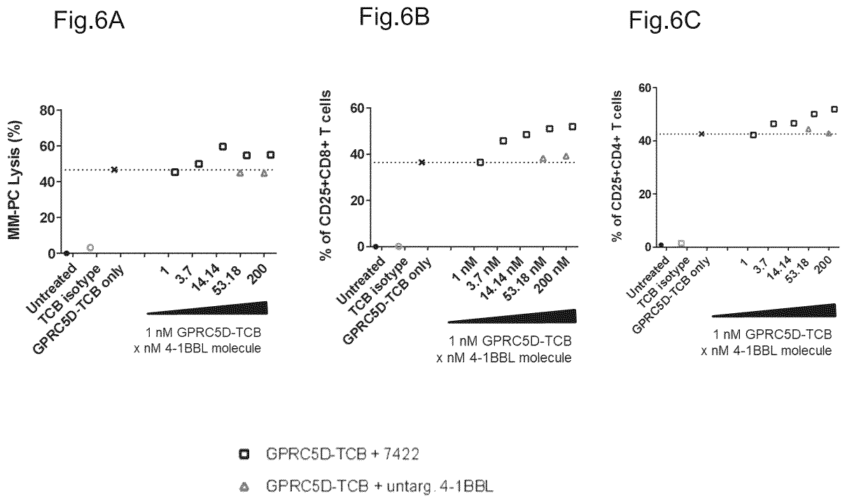

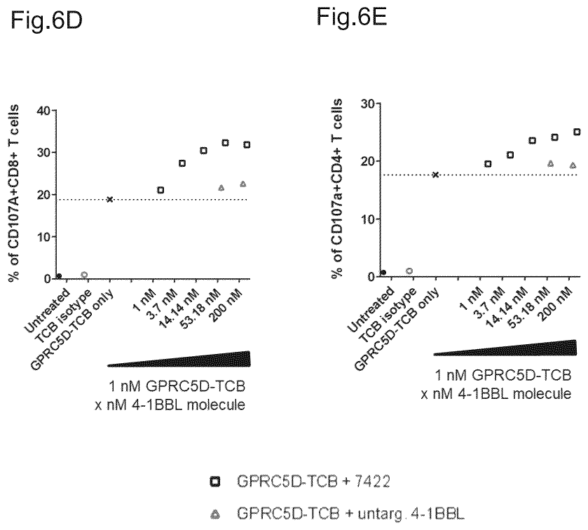

Results: FIG. 6A to FIG. 6E and Table 23 demonstrate that BCMA-4-1BBL antigen binding molecule P1AG7422 was able to significantly boost activation of CD8⁺ and CD4⁺ T cells (CD25 upregulation) as well as increase the level of the degranulation marker CD107a on top of the GPRC5D-targeted CD3 engager (GPRC5D-TCB) mediated activation and induction of degranulation. No activation on top of the TCB was induced in presence of 200 or 53 nM of the untargeted 4-1BBL, which demonstrates that the 4-1BBL molecules need to be crosslinked via the tumor antigen-targeting moiety to be active.

TABLE 23
Percent of tumor cell lysis, respective Up-regulation of the
activation marker CD25 or the degranulation marker
CD107a on CD8⁺ and CD4⁺ T cells upon incubation
of primary MM BM aspirate samples with the indicated
molecules for 96 hours, as assessed by flow cytometry

| | % | % | % | % | % |
|---|---|---|---|---|---|
| | Tumor | CD25⁺ | CD25⁺ | CD107a⁺ | CD107a⁺ |
| | Cell | CD8⁺ T | CD4⁺ T | CD8⁺ T | CD4⁺ T |
| Treatment | Lysis | cells | cells | cells | cells |
| untreated | 0 | 0.03 | 0.91 | 0.72 | 0.78 |
| TCB isotype | 3.15 | 0.09 | 1.5 | 0.98 | 1 |
| GPRC5D-TCB only | 46.65 | 36.5 | 42.57 | 18.83 | 17.63 |
| GPRC5D- | 44.96 | 38.25 | 44.48 | 21.71 | 19.64 |
| TCB + 53.18 | | | | | |
| nM Untarg. 4-1BBL | | | | | |
| GPRC5D-TCB + | 44.84 | 39.34 | 42.85 | 22.61 | 19.34 |
| 200 nM | | | | | |
| Untarg. 4-1BBL | | | | | |
| GPRC5D- | 45.24 | 36.45 | 42.18 | 21.09 | 19.53 |
| TCB + 1 nM | | | | | |
| BCMA-4-1BBL | | | | | |
| GPRC5D-TCB + | 49.89 | 45.78 | 46.43 | 27.42 | 21.06 |
| 3.7 nM | | | | | |
| BCMA-4-1BBL | | | | | |
| GPRC5D- | 59.56 | 48.55 | 46.62 | 30.44 | 23.55 |
| TCB + 14.14 | | | | | |
| nM BCMA-4-1BBL | | | | | |
| GPRC5D- | 54.54 | 51.06 | 50.04 | 32.28 | 24.1 |
| TCB + 53.18 | | | | | |
| nM BCMA-4-1BBL | | | | | |
| GPRC5D-TCB + | 54.99 | 52 | 51.82 | 31.84 | 25.07 |
| 200 nM | | | | | |
| BCMA-4-1BBL | | | | | |

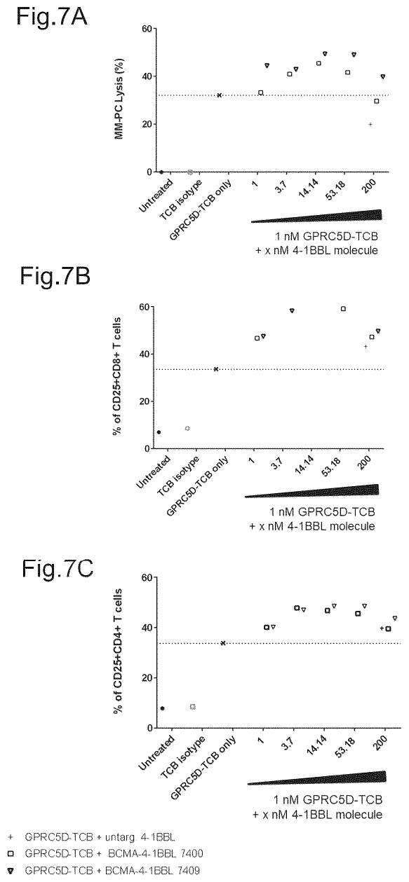

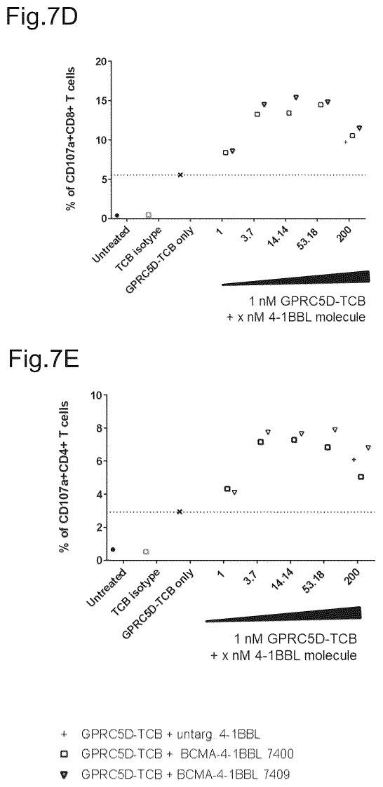

This could be demonstrated as well with another primary MM patient BM aspirate sample, as depicted in FIG. 7A to FIG. 7E and Table 24. Here, boosting of TCB-mediated lysis of primary malignant MM plasma cells or T cell activation (upregulation of CD25) could be observed in presence of 4.4 nM and higher concentrations of the BCMA-4-1BBL molecules P1AG7409 and P1AG7400, but no significant lysis could be observed in presence of the untargeted 4-1BBL at concentrations of up to even 40 and 120 nM.

TABLE 24
Percent of tumor cell lysis, respective Up-regulation of the
activation marker CD25 on CD8⁺ and CD4⁺ T cells upon
incubation of primary MM BM aspirate samples with the indicated
molecules for 96 hours, as assessed by flow cytometry

| | % Tumor | % CD25+ | %CD25+ |
|---|---|---|---|
| | Cell | CD8+ T | CD4+ T |
| Treatment | Lysis | cells | cells |
| untreated | 0 | 7.03 | 7.93 |
| TCB isotype | −29.44 | 8.67 | 8.53 |
| GPRC5D-TCB only | 32.13 | 33.58 | 33.8 |
| GPRC5D-TCB + 200 nM Untarg. | 19.93 | 43.2 | 39.72 |
| 4-1BBL | | | |
| GPRC5D-TCB + 1 nM BCMA-4- | 33.2 | 46.62 | 40.1 |
| 1BBL 7400 | | | |
| GPRC5D-TCB + 3.7 nM BCMA- | 40.92 | 61.98 | 47.79 |
| 4-1BBL 7400 | | | |
| GPRC5D-TCB + 14.14 nM | 45.39 | 60.71 | 46.78 |
| BCMA-4-1BBL 7400 | | | |
| GPRC5D-TCB + 53.18 nM | 41.61 | 59.07 | 45.56 |
| BCMA-4-1BBL 7400 | | | |
| GPRC5D-TCB + 200 nM BCMA- | 29.56 | 47.2 | 39.52 |
| 4-1BBL 7400 | | | |
| GPRC5D-TCB + 1 nM BCMA-4- | 44.34 | 47.41 | 40.19 |
| 1BBL 7409 | | | |
| GPRC5D-TCB + 3.7 nM BCMA- | 42.86 | 58.2 | 46.99 |
| 4-1BBL 7409 | | | |
| GPRC5D-TCB + 14.14 nM | 49.24 | 61.35 | 48.45 |
| BCMA-4-1BBL 7409 | | | |
| GPRC5D-TCB + 53.18 nM | 48.89 | 60.42 | 48.5 |
| BCMA-4-1BBL 7409 | | | |
| GPRC5D-TCB + 200 nM BCMA- | 39.74 | 49.57 | 43.61 |
| 4-1BBL | | | |

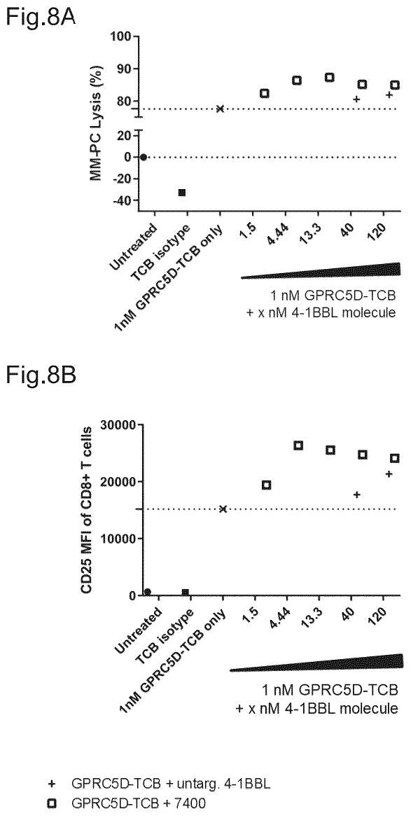

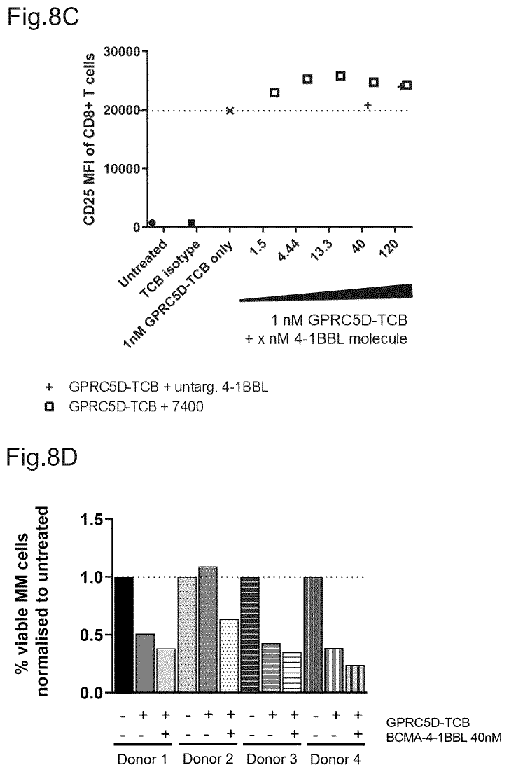

Another series of experiments was conducted using BM MNCs from primary MM patients after thawing. As indicated in FIG. 8A to FIG. 8D and Tables 25 and 26, 40 nM of BCMA-4-1BBL (P1AG7400) induced significant lysis of malignant MM plasma cells on top of the GPRC5D-targeted TCB in all four samples assessed. The maximal effect induced by the TCB, as well as by the combination of TCB and BCMA-4-1BBL differed between the MM patient samples, suggesting that the immune cell composition, the effector to target cell ratio and T cell fitness may impact the overall results.

TABLE 25
Percent of tumor cell lysis, respective Up-regulation of the activation
marker CD25 on CD8⁺ and CD4⁺ T cells upon incubation of
primary MM BM aspirate samples with the indicated molecules
for 96 hours, as assessed by flow cytometry

| | % Tumor Cell | CD25 MFI on | CD25 MFI on |
|---|---|---|---|
| Treatment | Lysis | CD8+ T cells | CD4+ T cells |
| untreated | 0.00 | 650.17 | 732.45 |
| TCB isotype | −32.93 | 541.47 | 700.76 |
| GPRC5D-TCB only | 77.57 | 15197.3 | 19857.16 |
| GPRC5D-TCB + 40 nM | 80.57 | 17703.97 | 20753.89 |
| Untarg. 4-1BBL | | | |
| GPRC5D-TCB + 120 nM | 81.89 | 21333.67 | 23928.77 |
| Untarg. 4-1BBL | | | |
| GPRC5D-TCB + 1.5 nM | 82.3 | 19415.77 | 22953.07 |
| BCMA-4-1BBL 7400 | | | |
| GPRC5D-TCB + 4.44 nM | 86.35 | 26367.28 | 25230.39 |
| BCMA-4-1BBL 7400 | | | |
| GPRC5D-TCB + 13.3 nM | 87.29 | 25549.06 | 25802.42 |
| BCMA-4-1BBL 7400 | | | |
| GPRC5D-TCB + 40 nM | 85.17 | 24739.75 | 24742.11 |
| BCMA-4-1BBL 7400 | | | |

TABLE 26
Increased lysis of primary malignant MM plasma cells upon
incubation of primary MM BM MNCs with BCMA-4-1BBL
(P1AG7400) for 96 hours, as assessed by flow cytometry

| | MM BM | MM BM | MM BM | MM BM |
|---|---|---|---|---|
| | MNCs, | MNCs, | MNCs, | MNCs, |
| | Donor 1 | Donor 2 | Donor 3 | Donor 4 |
| | % viable | % viable | % viable | % viable |
| | MM | MM | MM | MM |
| Treatment | cells | cells | cells | cells |
| untreated | 1 | 1 | 1 | 1 |
| 0.025 nM | 0.510519 | 1.090909 | 0.428571 | 0.387435 |
| GPRC5D-TCB | | | | |
| only | | | | |
| 0.025 nM | 0.382889 | 0.636364 | 0.351351 | 0.240838 |
| GPRC5D-TCB + | | | | |
| P1AG7400 | | | | |

Samples were normalized to Untreated cells (□ untreated cells = 1)

### Example 5

### In Vivo Functional Characterization of 4-1BBL Agonistic Antigen Binding Molecules Targeting BCMA

The efficacy studies described herein were aimed to understand the potency of the BCMA-4-1BBL antigen binding molecules in combination with an anti-GPRC5D/anti-CD3 bispecific antibody (GPRC5D x CD3).

Cell culture: Human NCI-H929 cells (obtained from Roche Nutley) were cultured in RPMI1640 high glucose medium containing 10% FCS, 2 mM L-Glutamine, 10 mM HEPES and 1 mM Sodiumpyruvate (37° C. at 5% CO₂). In order to generate tumor bearing mice, 50 microliters cell suspension (2.5×10⁶ cells) were co-injected with 50 μl Matrigel subcutaneously in the right flank of anaesthetized, humanized NSG mice.

Mouse model: Humanized NSG mice were provided by Jackson Laboratories, Sacramento USA. For engraftment, animals were irradiated (140cGy) and injected with CD34+ cord blood cells (9×10⁴ cells) from healthy donors (hematopoietic stem cells; HSC). After arrival animals were maintained for one week to get accustomed to new environment and for observation. Mice were maintained under specific pathogen-free conditions with daily cycles of 12 h light/12 h darkness according to committed guidelines (GV-Solas; Felasa; TierschG). Continuous health monitoring was carried out on a regular basis. Experimental study protocol was reviewed and approved by the local government (ROB-55.2-2532.Vet 03-20-170).

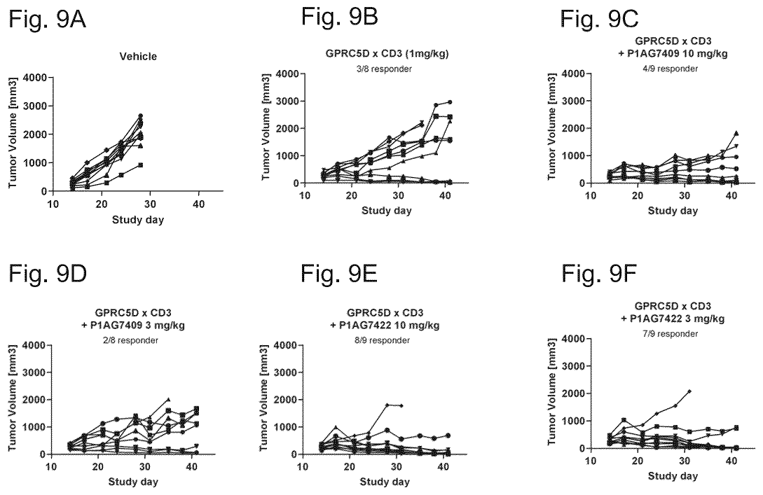

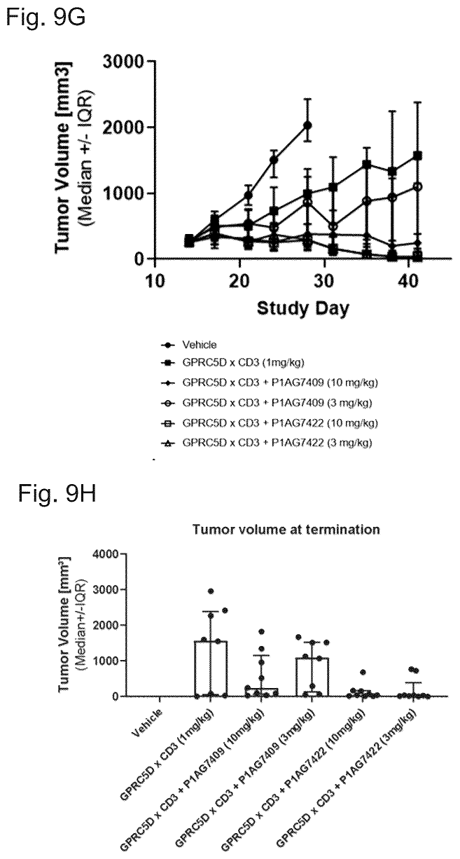

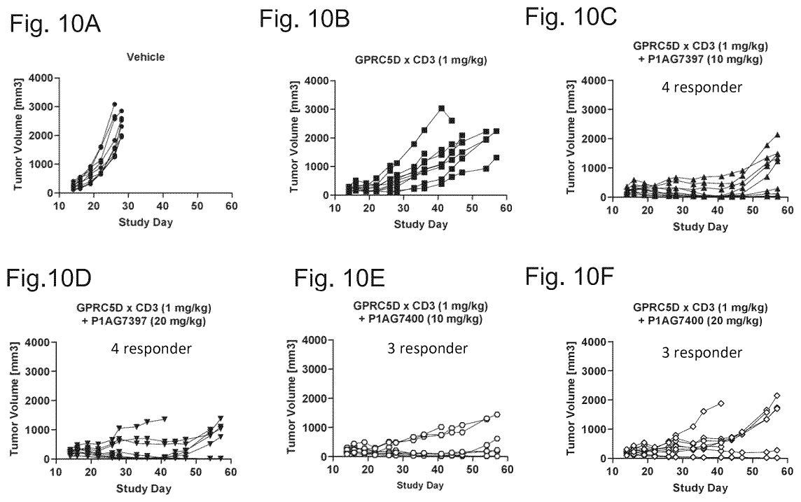

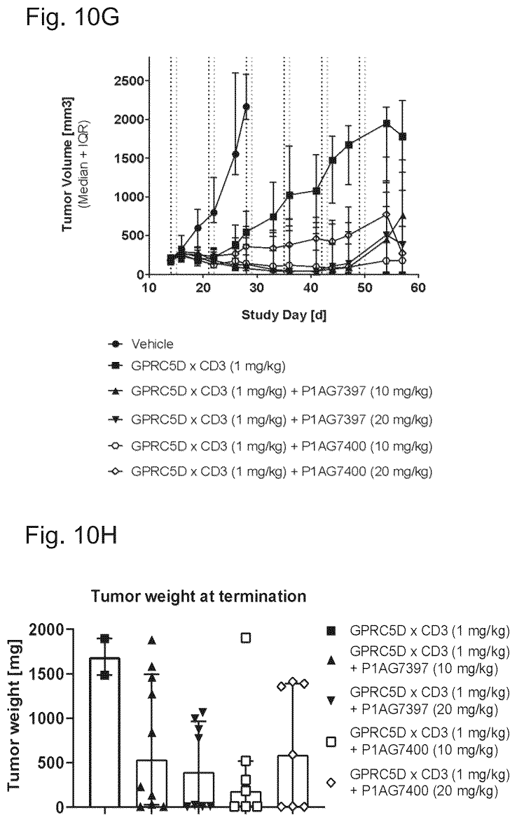

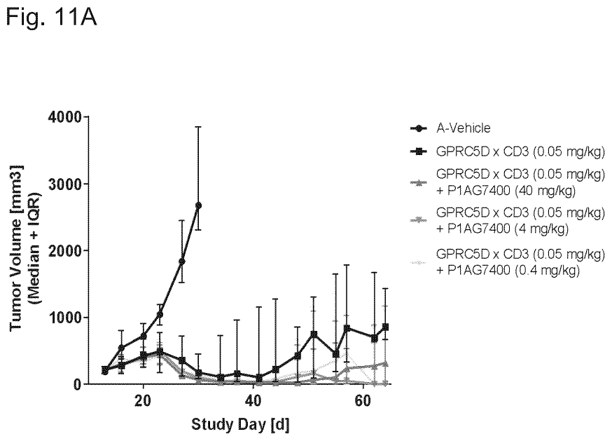

Randomization and treatment: When subcutaneous tumors reached a tumor volume of 200 mm³, humanized mice were randomized into different treatment groups based on tumor volume and body weight (n=10/group). All antibodies and vehicle (Histidin buffer) were administered intravenously (i.v.) once weekly. Costimulator molecules were injected 48 hours (FIG. 9A to FIG. 9H) or 24 hours (FIG. 10A to FIG. 10H) after GPRC5D x CD3 treatment.

Monitoring of animals: Animals were controlled daily for clinical symptoms and detection of adverse effects. Body weight and tumor growth (Caliper measurement) was monitored twice weekly. Animals were sacrificed according to the termination criteria or at the end of the experiment.

Statistics: Graphs were generated using GraphPad prism Software. A time-to-event analysis was performed using the internal tool DOPSa (based on the software R). The critical tumor volume was chosen as the maximum observed baseline tumor volume plus the standard deviation of the baseline tumor volume (rounded to the nearest multiple of ten). Study groups were compared using the Log-Rank test and p-values have been corrected for multiple testing using the Bonferroni-Holm method. Significant changes compared to the control group (GPRC5D x CD3) with p<0.05 were depicted with asterisks (* p<0.05, ** p<0.01, *** p<0.001).

Combination of GPRC5D x CD3 with BCMA-4-1BBL

In order to compare GPRC5D x CD3 monotherapy to combination with BCMA-4-1BBL molecules, NCI-H929 tumor-bearing animals were treated once weekly and tumor growth inhibition was monitored. The BCMA-4-1BBL costimulator molecules P1AG7422 and P1AG7409 were administered 48 hours after GPRC5D x CD3 treatment (P1AE3357). In summary, GPRC5D x CD3 monotherapy (1 mg/kg) induced a moderate tumor growth inhibition. Combination with P1AG7422 induced a significantly higher anti-tumoral efficacy with both concentrations (10 and 3 mg/kg) than the GPRC5D x CD3 monotherapy (FIG. 9A to FIG. 9H). The number of responders was highest in the treatment group of GPRC5D x CD3 with P1AG7422 (10 mg/kg; 8 responders) and showed lowest tumor volumes at termination.

In a follow up study, P1AG7397 and P1AG7400 were tested as combination partners for GPRC5D x CD3 ((P1AE3357) in two different doses (10 and 20 mg/kg; 24 hours after GPRC5D x CD3) (FIG. 10A to FIG. 10H). All combinations induced significantly higher tumor growth inhibition compared to GPRC5D x CD3 monotherapy (Table 28). P1AG7397 induced a slightly higher number of responders (n=4) compared to P1AG7400 (n=3) in combination with GPRC5D x CD3. Most homogeneous response was induced by the combination of GPRC5D x CD3 with 10 mg/kg P1AG7400. This is also shown with the tumor weights (FIG. 10H) at termination indicating more heterogeneous tumor weights and sizes after GPRC5D x CD3 combination with 10 and 20 mg/kg P1AG7397 or 20 mg/kg P1AG7400. Tumor volumes of last measurement are shown in Table 27.

TABLE 27
Tumor volume (mm³) at termination

| | GPRC5D × CD3 | GPRC5D × CD3 | | | GPRC5D × CD3 | | | GPRC5D × CD3 | | |
|---|---|---|---|---|---|---|---|---|---|---|
| | (1 mg/kg) + | (1 mg/kg) + | | | (1 mg/kg) + | | | (1 mg/kg) + | | |
| | P1AG7397 | P1AG7397 | | | P1AG7400 | | | P1AG7400 | | |
| Treatment | (10 mg/kg) | 20 mg/kg) | | | (10 mg/kg) | | | (20 mg/kg) | | |
| Group | Day | Day | Day | Day | Day | Day | Day | Day | Day | Day |
| Study day | 57 | 41 | 54 | 57 | 41 | 54 | 57 | 41 | 54 | 57 |
| Animal | 21 | | | 10.99 | | | 178.88 | | | 12.89 |
| X01 | | | | | | | | | | |
| Animal | 114.71 | | | 1026.7 | | 1280.5 | | | | 270.71 |
| X02 | | | | | | | | | | |
| Animal | 21.09 | 1354.8 | | | | | 10.08 | | | 1735.4 |
| X03 | | | | | | | | | | |
| Animal | 1330.1 | | | 1375.5 | | | 3.99 | 1877.2 | | |
| X04 | | | | | | | | | | |
| Animal | 2146.5 | | 999.46 | | 754.4 | | | | | 1698.3 |
| X05 | | | | | | | | | | |
| Animal | 292.27 | | | 15.5 | | | 616.85 | 390.99 | | |
| X06 | | | | | | | | | | |
| Animal | 28.82 | | | 751.22 | | | | | | 7.72 |
| X07 | | | | | | | | | | |
| Animal | 1475.6 | | | 14.74 | | | 19.9 | | | 9.4 |
| X08 | | | | | | | | | | |
| Animal | 1495.2 | | | 1100.9 | | | 225.75 | | 1650.8 | |
| X09 | | | | | | | | | | |
| Animal | 1228.6 | | | 9.16 | | | 1442 | | | 2147.9 |
| X10 | | | | | | | | | | |

TABLE 28
P-values of combination groups compared to GPRC5D x CD3

| Comparison to GPRC5D x CD3 | P-Value | Significance |
|---|---|---|
| P1AG7397 (10 mg/kg) | 0.0004 | *** |
| P1AG7397 (20 mg/kg) | 0.0053 | ** |
| P1AG7400 (10 mg/kg) | 0.0043 | ** |
| P1AG7400 (20 mg/kg) | 0.0203 | * |

Log-Rank Test p > 0.05*, p > 0.01**, p > 0.001***

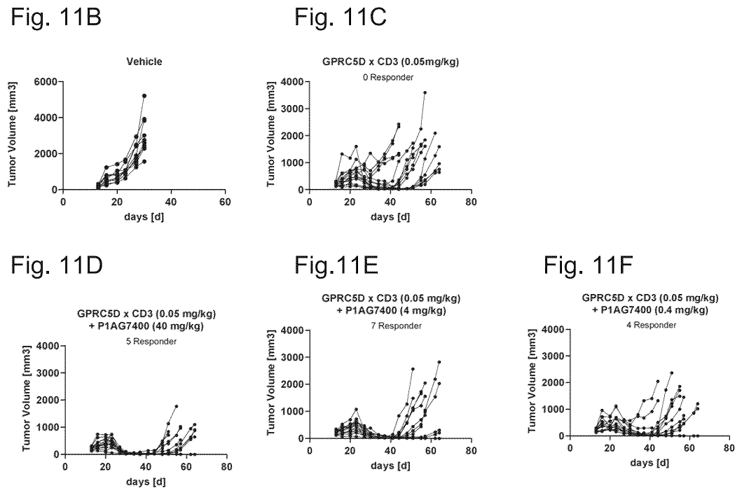

In the third experiment, P1AG7400 was tested in different doses (40, 4 and 0.4 mg/kg) in combination with GPRC5D x CD3 (0.05 mg/kg P1AE6625) and compared to the GPRC5D x CD3 monotherapy. P1AG7400 was injected 24 hours after GPRC5D x CD3. The GPRC5D x CD3 monotherapy induces strong tumor growth inhibition in the NCI-H929 model (FIG. 11A to FIG. 11F). However, tumors start to relapse after the 4th treatment cycle (Table 29). All three doses of P1AG7400 (40 and 10 mg/kg) inhibit the tumor regrowth. At termination of the experiment (day 64), 5, 7 and 4 responders were left in the P1AG7400 40, 4 and 0.4 mg/kg combination groups, respectively. In the GPRC5D x CD3 monotherapy group no responders were left.

TABLE 29
Timepoint of Tumor Escape

| | | After | After | After | After | |
|---|---|---|---|---|---|---|
| | No | 4th | 5th | 6th | 7th | |
| Group | Response | cycle | cycle | cycle | cycle | Responder |
| GPRC5D × CD3 | 4 | 1 | 5 | 3 | 2 | 0 |
| (0.05 mg/kg) | | | | | | |
| GPRC5D × CD3 | 0 | | 5 | 1 | 4 | 5 |
| (0.05 mg/kg) + | | | | | | |
| P1AG7400 | | | | | | |
| (40 mg/kg) | | | | | | |
| GPRC5D × CD3 | 0 | | 5 | 3 | 2 | 5 |
| (0.05 mg/kg) + | | | | | | |
| P1AG7400 | | | | | | |
| (4 mg/kg) | | | | | | |
| GPRC5D × CD3 | 2 | | 5 | 4 | | 4 |
| (0.05 mg/kg) + | | | | | | |
| P1AG7400 | | | | | | |
| (0.4 mg/kg) | | | | | | |

These results confirm that tumor growth inhibition induced by GPRC5D x CD3 in NCI-H929 tumor model is boosted by BCMA-41BBL.

### Example 6

### Pharmacological Properties of of 4-1BBL Agonistic Antigen Binding Molecules Targeting BCMA

6.1 PK Properties in huFcRn Transgenic Mice

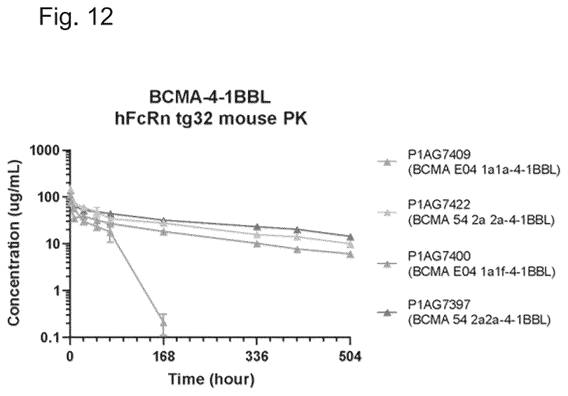

Transgenic mice carrying the human FcRn instead of the mouse FcRn are considered more predictive for the clearance in human than in wild-type mice (C57/B16). Female huFcRn Tg32 homozygous SCID mice (n=3 or 4) received a single IV bolus dose of P1AG740 or P1AG7397 at 5 mg/kg. Female huFcRn Tg32 homozygous BL6 mice (n=3 or 4) received a single IV bolus dose of P1AG7409 or P1AG7422, respectively, at 5 mg/kg. Blood samples were taken at 0.17, 7, 24, 48, 72, 168, 336, 408, and 504 hours after receiving the dose and processed to serum prior to bioanalysis. Serum samples were analyzed using an immunoassay with anti-human FCpan (CH2) or kCH capture and detection reagents. As shown in FIG. 12 and Table 30, all tested BCMA-4-1BBL antigen binding molecules have a similar plasma concentration-time profiles and clearance values ranging between 4 to 7 mL/kg/day in these mouse strains.

TABLE 30
Pharmacokinetic Data in hu FcRn transgenic mice

| | Cmax | Tmax | AUClast |
|---|---|---|---|
| Molecule | (μg/mL) | (hour) | (day*μg/mL) |
| P1AG7400 | 76.3 | 0.167 | 350 |
| P1AG7397 | 142 | 0.167 | 652 |
| P1AG7409 | 112 | 0.167 | 118 |
| P1AG7422 | 153 | 0.167 | 546 |

### 6.2 Off-Target Binding Assessment Assay

The Cell Microarray Technology (Charles River Laboratories) was used to screen for potential off-target binding interactions. BCMA-4-1BBL agonistic antigen binding molecules were screened for binding against fixed HEK293 cells expressing 6019 individual full-length human plasma membrane proteins and cell surface-tethered human secreted proteins, as well as a further 397 human heterodimers. Fluorescent images of the spotted cells were analyzed using ImageQuant software (GE healthcare, Version 8.2).

Table 31 reports the results of the screen as strong, medium, weak or no interaction after visual inspection of the spots intensity.

TABLE 31
Results of the Cell Microarray Assay

| | BCMA | 4-1BB | LDLR |
|---|---|---|---|
| TAPIR ID | interaction | Interaction | interaction |
| P1AE7397 | Strong | Strong | No |
| P1AG7409 | Strong | Strong | Weak/Medium |
| P1AG7422 | Strong | Strong | No |

As a conclusion, all antigen binding molecules showed a strong interaction with their primary targets, i.e. BCMA and 4-1BB. P1AG7409 showed weak or medium interactions with LDLR (low density lipoprotein receptor). No other interactions were detected for P1AG7397 and P1AG7422, indicating a high specificity of these molecules for their primary targets.

### 6.3 Immunogenicity Testing

### 6.3.1 MAPPs Assay

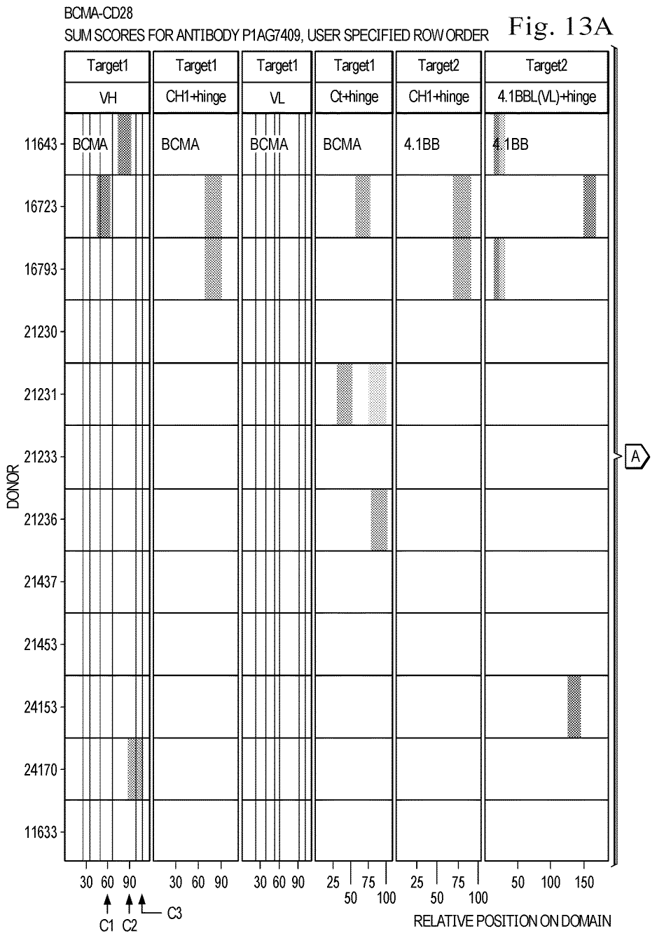

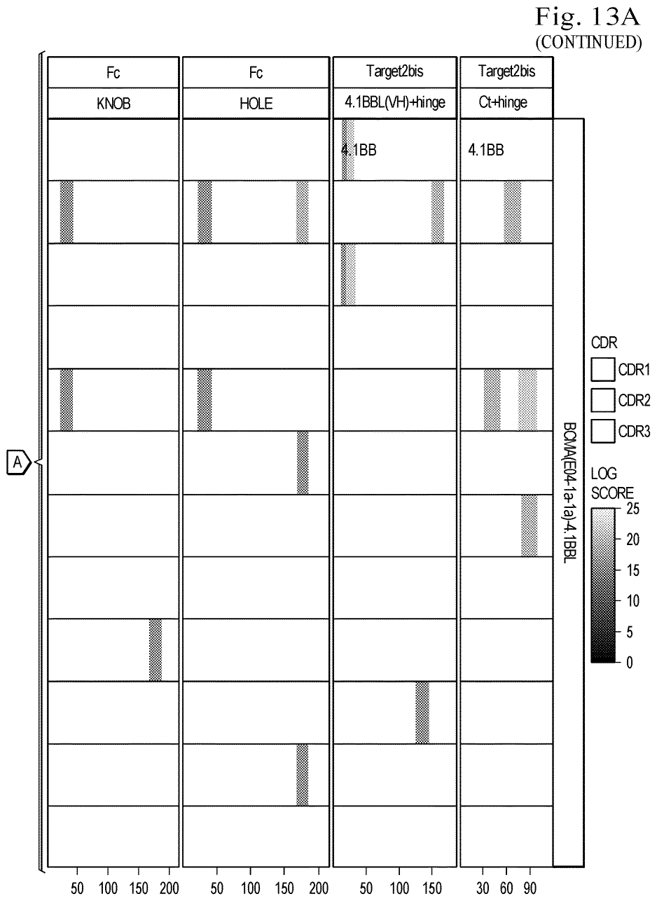

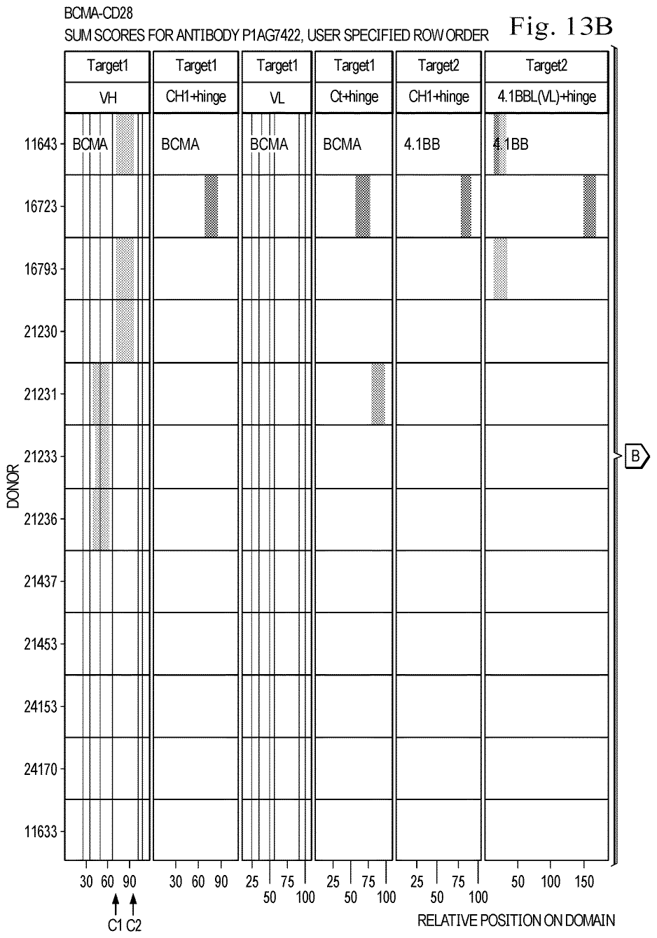

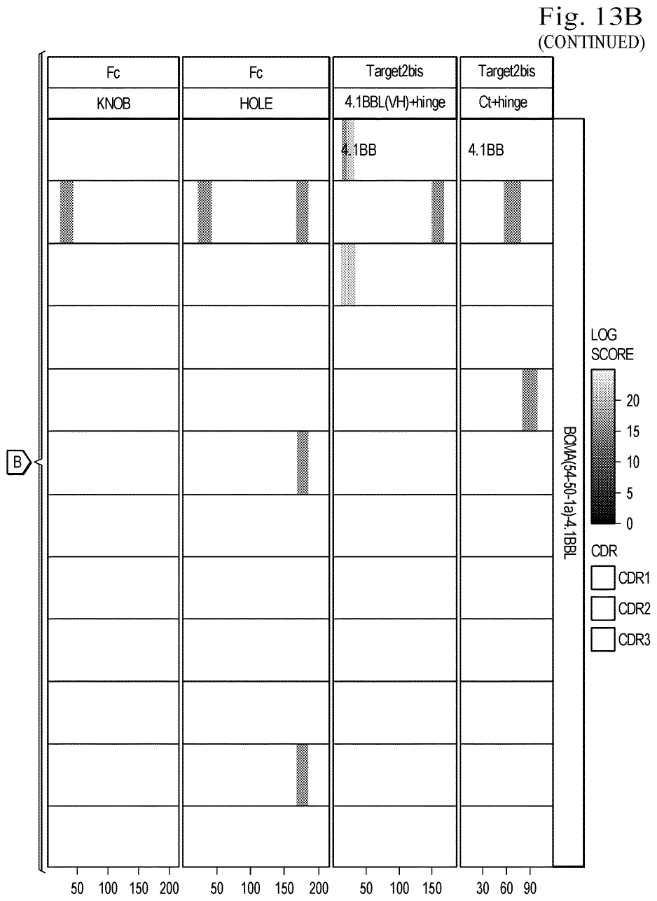

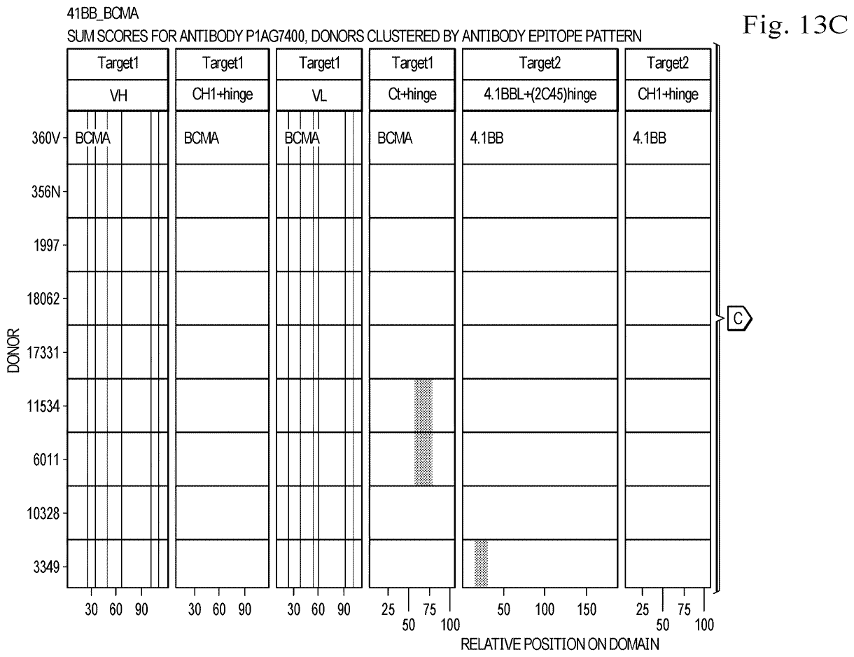

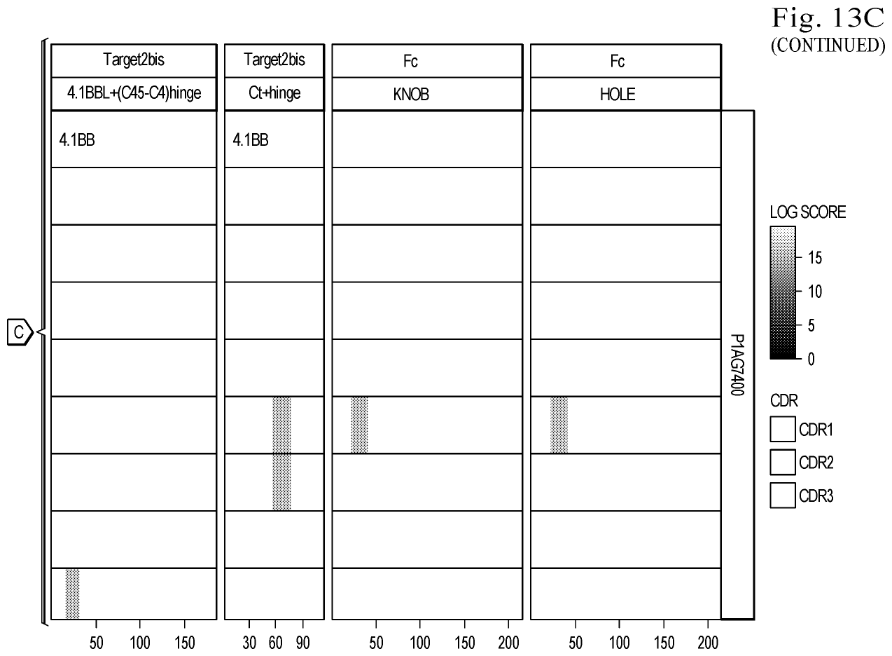

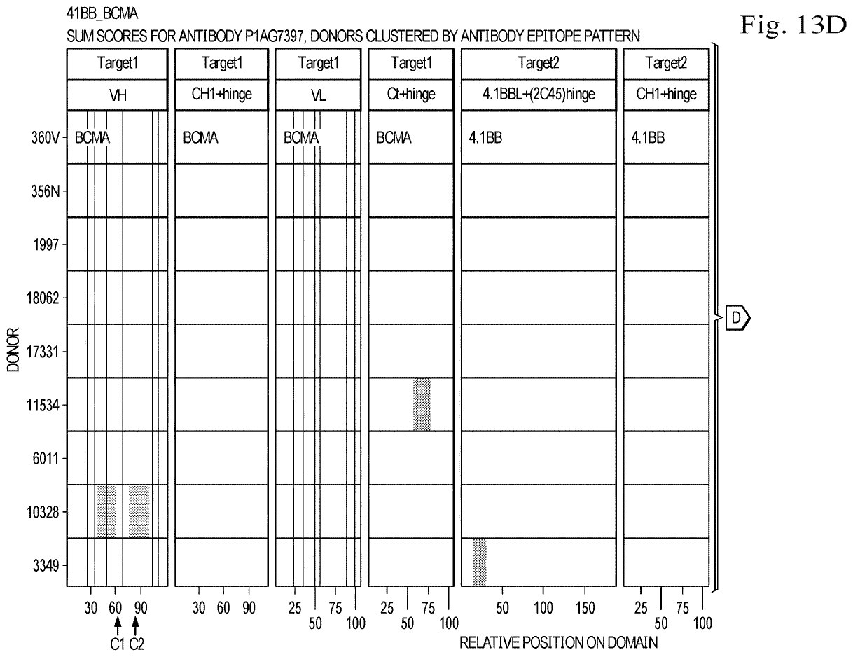

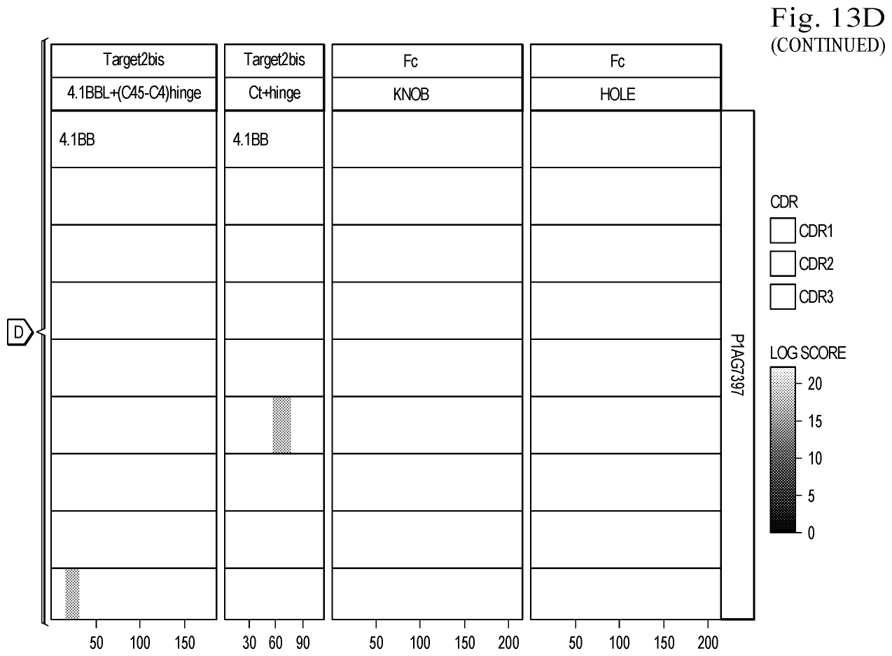

The MAPPs assay was used to identify potential T cell epitopes from the four BCMA-4-1BBL antigen binding molecules: P1AG7409, P1AG7422, P1AG7400 and P1AG7397 (FIG. 13A to FIG. 13D). Major histocompatibility complex-II (MHC-II)-Associated Peptide Proteomics (MAPPs) is a mass spectrometry-based approach to identify and relatively quantitate naturally processed and presented MHC-II-associated peptides that can potentially activate T cells and contribute to the immunogenicity of a drug.

Generation of Human Monocyte-Derived DCs and the Assay Procedure: Peripheral blood mononuclear cells (PBMCs) were isolated from buffy coats of healthy donors by gradient density centrifugation using Ficoll-Paque PLUS (GE Healthcare Europe GmbH, Glattbrugg, Switzerland). Monocytes were isolated by positive immunoselection using anti-CD14-coated microbeads and a magnetic separator (MACS, Miltenyi Biotech, Bergisch Gladbach, Germany). CD14⁺ cells were then cultured at a concentration of 0.3×10⁶ cells/mL in 100 mm ultra-low attachment culture dishes (Corning Inc., Corning, NY, USA) in serum-free Cellgro medium containing 1% GlutaMAX, 1% penicillin/streptomycin. Monocytes were differentiated into immature DCs with 50 ng/mL of GM-CSF and 5 ng/ml of IL-4 for 5 days at 37° C. with 5% CO₂ before being challenged with a test protein at 50 μg/mL in the presence of 1 μg/mL of lipopolysaccharide (LPS) from Salmonella abortus equi (Sigma-Aldrich Chemie GmbH, Buchs, Switzerland) for 24 h. Mature DCs were harvested, washed with phosphate-buffered saline (PBS) and the cell pellets were frozen at −80° C. prior to subsequent immunoprecipitation.

Isolation of HLA-DR-Presented Peptides: Cell pellets were lysed in 20 mM Tris-buffer solution pH 7.8 containing 1% (v/v) Triton X-100 and protease inhibitors (Roche Diagnostics GmbH, Mannheim, Germany) for 1 h at 4° C. on a ThermoMixer at 1100 rpm. The HLA-DR immune complexes were isolated by immunoprecipitation using biotin-conjugated anti-human HLA-DR antibodies (clone L243, RayBiotech). Lysates were incubated with the antibody on a rotator overnight at 4° C. Samples were washed five times with a buffer containing 20 mM N-(2-Hydroxyethyl) piperazine-N′-2-ethane sulfonic acid-NaOH (pH 7.9), 150 mM KCl, 1 mM MgCl₂, 0.2 mM CaCl₂, 0.2 mM ethylenediaminetetraacetate, 10% (v/v) glycerol, and 0.1% (v/v) Digitonin and five times with purified water. MHC-II peptides were eluted twice from HLA-DR molecules by adding 18 μL of 0.1% trifluoroacetic acid. The eluates were collected and kept at 4° C. prior to mass spectrometric analysis.

Data acquisition: Data acquisition and data processing were performed essentially as described in Steiner et al., J. Proteome Res. 2020, 19, 3792-3806. MHC-II peptide preparations obtained from about 3×10⁶ human monocyte-derived DCs (moDCs) per sample were separated on a nanocapillary liquid chromatography system (UltiMate 3000 RSLC, Thermo Scientific, CA, USA) using self-packed fused-silica C18 reversed phase column (75 μm i.d.×170 mm, ReproSil-Pur C18-AQ, 3 μm, Dr. Maisch GmbH) connected to a Q-Exactive HFX Orbitrap mass spectrometer (Thermo Scientific) via electrospray ionisation (LC-ESI-MS/MS). Samples (15 μL volume dissolved in 0.5% (v/v) formic acid in 2% (v/v) acetonitrile/water) were loaded for 2-3 min at 8 μL/min onto an Acclaim PepMap C18 trap column (100 μm i.d.×20 mm, Thermo Scientific) using a Vented Tee design. Peptides were then eluted at a flow rate of 250 nL/min using a nonlinear 39 min gradient of 2-45% B, followed by an 11 min column wash, and re-equilibration for 10 min [buffer A: 0.1% (v/v) formic acid in 2% (v/v) acetonitrile/water; buffer B: 0.1% (v/v) formic acid in acetonitrile]. MHC-II peptides were analysed by tandem MS using standard operating parameters. Survey scans (scanning range m/z 400-1650) were recorded in the Orbitrap mass analyzer at a resolution of 60,000, with the lock mass option enabled. Data-dependent MS/MS spectra of the 18 most abundant ions from the survey scan were recorded in the Orbitrap cell at a resolution of 15,000. Target ions selected for MS/MS were dynamically excluded for 7 s. Peptides were identified using the most updated PEAKS Studio version available at the time (version X Pro, Bioinformatics Solutions Inc., ON, Canada). The raw MS data were searched against the human protein database UniProtKB (http://www.uniprot.org, release 2015_10, approx. 88,500 TrEMBL and SwissProt entries containing the amino acid sequences of the test therapeutic proteins) with a mass tolerance of ±10 ppm for precursor ions and ±0.025 Da for fragment ions. Met-sulfoxide, Asn/Gln deamidation, and N-terminal pyroglutamylation were considered as differential modifications. Data were searched without enzyme specificity, and peptide results were reported at 1% specFDR cutoff. All LC-MS/MS runs of a given donor were processed batchwise, and the areas under the curve of identified features (2 min retention time shift tolerance; features reporting option: all) were exported in a tab-delimited table without further normalisation.

Statistical analysis: DataMAPPs is a Roche-created data analysis tool to help visualising otherwise complex mass spectrometry-derived data in the form of a heat map. The program is publicly available at https://www.R-project.org/and can be executed with a standard R installation. Please see reference (Steiner et al. 2020) for an extensive description of the package. DataMAPPs is invoked after processing the LC-MS/MS data with the PEAKS software and exporting results in a suitable tabular format. In short, the dataMAPPs processing pipeline consists of (1) data import and consistency check: sample annotation and peptide quantification (PEAKS output) files are read into memory and checked for consistency. Only peptides of length 10-30 amino acids are considered for analysis and mapped against the sequences of the studied biologics. Further filtering is performed to retain only the antibody-related signals that are specific to the studied molecules in a given LC-MS/MS analysis. A final step eliminates duplicate entries and sums signals from peptides identified with different charge states Peak area is transformed to the log 2 scale prior to processing; (2) data QC: the pipeline flags samples with a low number of identified peptides (cutoff typically set to half of the median peptide count for the respective donor) or low similarity (median Pearson correlation with all other samples from the same donor <0.8). Cutoffs can be modified by the user after inspecting a collection of QC plots, which allows for a suitable, data set-specific processing while being able to rerun the procedure in a reproducible way; (3) data normalisation: peptide abundance (based on peak area) is normalised based on an adjusted version of the GRSN (global rank-invariant set normalisation) procedure. (4) replicate aggregation: technical replicates (i.e. samples run for the same donor, same treatment, same dose) can be averaged to retain information on all peptides that were detected in at least one of the replicates; (5) peptide mapping: antibody-associated peptides are mapped on the tested biologic's protein amino acid sequences. In addition, peptides with neighbouring amino acid positions are binned to distinct epitope clusters (hotspots), whose abundance reflects the summed intensities of each of the constitutive MHC-II peptides; (6) data export and visualisation: the dataMAPPs workflow contains standard functionalities to generate several heatmap plots (epitope cluster or individual peptide level, per antibody or a global experiment summary) for a quick visualisation and comparison of the results

Results: The analysis of BCMA-4-1BBL antigen binding molecule P1AG7409 revealed two potential T cell epitopes in the non-germline regions (clusters C1 and C3). However, the frequency for these clusters appears to be low (1/10 donors).

The analysis of P1AG7422 revealed one binding cluster which contains any non-germline residues. In total 3/10 donors (35%) presented a potential immunogenic T cell epitope that could contribute to the immunogenicity of the compound.

The analysis of P1AG7400 did not reveal any binding clusters in the non-germline regions As a consequence, none of the donors presented a potential immunogenic T cell epitope that could contribute to the immunogenicity of the compound.

The analysis of P1AG7422 revealed two binding clusters. Among them, two clusters (C1 and C2) contained non-germline residues with frequencies of 1/10 and 3/10, respectively. In total 4/10 donors (40%) presented a potential immunogenic T cell epitope that could contribute to the immunogenicity of the compound.

### 6.3.2 DC: T Cell Assay (Epibase® DC: CD4 Re-Stimulation Assay Developed by Lonza)

T cell activation is an important part of the immune response to therapeutic proteins and is usually required for the development of anti-drug antibodies in the clinic. The DC-T cell assay was used to assess the capacity of four BCMA-4-1BBL antigen binding molecules: P1AG7409, P1AG7400, P1AG7397 and P1AG7422, to induce CD4⁺ T cells upon presentation of T cell epitopes by APCs.

Materials: Donors were recruited at Phase I clinical trial units in the UK. All samples were collected under an ethical protocol approved by a local REC (research ethics committee) and written informed consent was obtained from each donor prior to sample donation. All samples were stored according to the terms of Lonza's HTA (Human Tissue Authority) license for the use of samples in research. PBMCs from healthy donors were prepared from whole blood within six hours of blood withdrawal. Cells were cryopreserved in vapour phase nitrogen until use in the assays. The quality and functionality of each PBMC preparation was analyzed by seven days of activation with positive controls such as KLH to assess naïve T cell responses.

Keyhole limpet haemocyanin (KLH) was used as a technical control, reconstituted and stored at −80° C. in single use aliquots according to the manufacturer's recommendations under sterile conditions. Additionally, bevacizumab (Avastin®) was included as a positive benchmark protein. All samples were tested at a final concentration of 0.3 μM for the DC stimulation stage and for the APC re-stimulation stage.

Methods: Monocytes were isolated from frozen PBMC samples by magnetic bead selection and differentiated into immature DC (iDC) using GM-CSF and IL-4. iDC were then harvested, washed and loaded with each individual test protein/peptide for 4 hours at 37° C. A DC maturation cocktail containing TNFα and IL-1β was then added for a further 40-42 hours to activate/mature the DC (mDC). The expression of key DC surface markers (CD11c, CD14, CD40, CD80, CD83, CD86, CD209 and HLA-DR) at both the immature and mature stage were assessed by flow cytometry to ensure the DC were activated prior to T cell interaction. The mDCs were then co-cultured with autologous CD4⁺ T cells (isolated by magnetic bead selection) for 6 days at 37° C., 5% CO₂ in a humidified atmosphere. On day 6, autologous monocytes were isolated from PBMC using magnetic bead selection and loaded with the selected protein/peptide that were initially used to load the DC. After incubation at 37° C., 5% CO₂ in a humidified atmosphere for 4 hours, the monocytes were added to anti-IFNγ pre-coated FluoroSpot plates (Mabtech) along with the corresponding DC:CD4 co-culture in quadruplicate. The FluoroSpot plates were incubated for 40-42 hours at 37° C., 5% CO₂ in a humidified atmosphere. After incubation the FluoroSpot plates were developed using an in-house procedure and the spot-forming cells (SFC) per well assessed for each cytokine in each test condition.

Surface marker QC checks were performed on the monocyte derived DC at both the immature and mature stage to determine any possible influence of the test product on the DC differentiation and allows for the assessment of the quality of the DCs before subsequent co-culture with CD4+ T cells. Surface markers are assessed by flow cytometry using fluorescently labelled antibodies and the Guava® easyCyte™ 8HT flow cytometer.

Data analysis: Data management and statistical analysis has been performed in the R programming language (https://www.R-project.org/, v. 3.6.1). Data are transformed to a log 2 scale and a Generalized linear model (GLM) is applied to quantify SI (fold change and 95% CI). Adjustments are applied to the dataset (exponential type of heteroscedasticity adjusted, gaussian noise injection at low end of SFU scale, linear regression and extrapolation of each SI to a blank value of 0) and QC plot are generated (DC differentiation markers, reproducibility on compound and donor level, relative stimulation of donors).

To help for the immunogenicity risk assessment the Stimulation Index (SI) is calculated for each test condition in each donor (ratio between SFU/well and the matched blank). A positive donor response is counted if at least 2-fold SI change is established with p<0.05 (non-adjusted p-value from GLM). The number of positive donor responses to a treatment within the 30 healthy donor cohort gives the response rate relative to this treatment.

Results: The DC used in this assay were of high quality and expressed high levels of the T cell co-stimulatory molecules required for the activation of T cells. Based on the readouts of the DC-T cell assay, BCMA-4-1BBL antigen binding molecules P1AG7400 and P1AG739 were associated with a low risk of sequence-related immunogenicity for CD4⁺ T cell response (below the threshold of 10% as described in Siegel et al, Pharmaceutics 2022, 14 (12), 2672), whereas P1AG740 and P1AG7422 showed an incidence of T-cell activation (IFN-γ release) above the 10% threshold.

### 6.3.3 Integrated Risk for Immunogenicity

Administration of therapeutic antibodies may cause the formation of anti-drug antibodies (ADAs), which may negatively impact the safety of the therapeutic result (e.g., allergic reactions, immune complex-mediated diseases). The risk for the four BCMA-4-1BBL antigen binding molecules P1AG7409, P1AG7400, P1AG7397 and P1AG7422 to induce an unwanted immune response and the consequences for such a response were assessed through a combination of non-clinical assays (MAPPs and DC-T cell assay) as descibed herein before. Overall, the BCMA-4-1BBL antigen binding molecules can be ranked as follows, from the lowest risk of immunogenicity to the highest risk: P1AG7400, P1AG7397, P1AG7409 and P1AG7422.

1. An immunostimulatory antigen binding molecule that specifically binds to B cell maturation agent (BCMA), comprising
(a) a Fab molecule comprising
(i) a heavy chain variable region (V_{H} BCMA) comprising a heavy chain complementary determining region CDR-H1 of SEQ ID NO: 1 (GYTFTNYWMH), a CDR-H2 of SEQ ID NO: 2 (IIHPNSGSTNYNEKFQG), and a CDR-H3 of SEQ ID NO: 3 (GIYDYPFAY), and
(ii) a light chain variable region (V_{L} BCMA) selected from the group consisting of
(a) a VL comprising a light chain complementary determining region CDR-L1 of SEQ ID NO: 4 (RASESVSIHGTHLMH), a CDR-L2 of SEQ ID NO: 5 (AASSLQS) and a CDR-L3 of SEQ ID NO: 6 (QQSIEDPYT); and
(b) a VL comprising a light chain complementary determining region CDR-L1 of SEQ ID NO: 4 (RASESVSIHGTHLMH), a CDR-L2 of SEQ ID NO: 7 (AASNLES) and a CDR-L3 of SEQ ID NO: 6 (QQSIEDPYT), and
(c) a VL comprising a light chain complementary determining region CDR-L1 of SEQ ID NO: 4 (RASESVSIHGTHLMH), a CDR-L2 of SEQ ID NO: 8 (AASNLQS) and a CDR-L3 of SEQ ID NO: 6 (QQSIEDPYT),
(b) a first and a second polypeptide that are linked to each other by a disulfide bond, wherein the antigen binding molecule is characterized in that the first polypeptide comprises two ectodomains of 4-1BBL each comprising the amino acid sequence of SEQ ID NO:9 or SEQ ID NO:10 that are connected to each other by a peptide linker, and in that the second polypeptide comprises one ectodomain of 4-1BBL comprising the amino acid sequence of SEQ ID NO:9 or SEQ ID NO:10, and
(c) an IgG Fc domain.

2. The immunostimulatory antigen binding molecule of claim 1, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10.

3. The immunostimulatory antigen binding molecule of claim 1, wherein the Fab molecule comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16.

4. The immunostimulatory antigen binding molecule of claim 1, wherein the IgG Fc domain is an IgG1 Fc domain.

5. The immunostimulatory antigen binding molecule of claim 1, wherein the Fc comprises a modification promoting the association of the first and the second subunit of the Fc domain.

6. The immunostimulatory antigen binding molecule of claim 5, wherein the first subunit of the IgG Fc domain comprises the amino acid substitutions S354C and T366W (EU numbering) and the second subunit of the IgG Fc domain comprises the amino acid substitutions Y349C, T366S and Y407V (numbering according to Kabat EU index).

7. The immunostimulatory antigen binding molecule of claim 1, wherein the IgG Fc domain comprises one or more amino acid substitution that reduces binding to an Fc receptor and/or effector function.

8. The immunostimulatory antigen binding molecule of claim 1, wherein the IgG Fc domain is of human IgG1 subclass and comprises the amino acid mutations L234A, L235A and P329G (numbering according to Kabat EU index).

9. The immunostimulatory antigen binding molecule of claim 1, comprising
(a) a first polypeptide, comprising (ai) a first ectodomain of 4-1BBL or fragment thereof, fused at its C-terminus to the N-terminus of the second ectodomain of 4-1BBL or fragment thereof, (aii) a second ectodomain of 4-1BBL or fragment thereof, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL or fragment thereof, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
(d) a fourth polypeptide, comprising the light chain of the Fab molecule.

10. The immunostimulatory antigen binding molecule of claim 9, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

11. The immunostimulatory antigen binding molecule of claim 1, comprising
(a) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:24, or
(b) a first polypeptide comprising the amino acid sequence of SEQ ID NO:21, a second polypeptide comprising the amino acid sequence of SEQ ID NO:22, a third polypeptide comprising the amino acid sequence of SEQ ID NO:23 and a fourth polypeptide comprising the amino acid sequence of SEQ ID NO:25.

12. The immunostimulatory antigen binding molecule of claim 1, comprising (a) a first polypeptide comprising the amino acid sequence of SEQ ID NO:208, a second polypeptide comprising the amino acid sequence of SEQ ID NO:22, a third polypeptide comprising the amino acid sequence of SEQ ID NO:207 and a fourth polypeptide comprising the amino acid sequence of SEQ ID NO:24.

13. An immunostimulatory antigen binding molecule that specifically binds to B cell maturation agent (BCMA), comprising
(a) a Fab molecule comprising
(i) a heavy chain variable region (V_{L} BCMA) selected from the group consisting of
(a) a VH comprising a heavy chain complementary determining region CDR-H1 of SEQ ID NO: 26 (GFTFSNAWMD), a CDR-H2 of SEQ ID NO: 27 (QITAKSNNYATYYADSVKG), and a CDR-H3 of SEQ ID NO: 28 (DGYH); and
(b) a VH comprising a heavy chain complementary determining region CDR-H1 of SEQ ID NO: 26 (GFTFSNAWMD), a CDR-H2 of SEQ ID NO: 29 (QITAKSNNYATYYAAPVKG), and a CDR-H3 of SEQ ID NO: 28 (DGYH); and
(ii) a light chain variable region (V_{L} BCMA) comprising a light chain complementary determining region CDR-L1 of SEQ ID NO: 30 (RASEDIRNGLA), a CDR-L2 of SEQ ID NO: 31 (NANSLHT) and a CDR-L3 of SEQ ID NO: 32 (EDTSKYPYT),
(b) a first and a second polypeptide that are linked to each other by a disulfide bond, wherein the antigen binding molecule is characterized in that the first polypeptide comprises two ectodomains of 4-1BBL each comprising the amino acid sequence of SEQ ID NO:9 or SEQ ID NO:10 that are connected to each other by a peptide linker, and in that the second polypeptide comprises one ectodomain of 4-1BBL comprising the amino acid sequence of SEQ ID NO:9 or SEQ ID NO:10, and
(c) an IgG Fc domain.

14. The immunostimulatory antigen binding molecule of claim 13, wherein the immunostimulatory antigen binding molecule is characterized in that the first polypeptide comprises the amino acid sequence selected from the group consisting of SEQ ID NO:11, SEQ ID NO:12, SEQ ID NO:189 and SEQ ID NO:190 and in that the second polypeptide comprises the amino acid sequence of SEQ ID NO:9 or SEQ ID NO:10.

15. The immunostimulatory antigen binding molecule of claim 13, wherein the antigen binding domain comprises
(a) a V_{H} BCMA comprising an amino acid sequence of SEQ ID NO:33 (VH2a) and V_{L} BCMA comprising an amino acid sequence of SEQ ID NO:34 (VL2a), or
(b) a V_{H} BCMA comprising an amino acid sequence of SEQ ID NO:35 (VH1b) and V_{L} BCMA comprising an amino acid sequence of SEQ ID NO:36 (VL1a).

16. The immunostimulatory antigen binding molecule of claim 13, wherein the IgG Fc domain is an IgG1 Fc domain.

17. The immunostimulatory antigen binding molecule of claim 13, wherein the Fc domain is a human Fc domain.

18. The immunostimulatory antigen binding molecule of claim 13, wherein the IgG Fc domain comprises a modification promoting the association of the first and the second subunit of the Fc domain.

19. The immunostimulatory antigen binding molecule of claim 18, wherein the first subunit of the IgG Fc domain comprises the amino acid substitutions S354C and T366W (EU numbering) and the second subunit of the IgG Fc domain comprises the amino acid substitutions Y349C, T366S and Y407V (numbering according to Kabat EU index).

20. The immunostimulatory antigen binding molecule of claim 13, wherein the IgG Fc domain comprises one or more amino acid substitution that reduces binding to an Fc receptor and/or effector function.

21. The immunostimulatory antigen binding molecule of claim 13, wherein the IgG Fc domain is of human IgG1 subclass and comprises the amino acid mutations L234A, L235A and P329G (numbering according to Kabat EU index).

22. The immunostimulatory antigen binding molecule of claim 13, comprising
(a) a first polypeptide, comprising (ai) a first ectodomain of 4-1BBL or fragment thereof, fused at its C-terminus to the N-terminus of the second ectodomain of 4-1BBL or fragment thereof, (aii) a second ectodomain of 4-1BBL or fragment thereof, fused at its C-terminus to the N-terminus of a CL domain, (aiii) the CL domain, fused at its C-terminus to the N-terminus of one of the subunits (e.g. the first subunit) of the Fc domain, and (aiv) one of the subunits (e.g. the first subunit) of the Fc domain;
(b) a second polypeptide, comprising (bi) a third ectodomain of 4-1BBL or fragment thereof, fused at its C-terminus to the N-terminus of a CH1 domain, and (bii) the CH1 domain;
(c) a third polypeptide, comprising (ci) the heavy chain of the Fab molecule, fused at its C-terminus to the N-terminus of the other one of the subunits (e.g. the second subunit) of the Fc domain, and (cii) the other one of the subunits (e.g. the second subunit) of the Fc domain; and
(d) a fourth polypeptide, comprising the light chain of the Fab molecule.

23. The immunostimulatory antigen binding molecule of claim 22, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

24. The immunostimulatory antigen binding molecule of claim 13, comprising
(a) a first polypeptide comprising the amino acid sequence of SEQ ID NO:21, a second polypeptide comprising the amino acid sequence of SEQ ID NO:22, a third polypeptide comprising the amino acid sequence of SEQ ID NO:37 and a fourth polypeptide comprising the amino acid sequence of SEQ ID NO:38, or
(b) a first polypeptide comprising the amino acid sequence of SEQ ID NO:21, a second polypeptide comprising the amino acid sequence of SEQ ID NO:22, a third polypeptide comprising the amino acid sequence of SEQ ID NO:39 and a fourth polypeptide comprising the amino acid sequence of SEQ ID NO:40.

25. An antibody that specifically binds to BCMA, wherein the antibody comprises
(a) a V_{H} BCMA comprising an amino acid sequence of SEQ ID NO:13 and V_{L} BCMA comprising an amino acid sequence of SEQ ID NO:15, or
(b) a V_{H} BCMA comprising an amino acid sequence of SEQ ID NO:13 and V_{L} BCMA comprising an amino acid sequence of SEQ ID NO:16.

26. An antibody that specifically binds to BCMA, wherein the antibody comprises
(a) a V_{H} BCMA comprising an amino acid sequence of SEQ ID NO:33 (VH2a) and V_{L} BCMA comprising an amino acid sequence of SEQ ID NO:34 (VL2a), or
(b) a V_{H} BCMA comprising an amino acid sequence of SEQ ID NO:35 (VH1b) and V_{L} BCMA comprising an amino acid sequence of SEQ ID NO:36 (VL1a).

27. One or more isolated polynucleotide encoding the immunostimulatory antigen binding molecule of claim 1.

28. One or more vector, particularly expression vector, comprising the polynucleotide(s) of claim 27.

29. A host cell comprising the polynucleotide(s) of claim 27.

30. A method of producing an immunostimulatory antigen binding molecule that specifically binds to BCMA, comprising the steps of a) culturing the host cell of claim 28 under conditions suitable for the expression of the immunostimulatory antigen binding molecule and optionally b) recovering the immunostimulatory antigen binding molecule.

31. An immunostimulatory antigen binding molecule that specifically binds to BCMA produced by the method of claim 30.

32. A pharmaceutical composition comprising the immunostimulatory antigen binding molecule of claim 1 and at least one pharmaceutically acceptable excipient.

33. (canceled)

34. (canceled)

35. (canceled)

36. (canceled)

37. (canceled)

38. (canceled)

39. (canceled)

40. (canceled)

41. A method of treating a disease, particularly cancer, in an individual, comprising administering to said individual an effective amount of the immunostimulatory antigen binding molecule of claim 1.

42. The method of claim 41, further comprising administration in combination with a chemotherapeutic agent, radiation therapy and/or other agents for use in cancer immunotherapy, particularly in combination with a T-cell activating anti-CD3 bispecific antibody selected from an anti-GPRC5D/anti-CD3 antibody, an anti-BCMA/anti-CD3 antibody or an anti-FcRH5/anti-CD3 antibody.
